(19) United States
(12) Patent Application Publication
(10) Pub. No.: US 2010/0234220 A1
(43) Pub. Date: Sep. 16, 2010

(54) Oxooxetanes as Fungicidal Agents

(75) Inventors: Mazen Es-Sayed, Langenfeld (DE); Stefan Hillebrand, Neuss (DE); Welf-Burkhard Wiese, Langenfeld (DE); Astrid Ullmann-Koppold, Leverkusen (DE); Klaus Kunz, Düsseldorf (DE); Peter Schreier, Köln (DE); Martin Vaupel, Leichlingen (DE); Karl-Heinz Kuck, Langenfeld (DE); Ulrike Wachendorff-Neumann, Neuwied (DE); Kerstin Ilg, Köln (DE); Armin De Meijere, Göttingen (DE); Oleg Larionov, Cambridge, MA (US); Amos Mattes, Langenfeld (DE); Peter Dahmen, Neuss (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/517,517

(22) PCT Filed: Nov. 24, 2007
(86) PCT No.: PCT/EP2007/010227

§ 371 Date: Feb. 4, 2010

(30) Foreign Application Priority Data
Dec. 8, 2006 (DE) 10 2006 058 260.8
Dec. 13, 2006 (DE) 10 2006 058 623.9

(51) Int. Cl. A01C 1/06; A01N 43/20; A01P 3/00; C07D 305/12; C07D 405/12; A01N 43/66

(52) U.S. Cl. 504/100; 514/449; 549/328; 544/212; 514/245

(57) ABSTRACT

The present invention relates to compounds of the formula (I) below

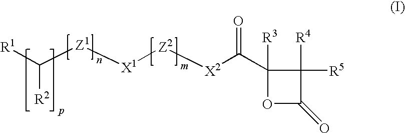

and to their agrochemically active salts and their use alone or in a mixture with other active compounds and/or auxiliaries and/or additives for controlling phytopathogenic fungi and other microorganisms in or on plants, to processes for preparing these compounds and mixtures, to intermediates of these processes and to seed treated with the compounds, salts or mixtures mentioned.

The invention relates to the use of oxooxetanecarboxylic acid derivatives for controlling phytopathogenic fungi, to novel oxooxetanecarboxylic acid derivatives and to processes for their preparation.

DE-A 101 13 045 describes ebelactone A, an oxooxetane, as fungicidally active compound for controlling plant pathogens. Furthermore, M. M. Mackeen et al., describe the fungicidal action of oxooxetanecarboxylic acid derivatives from the fruit Garcinia atroviridis (Zeitschrift fuer Naturforschung, C: Journal of Biosciences (2002), 57(3/4), 291-295).

Since the ecological and economical demands made on modern fungicides are increasing constantly, for example with respect to activity spectrum, toxicity, selectivity, application rate, formation of residues and favourable manufacture, and there can furthermore be problems, for example, with resistance, there is a constant need to develop novel fungicides which, at least in some areas, help to overcome the disadvantages mentioned.

Surprisingly, it has now been found that certain oxooxetanecarboxylic acid derivatives are highly suitable for controlling phytopathogenic fungi.

Some of these compounds are described in EP-A-1 166 781 as pharmaceutics for treating tumours, autoimmune diseases, inflammations, rheumatism, arthritis, asthma and Alzheimer's disease. However, that the compounds may be suitable for controlling phytopathogenic fungi cannot be derived from this publication.

Accordingly, the invention provides the use of compounds of the formula (I) or agrochemically active salts thereof for controlling phytopathogenic fungi and other microorganisms in or on plants,

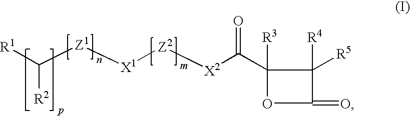

where the symbols and indices in the formula (I) have the following meanings:
m, n are identical or different and are a number from 0 to 5;
p is 0 or 1;
R¹ is H, substituted or unsubstituted alkyl, substituted or unsubstituted cycloalkyl, substituted or unsubstituted arylalkyl, substituted or unsubstituted aryl, or
R¹ is NR⁶R⁷,
R⁶ is H, substituted or unsubstituted alkyl, substituted or unsubstituted heterocyclyl, or substituted or unsubstituted arylalkyl, and
R⁷ is H, substituted or unsubstituted alkyl, substituted or unsubstituted arylalkyl, or CWR⁸ in which
R⁸ is H, substituted or unsubstituted alkyl, substituted or unsubstituted alkylamino, substituted or unsubstituted alkoxy, substituted or unsubstituted aryl, substituted or unsubstituted heterocyclyl, substituted or unsubstituted arylalkyl, substituted or unsubstituted arylalkylamino or substituted or unsubstituted arylalkyloxy, and
W is O or S or
R¹ is

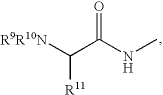

where
R⁹ is H, substituted or unsubstituted alkyl or substituted or unsubstituted arylalkyl,
R¹⁰ is H, substituted or unsubstituted alkyl, substituted or unsubstituted arylalkyl, CWR⁸, where W and R⁸ have the meanings given above, substituted or unsubstituted alkylsulphonyl, substituted or unsubstituted arylsulphonyl, or PW(R¹²)₂,
   in which
W has the meanings given above and
R¹² are identical or different and are substituted or unsubstituted alkyl or substituted or unsubstituted aryl;
   or
R⁹ and R¹⁰ together are

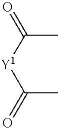

in which
Y¹ is substituted or unsubstituted alkylene or substituted or unsubstituted arylene; and
R¹¹ is H, substituted or unsubstituted alkyl, or substituted or unsubstituted arylalkyl;
R² is H, C₁-C₄-alkyl, COR¹³ in which
R¹³ is OH, substituted or unsubstituted alkoxy, substituted or unsubstituted alkenyloxy, substituted or unsubstituted arylalkyloxy, substituted or unsubstituted cycloalkyloxy, substituted or unsubstituted aryloxyalkoxy or NR¹⁴R¹⁵ in which
R¹⁴ is H, substituted or unsubstituted alkyl, substituted or unsubstituted aryl,
R¹⁵ is substituted or unsubstituted alkyl, substituted or unsubstituted arylalkyl, substituted or unsubstituted alkoxycarbonylalkyl, amino, substituted or unsubstituted alkylamino or substituted or unsubstituted arylamino, or
R¹⁴ and R¹⁵ together with the adjacent nitrogen atom form a substituted or unsubstituted heterocycle, or
R¹³ is CH₂OR¹⁶ in which
R¹⁶ is H, substituted or unsubstituted alkyl, substituted or unsubstituted arylalkyl, or
R² is substituted or unsubstituted alkanoyl, substituted or unsubstituted arylcarbonyl or SiR₃¹⁷ in which
R¹⁷ are identical or different and are substituted or unsubstituted alkyl or substituted or unsubstituted aryl; or
R¹ and R² together form a group of the formula

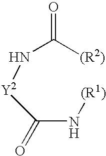

in which
Y² is a substituted or unsubstituted alkylene group;
R³, R⁴, R⁵ are identical or different and are H, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heterocyclyl or substituted or unsubstituted arylalkyl;
X¹ is a bond, substituted or unsubstituted alkylene, substituted or unsubstituted cycloalkylene, substituted or unsubstituted alkenylene or substituted or unsubstituted arylene;
X² is O, S, NR¹⁸, N(OR¹⁸), NR¹⁸O¹⁸ or NR¹⁸NH, where
R¹⁸ is H, substituted or unsubstituted alkyl, unsubstituted or substituted C₁-C₄-alkyl-C(═O), C₁-C₄-alkyl-OC(═O), unsubstituted or substituted C₁-C₄-alkoxy-(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo(C₁-C₃-alkyl)carbonyl-C₁-C₃-alkyl, halo(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl, (C₃-C₈-halocycloalkyl)-carbonyl having in each case 1 to 9 fluorine, chlorine and/or bromine atoms, or —CH₂—C≡C—R¹⁹, —CH₂—CH═CH—R¹⁹, —CH═C═CH—R¹⁹, —C(═O)C(═O)R¹⁹, —CON(R¹⁹)₂, —CH₂N(R¹⁹)₂, C₁-C₄-trialkylsilyl, C₁-C₄-dialkylmonophenylsilyl or substituted or unsubstituted arylalkyl in which
R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano,
Z¹, Z² are identical or different and are CH₂, CHCH₃, CHF, CF₂, CHCl, CCl₂, CH₂—CH(CH₃), CH(CH₃)—CH₂ or
Z² is —O—CH₂—CH₂, —O—CH(CH₃)—CH₂, —O—CH₂—CHCH₃.

In a second embodiment, the invention relates to the abovementioned use of compounds of the formula (I) or agrochemically active salts thereof

where
m and n independently of one another are an integer from 0 to 3; and
P is either 0 or 1;
   and
R¹ is selected from the group consisting of H, (C₁-C₈)-alkyl, (C₃-C₈)-cycloalkyl, (C₆-C₁₀)-aryl and (C₆-C₁₀)-aryl-(C₁-C₄)-alkyl;
   or
R¹ is NR⁶R⁷ in which
R⁶ is selected from the group consisting of H, (C₁-C₈)-alkyl, five- to ten-membered saturated, unsaturated or aromatic mono- or bicyclic heterocyclyl which contains one to four heteroatoms selected from the group consisting of O, N and S, and (C₆-C₁₂)-aryl-(C₁-C₄)-alkyl; and
R⁷ is selected from the group consisting of H, (C₁-C₈)-alkyl, (C₆-C₁₂)-aryl-(C₁-C₄)-alkyl and CWR⁸ in which
R⁸ is selected from the group consisting of H, (C₁-C₈)-alkyl, (C₁-C₈)-alkylamino, (C₁-C₈)-alkoxy, (C₆-C₁₂)-aryl, 4 to 8-membered heterocyclyl, (C₆-C₁₂)-aryl-(C₁-C₄)-alkyl, (C₆-C₁₂)-aryl-(C₁-C₄)-alkylamino and (C₆-C₁₂)-aryl-(C₁-C₄)-alkyloxy; and
W is either O or S;
   or
R¹ is

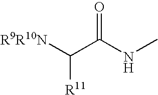

in which
R⁹ is selected from the group consisting of H, (C₁-C₈)-alkyl and (C₆-C₁₂)-aryl-(C₁-C₄)-alkyl; and
R¹⁰ is selected from the group consisting of H, (C₁-C₈)-alkyl, (C₆-C₁₂)-aryl-(C₁-C₄)-alkyl, CWR⁸ in which W and R⁸ are as defined above, (C₁-C₈)-alkylsulphonyl, (C₆-C₁₂)-arylsulphonyl and PW(R¹²)₂ in which
W is as defined above; and
R¹² are identical or different and are selected from the group consisting of (C₁-C₈)-alkyl and (C₆-C₁₂)-aryl; or
R⁹ and R¹⁰ together are

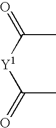

in which
Y¹ is (C₁-C₈)-alkylene or (C₆-C₁₂)-arylene; and
R¹¹ is selected from the group consisting of H, (C₁-C₈)-alkyl and (C₆-C₁₂)-aryl-(C₁-C₄)-alkyl; and
R² is H, C₁-C₄-alkyl or COR¹³ in which
R¹³ is selected from the group consisting of OH, (C₁-C₈)-alkoxy, (C₂-C₈)-alkenyloxy, (C₆-C₁₂)-aryl-(C₁-C₄)-alkoxy, (C₃-C₈)-cycloalkyloxy, (C₆-C₁₂)-aryloxy-(C₁-C₄)-alkoxy and NR¹⁴R¹⁵ in which
R¹⁴ is selected from the group consisting of H, (C₁-C₈)-alkyl and (C₆-C₁₂)-aryl; and
R¹⁵ is selected from the group consisting of (C₁-C₈)-alkyl, (C₆-C₁₂)-aryl-(C₁-C₄)-alkyl, (C₁-C₈)-alkoxycarbonyl-(C₁-C₄)-alkyl, amino, (C₁-C₈)-alkylamino and (C₆-C₁₂)-arylamino; or
R¹⁴ and R¹⁵ together with the adjacent nitrogen atom form a 4- to 8-membered heterocycle; or
R¹³ is CH₂OR¹⁶ in which
R¹⁶ is selected from the group consisting of H, (C₁-C₈)-alkyl and (C₆-C₁₀)-aryl-(C₁-C₄)-alkyl;
   or
R² is selected from the group consisting of (C₁-C₈)-alkanoyl, (C₆-C₁₂)-arylcarbonyl and Si(R¹⁷)₃ in which
R¹⁷ are all identical or different and are selected from the group consisting of (C₁-C₄)-alkyl and phenyl;
or
R¹ and R² together form a group of the formula

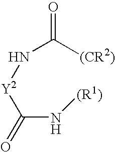

in which
Y² is a (C₁-C₄)-alkylene group;
   and
R³, R⁴ and R⁵ independently of one another are selected from the group consisting of H, (C₁-C₈)-alkyl, (C₂-C₈)-alkenyl, (C₂-C₈)-alkynyl, (C₃-C₈)-cycloalkyl, (C₃-C₆)-cycloalkenyl, (C₆-C₁₂)-aryl, a five- to ten-membered saturated, unsaturated or aromatic mono- or bicyclic heterocyclyl which contains one to four heteroatoms selected from the group consisting of O, N and S, and (C₆-C₁₂)-aryl-(C₁-C₄)-alkyl;
   and
X¹ is selected from the group consisting of a bond, (C₁-C₄)-alkylene, (C₃-C₆)-cycloalkylene, (C₂-C₄)-alkenylene and (C₆-C₁₂)-arylene;
   and
X² is selected from the group consisting of O, S, NR¹⁸, N(OR¹⁸), NR¹⁸O and NR¹⁸NH in which
R¹⁸ is selected from the group consisting of
H, (C₁-C₈)-alkyl, C₁-C₄-alkyl-C(═O), C₁-C₄-alkyl-OC(═O), C₁-C₄-alkoxy-(C₁-C₄)alkyl, C₁-C₆-alkenyl, C₁-C₆-alkynyl, C₁-C₆-alkylsulphinyl, C₁-C₆-alkylsulphonyl and C₃-C₈-cycloalkyl;
C₁-C₆-haloalkyl, C₁-C₄-haloalkylsulphinyl, C₁-C₄-haloalkylsulphonyl, halo-C₁-C₄-alkoxy-C₁-C₄-alkyl and C₃-C₈-halocycloalkyl having in each case 1 to 9 fluorine, chlorine and/or bromine atoms;
formyl, formyl-C₁-C₃-alkyl, (C₁-C₃-alkyl)carbonyl-C₁-C₃-alkyl and (C₁-C₃-alkoxy)carbonyl-C₁-C₃-alkyl;
halo(C₁-C₃-alkyl)carbonyl-C₁-C₃-alkyl and halo(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl and (C₃-C₈-cycloalkyl)carbonyl;
(C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)-carbonyl and (C₃-C₈-halocycloalkyl)carbonyl having in each case 1 to 9 fluorine, chlorine and/or bromine atoms; and
—CH₂—C≡C—R¹⁹, —CH₂—CH═CH—R¹⁹, —CH═C═CH—R¹⁹, —C(═O)C(═O)R¹⁹,
—CON(R¹⁹)₂, —CH₂N(R¹⁹)₂, C₁-C₄-trialkylsilyl, C₁-C₄-dialkylmonophenylsilyl and (C₆-C₁₂)-aryl-(C₁-C₄)-alkyl in which
R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano.

In a third embodiment, the invention relates to the abovementioned use of compounds of the formula (I) or agrochemically active salts thereof

where
m and n independently of one another are 0, 1 or 2;
   and
p is either 0 or 1;
   and
R¹ is selected from the group consisting of H, (C₁-C₈)-alkyl, (C₃-C₈)-cycloalkyl, phenyl, phenylethyl and benzyl;
   or
R¹ is NR⁶R⁷ in which
R⁶ is selected from the group consisting of H, (C₁-C₄)-alkyl, five- or six-membered saturated, unsaturated or aromatic monocyclic heterocyclyl which contains one to three heteroatoms selected from the group consisting of O, N and S, and benzyl; and
R⁷ is selected from the group consisting of H, (C₁-C₄)-alkyl, benzyl and CWR⁸ in which
R⁸ is selected from the group consisting of H, (C₁-C₄)-alkyl, (C₁-C₄)-alkylamino, (C₁-C₄)-alkoxy, phenyl, 4- to 6-membered heterocyclyl, benzyl, phenyl-(C₁-C₄)-alkylamino and phenyl-(C₁-C₄)-alkyloxy; and
W is either O or S;
or
R¹ is

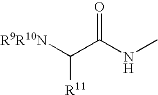

in which
R⁹ is selected from the group consisting of H, (C₁-C₈)-alkyl and benzyl; and
R¹⁰ is selected from the group consisting of H, (C₁-C₈)-alkyl, benzyl and CWR⁸ in which W and R⁸ are as defined above, (C₁-C₄)-alkylsulphonyl, phenylsulphonyl and PW(R¹²)₂ in which
W is as defined above; and
R¹² is (C₁-C₄)-alkyl or phenyl; or
R⁹ and R¹⁰ together are

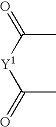

in which
Y¹ is (C₁-C₄)-alkylene or phenylene; and
R¹¹ is selected from the group consisting of H, (C₁-C₄)-alkyl and benzyl;
   and
R² is H, C₁-C₄-alkyl or COR¹³ in which
R¹³ is selected from the group consisting of OH, (C₁-C₄)-alkoxy, (C₂-C₄)-alkenyloxy, benzyloxy, (C₃-C₆)-cycloalkyloxy, phenoxy-(C₁-C₄)-alkoxy and NR¹⁴R¹⁵ in which
R¹⁴ is selected from the group consisting of H, (C₁-C₄)-alkyl and phenyl; and
R¹⁵ is selected from the group consisting of (C₁-C₄)-alkyl, benzyl, (C₁-C₄)-alkoxycarbonyl-(C₁-C₄)-alkyl, amino, (C₁-C₄)-alkylamino and phenylamino; or
R¹⁴ and R¹⁵ together with the adjacent nitrogen atom form a 4- to 6-membered heterocycle; or
R¹³ is CH₂OR¹⁶ in which
R¹⁶ is selected from the group consisting of H, (C₁-C₄)-alkyl and benzyl;
   or
R² is selected from the group consisting of (C₁-C₄)-alkanoyl, phenyl and Si(R¹⁷)₃ in which
R¹⁷ are all identical or different and are selected from the group consisting of (C₁-C₄)-alkyl and phenyl;
   and
R³, R⁴ and R⁵ independently of one another are selected from the group consisting of H, (C₁-C₈)-alkyl, (C₂-C₆)-alkenyl, (C₂-C₆)-alkynyl, (C₃-C₆)-cycloalkyl, (C₃-C₆)-cycloalkenyl, phenyl, a five or six-membered saturated, unsaturated or aromatic monocyclic heterocyclyl which contains one to four heteroatoms selected from the group consisting of O, N and S, and benzyl;
   and
X¹ is selected from the group consisting of a bond, (C₁-C₄)-alkylene, (C₃-C₆)-cycloalkylene, (C₂-C₄)-alkenylene and phenylene;
   and
X² is selected from the group consisting of O, S, NR¹⁸, N(OR¹⁸) and NR¹⁸O in which
R¹⁸ is selected from the group consisting of
H, (C₁-C₄)-alkyl, C₁-C₄-alkyl-C(═O), C₁-C₄-alkyl-OC(═O), C₁-C₄-alkoxy-(C₁-C₄)alkyl, C₂-C₄-alkenyl, C₂-C₄-alkynyl, C₁-C₄-alkylsulphinyl, C₁-C₄-alkylsulphonyl and C₃-C₆-cycloalkyl;
C₁-C₄-haloalkyl, C₁-C₄-haloalkylsulphinyl, C₁-C₄-haloalkylsulphonyl, halo-C₁-C₄-alkoxy-C₁-C₄-alkyl and C₃-C₆-halocycloalkyl having in each case 1 to 4 fluorine, chlorine and/or bromine atoms;
formyl, formyl-C₁-C₃-alkyl, (C₁-C₃-alkyl)carbonyl-C₁-C₃-alkyl and (C₁-C₃-alkoxy)carbonyl-C₁-C₃-alkyl;
halo(C₁-C₃-alkyl)carbonyl-C₁-C₃-alkyl and halo(C₁-C₃-alkoxy)carbonyl-C₁-C₃-alkyl having in each case 1 to 6 fluorine, chlorine and/or bromine atoms;
(C₁-C₄-alkyl)carbonyl, (C₁-C₄-alkoxy)carbonyl, (C₁-C₄-alkylthio)carbonyl, (C₁-C₄-alkoxy-C₁-C₄-alkyl)carbonyl, (C₃-C₄-alkenyloxy)carbonyl and (C₃-C₄-alkynyloxy)carbonyl, (C₃-C₆-cycloalkyl)carbonyl;
(C₁-C₄-haloalkyl)carbonyl, (C₁-C₄-haloalkylthio)carbonyl, (C₁-C₄-haloalkoxy)carbonyl, (C₃-C₄-haloalkenyloxy)carbonyl, (C₃-C₄-haloalkynyloxy)carbonyl, (halo-C₁-C₄-alkoxy-C₁-C₄-alkyl)-carbonyl and (C₃-C₆-halocycloalkyl)carbonyl having in each case 1 to 4 fluorine, chlorine and/or bromine atoms; and
—CH₂—C≡C—R¹⁹, —CH₂—CH═CH—R¹⁹, —C(═O)C(═O)R¹⁹, —CON(R¹⁹)₂, C₁-C₄-trialkylsilyl, C₁-C₄-dialkylmonophenylsilyl and benzyl in which
R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano.

In a fourth embodiment, the invention relates to the abovementioned use of compounds of the formula (I) or agrochemically active salts thereof where in the abovementioned embodiments 1 to 3 the radicals alkyl, cycloalkyl, arylalkyl or arylalkyl, aryl, heterocyclyl or heterocycle, alkylamino, alkoxy, aryl, arylalkylamino, arylalkyloxy or arylalkoxy, alkylsulphonyl, arylsulphonyl, alkylene, arylene, alkenyloxy, cycloalkyloxy, aryloxyalkoxy, alkoxycarbonylalkyl, arylamino, alkanoyl, arylcarbonyl or arylcarbonyl, alkenyl, alkynyl, cycloalkenyl, cycloalkylene, alkenylene, C₁-C₄-alkyl-C(═O), C₁-C₄-alkoxy(C₁-C₄)alkyl, C₁-C₆-alkenyl and C₁-C₆-alkynyl independently of one another are unsubstituted or substituted.

In a fifth embodiment, the radicals mentioned in the fourth embodiment are substituted by one or more substituents selected from the group consisting of F, Cl, Br, I, OH, SH, CN, NO₂, NH₂, (C₁-C₄)-alkyl, (C₂-C₄)-alkenyl, (C₂-C₄)-alkynyl, (C₃-C₆)-cycloalkyl, (C₃-C₆)-cycloalkenyl, (C₁-C₄)-haloalkyl, (C₂-C₄)-haloalkenyl, (C₂-C₄)-haloalkynyl, (C₃-C₆)-halocycloalkyl, (C₁-C₄)-alkoxy, (C₂-C₄)-alkenoxy, (C₂-C₄)-haloalkynoxy, (C₃-C₆)-halocycloalkyloxy, SO₀₋₂—(C₁-C₄)-alkyl, SO₀₋₂—(C₂-C₄)-alkenyl, SO₀₋₂—(C₂-C₄)-alkynyl, SO₀₋₂—(C₃-C₆)-cycloalkyl, SO₀₋₂—(C₁-C₄)-haloalkyl, SO₀₋₂—(C₂-C₄)-haloalkenyl, SO₀₋₂—(C₂-C₄)-haloalkynyl, SO₀₋₂—(C₂-C₆)-halocycloalkyl, carboxyl, (C₁-C₄)-alkoxycarbonyl, carbamoyl, (C₁-C₄)-mono- and (C₁-C₄)-dialkylamino, (C₁-C₄)-mono- and (C₁-C₄)-dialkylaminocarbonyl, (C₁-C₄)-mono- and (C₁-C₄)-dialkylaminosulphonyl, hydroxyimino-(C₁-C₄)-alkyl, (C₁-C₄)-alkyl-(C₁-C₄)-alkoxyimino-(C₁-C₆)-alkyl, Si(C₁-C₄)₃, phenyl, and phenoxy, where the phenyl and phenoxy radicals are unsubstituted or substituted by 1 to 5 of the substituents mentioned. Preferably, the abovementioned radicals of the previous embodiment are independently of one another unsubstituted or substituted by one or more substituents selected from the group consisting of F, Cl, Br, I, OH, SH, CN, NO₂, NH₂, (C₁-C₄)-alkyl, (C₂-C₄)-alkenyl, (C₃-C₆)-cycloalkyl, (C₃-C₆)-cycloalkenyl, (C₁-C₄)-haloalkyl, (C₂-C₄)-haloalkenyl, (C₃-C₆)-halocycloalkyl, (C₁-C₄)-alkoxy, (C₂-C₄)-alkenoxy, (C₃-C₆)-halocycloalkyloxy, SO₀₋₂—(C₁-C₄)-alkyl, SO₀₋₂—(C₂-C₄)-alkenyl, SO₀₋₂—(C₃-C₆)-cycloalkyl, SO₀₋₂—(C₁-C₄)-haloalkyl, SO₀₋₂—(C₂-C₄)-haloalkenyl, SO₀₋₂—(C₃-C₆)-halocycloalkyl, carboxyl, (C₁-C₄)-alkoxycarbonyl, carbamoyl, (C₁-C₄)-mono- or dialkylamino, (C₁-C₄)-mono- or -dialkylaminocarbonyl, (C₁-C₄)-mono- or -dialkylaminosulphonyl, phenyl and phenoxy, where the phenyl and phenoxy radicals are unsubstituted or substituted by 1 to 5 of the substituents mentioned.

Furthermore, in a sixth embodiment the invention relates to the use, mentioned in the first embodiment, of compounds of the formula (I) or agrochemically active salts thereof where
m is 0;
   and
n is either 0, 1 or 2;
   and
p is 0 or 1;
   and
R¹ is selected from the group consisting of H, straight-chain or branched (C₁-C₆)-alkyl, (C₅-C₆)-cycloalkyl, phenyl, benzyl, 1-phenylethyl and 2-phenylethyl, where aromatic groups are unsubstituted or substituted by one or more radicals selected from the group consisting of halogen, cyano, (C₁-C₄)-alkyl, (C₁-C₄)-haloalkyl, (C₁-C₄)-alkoxy, (C₁-C₄)-haloalkoxy, SO₀₋₂(C₁-C₄)-alkyl and phenoxy, where the phenoxy radical is unsubstituted or substituted by one or more of the radicals mentioned;
   or
R¹ is NR⁶R⁷ in which
R⁶ is H or six-membered aromatic heterocyclyl, where heterocyclyl contains one to three nitrogen atoms and is unsubstituted or substituted by one or more radicals selected from the group consisting of halogen, (C₁-C₄)-alkyl, (C₁-C₄)-haloalkyl, (C₁-C₄)-alkoxy, benzyloxy and (C₁-C₄)-haloalkoxy; and
R⁷ is H or CORE in which
R⁸ is selected from the group consisting of H, unsubstituted or substituted (C₁-C₄)-alkyl, unsubstituted or substituted (C₁-C₄)-alkylamino, unsubstituted or substituted (C₁-C₄)-alkoxy, unsubstituted or substituted phenyl and unsubstituted or substituted benzyloxy;
   or
R¹ is

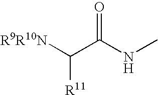

in which
R⁹ is selected from the group consisting of H, straight-chain or branched (C₁-C₄)-alkyl and unsubstituted or substituted benzyl; and
R¹⁰ is selected from the group consisting of H, CH₃, C₂H₅, C₃H₇, C₄H₉, C₅H₁₁, benzyl and COR⁸, where the alkyl groups are straight-chain or branched, and where the benzyl radical is unsubstituted or substituted by one or more, preferably 1 to 3, radicals selected from the group consisting of F, Cl, CH₃, i-C₃H₇, t-C₄—H₉, OCH₃, OC₂H₅, O-i-C₃H₇ and CF₃, and where R⁸ has the meanings given above; and
R¹¹ is selected from the group consisting of H, CH₃, C₂H₅ and benzyl;
   and
R² is selected from the group consisting of H, CH₃, C₂H₅ and COR¹³ in which
R¹³ is selected from the group consisting of OH, O-t-C₄H₃ and benzyloxy;
   and
R³ is selected from the group consisting of H, (C₁-C₄)-alkyl and (C₁-C₄)-haloalkyl;
   and
R⁴ is selected from the group consisting of (C₁-C₆)-alkyl, (C₂-C₄)-alkenyl, (C₂-C₄)-alkynyl, (C₁-C₆)-haloalkyl, cyclopropyl, cyclopentyl, cyclohexyl, 1-(C₅-C₇)-cycloalkenylethyl, (C₅-C₇)-cycloalkenylmethyl, phenyl, benzyl, 1-phenylethyl, hydroxybenzyl, 1-hydroxymethyl-(C₅-C₇)-cycloalkenyl, (C₂-C₃)-haloalkenylmethyl, 1-(C₂-C₃)-haloalkenylethyl, hydroxy-(C₁-C₆)-alkyl, 1-(C₃-C₇)-cycloalkylethyl, (C₃-C₇)-cycloalkylmethyl, 1-ethyl-(C₃-C₆)-cycloalkyl, 1-methyl-(C₃-C₆)-cycloalkyl, where the alkyl groups are straight-chain or branched, and where aromatic radicals are unsubstituted or substituted by one or more, preferably 1 to 3, radicals selected from the group consisting of F, Cl, t-C₄—H₉, CH₃, OCH₃, CF₃ and OCF₃;
   and
R⁵ is H;
   and
X¹ is selected from the group consisting of a bond, straight-chain or branched (C₁-C₄)-alkylene, or (C₃-C₄)-cycloalkylene;
   and
X² is selected from the group consisting of O, NR¹⁸, N(OR¹⁸) and NR¹⁸O in which
R¹⁸ is selected from the group consisting of
H, methyl, ethyl, propyl, 1-methylethyl, butyl, 1-methylpropyl, 2-methylpropyl, 1,1-dimethylethyl and benzyl;
CF₃, CF₂H, CCl₃, C₂F₅, C₃F₇ and CF(CF₃)₂;
acetyl, C₂H₅C(═O), C₃H₇C(═O), C₄H₉C(═O), CF₃C(═O), C₂F₅C(═O), CH₃C(═O), C₂H₅C(═O), C₃H₇C(═O), C₃H₇OC(═O), C₄H₉C(═O), CF₃C(═O), CCl₃OC(═O), and C₂F₅OC(═O);
CH₂OCH₃ and C₂H₄OCH₃;
CH═CH₂, CH₂CH═CH₂, C≡CH and CH₂C≡CH;
SOCH₃, SOC₂H₅, SOC₃H₇, SO₂CH₃, SO₂C₂H₅, SO₂C₃H₇ and SO₂CF₃;
cyclopropyl, cyclobutyl, cyclopentyl and cyclohexyl;
CH₂OCF₃ and C₂H₄OCF₃;
C(═O)NH₂; and
SiMe₃, SiMe₂tBu and SiMe₂Ph;
   and
Z¹ and Z² are CH₂.

Additionally, in a seventh embodiment the invention relates to the use, mentioned in the first embodiment, of compounds of the formula (I) or agrochemically active salts thereof where
m is 0;
   and
n is 0, 1 or 2;
   and
p 0 or 1 is;
   and
R¹ is selected from the group consisting of H, CH₃, C₂H₅, C₃H₇, C₄H₉, C₅H₁₁, cyclohexyl, phenyl, benzyl, 1-phenylethyl and 2-phenylethyl, where the alkyl groups are straight-chain or branched, and where aromatic radicals are unsubstituted or substituted by one or more, preferably 1 to 3, radicals selected from the group consisting of F, Cl, I, cyano, CH₃, i-C₃H₇, t-C₄—H₉, OCH₃, OC₂H₅, O-i-C₃H₇, O-t-C₄H₉, CF₃, OCF₃, OCHF₂, SO₂CH₃ and O-phenyl, where O-phenyl is unsubstituted or substituted by 1 to 3 radicals selected from the group consisting of F, Cl, CH₃ and OCH₃;

### Or

R¹ is NR⁶R⁷ in which
R⁶ is selected from the group consisting of H, pyridazinyl, pyrimidinyl, pyridinyl, pyrazinyl and 1,3,5-triazinyl, in each case unsubstituted or substituted by one or two radicals selected from the group consisting of CH₃, i-C₃H₇, t-C₄—H₉, OCH₃, OC₂H₅, O-i-C₃H₇, CF₃ and benzyloxy;
   and
R⁷ is H or CORE; and
R⁸ is selected from the group consisting of benzyloxy, O-t-C₄H₉ and 9-H-fluoren-9-ylmethoxy;

### Or

R¹ is

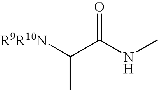

in which
R⁹ is selected from the group consisting of H, CH₃, C₂H₅ and benzyl; and
R¹⁰ is selected from the group consisting of H, CH₃, C₂H₅ and COR⁸, where R⁸ has the meanings given above;
   and
R² is selected from the group consisting of H, CH₃ and COR¹³ in which
R¹³ is selected from the group consisting of OH, O-t-C₄H₉ and benzyloxy;
   and
R³ is selected from the group consisting of H, (C₁-C₄)-alkyl and (C₁-C₄)-haloalkyl;
   and
R⁴ is selected from the group consisting of CH₃, C₂H₅, C₃H₇, C₄H₉, C₅H₁₁, cyclopropyl, cyclohexyl, 1-ethylcyclopropyl, phenyl, benzyl and 1-phenylethyl, where the alkyl groups are straight-chain or branched, and where aromatic radicals are unsubstituted or substituted by one or two radicals selected from the group consisting of F, Cl, t-C₄—H₉, CH₃, OCH₃, CF₃ and OCF₃;
   and
R⁵ is H;
   and
X¹ is selected from the group consisting of a bond, CH₂, CH₂—CH₂ and cyclopropylene;
   and
X² is NR¹⁸ in which
R¹⁸ is selected from the group consisting of
H, methyl, ethyl, propyl, 1-methylethyl, butyl and benzyl;
CF₃, CF₂H and C₂F₅;
acetyl, C₂H₅C(═O), CF₃C(═O), CH₃OC(═O), C₂H₅C(═O), C₃H₇OC(═O), C₃H₇OC(═O),
C₄H₉C(═O) and CF₃C(═O);
CH₂OCH₃ and C₂H₄OCH₃;
CH₂CH═CH₂ and CH₂C≡CH;
SO₂CH₃, SO₂C₂H₅ and SO₂CF₃;
CH₂OCF₃ and C₂H₄OCF₃;
C(═O)NH₂; and
SiMe₃ and
Z¹ and Z² are CH₂.

Furthermore, in an eighth embodiment the invention relates to the use, mentioned in the first embodiment, of compounds of the formula (I) or agrochemically active salts thereof, and also to the compounds themselves, where
(a) R³ is selected from the group consisting of alkyl, alkenyl and arylalkyl, where the abovementioned radicals are substituted by one or more substituents selected from the substituents as defined in claims 1 to 6 or unsubstituted, and
   X² is NR¹⁸;
or
(b) R⁴ and R⁵ are selected from the group consisting of alkyl, alkenyl, alkynyl, cycloalkyl, cycloalkenyl, aryl, heterocyclyl and arylalkyl, where the abovementioned radicals are substituted by one or more substituents selected from the substituents as defined in claims 1 to 6 or unsubstituted, and
   X² is NR¹⁸;
or
(c) R¹ is selected from the group consisting of alkyl, cycloalkyl, arylalkyl and aryl, where the abovementioned radicals are substituted by one or more substituents selected from the substituents as defined in claims 1 to 6 or unsubstituted, and
   X² is NR¹⁸;
or
(d) X² is selected from the group consisting of S, N(OR¹⁸), NR¹⁸O and NR¹⁸NH;
or
(e) X² is oxygen, and
   R⁴ comprises at least 2 carbon atoms, and
   with the proviso that, if
   (i) R¹=R³=R⁵=H, X¹ is a bond and m=n=p=0, then R⁴ is not n-propyl, sec-butyl, isobutyl or 2-methyl-1-propen-3-yl;
   (ii) R¹=R³=H, R⁵=n-propyl, X¹ is a bond and m=n=p=0, then R⁴ is not n-propyl;
   (iii) R¹=methyl, R³=R⁵=H, X¹ is a bond and m=n=p=0, then R⁴ is not ethyl;
   (iv) R¹=ethyl, R³=trifluoromethyl, R⁵=H, X¹ is a bond and m=n=p=0, then R⁴ is not 1-phenylprop-3-yl;
   (v) R¹=benzyl, R³=R⁵=H, X¹ is a bond and m=n=p=0, then R⁴ is not ethyl, isopropyl, isobutyl or 2-methyl-1-propen-3-yl;
   (vi) R¹=tert-butyl, R³=R⁵=H, X¹ is a bond and m=n=p=0, then R⁴ is not sec-butyl, isobutyl, 1-penten-5-Y¹ or naphth-2-ylmethyl;
   (vii) R³=H, R⁴=1-propen-3-yl, R⁵=methyl, X¹ is a bond and m=n=p=0, then R¹ is not benzyl or n-butyl;
   (viii) R¹=benzyl, R⁴=isobutyl, R⁵=H, X¹ is a bond and m=n=p=0, then R³ is not benzyloxycarbonyl or acetyloxymethyl;
   (ix) R¹=methyl, R³=methoxycarbonyl, R⁵=H, X¹ is a bond and m=n=p=0, then R⁴ is not methoxycarbonylmethyl; or
   (x) R¹=ethyl, R³=ethoxycarbonyl, R⁵=H, X¹ is a bond and m=n=p=0, then R⁴ is not ethoxycarbonylmethyl.

The invention furthermore relates to a process for preparing a compound according to formula (I)

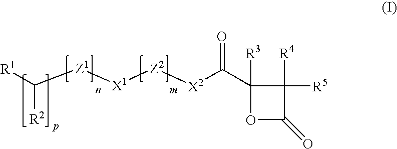

as defined in one of the embodiments 1 to 8 mentioned above, which comprises reacting a compound according to formula (II)

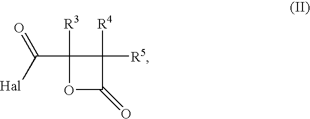

with a compound of the formula (IV)

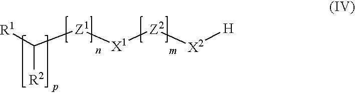

in the presence of a suitable base, where the symbols R¹, R², R³, R⁴, R⁵, X¹, X², Z¹, Z², m, n and p given in the formulae (II) and (IV) mentioned are as defined in one of the embodiments 1 to 8 mentioned above, and Hal is selected from the group consisting of F, Cl, Br and I.

The invention also relates to a process for preparing a compound according to formula (II)

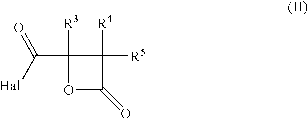

as defined above, which comprises reacting a compound according to formula (III)

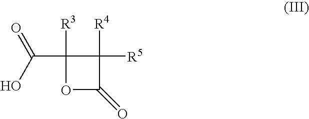

with a suitable halogenating agent, where the symbols R³, R⁴ and R⁵ given in formula (III) are as defined in one of the embodiments 1 to 8 mentioned above.

Preference is given to processes mentioned above in which the Hal mentioned in formula (II) is a Cl, and/or X² is either O or NR¹⁸, where R¹⁸ is as defined in one of the embodiments 1 to 8 mentioned above.

The invention also relates to a further process for preparing a compound of the formula (I)

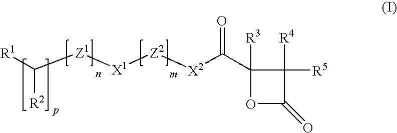

as defined in one of the embodiments 1 to 8 mentioned above, which comprises reacting a compound of the formula (VIII)

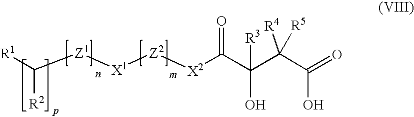

with a suitable condensing agent, where the symbols R¹, R², R³, R⁴, R⁵, X¹, X², Z¹, Z², m, n and p given in the formula (VIII) mentioned are as defined in one of the embodiments 1 to 8 mentioned above,
and also to a process for preparing a compound of the formula (VIII)

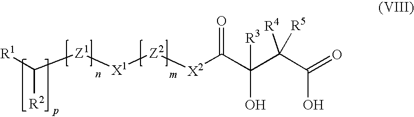

as defined above, which comprises reacting a compound of the formula (VII)

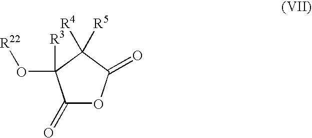

with a compound of the formula (IV)

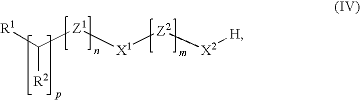

where the symbols R¹, R², R³, R⁴, R⁵, X¹, X², Z¹, Z², m, n and p given in the formulae (VII) and (IV) mentioned are as defined in one of the embodiments 1 to 8 mentioned above, and R²² is selected from the group consisting of H, SO₂R²³ or COR²³, where R²³ is unsubstituted or halogen-substituted (C₁-C₄)-alkyl, or unsubstituted or halogen-, (C₁-C₄)-alkyl- or (C₁-C₄)-haloalkyl-substituted aryl. Preferably

R²² is selected from the group consisting of H, SO₂R²³ and COR²³, where R²³ is unsubstituted or fluorine-substituted (C₁-C₄)-alkyl, or unsubstituted or fluorine-, chlorine-, methyl-, ethyl- or trifluoromethyl-substituted phenyl; particularly preferably, R²² is COR²³, where R²³ is unsubstituted or fluorine-substituted (C₁-C₄)-alkyl, or unsubstituted fluorine-, chlorine-, methyl-, ethyl- or trifluoromethyl-substituted phenyl; and especially preferably, R²² is COR²³, where R²³ is (C₁-C₄)-alkyl or (C₁-C₄)-fluoroalkyl; and R²² is especially preferably acetyl or trifluoroacetyl.

The invention furthermore relates to a process for preparing a compound according to formula (VII)

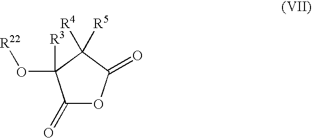

as defined in the previous process, which comprises reacting a compound according to formula (VI)

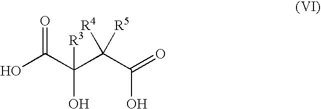

with an anhydride former such as ethyl chloroformate, methyl chloroformate, isopropyl chloroformate, isobutyl chloroformate, p-toluenesulphonyl chloride, methanesulphonyl chloride, acetic anhydride or trifluoroacetic anhydride, where the symbols R³, R⁴ and R⁵ given in the formula (VI) mentioned are as defined in one of the embodiments 1 to 8 mentioned above, and to a process for preparing a compound of the formula (VI)

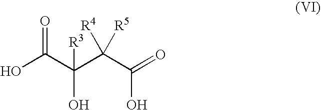

which comprises reacting a compound of the formula (V)

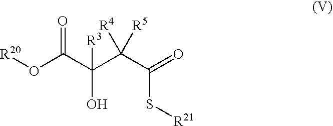

with a suitable base in an aqueous medium, where the symbols R³, R⁴ and R⁵ given in the formula (V) mentioned are as defined in one of the embodiments 1 to 8 mentioned above, R²⁰ is selected from the group consisting of branched and straight-chain alkyl and arylalkyl radicals, and R²¹ is selected from the group consisting of branched and straight-chain alkyl, arylalkyl and aryl radicals. Preferably, R²⁰ is selected from the group consisting of branched and straight-chain alkyl radicals, particularly preferably a straight-chain alkyl radical and very particularly preferably ethyl; and/or R²¹ is selected from the group consisting of branched and straight-chain alkyl and aryl radicals, particularly preferably a straight-chain aryl radical and very particularly preferably phenyl.

The invention also relates to compounds of the formula (II) as defined in the processes mentioned above.

The invention also relates to compounds of the formula (VIIa)

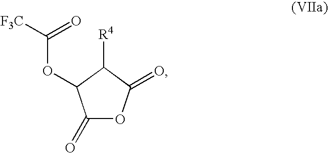

in which R⁴ is selected from the group consisting of (C₂-C₈)-alkyl, (C₂-C₈)-alkenyl, (C₂-C₈)-alkynyl, (C₃-C₈)-cycloalkyl, (C₃-C₆)-cycloalkenyl, (C₆-C₁₂)-aryl, five- to ten-membered saturated, unsaturated or aromatic mono- or bicyclic heterocyclyl which contains one to four heteroatoms selected from the group consisting of O, N and S, and (C₆-C₁₂)-aryl-(C₁-C₄)-alkyl. Preferably, the radicals defined as R⁴ are unsubstituted or substituted by one or more of the substituents mentioned above.

In a further embodiment, the invention relates to the use of a mixture comprising at least one of the compounds defined in one of the embodiments 1 to 8 mentioned above and/or at least one of the agrochemically active salts thereof and a further active compound selected from the group consisting of insecticides, attractants, sterilants, bactericides, acaricides, nematicides, fungicides, growth regulators, herbicides, safeners, fertilizers or semiochemicals for controlling phytopathogenic fungi and other microorganisms in or on plants.

In a particular embodiment, the invention relates to the abovementioned use or the use according to one of the embodiments 1 to 8 mentioned above for treating plants or for treating seed of plants, preferably of transgenic plants.

The invention also relates to a composition for controlling phytopathogenic fungi and other microorganisms in or on plants or in and/or on seed of plants, where the composition mentioned comprises at least one compound as defined in one of the embodiments 1 to 8 mentioned above and/or at least one of the agrochemically active salts thereof or a mixture as defined above and agrochemically customary auxiliaries and/or additives. Preferably, the auxiliaries and/or additives are selected from the group consisting of extenders and surfactants.

The invention also relates to a process for preparing such a composition, which process comprises mixing at least one compound as defined in one of the embodiments 1 to 8 mentioned above and/or at least one of the agrochemically active salts thereof or a mixture as defined above with agrochemically customary auxiliaries and/or additives; preferably, the auxiliaries and/or additives are selected from the group consisting of extenders and surfactants.

The invention furthermore relates to a method for controlling phytopathogenic fungi and other microorganisms in or on plants or in and/or on seed of plants, which method comprises bringing the fungi or microorganisms mentioned into direct or indirect contact with at least one compound as defined in one of the embodiments 1 to 8 mentioned above and/or at least one of the agrochemically active salts thereof or a mixture as defined above or a composition as defined above.

The invention also relates to seed treated with at least one compound as defined in one of the embodiments 1 to 8 mentioned above and/or at least one of the agrochemically active salts thereof or a mixture as defined above or a composition as defined above. Preferably, the seed is selected from seed of transgenic plants.

The compounds of the formula (I) are highly suitable for controlling unwanted microorganisms. They show in particular strong fungicidal activity and can be used both in crop protection and in the protection of materials.

The compounds of the formula (I) can be present both in pure form and as mixtures of various possible isomeric forms, in particular of stereoisomers, such as E and Z, threo- and erythro-, and also optical isomers, such as R and S isomers or atropisomers, and, if appropriate, also of tautomers. The use according to the invention comprises both the pure isomers and mixtures thereof.

Depending on the nature of the substituents defined above, the compounds of the formula (I) have acidic or basic properties and may be able to form salts, if appropriate also inner salts. If the compounds of the formula (I) carry hydroxyl groups, carboxyl groups or other groups which induce acidic properties, these compounds can be reacted with bases to salts. Suitable bases are, for example, hydroxides, carbonates, bicarbonates of the alkali metals and alkaline earth metals, in particular those of sodium, potassium, magnesium and calcium, furthermore ammonia, primary, secondary and tertiary amines having (C₁-C₄-)-alkyl radicals or arylalkyl radicals, mono-, di- and trialkanolamines of (C₁-C₄)-alkanols, choline and also chlorocholine. If the compounds of the formula (I) carry amino groups, alkylamino groups or other groups which induce basic properties, these compounds can be reacted with acids to salts. Suitable acids are, for example, mineral acids, such as hydrochloric acid, sulphuric acid and phosphoric acid, organic acids, such as acetic acid or oxalic acid, and acidic salts, such as NaHSO₄ and KHSO₄. The salts obtained in this manner also have fungicidal properties.

According to the invention, W is (═O) or (═S), unless indicated otherwise.

The formula (I) provides a general definition of the compounds used according to the invention.

Preferably, the symbols and indices in the formula (I) have the following meanings:
m, n are preferably identical or different and are a number from 0 to 3.
p is preferably 0 or 1.
R¹ is preferably H, unsubstituted or substituted (C₁-C₈)-alkyl, unsubstituted or substituted (C₃-C₈)-cycloalkyl, unsubstituted or substituted (C₆-C₁₀)-aryl or unsubstituted or substituted (C₆-C₁₀)-aryl-(C₁-C₄)-alkyl,
   or
R¹ is preferably NR⁶R⁷, where
R⁶ is H, unsubstituted or substituted (C₁-C₈)-alkyl, substituted or unsubstituted five- to ten-membered saturated, unsaturated or aromatic mono- or bicyclic heterocyclyl which contains one to four heteroatoms selected from the group consisting of O, N and S or unsubstituted or substituted (C₆-C₁₂)-aryl-(C₁-C₄)-alkyl, and
R⁷ is H, unsubstituted or substituted (C₁-C₈)-alkyl, unsubstituted or substituted (C₆-C₁₂)-aryl-(C₁-C₄)-alkyl or CWR⁸ in which
R⁸ is H, unsubstituted or substituted (C₁-C₈)-alkyl, unsubstituted or substituted (C₁-C₈)-alkylamino, unsubstituted or substituted (C₁-C₈)-alkoxy, unsubstituted or substituted (C₆-C₁₂)-aryl, unsubstituted or substituted 4- to 8-membered heterocyclyl, unsubstituted or substituted (C₆-C₁₂)-aryl-(C₁-C₄)-alkyl, unsubstituted or substituted (C₆-C₁₂)-aryl-(C₁-C₄-alkylamino or unsubstituted or substituted (C₆-C₁₂)-aryl-(C₁-C₄)-alkyloxy, and
W is O or S, or
R¹ is preferably

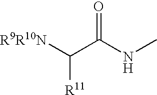

where
   R⁹ is H, unsubstituted or substituted (C₁-C₈)-alkyl or unsubstituted or substituted (C₆-C₁₂)-aryl-(C₁-C₄)-alkyl,
   R¹⁰ is H, unsubstituted or substituted (C₁-C₈)-alkyl or unsubstituted or substituted (C₆-C₁₂)-aryl-(C₁-C₄)-alkyl, CWR⁸, where W and R⁸ have the meanings given above, unsubstituted or substituted (C₁-C₈)-alkylsulphonyl, unsubstituted or substituted (C₆-C₁₂)-arylsulphonyl, or PWR₂¹²,
      in which
      W has the meanings given above and
      R¹² is unsubstituted or substituted (C₁-C₈)-alkyl or unsubstituted or substituted (C₆-C₁₂)-aryl, or
      R⁹ and R¹⁰ together are

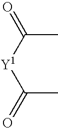

in which
Y¹ is unsubstituted or substituted (C₁-C₈)-alkylene or unsubstituted or substituted (C₆-C₁₂)-arylene and
R¹¹ is H, unsubstituted or substituted (C₁-C₈)-alkyl or unsubstituted or substituted (C₆-C₁₂)-aryl-(C₁-C₄)-alkyl.
R² is preferably H, C₁-C₄-alkyl, COR¹³ in which
   R¹³ is OH, unsubstituted or substituted (C₁-C₈)-alkoxy, unsubstituted or substituted (C₂-C₈)-alkenyloxy, unsubstituted or substituted (C₆-C₁₂)-aryl-(C₁-C₄)-alkoxy, unsubstituted or substituted (C₃-C₈)-cycloalkyloxy, unsubstituted or substituted (C₆-C₁₂)-aryloxy-(C₁-C₄)-alkoxy or NR¹⁴R¹⁵ in which
      R¹⁴ is H, unsubstituted or substituted (C₁-C₈)-alkyl or unsubstituted or substituted (C₆-C₁₂)-aryl,
      R¹⁵ is unsubstituted or substituted (C₁-C₈)-alkyl, unsubstituted or substituted (C₆-C₁₂)-aryl-(C₁-C₄)-alkyl, unsubstituted or substituted (C₁-C₈)-alkoxycarbonyl-(C₁-C₄)-alkyl, amino, unsubstituted or substituted (C₁-C₈)-alkylamino or unsubstituted or substituted (C₆-C₁₂)-arylamino, or
      R¹⁴ and R¹⁵ together with the adjacent nitrogen atom form an unsubstituted or substituted 4- to 8-membered heterocycle, or
   R¹³ is CH₂OR¹⁶ in which
      R¹⁶ is H, unsubstituted or substituted (C₁-C₈)-alkyl or unsubstituted or substituted (C₆-C₁₀)-aryl-(C₁-C₄)-alkyl, or
R² is preferably unsubstituted or substituted (C₁-C₈)-alkanoyl, unsubstituted or substituted (C₆-C₁₂)-arylcarbonyl, or SiR₃¹⁷ in which
   R¹⁷ are identical or different and are unsubstituted or substituted (C₁-C₄)-alkyl or unsubstituted or substituted phenyl, or
R¹ and R² together preferably form a group of the formula

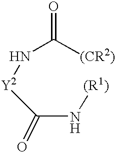

in which
   Y² is an unsubstituted or substituted (C₁-C₄)-alkylene group.
R³, R⁴, R⁵ are preferably identical or different and are H, unsubstituted or substituted (C₁-C₈)-alkyl, unsubstituted or substituted (C₂-C₈)-alkenyl, substituted or unsubstituted (C₂-C₈)-alkynyl, substituted or unsubstituted (C₃-C₈)-cycloalkyl, substituted or unsubstituted (C₃-C₆)-cycloalkenyl, substituted or unsubstituted (C₆-C₁₂)-aryl, a substituted or unsubstituted five- to ten-membered saturated, unsaturated or aromatic mono- or bicyclic heterocyclyl which contains one to four heteroatoms selected from the group consisting of O, N and S or unsubstituted or substituted (C₆-C₁₂)-aryl-(C₁-C₄)-alkyl.
X¹ is preferably a bond, unsubstituted or substituted (C₁-C₄)-alkylene, unsubstituted or substituted (C₃-C₆)-cycloalkylene, unsubstituted or substituted (C₂-C₄)-alkenylene or unsubstituted or substituted (C₆-C₁₂)-arylene.
X² is preferably O, S, NR¹⁸, N(OR¹⁸), NR¹⁸O or NR¹⁸NH, where
   R¹⁸ is H, unsubstituted or substituted (C₁-C₈)-alkyl, unsubstituted or substituted C₁-C₄-alkyl-C(═O), C₁-C₄-alkyl-OC(═O), unsubstituted or substituted C₁-C₄-alkoxy-(C₁-C₄)alkyl, unsubstituted or substituted C₁-C₆-alkenyl, unsubstituted or substituted C₁-C₆-alkynyl, C₁-C₆-alkylsulphinyl, C₁-C₆-alkylsulphonyl, C₃-C₈-cycloalkyl; C₁-C₆-haloalkyl, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkylthio)carbonyl, (C₁-C₆-haloalkoxy)carbonyl, (C₃-C₆-haloalkenyloxy)-carbonyl, (C₃-C₆-haloalkynyloxy)carbonyl, (halo-C₁-C₄-alkoxy-C₁-C₄-alkyl)carbonyl, (C₃-C₈-halocycloalkyl)carbonyl having in each case 1 to 9 fluorine, chlorine and/or bromine atoms, or —CH₂—C≡C—R¹⁹, —CH₂—CH═CH—R¹⁹, —CH═C═CH—R¹⁹, —C(═O)C(═O)R¹⁹, —CH₂N(R¹⁹)₂, C₁-C₄-trialkylsilyl, C₁-C₄-dialkylmonophenylsilyl and (C₆-C₁₂)-aryl-(C₁-C₄)-alkyl in which
   R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano.

“Substituted” means preferably substituted by one or more substituents from the group consisting of F, Cl, Br, I (halogen), OH, SH, CN, NO₂, NH₂, (C₁-C₄)-alkyl, (C₂-C₄)-alkenyl, (C₂-C₄)-alkynyl, (C₃-C₆)-cycloalkyl, (C₃-C₆)-cycloalkenyl, (C₁-C₄)-haloalkyl, (C₂-C₄)-haloalkenyl, (C₂-C₄)-haloalkynyl, (C₃-C₆)-halocycloalkyl, (C₁-C₄)-alkoxy, (C₂-C₄)-alkenoxy, (C₂-C₄)-haloalkynoxy, (C₃-C₆)-halocycloalkyloxy, SO₀₋₂—(C₂-C₄)-alkenyl, SO₀₋₂—(C₂-C₄)-alkynyl, SO₀₋₂—(C₃-C₆)-cycloalkyl, SO₀₋₂(C₁-C₄)-haloalkyl, SO₀₋₂—(C₂-C₄)-haloalkenyl, SO₀₋₂—(C₂-C₄)-haloalkynyl, SO₀₋₂—(C₂-C₆)-halocycloalkyl, carboxyl, (C₁-C₄)-alkoxycarbonyl, carbamoyl, (C₁-C₄)-mono- or -dialkylamino, (C₁-C₄)-mono- or -dialkylaminocarbonyl, (C₁-C₄)-mono- or -dialkylaminosulphonyl, hydroxyimino-(C₁-C₄)-alkyl, (C₁-C₄)-alkyl-(C₁-C₄)-alkoxyimino-(C₁-C₆)-alkyl, Si(C₁-C₄)₃, phenyl, phenoxy, where the phenyl and phenoxy radicals are unsubstituted or substituted by 1 to 5 of the groups mentioned.

Preferred substituents are furthermore (C₃-C₄)-alkynyloxy, CF₃, methyl, ethyl, n-propyl, isopropyl, n-butyl, tert-butyl, isobutyl

Preference is given to compounds of the formula (I) in which the symbols and indices have the preferred meanings mentioned.

Particularly preferably, the symbols and indices in the formula (I) have the following meanings:
m, n are particularly preferably identical or different and are a number 0, 1 or 2.
p is particularly preferably 0 or 1.
R¹ is particularly preferably H, unsubstituted or substituted (C₁-C₈)-alkyl, unsubstituted or substituted (C₃-C₈)-cycloalkyl, unsubstituted or substituted phenyl, unsubstituted or substituted benzyl or unsubstituted or substituted phenylethyl,
R¹ is furthermore particularly preferably unsubstituted or substituted phenylpropyl or unsubstituted or substituted phenylbutyl or unsubstituted or substituted naphthylmethyl or unsubstituted or substituted naphthylethyl,
   or
R¹ is particularly preferably NR⁶R⁷, where
   R⁶ is H, unsubstituted or substituted (C₁-C₄)-alkyl, substituted or unsubstituted five- or six-membered saturated, unsaturated or aromatic monocyclic heterocyclyl which contains one to three heteroatoms selected from the group consisting of O, N and S, or unsubstituted or substituted benzyl, and
   R⁷ is H, unsubstituted or substituted (C₁-C₄)-alkyl, unsubstituted or substituted benzyl or CWR⁸ in which
   R⁸ is H, unsubstituted or substituted (C₁-C₄)-alkyl, unsubstituted or substituted (C₁-C₄)-alkylamino, unsubstituted or substituted (C₁-C₄)-alkoxy, unsubstituted or substituted phenyl, unsubstituted or substituted 4- to 6-membered heterocyclyl, unsubstituted or substituted benzyl, unsubstituted or substituted phenyl-(C₁-C₄)-alkylamino or unsubstituted or substituted phenyl-(C₁-C₄)-alkyloxy, and
   W is O or S, or
R¹ is particularly preferably

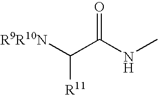

where
   R⁹ is H, unsubstituted or substituted (C₁-C₈)-alkyl or unsubstituted or substituted benzyl,
   R¹⁰ is H, unsubstituted or substituted (C₁-C₈)-alkyl or unsubstituted or substituted benzyl, CWR⁸ where W and R⁸ have the meanings given above, unsubstituted or substituted (C₁-C₄)-alkylsulphonyl, unsubstituted or substituted phenylsulphonyl, or PWR₂¹²,
   in which
      W has the meanings given above and
      R¹² is unsubstituted or substituted (C₁-C₄)-alkyl or unsubstituted or substituted phenyl, or
      R⁹ and R¹⁰ together are

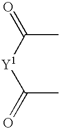

in which
Y¹ is unsubstituted or substituted (C₁-C₄)-alkylene or unsubstituted or substituted phenylene and
R¹¹ is H, unsubstituted or substituted (C₁-C₄)-alkyl or unsubstituted or substituted benzyl.
R² is particularly preferably H, C₁-C₄-alkyl, COR¹³ in which
   R¹³ is OH, unsubstituted or substituted (C₁-C₄)-alkoxy, unsubstituted or substituted (C₂-C₄)-alkenyloxy, unsubstituted or substituted benzyloxy, unsubstituted or substituted (C₃-C₆)-cycloalkyloxy, unsubstituted or substituted phenoxy-(C₁-C₄)-alkoxy or NR¹⁴R¹⁵ in which
      R¹⁴ is H, unsubstituted or substituted (C₁-C₄)-alkyl or unsubstituted or substituted phenyl,
      R¹⁵ unsubstituted or substituted (C₁-C₄)-alkyl, unsubstituted or substituted benzyl, unsubstituted or substituted (C₁-C₄)-alkoxycarbonyl-(C₁-C₄)-alkyl, amino, unsubstituted or substituted (C₁-C₄)-alkylamino or unsubstituted or substituted phenylamino, or
         R¹⁴ and R¹⁵ together with the adjacent nitrogen atom form an unsubstituted or substituted 4- to 6-membered heterocycle, or
      R¹³ is CH₂OR¹⁶ in which
      R¹⁶ is H, unsubstituted or substituted (C₁-C₄)-alkyl or unsubstituted or substituted benzyl, or
R² is particularly preferably unsubstituted or substituted (C₁-C₄)-alkanoyl, unsubstituted or substituted phenyl, or SiR₃¹⁷ in which
   R¹⁷ is identical or different unsubstituted or substituted (C₁-C₄)-alkyl or unsubstituted or substituted phenyl, or
R³, R⁴, R⁵ are particularly preferably identical or different H, unsubstituted or substituted (C₁-C₈)-alkyl, unsubstituted or substituted (C₂-C₆)-alkenyl, substituted or unsubstituted (C₂-C₆)-alkynyl, substituted or unsubstituted (C₃-C₆)-cycloalkyl, substituted or unsubstituted (C₃-C₆)-cycloalkenyl, substituted or unsubstituted phenyl, substituted or unsubstituted five- or six-membered saturated, unsaturated or aromatic monocyclic heterocyclyl which contains one to four heteroatoms selected from the group consisting of O, N and S or unsubstituted or substituted benzyl.
X¹ is particularly preferably a bond, unsubstituted or substituted (C₁-C₄)-alkylene, unsubstituted or substituted (C₃-C₆)-cycloalkylene, unsubstituted or substituted (C₂-C₄)-alkenylene or unsubstituted or substituted phenylene.
X² is particularly preferably O, S, NR¹⁸, N(OR¹⁸) or NR¹⁸O, where
   R¹⁸ is H, unsubstituted or substituted (C₁-C₄)-alkyl, unsubstituted or substituted C₁-C₄-alkyl-C(═O), C₁-C₄-alkyl-OC(═O), unsubstituted or substituted C₁-C₄-alkoxy-(C₁-C₄)-alkyl, unsubstituted or substituted C₂-C₄-alkenyl, unsubstituted or substituted C₂-C₄-alkynyl, C₁-C₄-alkylsulphinyl, C₁-C₄-alkylsulphonyl, C₃-C₆-cycloalkyl; C₁-C₄-haloalkyl, C₁-C₄-haloalkylsulphinyl, C₁-C₄-haloalkylsulphonyl, halo-C₁-C₄-alkoxy-C₁-C₄-alkyl, C₃-C₆-halocycloalkyl having in each case 1 to 4 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 6 fluorine, chlorine and/or bromine atoms; (C₁-C₄-alkyl)carbonyl, (C₁-C₄-alkoxy)carbonyl, (C₁-C₄-alkylthio)carbonyl, (C₁-C₄-alkoxy-C₁-C₄-alkyl)carbonyl, (C₃-C₄-alkenyloxy)carbonyl, (C₃-C₄-alkynyloxy)carbonyl, (C₃-C₆-cycloalkyl)carbonyl; (C₁-C₄-haloalkyl)carbonyl, (C₁-C₄-haloalkylthio)carbonyl, (C₁-C₄-haloalkoxy)carbonyl, (C₃-C₄-haloalkenyloxy)carbonyl, (C₃-C₄-haloalkynyloxy)carbonyl, (halo-C₁-C₄-alkoxy-C₁-C₄-alkyl)carbonyl, (C₃-C₆-halocycloalkyl)carbonyl having in each case 1 to 4 fluorine, chlorine and/or bromine atoms, or —CH₂—C≡C—R¹⁹, —CH₂—CH═CH—R¹⁹, —CH═C═CH—R¹⁹, —C(═O)C(═O)R¹⁹, —CON(R¹⁹)₂, —CH₂N(R¹⁹)₂, C₁-C₄-trialkylsilyl, C₁-C₄-dialkylmonophenylsilyl or unsubstituted or substituted benzyl in which
   R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano.

“Substituted” means particularly preferably substituted by one or more substituents from the group consisting of F, Cl, Br, I, OH, SH, CN, NO₂, NH₂, (C₁-C₄)-alkyl, (C₂-C₄)-alkenyl, (C₃-C₆)-cycloalkyl, (C₃-C₆)-cycloalkenyl, (C₁-C₄)-haloalkyl, (C₂-C₄)-haloalkenyl, (C₃-C₆)-halocycloalkyl, (C₁-C₄)-alkoxy, (C₂-C₄)-alkenoxy, (C₃-C₆)-halocycloalkyloxy, SO₀₋₂—(C₁-C₄)-alkyl, SO₀₋₂—(C₂-C₄)-alkenyl, SO₀₋₂(C₃-C₆)-cycloalkyl, SO₀₋₂—(C₁-C₄)-haloalkyl, SO₀₋₂—(C₂-C₄)-haloalkenyl, SO₀₋₂—(C₃-C₆)-halocycloalkyl, carboxyl, (C₁-C₄)-alkoxycarbonyl, carbamoyl, (C₁-C₄)-mono- or -dialkylamino, (C₁-C₄)-mono- or -dialkylaminocarbonyl, (C₁-C₄)-mono- or -dialkylaminosulphonyl, phenyl and phenoxy, where the phenyl and phenoxy radicals are unsubstituted or substituted by 1 to 5 of the groups mentioned.

Particularly preferred substituents are also (C₃-C₄)-alkynyloxy, CF₃, methyl, ethyl, n-propyl, isopropyl, n-butyl, tert-butyl, isobutyl.

Particular preference is given to compounds of the formula (I) in which the symbols and indices have the particularly preferred meanings mentioned.

Very particularly preferably, the symbols and indices in the formula (I) have the following meanings:
m is very particularly preferably 0.
n is very particularly preferably 0, 1 or 2.
p is very particularly preferably 0 or 1.
R¹ is very particularly preferably H, straight-chain or branched (C₁-C₆)-alkyl, (C₅-C₆)-cycloalkyl, phenyl, benzyl, 1-phenylethyl, 2-phenylethyl, where aromatic groups are unsubstituted or substituted by one or more radicals from the group consisting of halogen, cyano, (C₁-C₄)-alkyl, (C₁-C₄)-haloalkyl, (C₁-C₄)-alkoxy, (C₃-C₄)-alkynyloxy, (C₁-C₄)-haloalkoxy, SO₀₋₂(C₁-C₄)-alkyl and phenoxy, where the phenoxy radical is unsubstituted or substituted by one or more of the radicals mentioned,
R¹ is furthermore very particularly preferably 1-phenyl-2-trifluoroethyl, 1-methyl-1-phenylethyl, 1-phenylpropyl, 2-phenylpropyl, 2-methyl-2-phenylpropyl, 3-phenylpropyl, 1-methyl-3-phenylpropyl, 2-methyl-3-phenylpropyl, 2-phenylbutyl, 2-ethyl-2-phenylbutyl, naphthalenylmethyl or 1-naphthalenylethyl and substituted cycloalkyl which is substituted by one or more radicals from the group consisting of halogen, cyano, (C₁-C₄)-alkyl, (C₁-C₄)-haloalkyl, (C₁-C₄)-alkoxy, (C₃-C₄)-alkynyloxy, (C₁-C₄)-haloalkoxy, SO₀₋₂(C₁-C₄)-alkyl and phenoxy, where the phenoxy radical is unsubstituted or substituted by one or more of the radicals mentioned,

### Or

R¹ is very particularly preferably NR⁶R⁷, where
   R⁶ is H or six-membered aromatic heterocyclyl which contains one to three nitrogen atoms and is unsubstituted or substituted by one or more radicals from the group consisting of halogen, (C₁-C₄)-alkyl, (C₁-C₄)-haloalkyl, (C₁-C₄)-alkoxy, (C₁-C₄)-haloalkoxy, benzyloxy,
   R⁷ is H or COR⁸,
   R⁸ is H, unsubstituted or substituted (C₁-C₄)-alkyl, unsubstituted or substituted (C₁-C₄)-alkylamino, unsubstituted or substituted (C₁-C₄)-alkoxy, unsubstituted or substituted phenyl or unsubstituted or substituted benzyloxy,
   or
R¹ is very particularly preferably

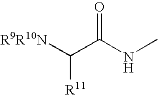

where
   R⁹ is H, straight-chain or branched (C₁-C₄)-alkyl or unsubstituted or substituted benzyl,
   R¹⁰ is H, CH₃, C₂H₅, C₃H₇, C₄H₉, C₅H₁₁, benzyl or COR⁸, where the alkyl groups are straight-chain or branched, the benzyl radical is unsubstituted or substituted by one or more, preferably 1 to 3, radicals from the group consisting of F, Cl, CH₃, i-C₃H₇, t-C₄—H₉, OCH₃, OC₂H₅, O-i-C₃H₇, CF₃ and R⁸ has the meanings given above,
   R¹¹ is H, CH₃, C₂H₅ or benzyl.
R² is very particularly preferably H, CH₃, C₂H₅ or COR¹³, where
   R¹³ is OH, O-t-C₄H₃ or benzyloxy.
R³ is very particularly preferably H, (C₁-C₄)-alkyl or (C₁-C₄)-haloalkyl.
R⁴ is very particularly preferably (C₁-C₆)-alkyl, (C₂-C₄)-alkenyl, (C₂-C₄)-alkynyl, (C₁-C₆)-haloalkyl, cyclopropyl, cyclopentyl, cyclohexyl, 1-(C₅-C₇)-cycloalkenylethyl, (C₅-C₇)-cycloalkenylmethyl, phenyl, benzyl, 1-phenylethyl, hydroxybenzyl, 1-hydroxymethyl-(C₅-C₇)-cycloalkenyl, (C₂-C₃)-haloalkenylmethyl, 1-(C₂-C₃)-haloalkenylethyl, hydroxy-(C₁-C₆)-alkyl, 1-(C₃-C₇)-cycloalkylethyl, (C₃-C₇)-cycloalkylmethyl, 1-ethyl-(C₃-C₆)-cycloalkyl, 1-methyl-(C₃-C₆)-cycloalkyl, where the alkyl groups are straight-chain or branched and where aromatic radicals are unsubstituted or substituted by one or more, preferably 1 to 3, radicals selected from the group consisting of F, Cl, t-C₄H₉, CH₃, OCH₃, CF₃, OCF₃.
R⁵ is very particularly preferably H.
X¹ is very particularly preferably a bond, straight-chain or branched (C₁-C₄)-alkylene, or (C₃-C₄)-cycloalkylene.
   X¹⁸ is very particularly preferably O, NR¹⁸, N(OR¹⁸) or NR¹⁸O, where
   R¹⁸ is H, methyl, ethyl, propyl, 1-methylethyl, butyl, 1-methylpropyl, 2-methylpropyl, 1,1-dimethylethyl, benzyl, CF₃, CF₂H, CCl₃, C₂F₅, C₃F₇, CF(CF₃)₂, acetyl, C₂H₅C(═O), C₃H₇C(═O), C₄H₉C(═O), CF₃C(═O), C₂F₅C(═O), CH₃C(═O), C₂H₅OC(═O), C₃H₇OC(═O), C₃H₇OC(═O), C₄H₉OC(═O), CF₃OC(═O), CCl₃OC(═O), C₂F₅C(═O), CH₂OCH₃; C₂H₄OCH₃, CH═CH₂, CH₂CH═CH₂, C═CH, CH₂C≡CH, SOCH₃, SOC₂H₅, SOC₃H₇, SO₂CH₃, SO₂C₂H₅, SO₂C₃H₇, SO₂CF₃, cyclopropyl, cyclobutyl, cyclopentyl, cyclohexyl; CH₂OCF₃; C₂H₄OCF₃, C═ONH₂, SiMe₃, SiMe₂tBu or SiMe₂Ph.
Z¹, Z² are very particularly preferably CH₂.

Very particular preference is given to compounds of the formula (I) in which the symbols and indices have the very particularly preferred meanings.

Especially preferably, the symbols and indices in the formula (I) have the following meanings:
m is especially preferably 0.
n is especially preferably 0, 1 or 2.
P is especially preferably 0 or 1.
R¹ is especially preferably H, CH₃, C₂H₅, C₃H₇, C₄H₉, C₅H₁₁, cyclohexyl, phenyl, benzyl, 1-phenylethyl, where the alkyl groups are straight-chain or branched and where aromatic radicals are unsubstituted or substituted by one or more, preferably 1 to 3, radicals from the group consisting of F, Cl, I, cyano, CH₃, i-C₃H₇, t-C₄—H₉, OCH₃, OC₂H₅, O-i-C₃H₇, O-t-C₄H₉, CF₃, OCF₃, OCHF₂, OCH₂C≡CH, SO₂CH₃, O-phenyl, where the last-mentioned group is unsubstituted or substituted by 1 to 3 radicals from the group consisting of F, Cl, CH₃, OCH₃,
R¹ is furthermore especially preferably 1-phenyl-2-trifluoroethyl, 1-methyl-1-phenylethyl, 1-phenylpropyl, 2-phenylpropyl, 2-methyl-2-phenylpropyl, 3-phenylpropyl, 1-methyl-3-phenylpropyl, 2-methyl-3-phenylpropyl, 2-phenylbutyl, 2-ethyl-2-phenylbutyl, naphthalen-2-ylmethyl or 1-naphthalen-2-ylethyl where the alkyl groups are straight-chain or branched and substituted cycloalkyl which is substituted by one or more, preferably 1 to 3, radicals selected from the group consisting of F, Cl, I, cyano, CH₃, i-C₃H₇, t-C₄—H₉, OCH₃, OC₂H₅, O-i-C₃H₇, O-t-C₄H₉, CF₃, OCF₃, OCHF₂, OCH₂C≡CH, SO₂CH₃, O-phenyl, where the last-mentioned group is unsubstituted or substituted by 1 to 3 radicals from the group consisting of F, Cl, CH₃, OCH₃,

### Or

R¹ is especially preferably NR⁶R⁷, where
   R⁶ is H, pyridazinyl, pyrimidinyl, pyridinyl, pyrazinyl or 1,3,5-triazinyl, in each case unsubstituted or substituted by one or two radicals from the group consisting of CH₃, i-C₃H₇, t-C₄—H₉, OCH₃, OC₂H₅, O-i-C₃H₇, CF₃, benzyloxy,
   R⁷ is H or COR⁸,
   R⁸ is benzyloxy, O-t-C₄H₉ or 9-H-fluoren-9-ylmethoxy,

### Or

R¹ is especially preferably

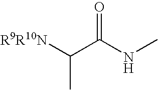

where
   R⁹ is H, CH₃, C₂H₅ or benzyl,
   R¹⁰ is H, CH₃, C₂H₅ or COR⁸, where R⁸ has the meanings given above.
R² is especially preferably H, CH₃ or COR¹³, where
   R¹³ is OH, O-t-C₄H₉ or benzyloxy.
R³ is especially preferably H, (C₁-C₄)-alkyl or (C₁-C₄)-haloalkyl.
R⁴ is especially preferably CH₃, C₂H₅, C₃H₇, C₄H₉, C₅H₁₁, cyclopropyl, cyclohexyl, 1-ethylcyclopropyl, phenyl, benzyl, 1-phenylethyl, where the alkyl groups are straight-chain or branched and where aromatic radicals are unsubstituted or substituted by one or two radicals from the group consisting of F, Cl, t-C₄—H₉, CH₃, OCH₃, CF₃, OCF₃.
R⁵ is especially preferably H.
X¹ is especially preferably a bond, CH₂, CH₂—CH₂ or cyclopropylene.
X² is especially preferably NR¹⁸, where
   R¹⁸ is H, methyl, ethyl, propyl, 1-methylethyl, butyl, benzyl, CF₃, CF₂H, C₂F₅, acetyl, C₂H₅C(═O), CF₃C(═O), CH₃OC(═O), C₂H₅OC(═O), C₃H₇OC(═O), C₃H₇OC(═O), C₄H₉C(═O), CF₃C(═O), CH₂OCH₃; C₂H₄OCH₃, CH₂CH═CH₂, CH₂C≡CH, SO₂CH₃, SO₂C₂H₅, SO₂CF₃, CH₂OCF₃; C₂H₄OCF₃, C═ONH₂ or SiMe₃.
Z¹, Z² are especially preferably CH₂.

Especially preferred are compounds of the formula (I) in which the symbols and indices have the especially preferred meanings.

Some of the compounds of the formula (I) are known, and some are novel.

Accordingly, the invention also provides the following groups of compounds of the formula (I):
a) compounds of the formula (I) in which
R³ is substituted or unsubstituted alkyl, substituted or unsubstituted alkenyl or substituted or unsubstituted arylalkyl, where preferred, particularly preferred, very particularly preferred and especially preferred meanings are evident from the meanings mentioned above, and
X² is NR¹⁸
and the other symbols and indices have the meanings given above; and
b) compounds of the formula (I) in which
R⁴ and R⁵ are identical or different and are substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heterocyclyl or substituted or unsubstituted arylalkyl, where preferred, particularly preferred, very particularly preferred and especially preferred meanings are evident from the meanings mentioned above, and
X² is NR¹⁸
and the other symbols and indices have the meaning given above; and
c) compounds of the formula (I) in which
R¹ is substituted or unsubstituted alkyl, substituted or unsubstituted cycloalkyl, substituted or unsubstituted arylalkyl or substituted or unsubstituted aryl, where preferred, particularly preferred, very particularly preferred and especially preferred meanings are evident from the meanings mentioned above, and
X² is NR¹⁸
and the other symbols and indices have the meaning given above; and
d) compounds of the formula (I) in which
X² is S, N(OR¹⁸), NR¹⁸O or NR¹⁸NH
and the other symbols and indices have the meaning given above;
e) compounds of the formula (I) in which
X² is oxygen and
R⁴ comprises at least 2 carbon atoms, where preferred, particularly preferred, very particularly preferred and especially preferred meanings are evident from the meanings mentioned above,
and the other symbols and indices have the meaning given above, with the proviso that, if
   R¹=R³=R⁵=H, X¹ is a bond and m=n=p=0, then R⁴ is not n-propyl, sec-butyl, isobutyl or 2-methyl-1-propen-3-yl;
   R¹=R³=H, R⁵=n-propyl, X¹ is a bond and m=n=p=0, then R⁴ is not n-propyl;
   R¹=methyl, R³=R⁵=H, X¹ is a bond and m=n=p=0, then R⁴ is not ethyl;
   R¹=ethyl, R³=trifluoromethyl, R⁵=H, X¹ is a bond and m=n=p=0, then R⁴ is not 1-phenylprop-3-yl;
   R¹=benzyl, R³=R⁵=H, X¹ is a bond and m=n=p=0, then R⁴ is not ethyl, isopropyl, isobutyl or 2-methyl-1-propen-3-yl; or
   R¹=tert-butyl, R³=R⁵=H, X¹ is a bond and m=n=p=0, then R⁴ is not sec-butyl, isobutyl, 1-penten-5-Y¹ or naphth-2-ylmethyl; or
   R³=H, R⁴=1-propen-3-yl, R⁵=methyl, X¹ is a bond and m=n=p=0, then R¹ is not benzyl or n-butyl; or
   R¹=benzyl, R⁴=isobutyl, R⁵=H, X¹ is a bond and m=n=p=0, then R³ is not benzyloxycarbonyl or acetyloxymethyl; or
   R¹=methyl, R³=methoxycarbonyl, R⁵=H, X¹ is a bond and m=n=p=0, then R⁴ is not methoxycarbonylmethyl; or
   R¹=ethyl, R³=ethoxycarbonyl, R⁵=H, X¹ is a bond and m=n=p=0, then R⁴ is not ethoxycarbonylmethyl.

Preferred, particularly preferred, very particularly preferred and especially preferred meanings are evident from the meanings mentioned above.

The compounds of the formula (I) can be prepared by known methods familiar to the person skilled in the art as described, for example, in Org. Lett., 2004, 6 (13), 2153-56, in Chem. Commun. 2004, 510 or by Kumaraswamy in J. Org. Chem. 2006, 71(1), 337-340.

Reference may be made in particular to EP-A-1 166 781 and the preparation methods and procedures given therein for compounds of the formula (I); this publication is incorporated into the description by reference.

However, compounds of the formula (I) can also be obtained by other routes hitherto unknown, as described in more detail in processes a) and b), for example.

### Process (a):

Compounds of the formula (I) can be prepared by reacting the acid chlorides (II) with appropriate nucleophiles of the formula (IV).

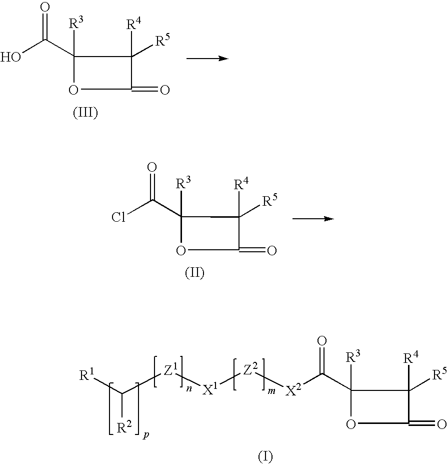

The acid chlorides (II) can be prepared from the acid (III) by various methods known to the person skilled in the art (for example Organikum, 21st edition, Wiley-VCH, 2001) and are generally reacted further without purification.

Using, for example, (2R,3S)-3-[(1S)-1-methylpropyl]-4-oxooxetane-2-carbonyl chloride and 4-chlorobenzylamine as starting materials and a base, the course of the process (a) according to the invention can be illustrated by the reaction equation below:

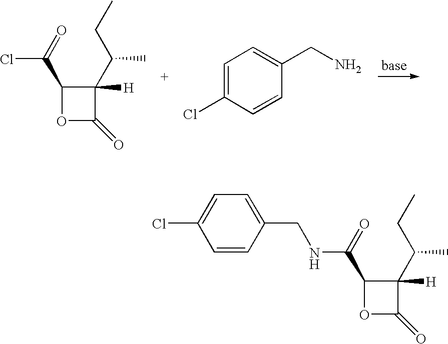

The formula (II) provides a general definition of the carbonyl halides required as starting materials for carrying out the process (a) according to the invention. In this formula (II), R³, R⁴ and R⁵ preferably, particularly preferably, very particularly preferably and especially preferably have those meanings which have already been mentioned in connection with the description of the compounds of the formula (I) according to the invention as being preferred, particularly preferred etc. for these radicals.

Process (a) also forms part of the subject matter of the present invention.

The carbonyl halides of the formula (II) are novel and can be prepared by known processes (Organikum, 21st edition, Wiley-VCH, 2001) from the known carboxylic acids or analogues thereof (lit.: Armstrong, Scutt, Chem. Commun. 2004, 510). In this formula (II), R³, R⁴ and R⁵ preferably, particularly preferably, very particularly preferably and especially preferably have those meanings which have already been mentioned in connection with the description of the compounds of the formula (I) according to the invention as being preferred, particularly preferred etc. for these radicals.

The formula (N)

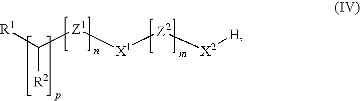

provides a general definition of the nucleophiles furthermore required as starting materials for carrying out the process (a) according to the invention. In this formula (N), R¹, R², X¹, X², Z¹, Z² and m, n and p preferably, particularly preferably, very particularly preferably and especially preferably have those meanings which have already been mentioned in connection with the description of the compounds of the formula (I) according to the invention as being preferred, particularly preferred etc. for these radicals.

The nucleophiles of the formula (N) are known and/or can be prepared by known processes (Houben-Weyl, Methoden der organischen Chemie [Methods of Organic Chemistry]; EP-A-1 166 781).

Suitable diluents for carrying out the process (a) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane 1,2-diethoxyethane or anisole; ketones, such as acetone, butanone, methyl isobutyl ketone or cyclohexanone; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide, or mixtures thereof with water.

If appropriate, the process (a) according to the invention is carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (a) according to the invention, the reaction temperatures can be varied over a relatively wide range. In general, the process is carried out at temperatures of from 0° C. to 150° C., preferably at temperatures of from 20° C. to 110° C., very particularly preferably at temperatures of 20° C.-50° C.

For carrying out the process (a) according to the invention for preparing the compounds of the formula (I), in general from 0.2 to 5 mol, preferably from 0.5 to 2 mol of nucleophile of the formula (IV) are employed per mole of the carbonyl halide of the formula (II). Work-up is carried out by customary methods.

In general, the process (a) according to the invention is carried out under atmospheric pressure. However, it is also possible to operate under elevated or reduced pressure in general between 0.1 bar and 10 bar.

### Process (b):

Compounds of the formula (I) can be prepared in a multi-step reaction from the alkyl (2R)-hydroxy-(4S)-methyl-(3S)-arylsulphanylcarbonylhexanoates (V).

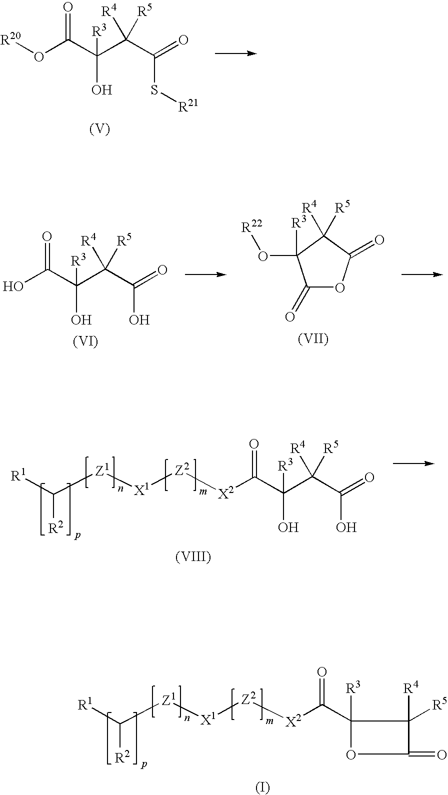

The compounds of the general formulae (V), (VI), (VII) and (VIII) can be prepared by methods known to the person skilled in the art (lit.: Org. Lett., 2004, 6 (13), 2153-56, Organikum, 21st edition, Wiley-VCH, 2001 or Houben-Weyl, Methoden der organischen Chemie) and are generally reacted further after purification by extraction or chromatography or without purification.

Using, for example, ethyl (2R)-hydroxy-(4S)-methyl-(3S)-phenylsulphanylcarbonylhexanoate and tert-butylbenzylamine as starting materials, the course of the process (b) according to the invention can be illustrated by the reaction equation below:

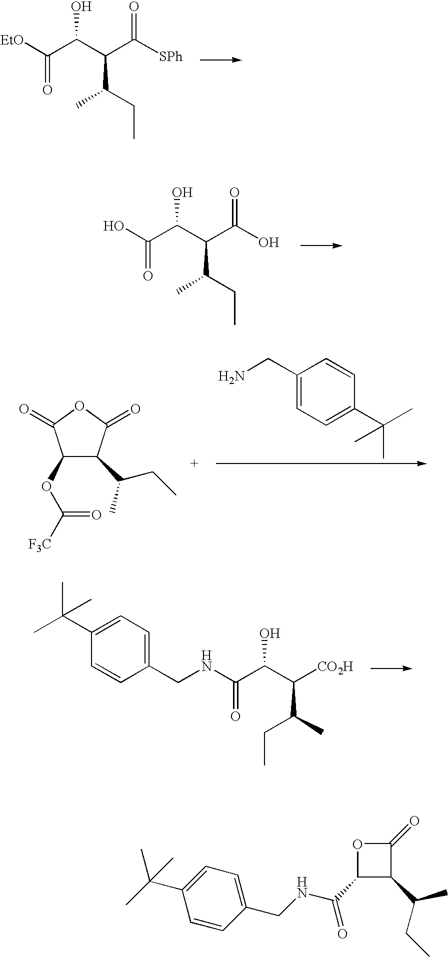

The formulae (V), (VI), (VII) and (VIII) provide a general definition of the compounds required as starting material for carrying out the process (b) according to the invention. In the formulae (V), (VI), (VII) and (VIII), R³, R⁴ and R⁵ preferably, particularly preferably, very particularly preferably and especially preferably have those meanings which have already been mentioned in connection with the description of the compounds of the formula (I) according to the invention as being preferred, particularly preferred etc. for these radicals. R²⁰ preferably represents branched and straight-chain alkyl and arylalkyl radicals, particularly preferably branched and straight-chain alkyl radicals, very particularly preferably straight-chain alkyl radicals and especially preferably ethyl. R²¹ preferably represents branched and straight-chain alkyl, arylalkyl and aryl radicals, particularly preferably branched and straight-chain alkyl and aryl radicals, very particularly preferably straight-chain aryl radicals and especially preferably phenyl. R²² represents H, COR²³, where R²³ is unsubstituted or substituted (C₁-C₄)-alkyl, unsubstituted or substituted phenyl, unsubstituted or substituted 4 to 6-membered heterocyclyl or unsubstituted or substituted benzyl; substituted or unsubstituted alkylsulphonyl, substituted or unsubstituted arylsulphonyl, preferably COR²³, where R²³ is unsubstituted or substituted (C₁-C₄)-alkyl or unsubstituted or substituted phenyl; substituted or unsubstituted alkylsulphonyl, substituted or unsubstituted arylsulphonyl, particularly preferably COR²³, where R²³ is unsubstituted or substituted (C₁ C₄)-alkyl or unsubstituted or substituted phenyl; very particularly preferably COR²³, where R²³ is (C₁ C₄)-alkyl or (C₁ C₄)-haloalkyl; especially preferably acetyl or trifluoroacetyl.

Process (b) also forms part of the subject matter of the present invention.

The (2R)-hydroxy-(4S)-methyl-(3S)-carboxyhexanoic acid of the formula (V) required in the example reaction can be prepared by basic hydrolysis according to known processes (Organikum, 21st edition, Wiley-VCH, 2001) from the known ethyl (2R)-hydroxy-(4S)-methyl-(3S)-phenylsulphanylcarbonylhexanoate (lit.: de Meijere, Larinonov, Org. Lett., 2004, 6 (13), 2153-56).

Suitable diluents for carrying out the hydrolysis in the process (b) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane 1,2-diethoxyethane or anisole; ketones, such as acetone, butanone, methyl isobutyl ketone or cyclohexanone; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide, or mixtures thereof with water. It is also possible to use aliphatic, alicyclic or aromatic alkohols, such as, for example, ethanol or methanol or mixtures thereof with water.

If appropriate, the hydrolysis according to process (b) according to the invention is carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, diisopropylethylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the hydrolysis according to the process (b) according to the invention, the reaction temperatures can be varied over a relatively wide range. In general, the process is carried out at temperatures of from 0° C. to 150° C., preferably at temperatures of from 20° C. to 110° C., very particularly preferably at temperatures of from 20° C. to 80° C.

For carrying out the hydrolysis according to process (b) according to the invention for preparing the compounds of the formula (VI), generally from 2 to 40 mol, preferably from 2 to 18 mol, of base are employed per mole of the thioester of the formula (V). Work-up is carried out by customary methods.

In the example reaction, the dicarboxylic acid (VI) is then cyclized in trifluoroacetic anhydride to give the protected (2R)-trifluoroacetoxy-(4S)-methyl-(3S)-carboxyhexanoic anhydride.

The cyclization according to the process (b) according to the invention is either carried out in the absence of a solvent, or any inert organic solvent may be used as diluent. These solvents preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide, or mixtures thereof with water.

The cyclization by the process (b) according to the invention is, if appropriate, carried out in the presence of a suitable condensing agent. Suitable condensing agents are all condensing agents customarily used for such reactions. Acid halide formers, such as phosgene, phosphorus tribromide, phosphorus trichloride, phosphorus pentachloride, phosphorus oxychloride or thionyl chloride; anhydride formers, such as ethyl chloroformate, methyl chloroformate, isopropyl chloroformate, isobutyl chloroformate, p-toluenesulphonyl chloride, methanesulphonyl chloride, acetic anhydride or trifluoroacetic anhydride; carbodiimides, such as N,N′-dicyclohexylcarbodiimide (DCC) or 1-ethyl-3-(3-dimethylaminopropyl)carbodiimide (EDCI); uronium salts, such as O-(7-azabenzotriazol-1-yl)-N,N,N′,N′-tetramethyluronium hexafluorophosphate (HATU); or other customary condensing agents, such as phosphorus pentoxide, polyphosphoric acid, N,N′-carbonyldiimidazole, 2-ethoxy-N-ethoxycarbonyl-1,2-dihydroquinoline (EEDQ), triphenylphosphine/carbon tetrachloride, bromotripyrrolidinophosphonium hexafluorophosphate or bis(2-oxo-3-oxazolidinyl)phosphinic chloride (BOP chloride), may be mentioned by way of example.

When carrying out the cyclization according to the process (b) according to the invention, the reaction temperatures can be varied over a relatively wide range. In general, the process is carried out at temperatures of from −50° C. to 100° C., preferably at temperatures of from −30° C. to 50° C., very particularly preferably at temperatures of from −10° C. to 30° C.

For carrying out the cyclization according to the process (b) according to the invention for preparing the compounds of the formula (VII), in general from 2 to 30 mol, preferably from 2 to 20 mol, of condensing agent are employed per mole of the compound of the general formula (VI). Work-up is carried out by customary methods.

The formula (N)

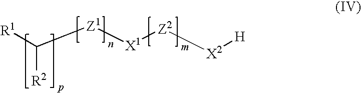

provides a general definition of the nucleophiles furthermore required as starting materials for carrying out the process (b) according to the invention. In this formula (N), R¹, R², X¹, X², Z¹, Z² and m, n and p preferably, particularly preferably, very particularly preferably and especially preferably have those meanings which have already been mentioned in connection with the description of the compounds of the formula (I) according to the invention as preferred, particularly preferred etc. for these radicals and this index.

The nucleophiles of the formula (IV) are known and/or can be prepared by known processes (EP-A-1 166 781; Houben-Weyl, Methoden der organischen Chemie).

In the example reaction, the anhydride of the general formula (VII) is reacted with the tert-butylbenzylamine, and the hydroxycarboxylic acid of the general formula (VIII) is then released under basic conditions.

Suitable diluents for carrying out the process (b) according to the invention for preparing compounds of the general (VIII) are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane 1,2-diethoxyethane or anisole; ketones, such as acetone, butanone, methyl isobutyl ketone or cyclohexanone; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide, or mixtures thereof with water. Suitable solvents for the basic aftertreatment are the same inert solvents, aliphatic or aromatic alcohols, such as, for example, methanol or ethanol and mixtures thereof with water.

If appropriate, the preparation of the compounds of the general formula (VIII) according to process (b) according to the invention is carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When preparing the compounds of the general formula (VIII) according to the process (b) according to the invention, the reaction temperatures can be varied over a relatively wide range. In general, the process is carried out at temperatures of from −50° C. to 100° C., preferably at temperatures of from −30° C. to 50° C., very particularly preferably at temperatures of from −10° C. to 30° C.

For preparing the compounds of the general formula (VIII) according to the process (b) according to the invention, in general from 0.2 to 5 mol, preferably from 0.5 to 2 mol, of the amine of the formula (IV) are employed per mole of the compound of the formula (VII). Work-up is carried out by customary methods.

Finally, the hydroxycarboxylic acids of the general formula (VIII) are lactonized using coupling agents, for example using O-(7-azabenzotriazol-1-yl)-N,N,N′,N′-tetramethyluronium hexafluorophosphate, to give the compound of the general formula (I).

Suitable diluents for carrying out the lactonization according to process (b) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as, for example, diethyl ether, diisopropyl ether, methyl t-butyl ether, methyl t-amyl ether, dioxane, tetrahydrofuran, 1,2-dimethoxyethane 1,2-diethoxyethane or anisole; ketones, such as acetone, butanone, methyl isobutyl ketone or cyclohexanone; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide, or mixtures thereof with water.

The lactonization according to process (b) according to the invention is, if appropriate, carried out in the presence of a suitable acid acceptor. Suitable acid acceptors are all customary inorganic or organic bases. These include, for example, alkali metal or alkaline earth metal hydrides, hydroxides, amides, alkoxides, acetates, carbonates or bicarbonates, such as, for example, sodium hydride, sodium amide, lithium diisopropylamide, sodium methoxide, sodium ethoxide, potassium tertbutoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, diisoproylethylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The lactonization according to process (b) according to the invention is, if appropriate, carried out in the presence of a suitable condensing agent. Suitable condensing agents are all condensing agents customarily used for such reactions. Acid halide formers, such as phosgene, phosphorus tribromide, phosphorus trichloride, phosphorus pentachloride, phosphorus oxychloride or thionyl chloride; anhydride formers, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or 1-ethyl-3-(3-dimethylaminopropyl)carbodiimide (EDCI); uronium salts, such as O-(7-azabenzotriazol-1-yl)-N,N,N′,N′-tetramethyluronium hexafluorophosphate (HATU); or other customary condensing agents, such as phosphorus pentoxide, polyphosphoric acid, N,N′-carbonyldiimidazole, 2-ethoxy-N-ethoxycarbonyl-1,2-dihydroquinoline (EEDQ), triphenylphosphine/carbon tetrachloride, bromotripyrrolidinophosphonium hexafluorophosphate or bis(2-oxo-3-oxazolidinyl)phosphinic chloride (BOP chloride), may be mentioned by way of example.

The lactonization according to process (b) according to the invention is, if appropriate, carried out in the presence of a catalyst. 4-Dimethylaminopyridine, 1-hydroxybenzotriazole, 1-hydroxy-7-azabenzotriazole or dimethylformamide may be mentioned by way of example.

When carrying out the lactonization by the process (b) according to the invention, the reaction temperatures may be varied within a relatively wide range. In general, the process is carried out at temperatures of from 0° C. to 150° C., preferably at temperatures of from 20° C. to 110° C., very particularly preferably at temperatures of from 20° C. to 50° C.

For carrying out the lactonization of the process (b) according to the invention for preparing the compounds of the formula (I), in general from 0.2 to 5 mol, preferably from 0.5 to 2 mol of coupling agent and from 0.2 bis 10 mol, preferably from 0.5 to 8 mol of base are employed per mole of the compound of the formula (VIII). Work-up is carried out by customary methods.

The process (b) according to the invention is generally carried out under atmospheric pressure. However, it is also possible to operate under elevated or reduced pressure—in general between 0.1 bar and 10 bar.

The active compounds according to the invention have potent microbicidal activity and can be employed for controlling unwanted microorganisms, such as fungi and bacteria, in crop protection and in the protection of materials.

Fungicides can be employed in crop protection for controlling, for example, Plasmodiophoromycetes, Oomycetes, Chytridiomycetes, Zygomycetes, Ascomycetes, Basidiomycetes and Deuteromycetes.

Bactericides can be employed in crop protection for controlling, for example, Pseudomonadaceae, Rhizobiaceae, Enterobacteriaceae, Corynebacteriaceae and Streptomycetaceae.

The active compounds according to the invention have very good fungicidal properties and can be used for controlling phytopathogenic fungi, such as Plasmodiophoromycetes, Oomycetes, Chytridiomycetes, Zygomycetes, Ascomycetes, Basidiomycetes and Deuteromycetes, etc.

Some pathogens causing fungal and bacterial diseases which come under the generic names listed above may be mentioned as examples, but not by way of limitation:

Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
diseases caused by powdery mildew pathogens, such as, for example
Blumeria species such as, for example, Blumeria graminis;
Podosphaera species such as, for example, Podosphaera leucotricha;
Sphaerotheca species such as, for example, Sphaerotheca fuliginea;
Uncinula species such as, for example, Uncinula necator;
diseases caused by rust pathogens such as, for example,
Gymnosporangium species such as, for example, Gymnosporangium sabinae
Hemileia species such as, for example, Hemileia vastatrix;
Phakopsora species such as, for example, Phakopsora pachyrhizi and Phakopsora meibomiae;
Puccinia species such as, for example, Puccinia recondita;
Uromyces species such as, for example, Uromyces appendiculatus;
diseases caused by pathogens from the Oomycetes group such as, for example,
Bremia species such as, for example, Bremia lactucae;
Peronospora species such as, for example, Peronospora pisi or P. brassicae;
Phytophthora species such as, for example, Phytophthora infestans;
Plasmopara species such as, for example, Plasmopara viticola;
Pseudoperonospora species such as, for example, Pseudoperonospora humuli or Pseudoperonospora cubensis;
Pythium species such as, for example, Pythium ultimum;
leaf spot diseases and leaf wilts caused by, for example,
Alternaria species such as, for example, Alternaria solani;
Cercospora species such as, for example, Cercospora beticola;
Cladiosporum species such as, for example, Cladiosporium cucumerinum;
Cochliobolus species such as, for example, Cochliobolus sativus
(conidia form: Drechslera, syn: Helminthosporium);
Colletotrichum species such as, for example, Colletotrichum lindemuthanium;
Cycloconium species such as, for example, Cycloconium oleaginum;
Diaporthe species such as, for example, Diaporthe citri;
Elsinoe species such as, for example, Elsinoe fawcettii;
Gloeosporium species such as, for example, Gloeosporium laeticolor;
Glomerella species such as, for example, Glomerella cingulata;
Guignardia species such as, for example, Guignardia bidwelli;
Leptosphaeria species such as, for example, Leptosphaeria maculans;
Magnaporthe species such as, for example, Magnaporthe grisea;
Mycosphaerella species such as, for example, Mycosphaerelle graminicola;
Phaeosphaeria species such as, for example, Phaeosphaeria nodorum;
Pyrenophora species such as, for example, Pyrenophora teres;
Ramularia species such as, for example, Ramularia collo-cygni;
Rhynchosporium species such as, for example, Rhynchosporium secalis;
Septoria species such as, for example, Septoria apii;
Typhula species such as, for example, Typhula incarnata;
Venturia species such as, for example, Venturia inaequalis;
root and stem diseases caused by, for example,
Corticium species such as, for example, Corticium graminearum;
Fusarium species such as, for example, Fusarium oxysporum;
Gaeumannomyces species such as, for example, Gaeumannomyces graminis;
Rhizoctonia species such as, for example, Rhizoctonia solani;
Tapesia species such as, for example, Tapesia acuformis;
Thielaviopsis species such as, for example, Thielaviopsis basicola;
ear and panicle diseases (including maize cobs), caused by, for example,
Alternaria species such as, for example, Alternaria spp.;
Aspergillus species such as, for example, Aspergillus flavus;
Cladosporium species such as, for example, Cladosporium spp.;
Claviceps species such as, for example, Claviceps purpurea;
Fusarium species such as, for example, Fusarium culmorum;
Gibberella species such as, for example, Gibberella zeae;
Monographella species such as, for example, Monographella nivalis;
diseases caused by smuts such as, for example,
Sphacelotheca species such as, for example, Sphacelotheca reiliana;
Tilletia species such as, for example, Tilletia caries;
Urocystis species such as, for example, Urocystis occulta;
Ustilago species such as, for example, Ustilago nuda;
fruit rots caused by, for example,
Aspergillus species such as, for example, Aspergillus flavus;
Botrytis species such as, for example, Botrytis cinerea;
Penicillium species such as, for example, Penicillium expansum;
Sclerotinia species such as, for example, Sclerotinia sclerotiorum;
Verticilium species such as, for example, Verticilium alboatrum;
seed- and soil-borne rots and wilts, and seedling diseases, caused by, for example,
Fusarium species such as, for example, Fusarium culmorum;
Phytophthora species such as, for example, Phytophthora cactorum;
Pythium species such as, for example, Pythium ultimum;
Rhizoctonia species such as, for example, Rhizoctonia solani;
Sclerotium species such as, for example, Sclerotium rolfsii;
cankers, galls and witches' broom disease, caused by, for example,
Nectria species such as, for example, Nectria galligena;
wilts caused by, for example,
Monilinia species such as, for example, Monilinia laxa;
deformations of leaves, flowers and fruits, caused by, for example,
Taphrina species such as, for example, Taphrina deformans;
degenerative diseases of woody plants, caused by, for example,
Esca species such as, for example, Phaemoniella clamydospora;
diseases of inflorescences and seeds, caused by, for example,
Botrytis species such as, for example, Botrytis cinerea;
diseases of plant tubers, caused by, for example,
Rhizoctonia species such as, for example, Rhizoctonia solani.

The active compounds according to the invention also show a strong invigorating action in plants. Accordingly, they are suitable for mobilizing the internal defences of the plant against attack by unwanted microorganisms.

In the present context, plant-invigorating (resistance-inducing) compounds are to be understood as meaning those substances which are capable of stimulating the defence system of plants such that, when the treated plants are subsequently inoculated with unwanted microorganisms, they display substantial resistance to these microorganisms.

In the present case, unwanted microorganisms are to be understood as meaning phytopathogenic fungi and bacteria. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, and diseases in viticulture and in the cultivation of fruit and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably tackifiers, sizes, papers and boards, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes) and against slime organisms and algae.

Microorganisms of the following genera may be mentioned as examples:

Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

### Fungicides:

### 1. Nucleic Acid Synthesis Inhibition

benalaxyl, benalaxyl-M, bupirimate, chiralaxyl, clozylacon, dimethirimol, ethirimol, furalaxyl, hymexazol, metalaxyl-M, ofurace, oxadixyl, oxolinic acid

### 2. Mitosis and Cell Division Inhibition

benomyl, carbendazim, diethofencarb, fuberidazole, pencycuron, thiabendazole, thiophanate-methyl, zoxamide

### 3. Inhibition of the Respiratory Chain

### 3.1 Complex I

diflumetorim

### 3.2 Complex II

boscalid, carboxin, fenfuram, flutolanil, furametpyr, mepronil, oxycarboxin, penthiopyrad, thifluzamide

### 3.3 Complex III

azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin

### 3.4 Decouplers

dinocap, fluazinam

### 3.5 ATP Production Inhibition

fentin acetate, fentin chloride, fentin hydroxide, silthiofam

### 4. Amino Acid and Protein Biosynthesis Inhibition

andoprim, blasticidin-S, cyprodinil, kasugamycin, kasugamycin hydrochloride hydrate, mepanipyrim, pyrimethanil

### 5. Signal Transduction Inhibition

fenpiclonil, fludioxonil, quinoxyfen

### 6. Lipid and Membrane Synthesis Inhibition

chlozolinate, iprodione, procymidone, vinclozolin
pyrazophos, edifenphos, iprobenfos (IBP), isoprothiolane
tolclofos-methyl, biphenyl
iodocarb, propamocarb, propamocarb hydrochloride

### 7. Inhibition of Ergosterol Biosynthesis

fenhexamid,
azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazol, penconazole, propiconazole, prothioconazole, simeconazole, tebuconazole, tetraconazole, triadimefon, triadimenol, triticonazole, uniconazole, voriconazole, imazalil, imazalil sulphate, oxpoconazole, fenarimol, flurprimidol, nuarimol, pyrifenox, triforine, pefurazoate, prochloraz, triflumizole, viniconazole
aldimorph, dodemorph, dodemorph acetate, fenpropimorph, tridemorph, fenpropidin, spiroxamine,
naftifine, pyributicarb, terbinafine

### 8. Cell Wall Synthesis Inhibition

benthiavalicarb, bialaphos, dimethomorph, flumorph, iprovalicarb, polyoxins, polyoxorim, validamycin A

### 9. Melanin Biosynthesis Inhibition

capropamid, diclocymet, fenoxanil, phthalide, pyroquilon, tricyclazole

### 10. Resistance Induction

acibenzolar-S-methyl, probenazole, tiadinil

### 11. Multisite

captafol, captan, chlorothalonil, copper salts, such as: copper hydroxide, copper naphthenate, copper oxychloride, copper sulphate, copper oxide, oxine copper and Bordeaux mixture, dichlofluanid, dithianone, dodine, dodine free base, ferbam, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations comprising calcium polysulphide, thiram, tolylfluanid, zineb, ziram

### 12. Unknown

amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methylsulphate, diphenylamine, ethaboxam, ferimzone, flumetover, flusulfamide, fluopicolide, fluoroimide, hexachlorobenzene, 8-hydroxyquinoline sulphate, irumamycin, methasulfocarb, metrafenone, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrolnitrin, quintozene, tecloftalam, tecnazene, triazoxide, trichlamid, zarilamid and 2,3,5,6-tetrachloro-4-(methylsulphonyl)pyridine, N-(4-chloro-2-nitrophenyl)-N-ethyl-4-methylbenzenesulphonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]-pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]-methyl]thio]methyl]-α-(methoxymethylene)benzacetate, 4-chloro-α-propynyloxy-N-[2-[3-methoxy-4-(2-propynyloxy)phenyl]ethyl]benzacetamide, (2S)—N-[2-[4-[[3-(4-chlorophenyl)-2-propynyl]oxy]-3-methoxyphenyl]ethyl]-3-methyl-2-[(methylsulphonyl)amino]butanamide, 5-chloro-7-(4-methylpiperidin-1-yl)-6-(2,4,6-trifluorophenyl) [1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl) [1,2,4]triazolo[1,5-a]pyrimidine-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3-(1-fluoro-2-phenylethyl)oxy]phenyl]ethylidene]amino]oxy]methyl]-α-(methoxyimino)-N-methyl-α-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

### 1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 organophosphates (for example acephate, azamethiphos, azinphos (-methyl, -ethyl), bromophosethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-s-methyl, demeton-s-methylsulphone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-5-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))
2.2 oxadiazines (for example indoxacarb)

### 3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)
3.2 nicotine, bensultap, cartap

### 4. Acetylcholine Receptor Modulators

4.1 spinosyns (for example spinosad)

### 5. Antagonists of GABA-Gated Chloride Channels

5.1 cyclodiene organochlorines (for example camphechlor, chlordane, endosulfan, gamma-HCH, HCH, heptachlor, lindane, methoxychlor)
5.2 fiproles (for example acetoprole, ethiprole, fipronil, vaniliprole)

### 6. Chloride Channel Activators

6.1 mectins (for example abamectin, avermectin, emamectin, emamectin-benzoate, ivermectin, milbemectin, milbemycin)

### 7. Juvenile Hormone Mimetics

(for example diofenolan, epofenonane, fenoxycarb, hydroprene, kinoprene, methoprene, pyriproxifen, triprene)

### 8. Ecdyson Agonists/Disruptors

8.1 diacylhydrazines (for example chromafenozide, halofenozide, methoxyfenozide, tebufenozide)

### 9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)
9.2 buprofezin
9.3 cyromazine

### 10. Inhibitors of Oxidative Phosphorylation, ATP Disruptors

10.1 diafenthiuron
10.2 organotins (for example azocyclotin, cyhexatin, fenbutatin-oxide)

### 11. Decouplers of Oxidative Phosphorylation Acting by Interrupting the H-Proton Gradient

11.1 pyrroles (for example chlorfenapyr)
11.2 dinitrophenols (for example binapacryl, dinobuton, dinocap, DNOC)

### 12. Site-I Electron Transport Inhibitors

12.1 METIs (for example fenazaquin, fenpyroximate, pyrimidifen, pyridaben, tebufenpyrad, tolfenpyrad)
12.2 hydramethylnone
12.3 dicofol

### 13. Site-II Electron Transport Inhibitors

13.1 rotenone

### 14. Site-III Electron Transport Inhibitors

14.1 acequinocyl, fluacrypyrim
15. Microbial Disruptors of the Insect Gut Membrane Bacillus thuringiensis strains

### 16. Inhibitors of Fat Synthesis

16.1 tetronic acids (for example spirodiclofen, spiromesifen)
16.2 tetramic acids [for example 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-Y¹ ethyl ester, CAS Reg. No.: 382608-10-8) and carbonic acid, cis-3-(2,5-dimethylphenyl)-8-methoxy-2-oxo-1-azaspiro[4.5]dec-3-en-4-Y¹ ethyl ester (CAS Reg. No.: 203313-25-1)]

### 17. Carboxamides

(for example flonicamid)

### 18. Octopaminergic Agonists

(for example amitraz)

### 19. Inhibitors of Magnesium-Stimulated ATPase

(for example propargite)

### 20. Phthalamides

(for example N²-[1,1-dimethyl-2-(methylsulphonyl)ethyl]-3-iodo-N¹-[2-methyl-4-[1,2,2,2-tetrafluoro-1-(trifluoromethyl)ethyl]phenyl]-1,2-benzenedicarboxamide (CAS Reg. No.: 272451-65-7), flubendiamide)

### 21. Nereistoxin Analogues

(for example thiocyclam hydrogen oxalate, thiosultap-sodium)

### 22. Biologicals, Hormones or Pheromones

(for example azadirachtin, Bacillus spec., Beauveria spec., Codlemone, Metarrhizium spec., Paecilomyces spec., Thuringiensin, Verticillium spec.)
23. Active Compounds with Unknown or Unspecific Mechanisms of Action
23.1 fumigants (for example aluminium phosphide, methyl bromide, sulphuryl fluoride)
23.2 selective antifeedants (for example cryolite, flonicamid, pymetrozine)
23.3 mite growth inhibitors (for example clofentezine, etoxazole, hexythiazox)
23.4 amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin,
furthermore the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

In addition, the compounds of the formula (I) according to the invention also have very good antimycotic activity. They have a very broad antimycotic activity spectrum in particular against dermatophytes and yeasts, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, by the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are particularly emphasized are increased defence of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in each case can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

The invention is illustrated in more detail by the examples below, without being limited thereby.

### EXAMPLES

General procedures for the synthesis of belactone carboxylic acid derivatives:

### Example 1

### (3S)-(1S-Methylpropyl)-(2R)-(3,5-dimethoxybenzylaminocarbonyl)-4-oxooxetane

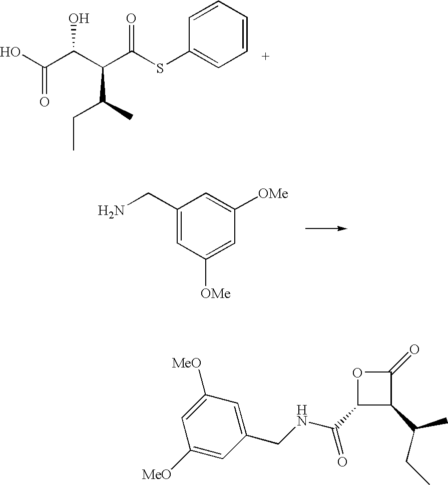

### (3S)-(1S-Methylpropyl)-(2R)-(3,5-dimethoxybenzylaminocarbonyl)-4-oxooxetane

A solution of 33.9 mg (0.22 mmol) of N-(3-dimethylaminopropyl)-N′-ethylcarbodiimide (EDC) in 1 ml DMF (dimethylformamide)/DCM (methylene chloride) (1:3) and 19.7 mg (0.12 mmol) of 3,5-dimethoxybenzylamine in 1 ml of DMF/DCM (1:3) were successively added dropwise to a solution, cooled to −30° C., of 40 mg (0.14 mmol) of ethyl (2R)-hydroxy-(4S)-methyl-(3S)-phenylsulphanylcarbonylhexanoate, 25.5 mg (0.19 mmol) of 1-hydroxy-7-azabenzotriazole (HOAt), 38.6 mg (0.32 mmol) of 2,4,6-trimethylpyridine in 3 ml of DMF/DCM (1:3). The mixture was stirred at −30° C. for 5 h, warmed to room temperature overnight and kept at this temperature for a further 18 h. This was followed by the addition of 10% strength aqueous citric acid, washing of the organic phase with water, sat. NaCl solution and drying of the organic phase over MgSO₄. The crude product was concentrated under reduced pressure and purified chromatographically (SiO₂, cyclohexane/ethyl acetae gradient).

This gives 5.9 mg (14.6%) of (3S)-(1S-methylpropyl)-(2R)-(3,5-dimethoxybenzylaminocarbonyl)-4-oxooxetane ¹H NMR (D3-acetonitrile, 400 MHz): δ=0.91 (t, 3H), 1.01 (d, 3H), 1.27 (m, 1H), 1.60 (m, 1H), 2.16 (m, 1H), 3.66 (dd, 1H), 3.75 (s, 6H), 4.31 (dd, 1H), 4.36 (dd, 1H), 4.72 (d, 1H), 6.38 (t, 1H), 6.44 (s, 2H), 7.37 (bs, NH)

Ethyl (2R)-hydroxy-(4S)-methyl-(3S)-phenylsulphanylcarbonylhexanoate was synthesized by procedures known from the literature (Org. Lett., 2004, 6 (13), 2153-56).

### Example 2

### Preparation of (2R,3S)—N-(4-chlorobenzyl)-3-[(1S)-1-methylpropyl]-4-oxooxetane-2-carboxamide

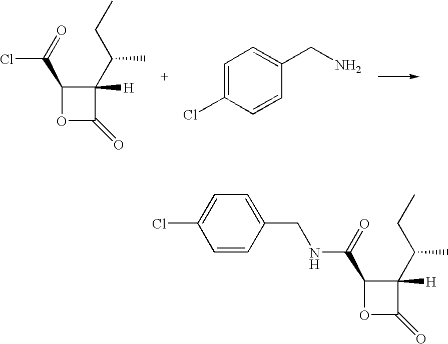

At room temperature, a solution of 52 mg (0.37 mmol) of 4-chlorobenzylamine in 2.5 ml of dioxane and then 0.05 ml (0.37 mmol) of triethylamine are added to a solution of 70 mg (0.37 mmol) of (2R,3S)-3-[(1S)-1-methylpropyl]-4-oxooxetane-2-carbonyl chloride in 2.5 ml of dioxane, and the mixture is stirred at room temperature for 20 h. The reaction solution is then filtered through silica gel, and the filter cake is washed with dichloromethane/methanol. If required, the crude product can be purified further by chromatography on silica gel using cyclohexane/ethyl acetate. Yield of (2R,3S)—N-(4-chlorobenzyl)-3-[(1S)-1-methylpropyl]-4-oxooxetane-2-carboxamide: 111 mg (80%). logP (HCO₂H)=3.04.

### Preparation of (2R,3S)-3-[(1S)-1-methylpropyl]-4-oxooxetane-2-carbonyl chloride

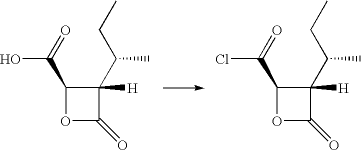

At room temperature, 0.7 ml (9.6 mmol) of thionyl chloride are added dropwise to a solution of 189 mg (1.1 mmol) of (2R,3S)-3-[(1S)-1-methylpropyl]-4-oxooxetane-2-carboxylic acid in 6.5 ml of dichloromethane, and the reaction solution is heated at reflux overnight. The volatile components are then removed under reduced pressure, and the (2R,3S)-3-[(1S)-1-methylpropyl]-4-oxooxetane-2-carbonyl chloride is directly reacted further without further purification.

(2R,3S)-3-[(1S)-1-Methylpropyl]-4-oxooxetane-2-carboxylic acid was prepared by methods known from the literature (Chem. Commun. 2004, 510-511).

### Example 3

### Preparation of (2R,3S)-3-[(1S)-1-methylpropyl]-4-oxo-N-(3,4,5-trimethoxyphenyl)oxetane-2-carboxamide

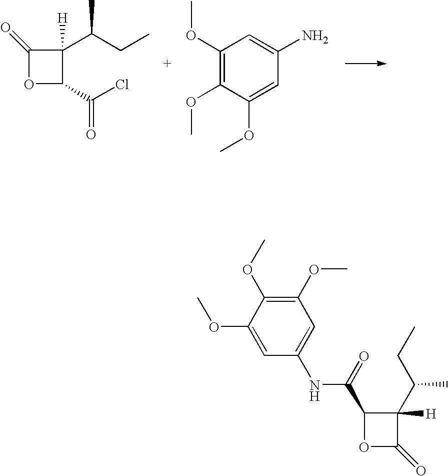

At room temperature, a solution of 57 mg (0.31 mmol) of 3,4,5-trimethoxyaniline in 2.5 ml of dioxane and then 0.04 ml (0.31 mmol) of triethylamine are added to a solution of 60 mg (0.31 mmol) of (2R,3S)-3-[(1S)-1-methylpropyl]-4-oxooxetane-2-carbonyl chloride in 2.5 ml of dioxane, and the mixture is stirred at room temperature for 20 h. The solvent is then removed, and the residue is taken up in diethyl ether and filtered through silica gel. The crude product obtained in this manner is purified further by chromatography on silica gel using cyclohexane/ethyl acetate. Yield of (2R,3S)-3-[(1S)-1-methylpropyl]-4-oxo-N-(3,4,5-trimethoxyphenyl)oxetane-2-carboxamide: 63 mg (58%). logP (HCO₂H)=2.50.

### Example 4

### Preparation of 4-tert-butylbenzyl 3S-(1S-methylpropyl)-4-oxooxetane-2R-carboxamide

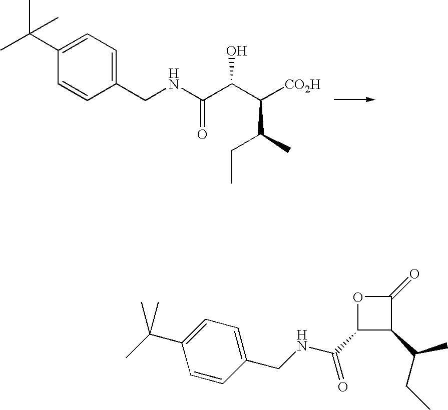

At 0° C., 0.2 g of diisopropylethylamine (DIPEA, 2.6 mmol) and 0.159 g of O-(7-azabenzotriazol-1-yl)-N,N,N′,N′-tetramethyluronium hexafluorophosphate (HATU, 0.42 mmol) were added to a solution of 0.118 g of N-4-tert-butylbenzyl-(2R)-hydroxy-(4S)-methyl-(3S)-carboxyhexanamide (0.35 mmol) in CH₂Cl₂, and the mixture was stirred at room temperature for 3 h. At this point in time, the reaction mixture homogenized to form a clear solution which was washed repeatedly with saturated aqueous KHSO₄. After each washing step, the aqueous phase was extracted with CH₂Cl₂, and the combined organic phases were dried over Na₂SO₄ and concentrated under reduced pressure. The crude product was purified chromatographically (SiO₂, cyclohexane/ethyl acetate gradient), giving 84 mg of (3S)-(1S-methylpropyl)-(2R)-(4-tert-butylbenzylaminocarbonyl)-4-oxooxetane (75.5%).

(3S)-(1S-Methylpropyl)-(2R)-(4-tert-butylbenzylaminocarbonyl)-4-oxooxetane: NMR (D3-Acetonitril, 400 MHz): δ=0.90 (t, 3H), 1.00 (d, 3H), 1.28 (m, 1H), 1.29 (s, 9H), 1.59 (m, 1H), 1.95 (m, 1H), 3.65 (dd, 1H), 4.33 (dd, 1H), 4.39 (dd, 1H), 4.68 (d, 1H), 7.20 (d, 2H), 7.36 (bs, NH), 7.37 (d, 2H)

### Preparation of N-4-tert-butylbenzyl-(2R)-hydroxy-(4S)-methyl-(3S)-carboxyhexanamide

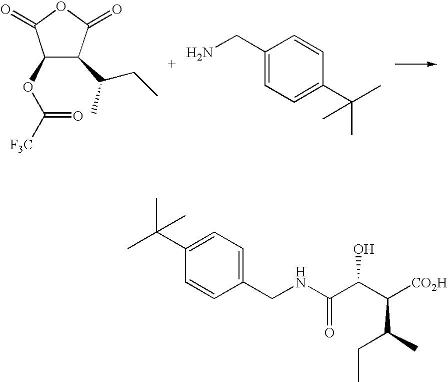

(2R)-Trifluoroacetoxy-(4S)-methyl-(3S)-carboxyhexanoic anhydride was dissolved in THF and, after cooling to from −5 to 0° C., a solution of 377 mg of 4-tert-butylbenzylamine (2.3 mmol) and 234 mg of triethylamine (2.3 mmol) in 1 ml of THF was added. After 30 min, the ice bath was removed, and the mixture was stirred at room temperature for 4 h. The reaction mixture was concentrated under reduced pressure, 1 ml of cold (0° C.) MeOH and 0.6 ml of 3 M NaOH were added on top of the residue and the mixture was stirred at room temperature for 1 h and concentrated at 35° C. under reduced pressure. The residue was acidified with 4 N HCl to pH 1 and extracted with CH₂Cl₂ (4×10 ml). The collected organic phases were dried over Na₂SO₄ and concentrated, and the crude product was purified by preparative LC (Kromasil 100-5 C18, 250×20 mm; A=2% strength HCOOH, B=acetonitrile, A/B=47/53% and after 9 minutes 39/61%, A/B isocratic, flow rate: 25 ml/min, UV=210 nm). This gave 120 mg of N-4-tert-butylbenzyl-(2R)-hydroxy-(4S)-methyl-(3S)-carboxyhexanamide (30.8%).

N-tert-Butylbenzyl-(2R)-hydroxy-(4S)-methyl-(3S)-carboxyhexanamide: ¹H-NMR (D3-acetonitrile, 400 MHz): δ=0.89 (t, 3H), 1.00 (d, 3H), 1.24 (m, 1H), 1.30 (s, 9H), 1.50 (m, 1H), 1.95 (m, 1H), 2.77 (dd, 1H), 4.29 (d, 1H), 4.36 (dd, 2H), 7.20 (d, 2H), 7.37 (d, 2H), 7.47 (bs, NH).

### Synthesis of (2R)-trifluoroacetoxy-(4S)-methyl-(3S)-carboxyhexanoic anhydride

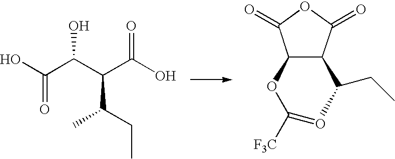

A solution of 220 mg of (2R)-hydroxy-(4S)-methyl-(3S)-carboxyhexanoic acid (0.157 mmol) and 4 ml of trifluoroacetic anhydride was stirred at from −5 to 0° C. (NaCl—ice bath) for 3 h. Excess trifluoroacetic anhydride was distilled off under high vacuum (0.05 mbar) at from ±5 to 0° C. over 2-3 h. Towards the end, the glass-like residue was occasionally shaken manually to remove as much trifluoroacetic anhydride and trifluoroacetic acid as possible. The (2R)-trifluoroacetoxy-(4S)-methyl-(3S)-carboxyhexanoic anhydride was vented with N₂ and immediately, without further purification, used for the next reaction.

### Preparation of (2R)-hydroxy-(4S)-methyl-(3S)-carboxyhexanoic acid

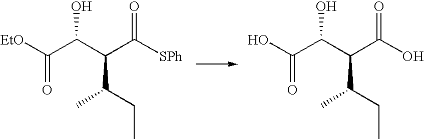

6 ml of 3 M aqueous NaOH were added to a solution of 420 mg of ethyl (2R)-hydroxy-(4S)-methyl-(3S)-phenylsulphanylcarbonylhexanoate (1.35 mmol) in MeOH, and the mixture was stirred vigorously at 65° C. for 16 h. The reaction mixture was initially concentrated under reduced pressure at 50° C. and then twice triturated with cyclohexane to remove the thiophenol. The viscous residue was cooled on an NaCl bath to from −5 to 0° C., and cold (−5 to 0° C.) 12 N HCl was added such that the reaction temperature remained below 40° C. After addition of CHCl₃, the mixture was stirred vigorously for 15 min, and Na₂SO₄ was then added in such an amount that the aqueous phase was absorbed completely. The mixture was stirred at 60° C. for 30 min and the organic phase was dried over Na₂SO₄, giving, after concentration under reduced pressure, 220 mg of (2R)-hydroxy-(4S)-methyl-(3S)-carboxyhexanoic acid (85%) as a white solid.

(2R)-Hydroxy-(4S)-methyl-(3S)-carboxyhexanoic acid: ¹H NMR (D6-DMSO, 400 MHz): δ=0.84 (t, 3H), 0.94 (d, 3H), 1.28 (m, 1H), 1.49 (m, 1H), 1.77 (m, 1H), 2.55 (m, 1H), 4.16 (dd, 1H)

Ethyl (2R)-hydroxy-(4S)-methyl-(3S)-phenylsulphanylcarbonylhexanoate was synthesized by procedures known from the literature (Org. Lett., 2004, 6 (13), 2153-56).

Using the methods stated above, it is or was also possible to obtain the beta-lactonecarboxylic acid derivatives of the formula

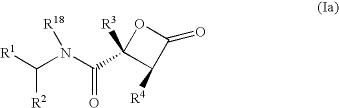

listed in Table 1 below.

TABLE 1

| Example | R⁴ | R³ | R¹⁸ | R² | R¹ | logP/Rt*) |
|---|---|---|---|---|---|---|
| 5 | 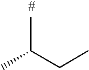 | H | H | H | 3-chlorophenyl | 2.87²⁾ |
| 5a | 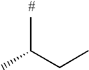 | H | H | H | 2-chlorophenyl | 2.95¹⁾ |
| 6 | 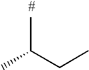 | H | H | H | 2-methylphenyl | |
| 7 | 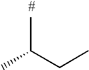 | H | H | H | 3-methylphenyl | 2.92¹⁾ |
| 8 | 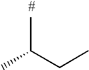 | H | H | H | 4-methylphenyl | |
| 9 | 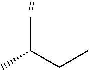 | H | H | H | 2-methoxyphenyl | |
| 10 | 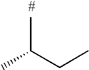 | H | H | H | 3-methoxyphenyl | 2.62¹⁾ |
| 11 | 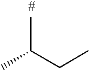 | H | H | H | 4-methoxyphenyl | 2.57¹⁾ |
| 12 | 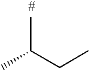 | H | H | H | 2,4-dichlorophenyl | 3.44¹⁾ |
| 13 | 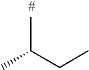 | H | H | H | 3,4-dichlorophenyl | |
| 14 | 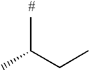 | H | H | H | 3,5-dichlorophenyl | 3.07²⁾ |
| 15 | 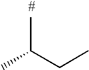 | H | H | H | 2,5-dichlorophenyl | |
| 16 | 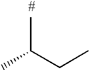 | H | H | H | 2,4,6-trichlorophenyl | |
| 17 | 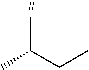 | H | H | H | 2-chloro-4-methylphenyl | |
| 18 | 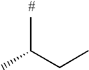 | H | H | H | 2-chloro-4-methoxy- phenyl | |
| 19 | 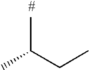 | H | H | H | 2,4-dimethylphenyl | |
| 20 | 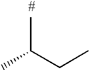 | H | H | H | 2,5-dimethylphenyl | |
| 21 | 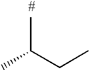 | H | H | H | 2,6-dimethylphenyl | |
| 22 | 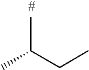 | H | H | H | 2,4,6-trimethylphenyl | |
| 23 | 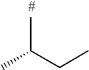 | H | H | H | 2-fluorophenyl | |
| 24 | 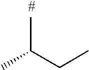 | H | H | H | 3-fluorophenyl | 2.70¹⁾ |
| 25 | 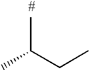 | H | H | H | 4-fluorophenyl | 2.62¹⁾ |
| 26 | 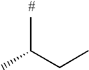 | H | H | H | 2-chloro-4-fluorophenyl | |
| 27 | 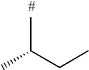 | H | H | H | 2,4-dimethoxyphenyl | |
| 28 | 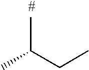 | H | H | H | 2,3-dimethoxyphenyl | |
| 29 | 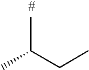 | H | H | H | 3,4-dimethoxyphenyl | |
| 30 | 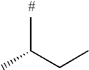 | H | H | H | 2,5-dimethoxyphenyl | |
| 31 | 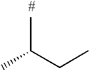 | H | H | H | 2-trifluoromethylphenyl | |
| 32 | 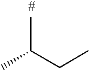 | H | H | H | 3-trifluoromethylphenyl | |
| 33 | 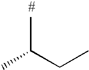 | H | H | H | 4-trifluoromethylphenyl | |
| 33a | 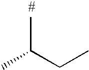 | H | H | H | 3-chloro-4-trifluoro- methylphenyl | 3.56¹⁾ |
| 34 | 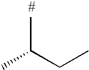 | H | H | H | 3-trifluoromethyl- 4-chlorophenyl | |
| 35 | 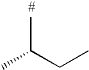 | H | H | H | 3-trifluoromethyl- 4-fluorophenyl | |
| 36 | 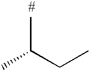 | H | H | H | 2-trifluoro- methoxyphenyl | |
| 37 | 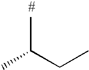 | H | H | H | 3-trifluoro- methoxyphenyl | 3.40¹⁾ |
| 38 | 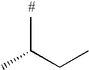 | H | H | H | 4-trifluoro- methoxyphenyl | |
| 39 | 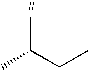 | H | H | H | 2-difluoro- methoxyphenyl | |
| 40 | 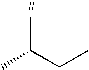 | H | H | H | 3-difluoro- methoxyphenyl | 2.83²⁾ |
| 41 | 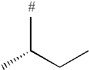 | H | H | H | 4-difluoro- methoxyphenyl | 2.93¹⁾ |
| 42 | 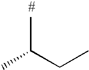 | H | H | H | 2-tert-butylphenyl | |
| 43 | 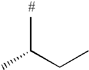 | H | H | H | 3-tert-butylphenyl | |
| 44 | 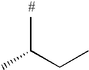 | H | H | H | 2-chlorophenyl | |
| 45 | 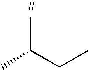 | H | H | H | 2-phenoxyphenyl | |
| 46 | 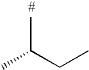 | H | H | H | 3-phenoxyphenyl | |
| 47 | 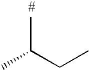 | H | H | H | 4-phenoxyphenyl | |
| 48 | 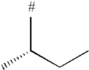 | H | H | H | 2-(4-methoxyphenoxy)- phenyl | 3.11²⁾ |
| 49 | 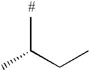 | H | H | H | 3-(4-methoxyphenoxy)- phenyl | |
| 50 | 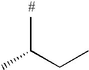 | H | H | H | 4-(4-methoxyphenoxy)- phenyl | |
| 51 | 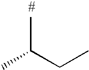 | H | H | H | 4-methanesulphonyl- phenyl | |
| 52 | 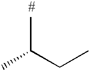 | H | H | H | 2-ethoxyphenyl | |
| 53 | 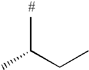 | H | H | H | 3-ethoxyphenyl | |
| 54 | 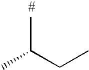 | H | H | H | 4-ethoxyphenyl | |
| 54a | 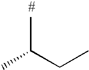 | H | H | H | 3-methoxy-4-(prop-2- yn-1-yloxy)phenyl | 3.24¹⁾ |
| 54b | 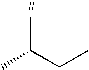 | H | H | H | 2,2-difluoro-1,3-benzo- dioxol-5-yl | 2.60¹⁾ |
| 55 | 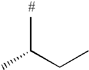 | H | H | H | methyl | |
| 56 | 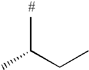 | H | H | H | ethyl | |
| 57 | 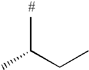 | H | H | H | n-propyl | |
| 58 | 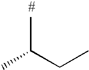 | H | H | H | n-butyl | |
| 59 | 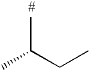 | H | H | H | n-pentyl | |
| 60 | 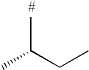 | H | H | H | benzyl | 2.75²⁾ |
| 61 | 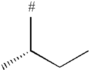 | H | H | H | 2-chlorobenzyl | |
| 62 | 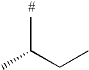 | H | H | H | 3-chlorobenzyl | |
| 63 | 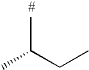 | H | H | H | 4-chlorobenzyl | 3.20¹⁾ |
| 64 | 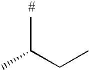 | H | H | H | 2-methylbenzyl | |
| 65 | 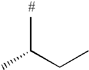 | H | H | H | 3-methylbenzyl | |
| 66 | 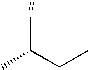 | H | H | H | 4-methylbenzyl | |
| 67 | 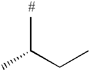 | H | H | H | 2-methoxybenzyl | 2.84²⁾ |
| 68 | 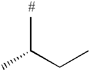 | H | H | H | 3-methoxybenzyl | 2.75²⁾ |
| 69 | 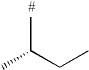 | H | H | H | 4-methoxybenzyl | 2.65¹⁾ |
| 70 | 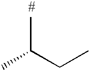 | H | H | H | 2,4-dichlorobenzyl | |
| 71 | 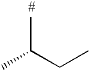 | H | H | H | 3,4-dichlorobenzyl | |
| 72 | 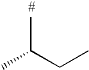 | H | H | H | 3,5-dichlorobenzyl | |
| 73 | 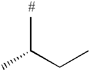 | H | H | H | 2,5-dichlorobenzyl | |
| 74 | 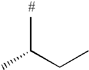 | H | H | H | 2,4,6-trichlorobenzyl | |
| 75 | 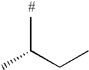 | H | H | H | 2-chloro-4-methylbenzyl | |
| 76 | 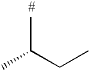 | H | H | H | 2-chloro-4-methoxy- benzyl | |
| 77 | 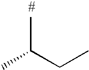 | H | H | H | 2,4-dimethylbenzyl | 3.37¹⁾ |
| 78 | 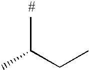 | H | H | H | 2,5-dimethylbenzyl | |
| 79 | 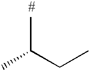 | H | H | H | 2,6-dimethylbenzyl | |
| 80 | 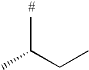 | H | H | H | 2,4,6-trimethylbenzyl | |
| 81 | 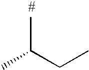 | H | H | H | 2-fluorobenzyl | |
| 82 | 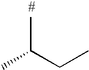 | H | H | H | 3-fluorobenzyl | 2.79²⁾ |
| 83 | 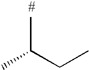 | H | H | H | 4-fluorobenzyl | 2.79²⁾ |
| 84 | 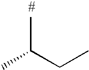 | H | H | H | 2-chloro-4-fluorobenzyl | |
| 85 | 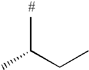 | H | H | H | 2,4-dimethoxybenzyl | |
| 86 | 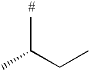 | H | H | H | 2,3-dimethoxybenzyl | 2.77²⁾ |
| 87 | 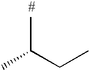 | H | H | H | 3,4-dimethoxybenzyl | 2.57²⁾ |
| 88 | 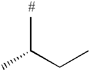 | H | H | H | 3,5-dimethoxybenzyl | |
| 89 | 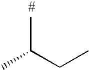 | H | H | H | 2,5-dimethoxybenzyl | 2.81²⁾ |
| 90 | 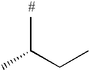 | H | H | H | 2-trifluoromethylbenzyl | |
| 91 | 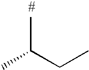 | H | H | H | 3-trifluoromethylbenzyl | 3.02²⁾ |
| 92 | 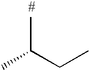 | H | H | H | 4-trifluoromethylbenzyl | 3.33¹⁾ |
| 93 | 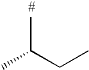 | H | H | H | 3-trifluoromethyl-4- chlorobenzyl | |
| 94 | 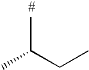 | H | H | H | 3-trifluoromethyl- 4-fluorobenzyl | |
| 95 | 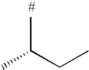 | H | H | H | 2-trifluoro- methoxybenzyl | |
| 96 | 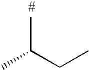 | H | H | H | 3-trifluoro- methoxybenzyl | 3.08²⁾ |
| 97 | 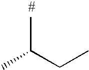 | H | H | H | 4-trifluoro- methoxybenzyl | |
| 98 | 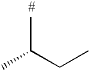 | H | H | H | 2-difluoro- methoxybenzyl | |
| 99 | 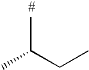 | H | H | H | 3-difluoro- methoxybenzyl | |
| 100 | 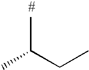 | H | H | H | 4-difluoro- methoxybenzyl | |
| 101 | 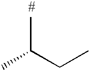 | H | H | H | 2-tert-butylbenzyl | |
| 102 | 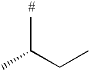 | H | H | H | 3-tert-butylbenzyl | |
| 103 | 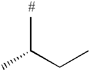 | H | H | H | 4-tert-butylbenzyl | 4.04¹⁾ |
| 104 | 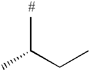 | H | H | H | 2-phenoxybenzyl | |
| 105 | 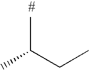 | H | H | H | 3-phenoxybenzyl | 3.18²⁾ |
| 106 | 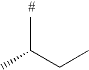 | H | H | H | 4-phenoxybenzyl | 3.17²⁾ |
| 107 | 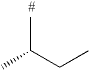 | H | H | H | 2-(4-methoxyphenoxy)- benzyl | |
| 108 | 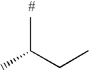 | H | H | H | 3-(4-methoxyphenoxy)- benzyl | |
| 109 | 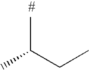 | H | H | H | 4-(4-methoxyphenoxy)- benzyl | |
| 110 | 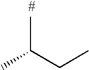 | H | H | H | 4-methanesulphonyl- benzyl | |
| 111 | 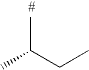 | H | H | H | 2-ethoxybenzyl | 3.01²⁾ |
| 112 | 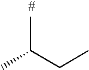 | H | H | H | 3-ethoxybenzyl | |
| 113 | 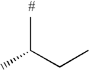 | H | H | H | 4-ethoxybenzyl | 2.88²⁾ |
| 114 | 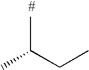 | H | H | H | phenethyl | 3.02¹⁾ |
| 115 | 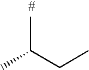 | H | H | H | 2-chlorophenethyl | |
| 116 | 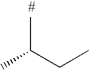 | H | H | H | 3-chlorophenethyl | |
| 117 | 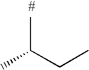 | H | H | H | 4-chlorophenethyl | 2.52¹⁾ |
| 118 | 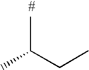 | H | H | H | 2-methylphenethyl | |
| 119 | 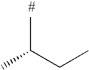 | H | H | H | 3-methylphenethyl | |
| 120 | 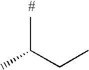 | H | H | H | 4-methylphenethyl | |
| 121 | 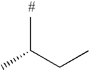 | H | H | H | 2-methoxyphenethyl | |
| 122 | 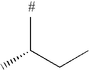 | H | H | H | 3-methoxyphenethyl | |
| 123 | 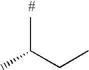 | H | H | H | 4-methoxyphenethyl | |
| 124 | 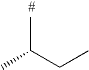 | H | H | H | 2,4-dichlorophenethyl | |
| 125 | 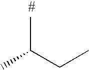 | H | H | H | 3,4-dichlorophenethyl | |
| 126 | 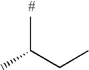 | H | H | H | 3,5-dichlorophenethyl | |
| 127 | 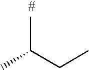 | H | H | H | 2,5-dichlorophenethyl | |
| 128 | 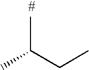 | H | H | H | 2,4,6-trichlorophenethyl | |
| 129 | 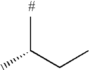 | H | H | H | 2-chloro- 4-methylphenethyl | |
| 130 | 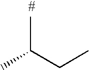 | H | H | H | 2-chloro-4-methoxy- phenethyl | |
| 131 | 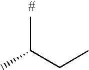 | H | H | H | 2,4-dimethylphenethyl | |
| 132 | 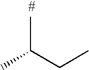 | H | H | H | 2,6-dimethylphenethyl | |
| 133 | 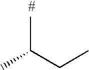 | H | H | H | 2,4,6-trimethylphenethyl | |
| 134 | 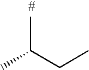 | H | H | H | 2-fluorophenethyl | |
| 135 | 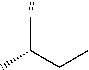 | H | H | H | 3-fluorophenethyl | |
| 136 | 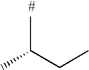 | H | H | H | 4-fluorophenethyl | |
| 137 | 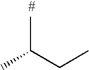 | H | H | H | 2-chloro- 4-fluorophenethyl | |
| 138 | 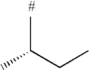 | H | H | H | 2,4-dimethoxyphenethyl | |
| 139 | 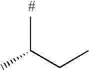 | H | H | H | 3,4-dimethoxyphenethyl | |
| 140 | 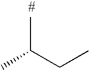 | H | H | H | 3,5-dimethoxyphenethyl | |
| 141 | 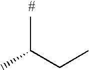 | H | H | H | 2,5-dimethoxyphenethyl | |
| 142 | 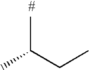 | H | H | H | 2-trifluoromethyl- phenethyl | |
| 143 | 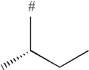 | H | H | H | 3-trifluoromethyl- phenethyl | |
| 144 | 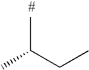 | H | H | H | 4-trifluoromethyl- phenethyl | 3.61¹⁾ |
| 144a | 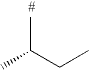 | H | H | H | 1-methyl-2-phenylethyl | |
| 144b | 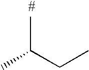 | H | H | H | 2-(3-chlorophenyl)- 1-methylethyl | |
| 144c | 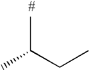 | H | H | H | 2-(4-chlorophenyl)- 1-methylethyl | |
| 144d | 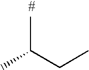 | H | H | H | 2-(3-trifluoromethyl)- 1-methylethyl | |
| 144e | 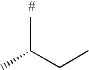 | H | H | H | 2-(4-trifluoromethyl)- 1-methylethyl | 3.97¹⁾ |
| 144f | 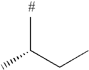 | H | H | H | 2-(4-tert-butylphenyl)- 1-methylethyl | 4.66¹⁾ |
| 144g | 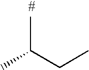 | H | H | H | 1-phenylethyl | |
| 144h | 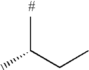 | H | H | H | 1-(4-methylphenyl)ethyl | |
| 144i | 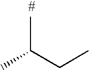 | H | H | H | 1-(4-chlorophenyl)ethyl | 3.52¹⁾ |
| 145 | 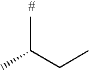 | H | H | H | 2-trifluoro- methoxyphenethyl | |
| 146 | 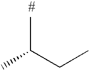 | H | H | H | 3-trifluoro- methoxyphenethyl | |
| 147 | 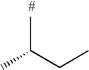 | H | H | H | 4-trifluoro- methoxyphenethyl | |
| 148 | 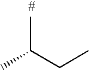 | H | H | H | 2-difluoro- methoxyphenethyl | |
| 149 | 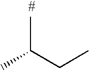 | H | H | H | 3-difluoro- methoxyphenethyl | |
| 150 | 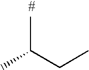 | H | H | H | 4-difluoro- methoxyphenethyl | |
| 151 | 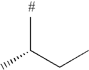 | H | H | H | 2-tert-butylphenethyl | |
| 152 | 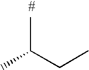 | H | H | H | 3-tert-butylphenethyl | |
| 153 | 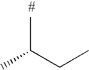 | H | H | H | 4-tert-butylphenethyl | |
| 154 | 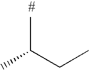 | H | H | H | tert-butoxycarbonyl | n.d.³⁾ |
| 155 | 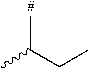 | H | H | H | phenyl | |
| 156 | 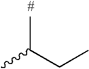 | H | H | H | 2-chlorophenyl | |
| 157 | 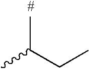 | H | H | H | 3-chlorophenyl | |
| 158 | 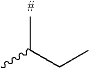 | H | H | H | 4-chlorophenyl | |
| 159 | 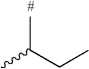 | H | H | H | 2-methylphenyl | |
| 160 | 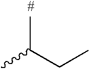 | H | H | H | 3-methylphenyl | |
| 161 | 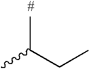 | H | H | H | 4-methylphenyl | |
| 162 | 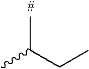 | H | H | H | 2-methoxyphenyl | |
| 163 | 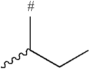 | H | H | H | 3-methoxyphenyl | |
| 164 | 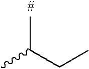 | H | H | H | 4-methoxyphenyl | |
| 165 | 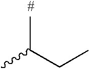 | H | H | H | 2,4-dichlorophenyl | |
| 166 | 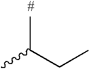 | H | H | H | 3,4-dichlorophenyl | |
| 167 | 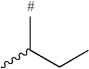 | H | H | H | 3,5-dichlorophenyl | |
| 168 | 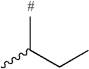 | H | H | H | 2,5-dichlorophenyl | |
| 169 | 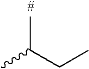 | H | H | H | 2,4,6-trichlorophenyl | |
| 170 | 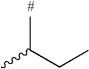 | H | H | H | 2-chloro-4-methylphenyl | |
| 171 | 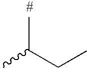 | H | H | H | 2-chloro-4-methoxy- phenyl | |
| 172 | 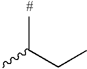 | H | H | H | 2,4-dimethylphenyl | |
| 173 | 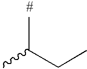 | H | H | H | 2,5-dimethylphenyl | |
| 174 | 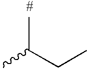 | H | H | H | 2,6-dimethylphenyl | |
| 175 | 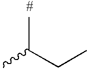 | H | H | H | 2,4,6-trimethylphenyl | |
| 176 | 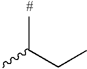 | H | H | H | 2-fluorophenyl | |
| 177 | 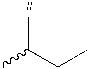 | H | H | H | 3-fluorophenyl | |
| 178 | 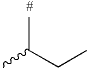 | H | H | H | 4-fluorophenyl | |
| 179 | 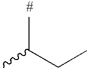 | H | H | H | 2-chloro-4-fluorophenyl | |
| 180 | 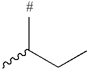 | H | H | H | 2,4-dimethoxyphenyl | |
| 181 | 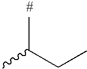 | H | H | H | 2,3-dimethoxyphenyl | |
| 182 | 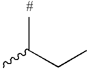 | H | H | H | 3,4-dimethoxyphenyl | |
| 183 | 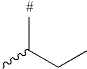 | H | H | H | 3,5-dimethoxyphenyl | |
| 184 | 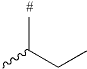 | H | H | H | 2,5-dimethoxyphenyl | |
| 185 | 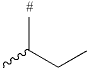 | H | H | H | 2-trifluoromethylphenyl | |
| 186 | 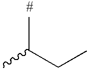 | H | H | H | 3-trifluoromethylphenyl | |
| 187 | 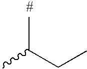 | H | H | H | 4-trifluoromethylphenyl | |
| 188 | 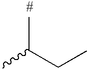 | H | H | H | 3-trifluoromethyl- 4-chlorophenyl | |
| 189 | 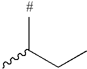 | H | H | H | 3-trifluoromethyl- 4-fluorophenyl | |
| 190 | 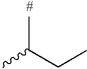 | H | H | H | 2-trifluoro- methoxyphenyl | |
| 191 | 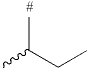 | H | H | H | 3-trifluoro- methoxyphenyl | |
| 192 | 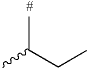 | H | H | H | 4-trifluoro- methoxyphenyl | |
| 193 | 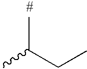 | H | H | H | 2-difluoro- methoxyphenyl | |
| 194 | 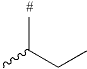 | H | H | H | 3-difluoro- methoxyphenyl | |
| 195 | 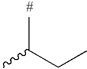 | H | H | H | 4-difluoro- methoxyphenyl | |
| 196 | 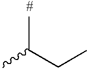 | H | H | H | 2-tert-butylphenyl | |
| 197 | 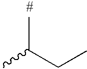 | H | H | H | 3-tert-butylphenyl | |
| 198 | 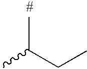 | H | H | H | 4-tert-butylphenyl | |
| 199 | 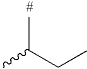 | H | H | H | 2-phenoxyphenyl | |
| 200 | 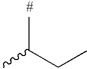 | H | H | H | 3-phenoxyphenyl | |
| 201 | 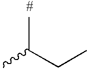 | H | H | H | 4-phenoxyphenyl | |
| 202 | 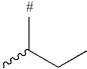 | H | H | H | 2-(4-methoxyphenoxy)- phenyl | |
| 203 | 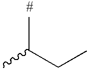 | H | H | H | 3-(4-methoxyphenoxy)- phenyl | |
| 204 | 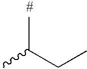 | H | H | H | 4-(4-methoxyphenoxy)- phenyl | |
| 205 | 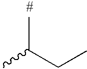 | H | H | H | 4-methanesulphonyl- phenyl | |
| 206 | 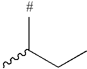 | H | H | H | 2-ethoxyphenyl | |
| 207 | 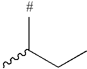 | H | H | H | 3-ethoxyphenyl | |
| 208 | 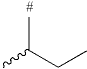 | H | H | H | 4-ethoxyphenyl | |
| 209 | 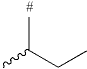 | H | H | H | methyl | |
| 210 | 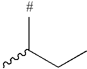 | H | H | H | ethyl | |
| 211 | 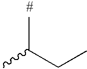 | H | H | H | n-propyl | |
| 212 | 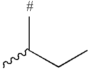 | H | H | H | n-butyl | |
| 213 | 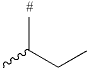 | H | H | H | n-pentyl | |
| 214 | 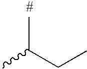 | H | H | H | benzyl | |
| 215 | 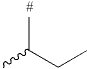 | H | H | H | 2-chlorobenzyl | |
| 216 | 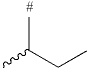 | H | H | H | 3-chlorobenzyl | |
| 217 | 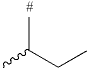 | H | H | H | 4-chlorobenzyl | |
| 218 | 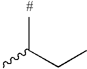 | H | H | H | 2-methylbenzyl | |
| 219 | 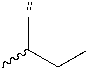 | H | H | H | 3-methylbenzyl | |
| 220 | 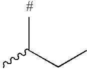 | H | H | H | 4-methylbenzyl | |
| 221 | 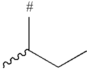 | H | H | H | 2-methoxybenzyl | |
| 222 | 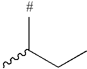 | H | H | H | 3-methoxybenzyl | |
| 223 | 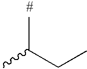 | H | H | H | 4-methoxybenzyl | |
| 224 | 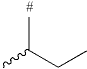 | H | H | H | 2,4-dichlorobenzyl | |
| 225 | 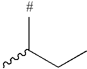 | H | H | H | 3,4-dichlorobenzyl | |
| 226 | 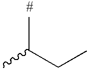 | H | H | H | 3,5-dichlorobenzyl | |
| 227 | 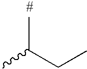 | H | H | H | 2,5-dichlorobenzyl | |
| 228 | 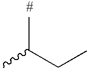 | H | H | H | 2,4,6-trichlorobenzyl | |
| 229 | 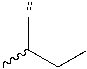 | H | H | H | 2-chloro-4-methylbenzyl | |
| 230 | 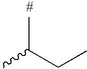 | H | H | H | 2-chloro-4-methoxy- benzyl | |
| 231 | 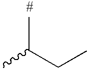 | H | H | H | 2,4-dimethylbenzyl | |
| 232 | 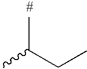 | H | H | H | 2,5-dimethylbenzyl | |
| 233 | 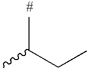 | H | H | H | 2,6-dimethylbenzyl | |
| 234 | 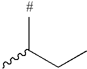 | H | H | H | 2,4,6-trimethylbenzyl | |
| 235 | 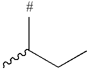 | H | H | H | 2-fluorobenzyl | |
| 236 | 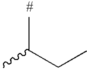 | H | H | H | 3-fluorobenzyl | |
| 237 | 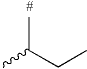 | H | H | H | 4-fluorobenzyl | |
| 238 | 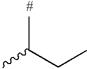 | H | H | H | 2-chloro-4-fluorobenzyl | |
| 239 | 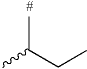 | H | H | H | 2,4-dimethoxybenzyl | |
| 240 | 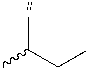 | H | H | H | 2,3-dimethoxybenzyl | |
| 241 | 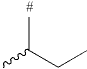 | H | H | H | 3,4-dimethoxybenzyl | |
| 242 | 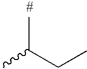 | H | H | H | 3,5-dimethoxybenzyl | |
| 243 | 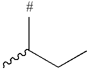 | H | H | H | 2,5-dimethoxybenzyl | |
| 244 | 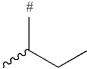 | H | H | H | 2-trifluoromethylbenzyl | |
| 245 | 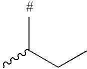 | H | H | H | 3-trifluoromethylbenzyl | |
| 246 | 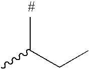 | H | H | H | 4-trifluoromethylbenzyl | |
| 247 | 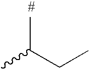 | H | H | H | 3-trifluoromethyl-4- chlorobenzyl | |
| 248 | 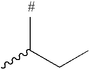 | H | H | H | 3-trifluoromethyl- 4-fluorobenzyl | |
| 249 | 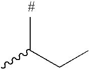 | H | H | H | 2-trifluoro- methoxybenzyl | |
| 250 | 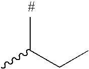 | H | H | H | 3-trifluoro- methoxybenzyl | |
| 251 | 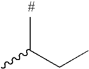 | H | H | H | 4-trifluoro- methoxybenzyl | |
| 252 | 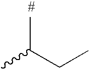 | H | H | H | 2-difluoro- methoxybenzyl | |
| 253 | 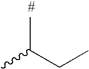 | H | H | H | 3-difluoro- methoxybenzyl | |
| 254 | 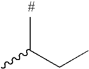 | H | H | H | 4-difluoro- methoxybenzyl | |
| 255 | 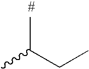 | H | H | H | 2-tert-butylbenzyl | |
| 256 | 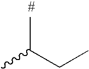 | H | H | H | 3-tert-butylbenzyl | |
| 257 | 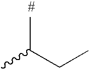 | H | H | H | 4-tert-butylbenzyl | |
| 258 | 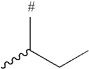 | H | H | H | 2-phenoxybenzyl | |
| 259 | 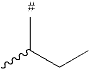 | H | H | H | 3-phenoxybenzyl | |
| 260 | 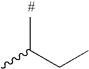 | H | H | H | 4-phenoxybenzyl | |
| 261 | 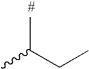 | H | H | H | 2-(4-methoxyphenoxy)- benzyl | |
| 262 | 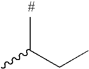 | H | H | H | 3-(4-methoxyphenoxy)- benzyl | |
| 263 | 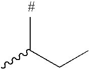 | H | H | H | 4-(4-methoxyphenoxy)- benzyl | |
| 264 | 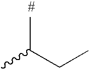 | H | H | H | 4-methanesulphonyl- benzyl | |
| 265 | 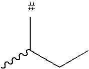 | H | H | H | 2-ethoxybenzyl | |
| 266 | 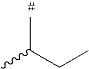 | H | H | H | 3-ethoxybenzyl | |
| 267 | 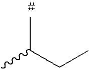 | H | H | H | 4-ethoxybenzyl | |
| 268 | 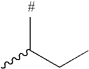 | H | H | H | phenethyl | |
| 269 | 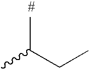 | H | H | H | 2-chlorophenethyl | |
| 270 | 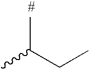 | H | H | H | 3-chlorophenethyl | |
| 271 | 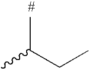 | H | H | H | 4-chlorophenethyl | |
| 272 | 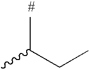 | H | H | H | 2-methylphenethyl | |
| 273 | 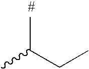 | H | H | H | 3-methylphenethyl | |
| 274 | 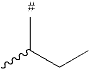 | H | H | H | 4-methylphenethyl | |
| 275 | 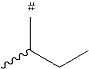 | H | H | H | 2-methoxyphenethyl | |
| 276 | 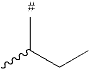 | H | H | H | 3-methoxyphenethyl | |
| 277 | 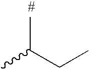 | H | H | H | 4-methoxyphenethyl | |
| 278 | 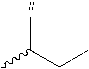 | H | H | H | 2,4-dichlorophenethyl | |
| 279 | 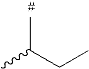 | H | H | H | 3,4-dichlorophenethyl | |
| 280 | 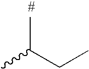 | H | H | H | 3,5-dichlorophenethyl | |
| 281 | 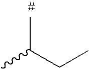 | H | H | H | 2,5-dichlorophenethyl | |
| 282 | 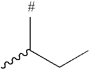 | H | H | H | 2,4,6-trichlorophenethyl | |
| 283 | 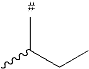 | H | H | H | 2-chloro- 4-methylphenethyl | |
| 284 | 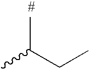 | H | H | H | 2-chloro-4-methoxy- phenethyl | |
| 285 | 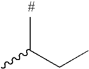 | H | H | H | 2,4-dimethylphenethyl | |
| 286 | 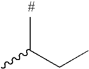 | H | H | H | 2,6-dimethylphenethyl | |
| 287 | 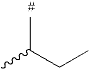 | H | H | H | 2,4,6-trimethylphenethyl | |
| 288 | 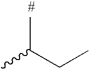 | H | H | H | 2-fluorophenethyl | |
| 289 | 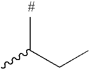 | H | H | H | 3-fluorophenethyl | |
| 290 | 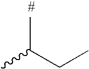 | H | H | H | 4-fluorophenethyl | |
| 291 | 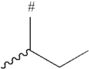 | H | H | H | 2-chloro- 4-fluorophenethyl | |
| 292 | 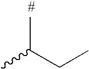 | H | H | H | 2,4-dimethoxyphenethyl | |
| 293 | 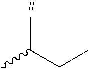 | H | H | H | 3,4-dimethoxyphenethyl | |
| 294 | 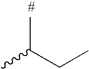 | H | H | H | 3,5-dimethoxyphenethyl | |
| 295 | 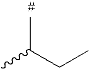 | H | H | H | 2,5-dimethoxyphenethyl | |
| 296 | 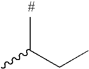 | H | H | H | 2-trifluoromethyl- phenethyl | |
| 297 | 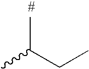 | H | H | H | 3-trifluoromethyl- phenethyl | |
| 298 | 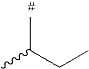 | H | H | H | 4-trifluoromethyl- phenethyl | |
| 299 | 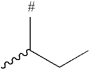 | H | H | H | 2-trifluoro- methoxyphenethyl | |
| 300 | 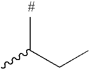 | H | H | H | 3-trifluoro- methoxyphenethyl | |
| 301 | 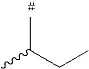 | H | H | H | 4-trifluoro- methoxyphenethyl | |
| 302 | 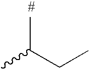 | H | H | H | 2-difluoro- methoxyphenethyl | |
| 303 | 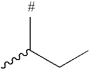 | H | H | H | 3-difluoro- methoxyphenethyl | |
| 304 | 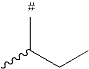 | H | H | H | 4-difluoro- methoxyphenethyl | |
| 305 | 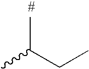 | H | H | H | 2-tert-butylphenethyl | |
| 306 | 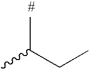 | H | H | H | 3-tert-butylphenethyl | |
| 307 | 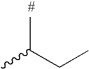 | H | H | H | 4-tert-butylphenethyl | |
| 308 | cyclohexyl | H | H | H | phenyl | |
| 309 | cyclohexyl | H | H | H | 2-chlorophenyl | |
| 310 | cyclohexyl | H | H | H | 3-chlorophenyl | |
| 311 | cyclohexyl | H | H | H | 4-chlorophenyl | |
| 312 | cyclohexyl | H | H | H | 2-methylphenyl | |
| 313 | cyclohexyl | H | H | H | 3-methylphenyl | |
| 314 | cyclohexyl | H | H | H | 4-methylphenyl | |
| 315 | cyclohexyl | H | H | H | 2-methoxyphenyl | |
| 316 | cyclohexyl | H | H | H | 3-methoxyphenyl | |
| 317 | cyclohexyl | H | H | H | 4-methoxyphenyl | |
| 318 | cyclohexyl | H | H | H | 2,4-dichlorophenyl | |
| 319 | cyclohexyl | H | H | H | 3,4-dichlorophenyl | |
| 320 | cyclohexyl | H | H | H | 3,5-dichlorophenyl | |
| 321 | cyclohexyl | H | H | H | 2,5-dichlorophenyl | |
| 322 | cyclohexyl | H | H | H | 2,4,6-trichlorophenyl | |
| 323 | cyclohexyl | H | H | H | 2-chloro-4-methylphenyl | |
| 324 | cyclohexyl | H | H | H | 2-chloro-4-methoxy- | |
| | | | | | phenyl | |
| 325 | cyclohexyl | H | H | H | 2,4-dimethylphenyl | |
| 326 | cyclohexyl | H | H | H | 2,5-dimethylphenyl | |
| 327 | cyclohexyl | H | H | H | 2,6-dimethylphenyl | |
| 328 | cyclohexyl | H | H | H | 2,4,6-trimethylphenyl | |
| 329 | cyclohexyl | H | H | H | 2-fluorophenyl | |
| 330 | cyclohexyl | H | H | H | 3-fluorophenyl | |
| 331 | cyclohexyl | H | H | H | 4-fluorophenyl | |
| 332 | cyclohexyl | H | H | H | 2-chloro-4-fluorophenyl | |
| 333 | cyclohexyl | H | H | H | 2,4-dimethoxyphenyl | |
| 334 | cyclohexyl | H | H | H | 2,3-dimethoxyphenyl | |
| 335 | cyclohexyl | H | H | H | 3,4-dimethoxyphenyl | |
| 336 | cyclohexyl | H | H | H | 3,5-dimethoxyphenyl | 1.59²⁾ |
| 337 | cyclohexyl | H | H | H | 2,5-dimethoxyphenyl | |
| 338 | cyclohexyl | H | H | H | 2-trifluoromethylphenyl | |
| 339 | cyclohexyl | H | H | H | 3-trifluoromethylphenyl | |
| 340 | cyclohexyl | H | H | H | 4-trifluoromethylphenyl | |
| 341 | cyclohexyl | H | H | H | 3-trifluoromethyl- | |
| | | | | | 4-chlorophenyl | |
| 342 | cyclohexyl | H | H | H | 3-trifluoromethyl- | |
| | | | | | 4-fluorophenyl | |
| 343 | cyclohexyl | H | H | H | 2-trifluoro- | |
| | | | | | methoxyphenyl | |
| 344 | cyclohexyl | H | H | H | 3-trifluoro- | |
| | | | | | methoxyphenyl | |
| 345 | cyclohexyl | H | H | H | 4-trifluoro- | |
| | | | | | methoxyphenyl | |
| 346 | cyclohexyl | H | H | H | 2-difluoro- | |
| | | | | | methoxyphenyl | |
| 347 | cyclohexyl | H | H | H | 3-difluoro- | |
| | | | | | methoxyphenyl | |
| 348 | cyclohexyl | H | H | H | 4-difluoro- | |
| | | | | | methoxyphenyl | |
| 349 | cyclohexyl | H | H | H | 2-tert-butylphenyl | |
| 350 | cyclohexyl | H | H | H | 3-tert-butylphenyl | |
| 351 | cyclohexyl | H | H | H | 4-tert-butylphenyl | |
| 352 | cyclohexyl | H | H | H | 2-phenoxyphenyl | |
| 353 | cyclohexyl | H | H | H | 3-phenoxyphenyl | |
| 354 | cyclohexyl | H | H | H | 4-phenoxyphenyl | |
| 355 | cyclohexyl | H | H | H | 2-(4-methoxyphenoxy)- | |
| | | | | | phenyl | |
| 356 | cyclohexyl | H | H | H | 3-(4-methoxyphenoxy)- | |
| | | | | | phenyl | |
| 357 | cyclohexyl | H | H | H | 4-(4-methoxyphenoxy)- | |
| | | | | | phenyl | |
| 358 | cyclohexyl | H | H | H | 4-methanesulphonyl- | |
| | | | | | phenyl | |
| 359 | cyclohexyl | H | H | H | 2-ethoxyphenyl | |
| 360 | cyclohexyl | H | H | H | 3-ethoxyphenyl | |
| 361 | cyclohexyl | H | H | H | 4-ethoxyphenyl | |
| 362 | cyclohexyl | H | H | H | methyl | 2.12¹⁾ |
| 363 | cyclohexyl | H | H | H | ethyl | |
| 364 | cyclohexyl | H | H | H | n-propyl | |
| 365 | cyclohexyl | H | H | H | n-butyl | |
| 366 | cyclohexyl | H | H | H | n-pentyl | 3.72¹⁾ |
| 367 | cyclohexyl | H | H | H | benzyl | |
| 368 | cyclohexyl | H | H | H | 2-chlorobenzyl | |
| 369 | cyclohexyl | H | H | H | 3-chlorobenzyl | |
| 370 | cyclohexyl | H | H | H | 4-chlorobenzyl | |
| 371 | cyclohexyl | H | H | H | 2-methylbenzyl | |
| 372 | cyclohexyl | H | H | H | 3-methylbenzyl | |
| 373 | cyclohexyl | H | H | H | 4-methylbenzyl | |
| 374 | cyclohexyl | H | H | H | 2-methoxybenzyl | |
| 375 | cyclohexyl | H | H | H | 3-methoxybenzyl | |
| 376 | cyclohexyl | H | H | H | 4-methoxybenzyl | |
| 377 | cyclohexyl | H | H | H | 2,4-dichlorobenzyl | |
| 378 | cyclohexyl | H | H | H | 3,4-dichlorobenzyl | |
| 379 | cyclohexyl | H | H | H | 3,5-dichlorobenzyl | |
| 380 | cyclohexyl | H | H | H | 2,5-dichlorobenzyl | |
| 381 | cyclohexyl | H | H | H | 2,4,6-trichlorobenzyl | |
| 382 | cyclohexyl | H | H | H | 2-chloro-4-methylbenzyl | |
| 383 | cyclohexyl | H | H | H | 2-chloro-4-methoxy- | |
| | | | | | benzyl | |
| 384 | cyclohexyl | H | H | H | 2,4-dimethylbenzyl | |
| 385 | cyclohexyl | H | H | H | 2,5-dimethylbenzyl | |
| 386 | cyclohexyl | H | H | H | 2,6-dimethylbenzyl | |
| 387 | cyclohexyl | H | H | H | 2,4,6-trimethylbenzyl | |
| 388 | cyclohexyl | H | H | H | 2-fluorobenzyl | |
| 389 | cyclohexyl | H | H | H | 3-fluorobenzyl | |
| 390 | cyclohexyl | H | H | H | 4-fluorobenzyl | |
| 391 | cyclohexyl | H | H | H | 2-chloro-4-fluorobenzyl | |
| 392 | cyclohexyl | H | H | H | 2,4-dimethoxybenzyl | |
| 393 | cyclohexyl | H | H | H | 2,3-dimethoxybenzyl | |
| 394 | cyclohexyl | H | H | H | 3,4-dimethoxybenzyl | |
| 395 | cyclohexyl | H | H | H | 3,5-dimethoxybenzyl | |
| 396 | cyclohexyl | H | H | H | 2,5-dimethoxybenzyl | |
| 397 | cyclohexyl | H | H | H | 2-trifluoromethylbenzyl | |
| 398 | cyclohexyl | H | H | H | 3-trifluoromethylbenzyl | |
| 399 | cyclohexyl | H | H | H | 4-trifluoromethylbenzyl | |
| 400 | cyclohexyl | H | H | H | 3-trifluoromethyl-4- | |
| | | | | | chlorobenzyl | |
| 401 | cyclohexyl | H | H | H | 3-trifluoromethyl- | |
| | | | | | 4-fluorobenzyl | |
| 402 | cyclohexyl | H | H | H | 2-trifluoro- | |
| | | | | | methoxybenzyl | |
| 403 | cyclohexyl | H | H | H | 3-trifluoro- | |
| | | | | | methoxybenzyl | |
| 404 | cyclohexyl | H | H | H | 4-trifluoro- | |
| | | | | | methoxybenzyl | |
| 405 | cyclohexyl | H | H | H | 2-difluoro- | |
| | | | | | methoxybenzyl | |
| 406 | cyclohexyl | H | H | H | 3-difluoro- | |
| | | | | | methoxybenzyl | |
| 407 | cyclohexyl | H | H | H | 4-difluoro- | |
| | | | | | methoxybenzyl | |
| 408 | cyclohexyl | H | H | H | 2-tert-butylbenzyl | |
| 409 | cyclohexyl | H | H | H | 3-tert-butylbenzyl | |
| 410 | cyclohexyl | H | H | H | 4-tert-butylbenzyl | |
| 411 | cyclohexyl | H | H | H | 2-phenoxybenzyl | |
| 412 | cyclohexyl | H | H | H | 3-phenoxybenzyl | |
| 413 | cyclohexyl | H | H | H | 4-phenoxybenzyl | |
| 414 | cyclohexyl | H | H | H | 2-(4-methoxyphenoxy)- | |
| | | | | | benzyl | |
| 415 | cyclohexyl | H | H | H | 3-(4-methoxyphenoxy)- | |
| | | | | | benzyl | |
| 416 | cyclohexyl | H | H | H | 4-(4-methoxyphenoxy)- | |
| | | | | | benzyl | |
| 417 | cyclohexyl | H | H | H | 4-methanesulphonyl- | |
| | | | | | benzyl | |
| 418 | cyclohexyl | H | H | H | 2-ethoxybenzyl | |
| 419 | cyclohexyl | H | H | H | 3-ethoxybenzyl | |
| 420 | cyclohexyl | H | H | H | 4-ethoxybenzyl | |
| 421 | cyclohexyl | H | H | H | phenethyl | 3.49¹⁾ |
| 422 | cyclohexyl | H | H | H | 2-chlorophenethyl | |
| 423 | cyclohexyl | H | H | H | 3-chlorophenethyl | |
| 424 | cyclohexyl | H | H | H | 4-chlorophenethyl | |
| 425 | cyclohexyl | H | H | H | 2-methylphenethyl | |
| 426 | cyclohexyl | H | H | H | 3-methylphenethyl | |
| 427 | cyclohexyl | H | H | H | 4-methylphenethyl | |
| 428 | cyclohexyl | H | H | H | 2-methoxyphenethyl | |
| 429 | cyclohexyl | H | H | H | 3-methoxyphenethyl | |
| 430 | cyclohexyl | H | H | H | 4-methoxyphenethyl | |
| 431 | cyclohexyl | H | H | H | 2,4-dichlorophenethyl | |
| 432 | cyclohexyl | H | H | H | 3,4-dichlorophenethyl | |
| 433 | cyclohexyl | H | H | H | 3,5-dichlorophenethyl | |
| 434 | cyclohexyl | H | H | H | 2,5-dichlorophenethyl | |
| 435 | cyclohexyl | H | H | H | 2,4,6-trichlorophenethyl | |
| 436 | cyclohexyl | H | H | H | 2-chloro- | |
| | | | | | 4-methylphenethyl | |
| 437 | cyclohexyl | H | H | H | 2-chloro-4-methoxy- | |
| | | | | | phenethyl | |
| 438 | cyclohexyl | H | H | H | 2,4-dimethylphenethyl | |
| 439 | cyclohexyl | H | H | H | 2,6-dimethylphenethyl | |
| 440 | cyclohexyl | H | H | H | 2,4,6-trimethylphenethyl | |
| 441 | cyclohexyl | H | H | H | 2-fluorophenethyl | |
| 442 | cyclohexyl | H | H | H | 3-fluorophenethyl | |
| 443 | cyclohexyl | H | H | H | 4-fluorophenethyl | |
| 444 | cyclohexyl | H | H | H | 2-chloro- | |
| | | | | | 4-fluorophenethyl | |
| 445 | cyclohexyl | H | H | H | 2,4-dimethoxyphenethyl | |
| 446 | cyclohexyl | H | H | H | 3,4-dimethoxyphenethyl | |
| 447 | cyclohexyl | H | H | H | 3,5-dimethoxyphenethyl | |
| 448 | cyclohexyl | H | H | H | 2,5-dimethoxyphenethyl | |
| 449 | cyclohexyl | H | H | H | 2-trifluoromethyl- | |
| | | | | | phenethyl | |
| 450 | cyclohexyl | H | H | H | 3-trifluoromethyl- | |
| | | | | | phenethyl | |
| 451 | cyclohexyl | H | H | H | 4-trifluoromethyl- | |
| | | | | | phenethyl | |
| 452 | cyclohexyl | H | H | H | 2-trifluoro- | |
| | | | | | methoxyphenethyl | |
| 453 | cyclohexyl | H | H | H | 3-trifluoro- | |
| | | | | | methoxyphenethyl | |
| 454 | cyclohexyl | H | H | H | 4-trifluoro- | |
| | | | | | methoxyphenethyl | |
| 455 | cyclohexyl | H | H | H | 2-difluoro- | |
| | | | | | methoxyphenethyl | |
| 456 | cyclohexyl | H | H | H | 3-difluoro- | |
| | | | | | methoxyphenethyl | |
| 457 | cyclohexyl | H | H | H | 4-difluoro- | |
| | | | | | methoxyphenethyl | |
| 458 | cyclohexyl | H | H | H | 2-tert-butylphenethyl | |
| 459 | cyclohexyl | H | H | H | 3-tert-butylphenethyl | |
| 460 | cyclohexyl | H | H | H | 4-tert-butylphenethyl | |
| 469 | isopropyl | H | H | H | 3-methoxyphenyl | |
| 470 | isopropyl | H | H | H | 4-methoxyphenyl | |
| 471 | isopropyl | H | H | H | 2,4-dichlorophenyl | |
| 472 | isopropyl | H | H | H | 3,4-dichlorophenyl | |
| 473 | isopropyl | H | H | H | 3,5-dichlorophenyl | |
| 474 | isopropyl | H | H | H | 2,5-dichlorophenyl | |
| 475 | isopropyl | H | H | H | 2,4,6-trichlorophenyl | |
| 476 | isopropyl | H | H | H | 2-chloro-4-methylphenyl | |
| 477 | isopropyl | H | H | H | 2-chloro-4-methoxy- | |
| | | | | | phenyl | |
| 478 | isopropyl | H | H | H | 2,4-dimethylphenyl | |
| 479 | isopropyl | H | H | H | 2,5-dimethylphenyl | |
| 480 | isopropyl | H | H | H | 2,6-dimethylphenyl | |
| 481 | isopropyl | H | H | H | 2,4,6-trimethylphenyl | |
| 482 | isopropyl | H | H | H | 2-fluorophenyl | |
| 483 | isopropyl | H | H | H | 3-fluorophenyl | |
| 484 | isopropyl | H | H | H | 4-fluorophenyl | |
| 485 | isopropyl | H | H | H | 2-chloro-4-fluorophenyl | |
| 486 | isopropyl | H | H | H | 2,4-dimethoxyphenyl | |
| 487 | isopropyl | H | H | H | 2,3-dimethoxyphenyl | |
| 488 | isopropyl | H | H | H | 3,4-dimethoxyphenyl | |
| 489 | isopropyl | H | H | H | 3,5-dimethoxyphenyl | 2.32¹⁾ |
| 490 | isopropyl | H | H | H | 2,5-dimethoxyphenyl | |
| 491 | isopropyl | H | H | H | 2-trifluoromethylphenyl | |
| 492 | isopropyl | H | H | H | 3-trifluoromethylphenyl | |
| 493 | isopropyl | H | H | H | 4-trifluoromethylphenyl | |
| 494 | isopropyl | H | H | H | 3-trifluoromethyl- | |
| | | | | | 4-chlorophenyl | |
| 495 | isopropyl | H | H | H | 3-trifluoromethyl- | |
| | | | | | 4-fluorophenyl | |
| 496 | isopropyl | H | H | H | 2-trifluoro- | |
| | | | | | methoxyphenyl | |
| 497 | isopropyl | H | H | H | 3-trifluoro- | |
| | | | | | methoxyphenyl | |
| 498 | isopropyl | H | H | H | 4-trifluoro- | |
| | | | | | methoxyphenyl | |
| 499 | isopropyl | H | H | H | 2-difluoro- | |
| | | | | | methoxyphenyl | |
| 500 | isopropyl | H | H | H | 3-difluoro- | |
| | | | | | methoxyphenyl | |
| 501 | isopropyl | H | H | H | 4-difluoro- | |
| | | | | | methoxyphenyl | |
| 502 | isopropyl | H | H | H | 2-tert-butylphenyl | |
| 503 | isopropyl | H | H | H | 3-tert-butylphenyl | |
| 504 | isopropyl | H | H | H | 4-tert-butylphenyl | |
| 505 | isopropyl | H | H | H | 2-phenoxyphenyl | |
| 506 | isopropyl | H | H | H | 3-phenoxyphenyl | |
| 507 | isopropyl | H | H | H | 4-phenoxyphenyl | |
| 508 | isopropyl | H | H | H | 2-(4-methoxyphenoxy)- | |
| | | | | | phenyl | |
| 509 | isopropyl | H | H | H | 3-(4-methoxyphenoxy)- | |
| | | | | | phenyl | |
| 510 | isopropyl | H | H | H | 4-(4-methoxyphenoxy)- | |
| | | | | | phenyl | |
| 511 | isopropyl | H | H | H | 4-methanesulphonyl- | |
| | | | | | phenyl | |
| 512 | isopropyl | H | H | H | 2-ethoxyphenyl | |
| 513 | isopropyl | H | H | H | 3-ethoxyphenyl | |
| 514 | isopropyl | H | H | H | 4-ethoxyphenyl | |
| 515 | isopropyl | H | H | H | methyl | |
| 516 | isopropyl | H | H | H | ethyl | |
| 517 | isopropyl | H | H | H | n-propyl | |
| 518 | isopropyl | H | H | H | n-butyl | |
| 519 | isopropyl | H | H | H | n-pentyl | |
| 520 | isopropyl | H | H | H | benzyl | |
| 521 | isopropyl | H | H | H | 2-chlorobenzyl | |
| 522 | isopropyl | H | H | H | 3-chlorobenzyl | |
| 523 | isopropyl | H | H | H | 4-chlorobenzyl | |
| 524 | isopropyl | H | H | H | 2-methylbenzyl | |
| 525 | isopropyl | H | H | H | 3-methylbenzyl | |
| 526 | isopropyl | H | H | H | 4-methylbenzyl | |
| 527 | isopropyl | H | H | H | 2-methoxybenzyl | |
| 528 | isopropyl | H | H | H | 3-methoxybenzyl | |
| 529 | isopropyl | H | H | H | 4-methoxybenzyl | |
| 530 | isopropyl | H | H | H | 2,4-dichlorobenzyl | |
| 531 | isopropyl | H | H | H | 3,4-dichlorobenzyl | |
| 532 | isopropyl | H | H | H | 3,5-dichlorobenzyl | |
| 533 | isopropyl | H | H | H | 2,5-dichlorobenzyl | |
| 534 | isopropyl | H | H | H | 2,4,6-trichlorobenzyl | |
| 535 | isopropyl | H | H | H | 2-chloro-4-methylbenzyl | |
| 536 | isopropyl | H | H | H | 2-chloro-4-methoxy- | |
| | | | | | benzyl | |
| 537 | isopropyl | H | H | H | 2,4-dimethylbenzyl | |
| 538 | isopropyl | H | H | H | 2,5-dimethylbenzyl | |
| 539 | isopropyl | H | H | H | 2,6-dimethylbenzyl | |
| 540 | isopropyl | H | H | H | 2,4,6-trimethylbenzyl | |
| 541 | isopropyl | H | H | H | 2-fluorobenzyl | |
| 542 | isopropyl | H | H | H | 3-fluorobenzyl | |
| 543 | isopropyl | H | H | H | 4-fluorobenzyl | |
| 544 | isopropyl | H | H | H | 2-chloro-4-fluorobenzyl | |
| 545 | isopropyl | H | H | H | 2,4-dimethoxybenzyl | |
| 546 | isopropyl | H | H | H | 2,3-dimethoxybenzyl | |
| 547 | isopropyl | H | H | H | 3,4-dimethoxybenzyl | |
| 548 | isopropyl | H | H | H | 3,5-dimethoxybenzyl | |
| 549 | isopropyl | H | H | H | 2,5-dimethoxybenzyl | |
| 550 | isopropyl | H | H | H | 2-trifluoromethylbenzyl | |
| 551 | isopropyl | H | H | H | 3-trifluoromethylbenzyl | |
| 552 | isopropyl | H | H | H | 4-trifluoromethylbenzyl | |
| 553 | isopropyl | H | H | H | 3-trifluoromethyl-4- | |
| | | | | | chlorobenzyl | |
| 554 | isopropyl | H | H | H | 3-trifluoromethyl- | |
| | | | | | 4-fluorobenzyl | |
| 555 | isopropyl | H | H | H | 2-trifluoro- | |
| | | | | | methoxybenzyl | |
| 556 | isopropyl | H | H | H | 3-trifluoro- | |
| | | | | | methoxybenzyl | |
| 557 | isopropyl | H | H | H | 4-trifluoro- | |
| | | | | | methoxybenzyl | |
| 558 | isopropyl | H | H | H | 2-difluoro- | |
| | | | | | methoxybenzyl | |
| 559 | isopropyl | H | H | H | 3-difluoro- | |
| | | | | | methoxybenzyl | |
| 560 | isopropyl | H | H | H | 4-difluoro- | |
| | | | | | methoxybenzyl | |
| 561 | isopropyl | H | H | H | 2-tert-butylbenzyl | |
| 562 | isopropyl | H | H | H | 3-tert-butylbenzyl | |
| 563 | isopropyl | H | H | H | 4-tert-butylbenzyl | |
| 564 | isopropyl | H | H | H | 2-phenoxybenzyl | |
| 565 | isopropyl | H | H | H | 3-phenoxybenzyl | |
| 566 | isopropyl | H | H | H | 4-phenoxybenzyl | |
| 567 | isopropyl | H | H | H | 2-(4-methoxyphenoxy)- | |
| | | | | | benzyl | |
| 568 | isopropyl | H | H | H | 3-(4-methoxyphenoxy)- | |
| | | | | | benzyl | |
| 569 | isopropyl | H | H | H | 4-(4-methoxyphenoxy)- | |
| | | | | | benzyl | |
| 570 | isopropyl | H | H | H | 4-methanesulphonyl- | |
| | | | | | benzyl | |
| 571 | isopropyl | H | H | H | 2-ethoxybenzyl | |
| 572 | isopropyl | H | H | H | 3-ethoxybenzyl | |
| 573 | isopropyl | H | H | H | 4-ethoxybenzyl | |
| 574 | isopropyl | H | H | H | phenethyl | |
| 575 | isopropyl | H | H | H | 2-chlorophenethyl | |
| 576 | isopropyl | H | H | H | 3-chlorophenethyl | |
| 577 | isopropyl | H | H | H | 4-chlorophenethyl | |
| 578 | isopropyl | H | H | H | 2-methylphenethyl | |
| 579 | isopropyl | H | H | H | 3-methylphenethyl | |
| 580 | isopropyl | H | H | H | 4-methylphenethyl | |
| 581 | isopropyl | H | H | H | 2-methoxyphenethyl | |
| 582 | isopropyl | H | H | H | 3-methoxyphenethyl | |
| 583 | isopropyl | H | H | H | 4-methoxyphenethyl | |
| 584 | isopropyl | H | H | H | 2,4-dichlorophenethyl | |
| 585 | isopropyl | H | H | H | 3,4-dichlorophenethyl | |
| 586 | isopropyl | H | H | H | 3,5-dichlorophenethyl | |
| 587 | isopropyl | H | H | H | 2,5-dichlorophenethyl | |
| 588 | isopropyl | H | H | H | 2,4,6-trichlorophenethyl | |
| 589 | isopropyl | H | H | H | 2-chloro- | |
| | | | | | 4-methylphenethyl | |
| 590 | isopropyl | H | H | H | 2-chloro-4-methoxy- | |
| | | | | | phenethyl | |
| 591 | isopropyl | H | H | H | 2,4-dimethylphenethyl | |
| 592 | isopropyl | H | H | H | 2,6-dimethylphenethyl | |
| 593 | isopropyl | H | H | H | 2,4,6-trimethylphenethyl | |
| 594 | isopropyl | H | H | H | 2-fluorophenethyl | |
| 595 | isopropyl | H | H | H | 3-fluorophenethyl | |
| 596 | isopropyl | H | H | H | 4-fluorophenethyl | |
| 597 | isopropyl | H | H | H | 2-chloro- | |
| | | | | | 4-fluorophenethyl | |
| 598 | isopropyl | H | H | H | 2,4-dimethoxyphenethyl | |
| 599 | isopropyl | H | H | H | 3,4-dimethoxyphenethyl | |
| 600 | isopropyl | H | H | H | 3,5-dimethoxyphenethyl | |
| 601 | isopropyl | H | H | H | 2,5-dimethoxyphenethyl | |
| 602 | isopropyl | H | H | H | 2-trifluoromethyl- | |
| | | | | | phenethyl | |
| 603 | isopropyl | H | H | H | 3-trifluoromethyl- | |
| | | | | | phenethyl | |
| 604 | isopropyl | H | H | H | 4-trifluoromethyl- | |
| | | | | | phenethyl | |
| 605 | isopropyl | H | H | H | 2-trifluoro- | |
| | | | | | methoxyphenethyl | |
| 606 | isopropyl | H | H | H | 3-trifluoro- | |
| | | | | | methoxyphenethyl | |
| 607 | isopropyl | H | H | H | 4-trifluoro- | |
| | | | | | methoxyphenethyl | |
| 608 | isopropyl | H | H | H | 2-difluoro- | |
| | | | | | methoxyphenethyl | |
| 609 | isopropyl | H | H | H | 3-difluoro- | |
| | | | | | methoxyphenethyl | |
| 610 | isopropyl | H | H | H | 4-difluoro- | |
| | | | | | methoxyphenethyl | |
| 611 | isopropyl | H | H | H | 2-tert-butylphenethyl | |
| 612 | isopropyl | H | H | H | 3-tert-butylphenethyl | |
| 613 | isopropyl | H | H | H | 4-tert-butylphenethyl | |
| 614 | isobutyl | H | H | H | phenyl | |
| 615 | isobutyl | H | H | H | 2-chlorophenyl | |
| 616 | isobutyl | H | H | H | 3-chlorophenyl | |
| 617 | isobutyl | H | H | H | 4-chlorophenyl | |
| 618 | isobutyl | H | H | H | 2-methylphenyl | |
| 619 | isobutyl | H | H | H | 3-methylphenyl | |
| 620 | isobutyl | H | H | H | 4-methylphenyl | |
| 621 | isobutyl | H | H | H | 2-methoxyphenyl | |
| 622 | isobutyl | H | H | H | 3-methoxyphenyl | |
| 623 | isobutyl | H | H | H | 4-methoxyphenyl | |
| 624 | isobutyl | H | H | H | 2,4-dichlorophenyl | |
| 625 | isobutyl | H | H | H | 3,4-dichlorophenyl | |
| 626 | isobutyl | H | H | H | 3,5-dichlorophenyl | |
| 627 | isobutyl | H | H | H | 2,5-dichlorophenyl | |
| 628 | isobutyl | H | H | H | 2,4,6-trichlorophenyl | |
| 629 | isobutyl | H | H | H | 2-chloro-4-methylphenyl | |
| 630 | isobutyl | H | H | H | 2-chloro-4-methoxy- | |
| | | | | | phenyl | |
| 631 | isobutyl | H | H | H | 2,4-dimethylphenyl | |
| 632 | isobutyl | H | H | H | 2,5-dimethylphenyl | |
| 633 | isobutyl | H | H | H | 2,6-dimethylphenyl | |
| 634 | isobutyl | H | H | H | 2,4,6-trimethylphenyl | |
| 635 | isobutyl | H | H | H | 2-fluorophenyl | |
| 636 | isobutyl | H | H | H | 3-fluorophenyl | |
| 637 | isobutyl | H | H | H | 4-fluorophenyl | |
| 638 | isobutyl | H | H | H | 2-chloro-4-fluorophenyl | |
| 639 | isobutyl | H | H | H | 2,4-dimethoxyphenyl | |
| 640 | isobutyl | H | H | H | 2,3-dimethoxyphenyl | |
| 641 | isobutyl | H | H | H | 3,4-dimethoxyphenyl | |
| 642 | isobutyl | H | H | H | 3,5-dimethoxyphenyl | 2.54¹⁾ |
| 643 | isobutyl | H | H | H | 2,5-dimethoxyphenyl | |
| 644 | isobutyl | H | H | H | 2-trifluoromethylphenyl | |
| 645 | isobutyl | H | H | H | 3-trifluoromethylphenyl | |
| 646 | isobutyl | H | H | H | 4-trifluoromethylphenyl | |
| 647 | isobutyl | H | H | H | 3-trifluoromethyl- | |
| | | | | | 4-chlorophenyl | |
| 648 | isobutyl | H | H | H | 3-trifluoromethyl- | |
| | | | | | 4-fluorophenyl | |
| 649 | isobutyl | H | H | H | 2-trifluoro- | |
| | | | | | methoxyphenyl | |
| 650 | isobutyl | H | H | H | 3-trifluoro- | |
| | | | | | methoxyphenyl | |
| 651 | isobutyl | H | H | H | 4-trifluoro- | |
| | | | | | methoxyphenyl | |
| 652 | isobutyl | H | H | H | 2-difluoro- | |
| | | | | | methoxyphenyl | |
| 653 | isobutyl | H | H | H | 3-difluoro- | |
| | | | | | methoxyphenyl | |
| 654 | isobutyl | H | H | H | 4-difluoro- | |
| | | | | | methoxyphenyl | |
| 655 | isobutyl | H | H | H | 2-tert-butylphenyl | |
| 656 | isobutyl | H | H | H | 3-tert-butylphenyl | |
| 657 | isobutyl | H | H | H | 4-tert-butylphenyl | |
| 658 | isobutyl | H | H | H | 2-phenoxyphenyl | |
| 659 | isobutyl | H | H | H | 3-phenoxyphenyl | |
| 660 | isobutyl | H | H | H | 4-phenoxyphenyl | |
| 661 | isobutyl | H | H | H | 2-(4-methoxyphenoxy)- | |
| | | | | | phenyl | |
| 662 | isobutyl | H | H | H | 3-(4-methoxyphenoxy)- | |
| | | | | | phenyl | |
| 663 | isobutyl | H | H | H | 4-(4-methoxyphenoxy)- | |
| | | | | | phenyl | |
| 664 | isobutyl | H | H | H | 4-methanesulphonyl- | |
| | | | | | phenyl | |
| 665 | isobutyl | H | H | H | 2-ethoxyphenyl | |
| 666 | isobutyl | H | H | H | 3-ethoxyphenyl | |
| 667 | isobutyl | H | H | H | 4-ethoxyphenyl | |
| 668 | isobutyl | H | H | H | methyl | |
| 669 | isobutyl | H | H | H | ethyl | |
| 670 | isobutyl | H | H | H | n-butyl | |
| 671 | isobutyl | H | H | H | n-butyl | |
| 672 | isobutyl | H | H | H | n-pentyl | |
| 673 | isobutyl | H | H | H | benzyl | |
| 674 | isobutyl | H | H | H | 2-chlorobenzyl | |
| 675 | isobutyl | H | H | H | 3-chlorobenzyl | |
| 676 | isobutyl | H | H | H | 4-chlorobenzyl | |
| 677 | isobutyl | H | H | H | 2-methylbenzyl | |
| 678 | isobutyl | H | H | H | 3-methylbenzyl | |
| 679 | isobutyl | H | H | H | 4-methylbenzyl | |
| 680 | isobutyl | H | H | H | 2-methoxybenzyl | |
| 681 | isobutyl | H | H | H | 3-methoxybenzyl | |
| 682 | isobutyl | H | H | H | 4-methoxybenzyl | |
| 683 | isobutyl | H | H | H | 2,4-dichlorobenzyl | |
| 684 | isobutyl | H | H | H | 3,4-dichlorobenzyl | |
| 685 | isobutyl | H | H | H | 3,5-dichlorobenzyl | |
| 686 | isobutyl | H | H | H | 2,5-dichlorobenzyl | |
| 687 | isobutyl | H | H | H | 2,4,6-trichlorobenzyl | |
| 688 | isobutyl | H | H | H | 2-chloro-4-methylbenzyl | |
| 689 | isobutyl | H | H | H | 2-chloro-4-methoxy- | |
| | | | | | benzyl | |
| 690 | isobutyl | H | H | H | 2,4-dimethylbenzyl | |
| 691 | isobutyl | H | H | H | 2,5-dimethylbenzyl | |
| 692 | isobutyl | H | H | H | 2,6-dimethylbenzyl | |
| 693 | isobutyl | H | H | H | 2,4,6-trimethylbenzyl | |
| 694 | isobutyl | H | H | H | 2-fluorobenzyl | |
| 695 | isobutyl | H | H | H | 3-fluorobenzyl | |
| 696 | isobutyl | H | H | H | 4-fluorobenzyl | |
| 697 | isobutyl | H | H | H | 2-chloro-4-fluorobenzyl | |
| 698 | isobutyl | H | H | H | 2,4-dimethoxybenzyl | |
| 699 | isobutyl | H | H | H | 2,3-dimethoxybenzyl | |
| 700 | isobutyl | H | H | H | 3,4-dimethoxybenzyl | |
| 701 | isobutyl | H | H | H | 3,5-dimethoxybenzyl | |
| 702 | isobutyl | H | H | H | 2,5-dimethoxybenzyl | |
| 703 | isobutyl | H | H | H | 2-trifluoromethylbenzyl | |
| 704 | isobutyl | H | H | H | 3-trifluoromethylbenzyl | |
| 705 | isobutyl | H | H | H | 4-trifluoromethylbenzyl | |
| 706 | isobutyl | H | H | H | 3-trifluoromethyl-4- | |
| | | | | | chlorobenzyl | |
| 707 | isobutyl | H | H | H | 3-trifluoromethyl- | |
| | | | | | 4-fluorobenzyl | |
| 708 | isobutyl | H | H | H | 2-trifluoro- | |
| | | | | | methoxybenzyl | |
| 709 | isobutyl | H | H | H | 3-trifluoro- | |
| | | | | | methoxybenzyl | |
| 710 | isobutyl | H | H | H | 4-trifluoro- | |
| | | | | | methoxybenzyl | |
| 711 | isobutyl | H | H | H | 2-difluoro- | |
| | | | | | methoxybenzyl | |
| 712 | isobutyl | H | H | H | 3-difluoro- | |
| | | | | | methoxybenzyl | |
| 713 | isobutyl | H | H | H | 4-difluoro- | |
| | | | | | methoxybenzyl | |
| 714 | isobutyl | H | H | H | 2-tert-butylbenzyl | |
| 715 | isobutyl | H | H | H | 3-tert-butylbenzyl | |
| 716 | isobutyl | H | H | H | 4-tert-butylbenzyl | |
| 717 | isobutyl | H | H | H | 2-phenoxybenzyl | |
| 718 | isobutyl | H | H | H | 3-phenoxybenzyl | |
| 719 | isobutyl | H | H | H | 4-phenoxybenzyl | |
| 720 | isobutyl | H | H | H | 2-(4-methoxyphenoxy)- | |
| | | | | | benzyl | |
| 721 | isobutyl | H | H | H | 3-(4-methoxyphenoxy)- | |
| | | | | | benzyl | |
| 722 | isobutyl | H | H | H | 4-(4-methoxyphenoxy)- | |
| | | | | | benzyl | |
| 723 | isobutyl | H | H | H | 4-methanesulphonyl- | |
| | | | | | benzyl | |
| 724 | isobutyl | H | H | H | 2-ethoxybenzyl | |
| 725 | isobutyl | H | H | H | 3-ethoxybenzyl | |
| 726 | isobutyl | H | H | H | 4-ethoxybenzyl | |
| 727 | isobutyl | H | H | H | phenethyl | |
| 728 | isobutyl | H | H | H | 2-chlorophenethyl | |
| 729 | isobutyl | H | H | H | 3-chlorophenethyl | |
| 730 | isobutyl | H | H | H | 4-chlorophenethyl | |
| 731 | isobutyl | H | H | H | 2-methylphenethyl | |
| 732 | isobutyl | H | H | H | 3-methylphenethyl | |
| 733 | isobutyl | H | H | H | 4-methylphenethyl | |
| 734 | isobutyl | H | H | H | 2-methoxyphenethyl | |
| 735 | isobutyl | H | H | H | 3-methoxyphenethyl | |
| 736 | isobutyl | H | H | H | 4-methoxyphenethyl | |
| 737 | isobutyl | H | H | H | 2,4-dichlorophenethyl | |
| 738 | isobutyl | H | H | H | 3,4-dichlorophenethyl | |
| 739 | isobutyl | H | H | H | 3,5-dichlorophenethyl | |
| 740 | isobutyl | H | H | H | 2,5-dichlorophenethyl | |
| 741 | isobutyl | H | H | H | 2,4,6-trichlorophenethyl | |
| 742 | isobutyl | H | H | H | 2-chloro- | |
| | | | | | 4-methylphenethyl | |
| 743 | isobutyl | H | H | H | 2-chloro-4-methoxy- | |
| | | | | | phenethyl | |
| 744 | isobutyl | H | H | H | 2,4-dimethylphenethyl | |
| 745 | isobutyl | H | H | H | 2,6-dimethylphenethyl | |
| 746 | isobutyl | H | H | H | 2,4,6-trimethylphenethyl | |
| 747 | isobutyl | H | H | H | 2-fluorophenethyl | |
| 748 | isobutyl | H | H | H | 3-fluorophenethyl | |
| 749 | isobutyl | H | H | H | 4-fluorophenethyl | |
| 750 | isobutyl | H | H | H | 2-chloro- | |
| | | | | | 4-fluorophenethyl | |
| 751 | isobutyl | H | H | H | 2,4-dimethoxyphenethyl | |
| 752 | isobutyl | H | H | H | 3,4-dimethoxyphenethyl | |
| 753 | isobutyl | H | H | H | 3,5-dimethoxyphenethyl | |
| 754 | isobutyl | H | H | H | 2,5-dimethoxyphenethyl | |
| 755 | isobutyl | H | H | H | 2-trifluoromethyl- | |
| | | | | | phenethyl | |
| 756 | isobutyl | H | H | H | 3-trifluoromethyl- | |
| | | | | | phenethyl | |
| 757 | isobutyl | H | H | H | 4-trifluoromethyl- | |
| | | | | | phenethyl | |
| 758 | isobutyl | H | H | H | 2-trifluoro- | |
| | | | | | methoxyphenethyl | |
| 759 | isobutyl | H | H | H | 3-trifluoro- | |
| | | | | | methoxyphenethyl | |
| 760 | isobutyl | H | H | H | 4-trifluoro- | |
| | | | | | methoxyphenethyl | |
| 761 | isobutyl | H | H | H | 2-difluoro- | |
| | | | | | methoxyphenethyl | |
| 762 | isobutyl | H | H | H | 3-difluoro- | |
| | | | | | methoxyphenethyl | |
| 763 | isobutyl | H | H | H | 4-difluoro- | |
| | | | | | methoxyphenethyl | |
| 764 | isobutyl | H | H | H | 2-tert-butylphenethyl | |
| 765 | isobutyl | H | H | H | 3-tert-butylphenethyl | |
| 766 | isobutyl | H | H | H | 4-tert-butylphenethyl | |
| 767 | benzyl | H | H | H | phenyl | |
| 768 | benzyl | H | H | H | 2-chlorophenyl | |
| 769 | benzyl | H | H | H | 3-chlorophenyl | |
| 770 | benzyl | H | H | H | 4-chlorophenyl | 3.00¹⁾ |
| 771 | benzyl | H | H | H | 2-methylphenyl | |
| 772 | benzyl | H | H | H | 3-methylphenyl | 2.89¹⁾ |
| 773 | benzyl | H | H | H | 4-methylphenyl | |
| 774 | benzyl | H | H | H | 2-methoxyphenyl | |
| 775 | benzyl | H | H | H | 3-methoxyphenyl | |
| 776 | benzyl | H | H | H | 4-methoxyphenyl | |
| 777 | benzyl | H | H | H | 2,4-dichlorophenyl | |
| 778 | benzyl | H | H | H | 3,4-dichlorophenyl | |
| 779 | benzyl | H | H | H | 3,5-dichlorophenyl | |
| 780 | benzyl | H | H | H | 2,5-dichlorophenyl | |
| 781 | benzyl | H | H | H | 2,4,6-trichlorophenyl | |
| 782 | benzyl | H | H | H | 2-chloro-4-methylphenyl | |
| 783 | benzyl | H | H | H | 2-chloro-4-methoxy- | |
| | | | | | phenyl | |
| 784 | benzyl | H | H | H | 2,4-dimethylphenyl | |
| 785 | benzyl | H | H | H | 2,5-dimethylphenyl | |
| 786 | benzyl | H | H | H | 2,6-dimethylphenyl | |
| 787 | benzyl | H | H | H | 2,4,6-trimethylphenyl | |
| 788 | benzyl | H | H | H | 2-fluorophenyl | |
| 789 | benzyl | H | H | H | 3-fluorophenyl | 2.63¹⁾ |
| 790 | benzyl | H | H | H | 4-fluorophenyl | |
| 791 | benzyl | H | H | H | 2-chloro-4-fluorophenyl | |
| 792 | benzyl | H | H | H | 2,4-dimethoxyphenyl | |
| 793 | benzyl | H | H | H | 2,3-dimethoxyphenyl | |
| 794 | benzyl | H | H | H | 3,4-dimethoxyphenyl | |
| 795 | benzyl | H | H | H | 3,5-dimethoxyphenyl | 2.65¹⁾ |
| 796 | benzyl | H | H | H | 2,5-dimethoxyphenyl | |
| 797 | benzyl | H | H | H | 2-trifluoromethylphenyl | |
| 798 | benzyl | H | H | H | 3-trifluoromethylphenyl | |
| 799 | benzyl | H | H | H | 4-trifluoromethylphenyl | |
| 800 | benzyl | H | H | H | 3-trifluoromethyl- | |
| | | | | | 4-chlorophenyl | |
| 801 | benzyl | H | H | H | 3-trifluoromethyl- | |
| | | | | | 4-fluorophenyl | |
| 802 | benzyl | H | H | H | 2-trifluoro- | |
| | | | | | methoxyphenyl | |
| 803 | benzyl | H | H | H | 3-trifluoro- | |
| | | | | | methoxyphenyl | |
| 804 | benzyl | H | H | H | 4-trifluoro- | |
| | | | | | methoxyphenyl | |
| 805 | benzyl | H | H | H | 2-difluoro- | |
| | | | | | methoxyphenyl | |
| 806 | benzyl | H | H | H | 3-difluoro- | |
| | | | | | methoxyphenyl | |
| 807 | benzyl | H | H | H | 4-difluoro- | |
| | | | | | methoxyphenyl | |
| 808 | benzyl | H | H | H | 2-tert-butylphenyl | |
| 809 | benzyl | H | H | H | 3-tert-butylphenyl | |
| 810 | benzyl | H | H | H | 4-tert-butylphenyl | 3.83¹⁾ |
| 811 | benzyl | H | H | H | 2-phenoxyphenyl | |
| 812 | benzyl | H | H | H | 3-phenoxyphenyl | |
| 813 | benzyl | H | H | H | 4-phenoxyphenyl | |
| 814 | benzyl | H | H | H | 2-(4-methoxyphenoxy)- | |
| | | | | | phenyl | |
| 815 | benzyl | H | H | H | 3-(4-methoxyphenoxy)- | |
| | | | | | phenyl | |
| 816 | benzyl | H | H | H | 4-(4-methoxyphenoxy)- | |
| | | | | | phenyl | |
| 817 | benzyl | H | H | H | 4-methanesulphonyl- | |
| | | | | | phenyl | |
| 818 | benzyl | H | H | H | 2-ethoxyphenyl | |
| 819 | benzyl | H | H | H | 3-ethoxyphenyl | |
| 820 | benzyl | H | H | H | 4-ethoxyphenyl | |
| 821 | benzyl | H | H | H | methyl | |
| 822 | benzyl | H | H | H | ethyl | |
| 823 | benzyl | H | H | H | n-butyl | |
| 824 | benzyl | H | H | H | n-butyl | |
| 825 | benzyl | H | H | H | n-pentyl | |
| 826 | benzyl | H | H | H | benzyl | |
| 827 | benzyl | H | H | H | 2-chlorobenzyl | |
| 828 | benzyl | H | H | H | 3-chlorobenzyl | 3.16¹⁾ |
| 829 | benzyl | H | H | H | 4-chlorobenzyl | |
| 830 | benzyl | H | H | H | 2-methylbenzyl | |
| 831 | benzyl | H | H | H | 3-methylbenzyl | |
| 832 | benzyl | H | H | H | 4-methylbenzyl | |
| 833 | benzyl | H | H | H | 2-methoxybenzyl | |
| 834 | benzyl | H | H | H | 3-methoxybenzyl | |
| 835 | benzyl | H | H | H | 4-methoxybenzyl | |
| 836 | benzyl | H | H | H | 2,4-dichlorobenzyl | |
| 837 | benzyl | H | H | H | 3,4-dichlorobenzyl | |
| 838 | benzyl | H | H | H | 3,5-dichlorobenzyl | |
| 839 | benzyl | H | H | H | 2,5-dichlorobenzyl | |
| 840 | benzyl | H | H | H | 2,4,6-trichlorobenzyl | |
| 841 | benzyl | H | H | H | 2-chloro-4-methylbenzyl | |
| 842 | benzyl | H | H | H | 2-chloro-4-methoxy- | |
| | | | | | benzyl | |
| 843 | benzyl | H | H | H | 2,4-dimethylbenzyl | |
| 844 | benzyl | H | H | H | 2,5-dimethylbenzyl | |
| 845 | benzyl | H | H | H | 2,6-dimethylbenzyl | |
| 846 | benzyl | H | H | H | 2,4,6-trimethylbenzyl | |
| 847 | benzyl | H | H | H | 2-fluorobenzyl | |
| 848 | benzyl | H | H | H | 3-fluorobenzyl | |
| 849 | benzyl | H | H | H | 4-fluorobenzyl | |
| 850 | benzyl | H | H | H | 2-chloro-4-fluorobenzyl | |
| 851 | benzyl | H | H | H | 2,4-dimethoxybenzyl | |
| 852 | benzyl | H | H | H | 2,3-dimethoxybenzyl | |
| 853 | benzyl | H | H | H | 3,4-dimethoxybenzyl | |
| 854 | benzyl | H | H | H | 3,5-dimethoxybenzyl | |
| 855 | benzyl | H | H | H | 2,5-dimethoxybenzyl | |
| 856 | benzyl | H | H | H | 2-trifluoromethylbenzyl | |
| 857 | benzyl | H | H | H | 3-trifluoromethylbenzyl | |
| 858 | benzyl | H | H | H | 4-trifluoromethylbenzyl | |
| 859 | benzyl | H | H | H | 3-trifluoromethyl-4- | |
| | | | | | chlorobenzyl | |
| 860 | benzyl | H | H | H | 3-trifluoromethyl- | |
| | | | | | 4-fluorobenzyl | |
| 861 | benzyl | H | H | H | 2-trifluoro- | |
| | | | | | methoxybenzyl | |
| 862 | benzyl | H | H | H | 3-trifluoro- | |
| | | | | | methoxybenzyl | |
| 863 | benzyl | H | H | H | 4-trifluoro- | |
| | | | | | methoxybenzyl | |
| 864 | benzyl | H | H | H | 2-difluoro- | |
| | | | | | methoxybenzyl | |
| 865 | benzyl | H | H | H | 3-difluoro- | |
| | | | | | methoxybenzyl | |
| 866 | benzyl | H | H | H | 4-difluoro- | |
| | | | | | methoxybenzyl | |
| 867 | benzyl | H | H | H | 2-tert-butylbenzyl | |
| 868 | benzyl | H | H | H | 3-tert-butylbenzyl | |
| 869 | benzyl | H | H | H | 4-tert-butylbenzyl | |
| 870 | benzyl | H | H | H | 2-phenoxybenzyl | |
| 871 | benzyl | H | H | H | 3-phenoxybenzyl | |
| 872 | benzyl | H | H | H | 4-phenoxybenzyl | |
| 873 | benzyl | H | H | H | 2-(4-methoxyphenoxy)- | |
| | | | | | benzyl | |
| 874 | benzyl | H | H | H | 3-(4-methoxyphenoxy)- | |
| | | | | | benzyl | |
| 875 | benzyl | H | H | H | 4-(4-methoxyphenoxy)- | |
| | | | | | benzyl | |
| 876 | benzyl | H | H | H | 4-methanesulphonyl- | |
| | | | | | benzyl | |
| 877 | benzyl | H | H | H | 2-ethoxybenzyl | |
| 878 | benzyl | H | H | H | 3-ethoxybenzyl | |
| 879 | benzyl | H | H | H | 4-ethoxybenzyl | |
| 880 | benzyl | H | H | H | phenethyl | |
| 881 | benzyl | H | H | H | 2-chlorophenethyl | |
| 882 | benzyl | H | H | H | 3-chlorophenethyl | |
| 883 | benzyl | H | H | H | 4-chlorophenethyl | |
| 884 | benzyl | H | H | H | 2-methylphenethyl | |
| 885 | benzyl | H | H | H | 3-methylphenethyl | |
| 886 | benzyl | H | H | H | 4-methylphenethyl | |
| 887 | benzyl | H | H | H | 2-methoxyphenethyl | |
| 888 | benzyl | H | H | H | 3-methoxyphenethyl | |
| 889 | benzyl | H | H | H | 4-methoxyphenethyl | |
| 890 | benzyl | H | H | H | 2,4-dichlorophenethyl | |
| 891 | benzyl | H | H | H | 3,4-dichlorophenethyl | |
| 892 | benzyl | H | H | H | 3,5-dichlorophenethyl | |
| 893 | benzyl | H | H | H | 2,5-dichlorophenethyl | |
| 894 | benzyl | H | H | H | 2,4,6-trichlorophenethyl | |
| 895 | benzyl | H | H | H | 2-chloro- | |
| | | | | | 4-methylphenethyl | |
| 896 | benzyl | H | H | H | 2-chloro-4-methoxy- | |
| | | | | | phenethyl | |
| 897 | benzyl | H | H | H | 2,4-dimethylphenethyl | |
| 898 | benzyl | H | H | H | 2,6-dimethylphenethyl | |
| 899 | benzyl | H | H | H | 2,4,6-trimethylphenethyl | |
| 900 | benzyl | H | H | H | 2-fluorophenethyl | |
| 901 | benzyl | H | H | H | 3-fluorophenethyl | |
| 902 | benzyl | H | H | H | 4-fluorophenethyl | |
| 903 | benzyl | H | H | H | 2-chloro- | |
| | | | | | 4-fluorophenethyl | |
| 904 | benzyl | H | H | H | 2,4-dimethoxyphenethyl | |
| 905 | benzyl | H | H | H | 3,4-dimethoxyphenethyl | |
| 906 | benzyl | H | H | H | 3,5-dimethoxyphenethyl | |
| 907 | benzyl | H | H | H | 2,5-dimethoxyphenethyl | |
| 908 | benzyl | H | H | H | 2-trifluoromethyl- | |
| | | | | | phenethyl | |
| 909 | benzyl | H | H | H | 3-trifluoromethyl- | |
| | | | | | phenethyl | |
| 910 | benzyl | H | H | H | 4-trifluoromethyl- | |
| | | | | | phenethyl | |
| 911 | benzyl | H | H | H | 2-trifluoro- | |
| | | | | | methoxyphenethyl | |
| 912 | benzyl | H | H | H | 3-trifluoro- | |
| | | | | | methoxyphenethyl | |
| 913 | benzyl | H | H | H | 4-trifluoro- | |
| | | | | | methoxyphenethyl | |
| 914 | benzyl | H | H | H | 2-difluoro- | |
| | | | | | methoxyphenethyl | |
| 915 | benzyl | H | H | H | 3-difluoro- | |
| | | | | | methoxyphenethyl | |
| 916 | benzyl | H | H | H | 4-difluoro- | |
| | | | | | methoxyphenethyl | |
| 917 | benzyl | H | H | H | 2-tert-butylphenethyl | |
| 918 | benzyl | H | H | H | 3-tert-butylphenethyl | |
| 919 | benzyl | H | H | H | 4-tert-butylphenethyl | |
| 920 | 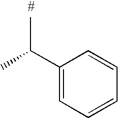 | H | H | H | phenyl | |
| 921 | 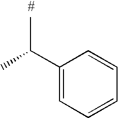 | H | H | H | 2-chlorophenyl | |
| 922 | 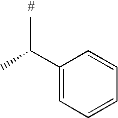 | H | H | H | 3-chlorophenyl | |
| 923 | 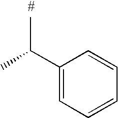 | H | H | H | 4-chlorophenyl | |
| 924 | 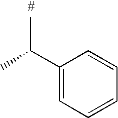 | H | H | H | 2-methylphenyl | |
| 925 | 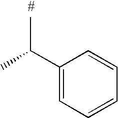 | H | H | H | 3-methylphenyl | |
| 926 | 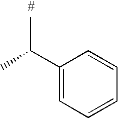 | H | H | H | 4-methylphenyl | |
| 927 | 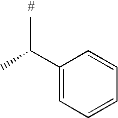 | H | H | H | 2-methoxyphenyl | |
| 928 | 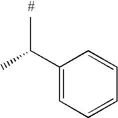 | H | H | H | 3-methoxyphenyl | |
| 929 | 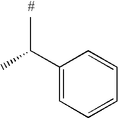 | H | H | H | 4-methoxyphenyl | |
| 930 | 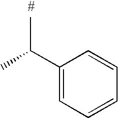 | H | H | H | 2,4-dichlorophenyl | |
| 931 | 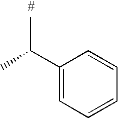 | H | H | H | 3,4-dichlorophenyl | |
| 932 | 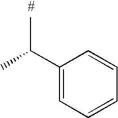 | H | H | H | 3,5-dichlorophenyl | |
| 933 | 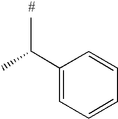 | H | H | H | 2,5-dichlorophenyl | |
| 934 | 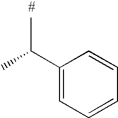 | H | H | H | 2,4,6-trichlorophenyl | |
| 935 | 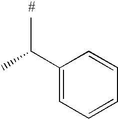 | H | H | H | 2-chloro-4-methylphenyl | |
| 936 | 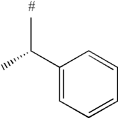 | H | H | H | 2-chloro-4-methoxy- phenyl | |
| 937 | 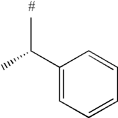 | H | H | H | 2,4-dimethylphenyl | |
| 938 | 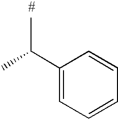 | H | H | H | 2,5-dimethylphenyl | |
| 939 | 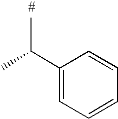 | H | H | H | 2,6-dimethylphenyl | |
| 940 | 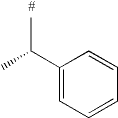 | H | H | H | 2,4,6-trimethylphenyl | |
| 941 | 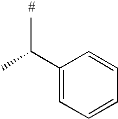 | H | H | H | 2-fluorophenyl | |
| 942 | 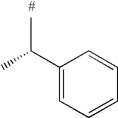 | H | H | H | 3-fluorophenyl | |
| 943 | 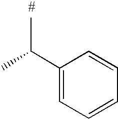 | H | H | H | 4-fluorophenyl | |
| 944 | 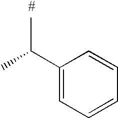 | H | H | H | 2-chloro-4-fluorophenyl | |
| 945 | 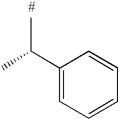 | H | H | H | 2,4-dimethoxyphenyl | |
| 946 | 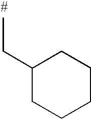 | H | H | H | phenyl | |
| 947 | 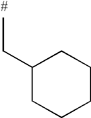 | H | H | H | 2-chlorophenyl | |
| 948 | 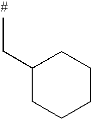 | H | H | H | 3-chlorophenyl | |
| 949 | 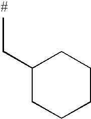 | H | H | H | 4-chlorophenyl | |
| 950 | 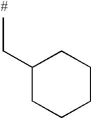 | H | H | H | 2-methylphenyl | |
| 951 | 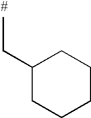 | H | H | H | 3-methylphenyl | |
| 952 | 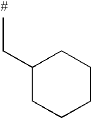 | H | H | H | 4-methylphenyl | |
| 953 | 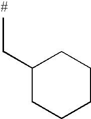 | H | H | H | 2-methoxyphenyl | |
| 954 | 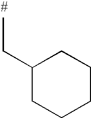 | H | H | H | 3-methoxyphenyl | |
| 955 | 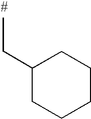 | H | H | H | 4-methoxyphenyl | |
| 956 | 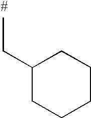 | H | H | H | 2,4-dichlorophenyl | |
| 957 | 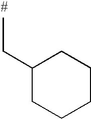 | H | H | H | 3,4-dichlorophenyl | |
| 958 | 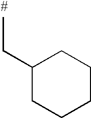 | H | H | H | 3,5-dichlorophenyl | |
| 959 | 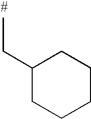 | H | H | H | 2,5-dichlorophenyl | |
| 960 | 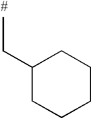 | H | H | H | 2,4,6-trichlorophenyl | |
| 961 | 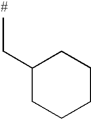 | H | H | H | 2-chloro-4-methylphenyl | |
| 962 | 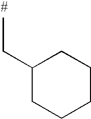 | H | H | H | 2-chloro-4-methoxy- phenyl | |
| 963 | 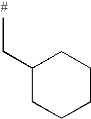 | H | H | H | 2,4-dimethylphenyl | |
| 964 | 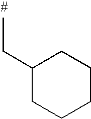 | H | H | H | 2,5-dimethylphenyl | |
| 965 | 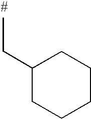 | H | H | H | 2,6-dimethylphenyl | |
| 966 | 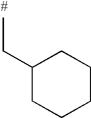 | H | H | H | 2,4,6-trimethylphenyl | |
| 967 | 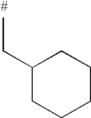 | H | H | H | 2-fluorophenyl | |
| 968 | 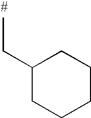 | H | H | H | 3-fluorophenyl | |
| 969 | 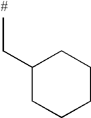 | H | H | H | 4-fluorophenyl | |
| 970 | 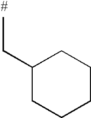 | H | H | H | 2-chloro-4-fluorophenyl | |
| 971 | 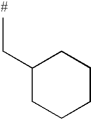 | H | H | H | 2,4-dimethoxyphenyl | |
| 972 | 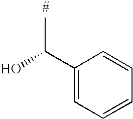 | H | H | H | phenyl | |
| 973 | 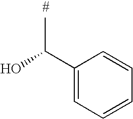 | H | H | H | 2-chlorophenyl | |
| 974 | 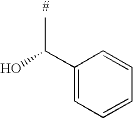 | H | H | H | 3-chlorophenyl | |
| 975 | 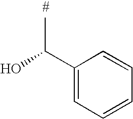 | H | H | H | 4-chlorophenyl | |
| 976 | 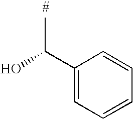 | H | H | H | 2-methylphenyl | |
| 977 | 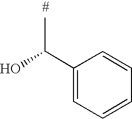 | H | H | H | 3-methylphenyl | |
| 978 | 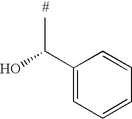 | H | H | H | 4-methylphenyl | |
| 979 | 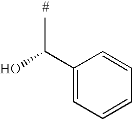 | H | H | H | 2-methoxyphenyl | |
| 980 | 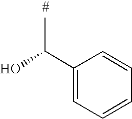 | H | H | H | 3-methoxyphenyl | |
| 981 | 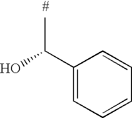 | H | H | H | 4-methoxyphenyl | |
| 982 | 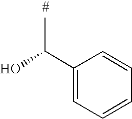 | H | H | H | 2,4-dichlorophenyl | |
| 983 | 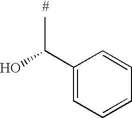 | H | H | H | 3,4-dichlorophenyl | |
| 984 | 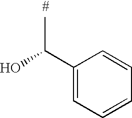 | H | H | H | 3,5-dichlorophenyl | |
| 985 | 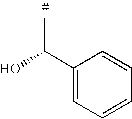 | H | H | H | 2,5-dichlorophenyl | |
| 986 | 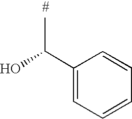 | H | H | H | 2,4,6-trichlorophenyl | |
| 987 | 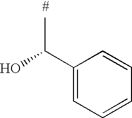 | H | H | H | 2-chloro-4-methylphenyl | |
| 988 | 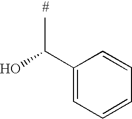 | H | H | H | 2-chloro-4-methoxy- phenyl | |
| 989 | 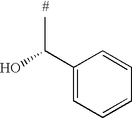 | H | H | H | 2,4-dimethylphenyl | |
| 990 | 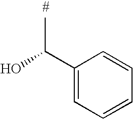 | H | H | H | 2,5-dimethylphenyl | |
| 991 | 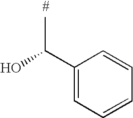 | H | H | H | 2,6-dimethylphenyl | |
| 992 | 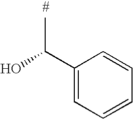 | H | H | H | 2,4,6-trimethylphenyl | |
| 993 | 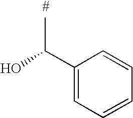 | H | H | H | 2-fluorophenyl | |
| 994 | 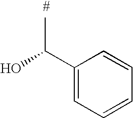 | H | H | H | 3-fluorophenyl | |
| 995 | 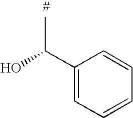 | H | H | H | 4-fluorophenyl | |
| 996 | 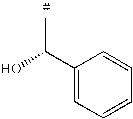 | H | H | H | 2-chloro-4-fluorophenyl | |
| 997 | 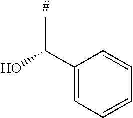 | H | H | H | 2,4-dimethoxyphenyl | |
| 998 | 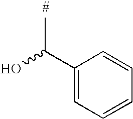 | H | H | H | phenyl | |
| 999 | 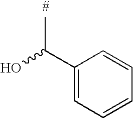 | H | H | H | 2-chlorophenyl | |
| 1000 | 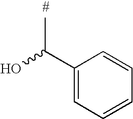 | H | H | H | 3-chlorophenyl | |
| 1001 | 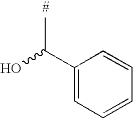 | H | H | H | 4-chlorophenyl | |
| 1002 | 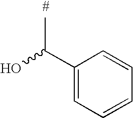 | H | H | H | 2-methylphenyl | |
| 1003 | 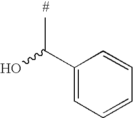 | H | H | H | 3-methylphenyl | |
| 1004 | 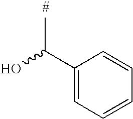 | H | H | H | 4-methylphenyl | |
| 1005 | 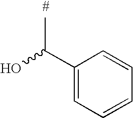 | H | H | H | 2-methoxyphenyl | |
| 1006 | 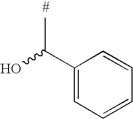 | H | H | H | 3-methoxyphenyl | |
| 1007 | 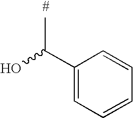 | H | H | H | 4-methoxyphenyl | |
| 1008 | 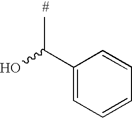 | H | H | H | 2,4-dichlorophenyl | |
| 1009 | 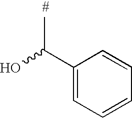 | H | H | H | 3,4-dichlorophenyl | |
| 1010 | 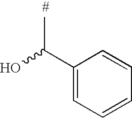 | H | H | H | 3,5-dichlorophenyl | |
| 1011 | 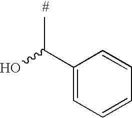 | H | H | H | 2,5-dichlorophenyl | |
| 1012 | 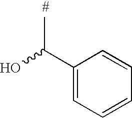 | H | H | H | 2,4,6-trichlorophenyl | |
| 1013 | 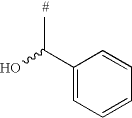 | H | H | H | 2-chloro-4-methylphenyl | |
| 1014 | 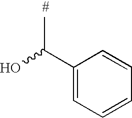 | H | H | H | 2-chloro-4-methoxy- phenyl | |
| 1015 | 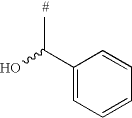 | H | H | H | 2,4-dimethylphenyl | |
| 1016 | 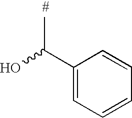 | H | H | H | 2,5-dimethylphenyl | |
| 1017 | 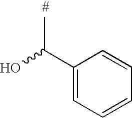 | H | H | H | 2,6-dimethylphenyl | |
| 1018 | 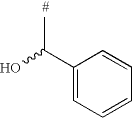 | H | H | H | 2,4,6-trimethylphenyl | |
| 1019 | 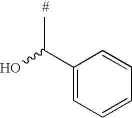 | H | H | H | 2-fluorophenyl | |
| 1020 | 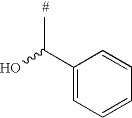 | H | H | H | 3-fluorophenyl | |
| 1021 | 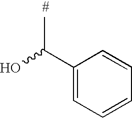 | H | H | H | 4-fluorophenyl | |
| 1022 | 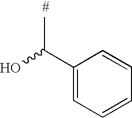 | H | H | H | 2-chloro-4-fluorophenyl | |
| 1023 | 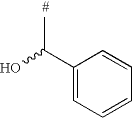 | H | H | H | 2,4-dimethoxyphenyl | |
| 1024 | 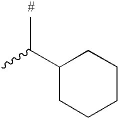 | H | H | H | phenyl | |
| 1025 | 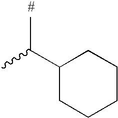 | H | H | H | 2-chlorophenyl | |
| 1026 | 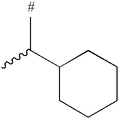 | H | H | H | 3-chlorophenyl | |
| 1027 | 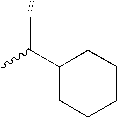 | H | H | H | 4-chlorophenyl | |
| 1028 | 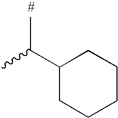 | H | H | H | 2-methylphenyl | |
| 1029 | 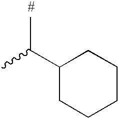 | H | H | H | 3-methylphenyl | |
| 1030 | 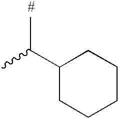 | H | H | H | 4-methylphenyl | |
| 1031 | 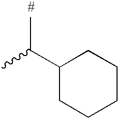 | H | H | H | 2-methoxyphenyl | |
| 1032 | 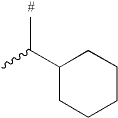 | H | H | H | 3-methoxyphenyl | |
| 1033 | 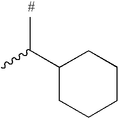 | H | H | H | 4-methoxyphenyl | |
| 1034 | 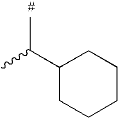 | H | H | H | 2,4-dichlorophenyl | |
| 1035 | 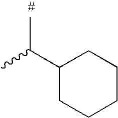 | H | H | H | 3,4-dichlorophenyl | |
| 1036 | 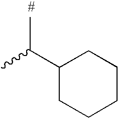 | H | H | H | 3,5-dichlorophenyl | |
| 1037 | 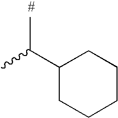 | H | H | H | 2,5-dichlorophenyl | |
| 1038 | 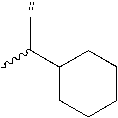 | H | H | H | 2,4,6-trichlorophenyl | |
| 1039 | 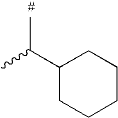 | H | H | H | 2-chloro-4-methylphenyl | |
| 1040 | 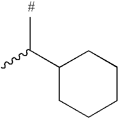 | H | H | H | 2-chloro-4-methoxy- phenyl | |
| 1041 | 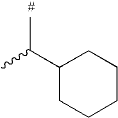 | H | H | H | 2,4-dimethylphenyl | |
| 1042 | 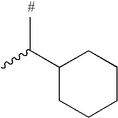 | H | H | H | 2,5-dimethylphenyl | |
| 1043 | 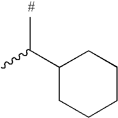 | H | H | H | 2,6-dimethylphenyl | |
| 1044 | 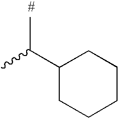 | H | H | H | 2,4,6-trimethylphenyl | |
| 1045 | 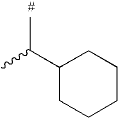 | H | H | H | 2-fluorophenyl | |
| 1046 | 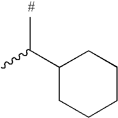 | H | H | H | 3-fluorophenyl | |
| 1047 | 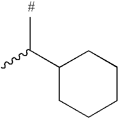 | H | H | H | 4-fluorophenyl | |
| 1048 | 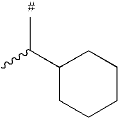 | H | H | H | 2-chloro-4-fluorophenyl | |
| 1049 | 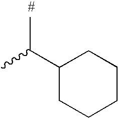 | H | H | H | 2,4-dimethoxyphenyl | |
| 1050 | 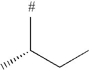 | CH₃ | H | H | phenyl | |
| 1051 | 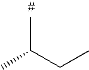 | CH₃ | H | H | 2-chlorophenyl | |
| 1052 | 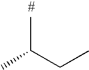 | CH₃ | H | H | 3-chlorophenyl | |
| 1053 | 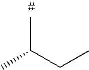 | CH₃ | H | H | 4-chlorophenyl | |
| 1054 | 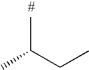 | CH₃ | H | H | 2-methylphenyl | |
| 1055 | 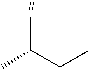 | CH₃ | H | H | 3-methylphenyl | |
| 1056 | 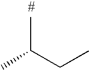 | CH₃ | H | H | 4-methylphenyl | |
| 1057 | 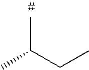 | CH₃ | H | H | 2-methoxyphenyl | |
| 1058 | 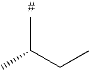 | CH₃ | H | H | 3-methoxyphenyl | |
| 1059 | 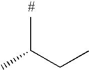 | CH₃ | H | H | 4-methoxyphenyl | |
| 1060 | 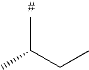 | CH₃ | H | H | 2,4-dichlorophenyl | |
| 1061 | 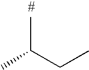 | CH₃ | H | H | 3,4-dichlorophenyl | |
| 1062 | 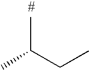 | CH₃ | H | H | 3,5-dichlorophenyl | |
| 1063 | 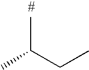 | CH₃ | H | H | 2,5-dichlorophenyl | |
| 1064 | 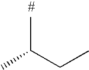 | CH₃ | H | H | 2,4,6-trichlorophenyl | |
| 1065 | 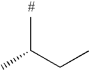 | CH₃ | H | H | 2-chloro-4-methylphenyl | |
| 1066 | 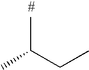 | CH₃ | H | H | 2-chloro-4-methoxy- phenyl | |
| 1067 | 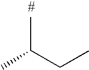 | CH₃ | H | H | 2,4-dimethylphenyl | |
| 1068 | 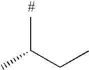 | CH₃ | H | H | 2,5-dimethylphenyl | |
| 1069 | 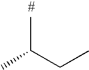 | CH₃ | H | H | 2,6-dimethylphenyl | |
| 1070 | 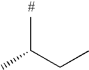 | CH₃ | H | H | 2,4,6-trimethylphenyl | |
| 1070a | 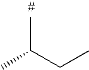 | CH₃ | H | H | 3,5-dimethoxyphenyl | |
| 1070b | 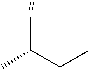 | CH₃ | H | H | 4-tert-butylphenyl | |
| 1071 | 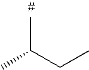 | CH₃ | H | H | methyl | 4.32¹⁾ |
| 1072 | 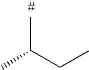 | CH₃ | H | H | ethyl | |
| 1073 | 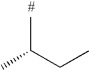 | CH₃ | H | H | n-propyl | |
| 1074 | 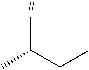 | CH₃ | H | H | n-butyl | |
| 1075 | 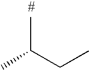 | CH₃ | H | H | n-pentyl | |
| 1076 | 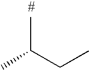 | H | CH₃ | H | phenyl | |
| 1077 | 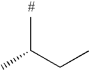 | H | CH₃ | H | 2-chlorophenyl | |
| 1078 | 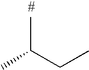 | H | CH₃ | H | 3-chlorophenyl | |
| 1079 | 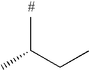 | H | CH₃ | H | 4-chlorophenyl | |
| 1080 | 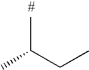 | H | CH₃ | H | 2-methylphenyl | |
| 1081 | 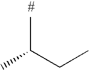 | H | CH₃ | H | 3-methylphenyl | 3.31¹⁾ |
| 1082 | 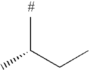 | H | CH₃ | H | 4-methylphenyl | |
| 1083 | 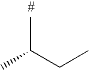 | H | CH₃ | H | 2-methoxyphenyl | |
| 1084 | 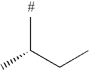 | H | CH₃ | H | 3-methoxyphenyl | |
| 1085 | 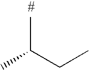 | H | CH₃ | H | 4-methoxyphenyl | |
| 1086 | 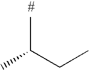 | H | CH₃ | H | 2,4-dichlorophenyl | 3.91¹⁾ |
| 1087 | 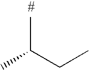 | H | CH₃ | H | 3,4-dichlorophenyl | |
| 1088 | 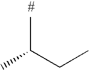 | H | CH₃ | H | 3,5-dichlorophenyl | |
| 1089 | 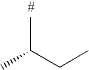 | H | CH₃ | H | 2,5-dichlorophenyl | |
| 1089a | 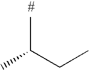 | H | CH₃ | H | 4-trifluorophenyl | 3.59¹⁾ |
| 1090 | 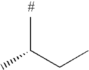 | H | CH₃ | H | 2,4,6-trichlorophenyl | |
| 1091 | 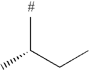 | H | CH₃ | H | 2-chloro-4-methylphenyl | |
| 1092 | 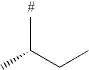 | H | CH₃ | H | 2-chloro-4-methoxy- phenyl | |
| 1093 | 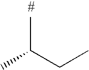 | H | CH₃ | H | 2,4-dimethylphenyl | |
| 1094 | 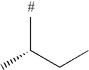 | H | CH₃ | H | 2,5-dimethylphenyl | |
| 1095 | 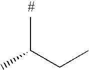 | H | CH₃ | H | 2,6-dimethylphenyl | |
| 1096 | 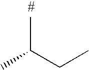 | H | CH₃ | H | 2,4,6-trimethylphenyl | |
| 1097 | 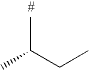 | H | CH₃ | H | methyl | |
| 1098 | 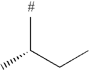 | H | CH₃ | H | ethyl | |
| 1099 | 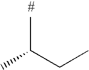 | H | CH₃ | H | n-propyl | |
| 1100 | 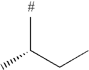 | H | CH₃ | H | n-butyl | |
| 1100a | H | H | CH₃ | H | 3,5-dimethoxyphenyl | 1.72¹⁾ |
| 1101 | 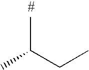 | H | CH₃ | H | n-pentyl | |
| 1102 | cyclohexyl | H | CH₃ | H | phenyl | |
| 1103 | cyclohexyl | H | CH₃ | H | 2-chlorophenyl | |
| 1104 | cyclohexyl | H | CH₃ | H | 3-chlorophenyl | |
| 1105 | cyclohexyl | H | CH₃ | H | 4-chlorophenyl | |
| 1106 | cyclohexyl | H | CH₃ | H | 2-methylphenyl | |
| 1107 | cyclohexyl | H | CH₃ | H | 3-methylphenyl | |
| 1108 | cyclohexyl | H | CH₃ | H | 4-methylphenyl | |
| 1109 | cyclohexyl | H | CH₃ | H | 2-methoxyphenyl | |
| 1110 | cyclohexyl | H | CH₃ | H | 3-methoxyphenyl | |
| 1111 | cyclohexyl | H | CH₃ | H | 4-methoxyphenyl | |
| 1112 | cyclohexyl | H | CH₃ | H | 2,4-dichlorophenyl | |
| 1113 | cyclohexyl | H | CH₃ | H | 3,4-dichlorophenyl | |
| 1114 | cyclohexyl | H | CH₃ | H | 3,5-dichlorophenyl | |
| 1115 | cyclohexyl | H | CH₃ | H | 2,5-dichlorophenyl | |
| 1116 | cyclohexyl | H | CH₃ | H | 2,4,6-trichlorophenyl | |
| 1117 | cyclohexyl | H | CH₃ | H | 2-chloro-4-methylphenyl | |
| 1118 | cyclohexyl | H | CH₃ | H | 2-chloro-4-methoxy- | |
| | | | | | phenyl | |
| 1119 | cyclohexyl | H | CH₃ | H | 2,4-dimethylphenyl | |
| 1120 | cyclohexyl | H | CH₃ | H | 2,5-dimethylphenyl | |
| 1121 | cyclohexyl | H | CH₃ | H | 2,6-dimethylphenyl | |
| 1122 | cyclohexyl | H | CH₃ | H | 2,4,6-trimethylphenyl | |
| 1123 | cyclohexyl | H | CH₃ | H | methyl | |
| 1124 | cyclohexyl | H | CH₃ | H | ethyl | |
| 1125 | cyclohexyl | H | CH₃ | H | n-propyl | n.d.⁴⁾ |
| 1126 | cyclohexyl | H | CH₃ | H | n-butyl | |
| 1127 | cyclohexyl | H | CH₃ | H | n-pentyl | |
| 1128 | 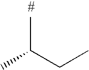 | H | benzyl | H | phenyl | |
| 1129 | cyclohexyl | H | benzyl | H | phenyl | 5.45¹⁾ |
| 1130 | 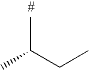 | H | H | CH₃ | phenyl | |
| 1131 | 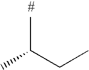 | H | H | CH₃ | 2-chlorophenyl | |
| 1132 | 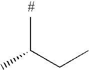 | H | H | CH₃ | 3-chlorophenyl | 3.22¹⁾ |
| 1133 | 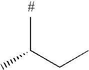 | H | H | CH₃ | 4-chlorophenyl | 3.27¹⁾ |
| 1134 | 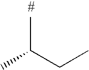 | H | H | CH₃ | 2-methylphenyl | |
| 1135 | 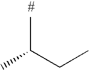 | H | H | CH₃ | 3-methylphenyl | |
| 1136 | 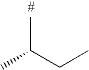 | H | H | CH₃ | 4-methylphenyl | |
| 1137 | 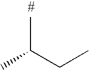 | H | H | CH₃ | 2-methoxyphenyl | |
| 1138 | 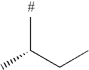 | H | H | CH₃ | 3-methoxyphenyl | |
| 1139 | 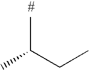 | H | H | CH₃ | 4-methoxyphenyl | 3.81¹⁾ |
| 1140 | 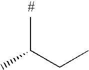 | H | H | CH₃ | 2,4-dichlorophenyl | |
| 1141 | 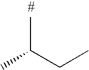 | H | H | CH₃ | 3,4-dichlorophenyl | |
| 1142 | 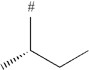 | H | H | CH₃ | 3,5-dichlorophenyl | |
| 1143 | 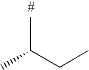 | H | H | CH₃ | 2,5-dichlorophenyl | |
| 1144 | 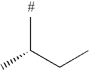 | H | H | CH₃ | 2,4,6-trichlorophenyl | |
| 1145 | 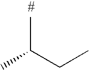 | H | H | CH₃ | 2-chloro-4-methylphenyl | |
| 1146 | 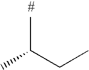 | H | H | CH₃ | 2-chloro-4-methoxy- phenyl | |
| 1147 | 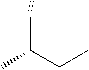 | H | H | CH₃ | 2,4-dimethylphenyl | |
| 1148 | 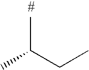 | H | H | CH₃ | 2,5-dimethylphenyl | |
| 1149 | 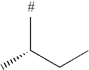 | H | H | CH₃ | 2,6-dimethylphenyl | |
| 1150 | 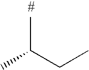 | H | H | CH₃ | 2,4,6-trimethylphenyl | |
| 1151 | 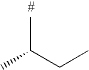 | H | H | CH₃ | 2-fluorophenyl | |
| 1152 | 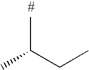 | H | H | CH₃ | 3-fluorophenyl | |
| 1153 | 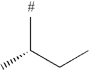 | H | H | CH₃ | 4-fluorophenyl | |
| 1154 | 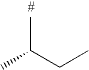 | H | H | CH₃ | 2-chloro-4-fluorophenyl | |
| 1155 | 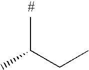 | H | H | CH₃ | 2,4-dimethoxyphenyl | |
| 1156 | 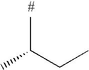 | H | H | CH₃ | 2,3-dimethoxyphenyl | |
| 1157 | 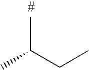 | H | H | CH₃ | 3,4-dimethoxyphenyl | |
| 1158 | 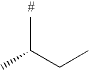 | H | H | CH₃ | 3,5-dimethoxyphenyl | |
| 1159 | 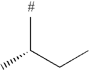 | H | H | CH₃ | 2,5-dimethoxyphenyl | |
| 1160 | 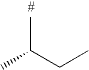 | H | H | CH₃ | 2-trifluoromethylphenyl | |
| 1161 | 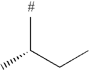 | H | H | CH₃ | 3-trifluoromethylphenyl | |
| 1162 | 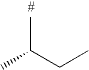 | H | H | CH₃ | 4-trifluoromethylphenyl | |
| 1163 | 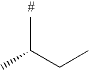 | H | H | CH₃ | 3-trifluoromethyl- 4-chlorophenyl | |
| 1164 | 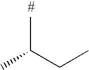 | H | H | CH₃ | 3-trifluoromethyl- 4-fluorophenyl | |
| 1165 | 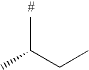 | H | H | CH₃ | 2-trifluoro- methoxyphenyl | |
| 1166 | 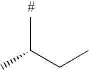 | H | H | CH₃ | 3-trifluoro- methoxyphenyl | |
| 1167 | 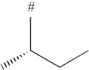 | H | H | CH₃ | 4-trifluoro- methoxyphenyl | |
| 1168 | 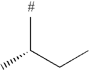 | H | H | CH₃ | 2-difluoro- methoxyphenyl | |
| 1169 | 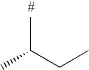 | H | H | CH₃ | 3-difluoro- methoxyphenyl | |
| 1170 | 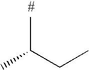 | H | H | CH₃ | 4-difluoro- methoxyphenyl | |
| 1171 | 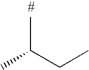 | H | H | CH₃ | 2-tert-butylphenyl | |
| 1172 | 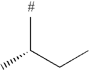 | H | H | CH₃ | 3-tert-butylphenyl | |
| 1173 | 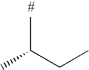 | H | H | CH₃ | 4-tert-butylphenyl | 4.10¹⁾ |
| 1174 | 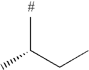 | H | H | CH₃ | 2-phenoxyphenyl | |
| 1175 | 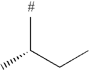 | H | H | CH₃ | 3-phenoxyphenyl | |
| 1176 | 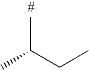 | H | H | CH₃ | 4-phenoxyphenyl | |
| 1177 | 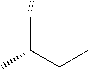 | H | H | CH₃ | 2-(4-methoxyphenoxy)- phenyl | |
| 1177a | 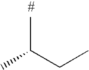 | H | H | CH₃ | naphthalen-2-yl | 3.37¹⁾ |
| 1177b | 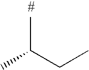 | H | H | C₂H₅ | 4-methoxyphenyl | 3.02¹⁾ |
| 1178 | 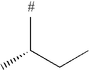 | H | H | CH₃ | 3-(4-methoxyphenoxy)- phenyl | |
| 1179 | 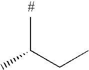 | H | H | CH₃ | 4-(4-methoxyphenoxy)- phenyl | |
| 1180 | 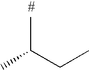 | H | H | CH₃ | 4-methanesulphonyl- phenyl | 2.13²⁾ |
| 1180a | 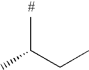 | H | H | CF₃ | phenyl | |
| 1180b | 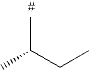 | H | H | CF₃ | 2-chlorophenyl | |
| 1180c | 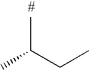 | H | H | CF₃ | 3-chlorophenyl | |
| 1180d | 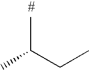 | H | H | CF₃ | 4-chlorophenyl | 2.86¹⁾ |
| 1180e | 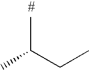 | H | H | CF₃ | 2-methylphenyl | |
| 1180f | 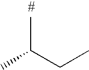 | H | H | CF₃ | 3-methylphenyl | |
| 1180g | 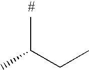 | H | H | CF₃ | 4-methylphenyl | |
| 1180h | 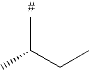 | H | H | CF₃ | 2-methoxyphenyl | |
| 1180i | 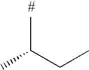 | H | H | CF₃ | 3-methoxyphenyl | |
| 1180j | 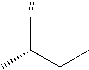 | H | H | CF₃ | 4-methoxyphenyl | 3.28¹⁾ |
| 1180k | isopropyl | H | H | CH₃ | phenyl | isopropyl |
| 1180l | isopropyl | H | H | CH₃ | 2-chlorophenyl | isopropyl |
| 1180m | isopropyl | H | H | CH₃ | 3-chlorophenyl | isopropyl |
| 1180n | isopropyl | H | H | CH₃ | 4-chlorophenyl | isopropyl |
| 1180o | isopropyl | H | H | CH₃ | 2-methylphenyl | isopropyl |
| 1180p | isopropyl | H | H | CH₃ | 3-methylphenyl | isopropyl |
| 1180q | isopropyl | H | H | CH₃ | 4-methylphenyl | isopropyl |
| 1180r | isopropyl | H | H | CH₃ | 2-methoxyphenyl | isopropyl |
| 1180s | isopropyl | H | H | CH₃ | 3-methoxyphenyl | isopropyl |
| 1180t | isopropyl | H | H | CH₃ | 4-methoxyphenyl | isopropyl |
| 1181 | isopropyl | H | H | CH₃ | phenyl | |
| 1182 | isopropyl | H | H | CH₃ | 2-chlorophenyl | |
| 1183 | isopropyl | H | H | CH₃ | 3-chlorophenyl | |
| 1184 | isopropyl | H | H | CH₃ | 4-chlorophenyl | |
| 1185 | isopropyl | H | H | CH₃ | 2-methylphenyl | |
| 1186 | isopropyl | H | H | CH₃ | 3-methylphenyl | |
| 1187 | isopropyl | H | H | CH₃ | 4-methylphenyl | |
| 1188 | isopropyl | H | H | CH₃ | 2-methoxyphenyl | |
| 1189 | isopropyl | H | H | CH₃ | 3-methoxyphenyl | |
| 1190 | isopropyl | H | H | CH₃ | 4-methoxyphenyl | |
| 1191 | isopropyl | H | H | CH₃ | 2,4-dichlorophenyl | |
| 1192 | isopropyl | H | H | CH₃ | 3,4-dichlorophenyl | |
| 1193 | isopropyl | H | H | CH₃ | 3,5-dichlorophenyl | |
| 1194 | isopropyl | H | H | CH₃ | 2,5-dichlorophenyl | |
| 1195 | isopropyl | H | H | CH₃ | 2,4,6-trichlorophenyl | |
| 1196 | isopropyl | H | H | CH₃ | 2-chloro-4-methylphenyl | |
| 1197 | isopropyl | H | H | CH₃ | 2-chloro-4-methoxy- | |
| | | | | | phenyl | |
| 1198 | isopropyl | H | H | CH₃ | 2,4-dimethylphenyl | |
| 1199 | isopropyl | H | H | CH₃ | 2,5-dimethylphenyl | |
| 1200 | isopropyl | H | H | CH₃ | 2,6-dimethylphenyl | |
| 1201 | isopropyl | H | H | CH₃ | 2,4,6-trimethylphenyl | |
| 1202 | isopropyl | H | H | CH₃ | 2-fluorophenyl | |
| 1203 | isopropyl | H | H | CH₃ | 3-fluorophenyl | |
| 1204 | isopropyl | H | H | CH₃ | 4-fluorophenyl | |
| 1205 | isopropyl | H | H | CH₃ | 2-chloro-4-fluorophenyl | |
| 1206 | isopropyl | H | H | CH₃ | 2,4-dimethoxyphenyl | |
| 1207 | isopropyl | H | H | CH₃ | 2,3-dimethoxyphenyl | |
| 1208 | isopropyl | H | H | CH₃ | 3,4-dimethoxyphenyl | |
| 1209 | isopropyl | H | H | CH₃ | 3,5-dimethoxyphenyl | |
| 1210 | isopropyl | H | H | CH₃ | 2,5-dimethoxyphenyl | |
| 1211 | isopropyl | H | H | CH₃ | 2-trifluoromethylphenyl | |
| 1212 | isopropyl | H | H | CH₃ | 3-trifluoromethylphenyl | |
| 1213 | isopropyl | H | H | CH₃ | 4-trifluoromethylphenyl | |
| 1214 | isopropyl | H | H | CH₃ | 3-trifluoromethyl- | |
| | | | | | 4-chlorophenyl | |
| 1215 | isopropyl | H | H | CH₃ | 3-trifluoromethyl- | |
| | | | | | 4-fluorophenyl | |
| 1216 | isopropyl | H | H | CH₃ | 2-trifluoro- | |
| | | | | | methoxyphenyl | |
| 1217 | isopropyl | H | H | CH₃ | 3-trifluoro- | |
| | | | | | methoxyphenyl | |
| 1218 | isopropyl | H | H | CH₃ | 4-trifluoro- | |
| | | | | | methoxyphenyl | |
| 1219 | isobutyl | H | H | CH₃ | phenyl | |
| 1220 | isobutyl | H | H | CH₃ | 2-chlorophenyl | |
| 1221 | isobutyl | H | H | CH₃ | 3-chlorophenyl | |
| 1222 | isobutyl | H | H | CH₃ | 4-chlorophenyl | |
| 1223 | isobutyl | H | H | CH₃ | 2-methylphenyl | |
| 1224 | isobutyl | H | H | CH₃ | 3-methylphenyl | |
| 1225 | isobutyl | H | H | CH₃ | 4-methylphenyl | |
| 1226 | isobutyl | H | H | CH₃ | 2-methoxyphenyl | |
| 1227 | isobutyl | H | H | CH₃ | 3-methoxyphenyl | |
| 1228 | isobutyl | H | H | CH₃ | 4-methoxyphenyl | |
| 1229 | isobutyl | H | H | CH₃ | 2,4-dichlorophenyl | |
| 1230 | isobutyl | H | H | CH₃ | 3,4-dichlorophenyl | |
| 1231 | isobutyl | H | H | CH₃ | 3,5-dichlorophenyl | |
| 1232 | isobutyl | H | H | CH₃ | 2,5-dichlorophenyl | |
| 1233 | isobutyl | H | H | CH₃ | 2,4,6-trichlorophenyl | |
| 1234 | isobutyl | H | H | CH₃ | 2-chloro-4-methylphenyl | |
| 1235 | isobutyl | H | H | CH₃ | 2-chloro-4-methoxy- | |
| | | | | | phenyl | |
| 1236 | isobutyl | H | H | CH₃ | 2,4-dimethylphenyl | |
| 1237 | isobutyl | H | H | CH₃ | 2,5-dimethylphenyl | |
| 1238 | isobutyl | H | H | CH₃ | 2,6-dimethylphenyl | |
| 1239 | isobutyl | H | H | CH₃ | 2,4,6-trimethylphenyl | |
| 1240 | isobutyl | H | H | CH₃ | 2-fluorophenyl | |
| 1241 | isobutyl | H | H | CH₃ | 3-fluorophenyl | |
| 1242 | isobutyl | H | H | CH₃ | 4-fluorophenyl | |
| 1243 | isobutyl | H | H | CH₃ | 2-chloro-4-fluorophenyl | |
| 1244 | isobutyl | H | H | CH₃ | 2,4-dimethoxyphenyl | |
| 1245 | isobutyl | H | H | CH₃ | 2,3-dimethoxyphenyl | |
| 1246 | isobutyl | H | H | CH₃ | 3,4-dimethoxyphenyl | |
| 1247 | isobutyl | H | H | CH₃ | 3,5-dimethoxyphenyl | |
| 1248 | isobutyl | H | H | CH₃ | 2,5-dimethoxyphenyl | |
| 1249 | isobutyl | H | H | CH₃ | 2-trifluoromethylphenyl | |
| 1250 | isobutyl | H | H | CH₃ | 3-trifluoromethylphenyl | |
| 1251 | isobutyl | H | H | CH₃ | 4-trifluoromethylphenyl | |
| 1252 | isobutyl | H | H | CH₃ | 3-trifluoromethyl- | |
| | | | | | 4-chlorophenyl | |
| 1253 | isobutyl | H | H | CH₃ | 3-trifluoromethyl- | |
| | | | | | 4-fluorophenyl | |
| 1254 | isobutyl | H | H | CH₃ | 2-trifluoro- | |
| | | | | | methoxyphenyl | |
| 1255 | isobutyl | H | H | CH₃ | 3-trifluoro- | |
| | | | | | methoxyphenyl | |
| 1256 | isobutyl | H | H | CH₃ | 4-trifluoro- | |
| | | | | | methoxyphenyl | |
| 1257 | isobutyl | H | H | CH₃ | 2-difluoro- | |
| | | | | | methoxyphenyl | |
| 1258 | isobutyl | H | H | CH₃ | 3-difluoro- | |
| | | | | | methoxyphenyl | |
| 1259 | isobutyl | H | H | CH₃ | 4-difluoro- | |
| | | | | | methoxyphenyl | |
| 1259a | benzyl | H | H | CH₃ | phenyl | |
| 1259b | benzyl | H | H | CH₃ | 3-chlorophenyl | |
| 1259c | benzyl | H | H | CH₃ | 4-chlorophenyl | 3.23¹⁾ |
| 1259d | benzyl | H | H | CH₃ | 4-methylphenyl | |
| 1260 | isobutyl | H | H | CH₃ | 2-tert-butylphenyl | |
| 1261 | isobutyl | H | H | CH₃ | 3-tert-butylphenyl | |
| 1262 | isobutyl | H | H | CH₃ | 4-tert-butylphenyl | |
| 1263 | isobutyl | H | H | CH₃ | 4-methanesulphonyl- | |
| | | | | | phenyl | |
| 1264 | 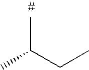 | H | H | CH₃ | phenyl | |
| 1265 | 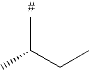 | H | H | CH₃ | 2-chlorophenyl | |
| 1266 | 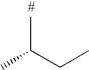 | H | H | CH₃ | 3-chlorophenyl | |
| 1267 | 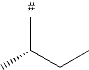 | H | H | CH₃ | 4-chlorophenyl | |
| 1268 | 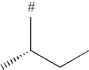 | H | H | CH₃ | 2-methylphenyl | |
| 1269 | 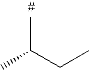 | H | H | CH₃ | 3-methylphenyl | |
| 1270 | 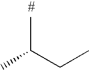 | H | H | CH₃ | 4-methylphenyl | |
| 1271 | 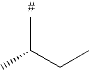 | H | H | CH₃ | 2-methoxyphenyl | |
| 1272 | 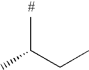 | H | H | CH₃ | 3-methoxyphenyl | |
| 1273 | 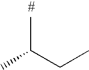 | H | H | CH₃ | 4-methoxyphenyl | |
| 1274 | 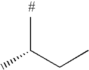 | H | H | CH₃ | 2,4-dichlorophenyl | |
| 1275 | 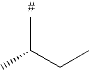 | H | H | CH₃ | 3,4-dichlorophenyl | |
| 1276 | 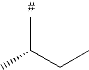 | H | H | CH₃ | 3,5-dichlorophenyl | |
| 1277 | 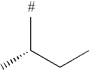 | H | H | CH₃ | 2,5-dichlorophenyl | |
| 1278 | 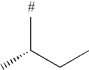 | H | H | CH₃ | 2,4,6-trichlorophenyl | |
| 1279 | 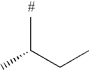 | H | H | CH₃ | 2-chloro-4-methylphenyl | |
| 1280 | 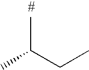 | H | H | CH₃ | 2-chloro-4-methoxy- phenyl | |
| 1281 | 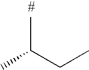 | H | H | CH₃ | 2,4-dimethylphenyl | |
| 1282 | 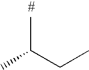 | H | H | CH₃ | 2,5-dimethylphenyl | |
| 1283 | 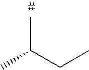 | H | H | CH₃ | 2,6-dimethylphenyl | |
| 1284 | 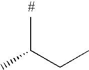 | H | H | CH₃ | 2,4,6-trimethylphenyl | |
| 1285 | 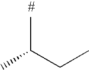 | H | H | H | phenyl | |
| 1286 | 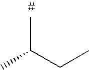 | H | H | H | 2-chlorophenyl | |
| 1287 | 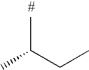 | H | H | H | 3-chlorophenyl | |
| 1288 | 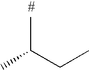 | H | H | H | 4-chlorophenyl | |
| 1289 | 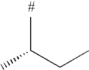 | H | H | H | 2-methylphenyl | |
| 1290 | 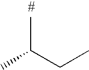 | H | H | H | 3-methylphenyl | |
| 1291 | 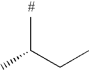 | H | H | H | 4-methylphenyl | |
| 1292 | 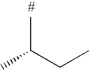 | H | H | H | 2-methoxyphenyl | |
| 1293 | 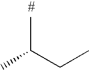 | H | H | H | 3-methoxyphenyl | |
| 1294 | 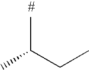 | H | H | H | 4-methoxyphenyl | |
| 1295 | 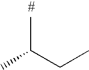 | H | H | H | 2,4-dichlorophenyl | |
| 1296 | 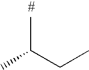 | H | H | H | 3,4-dichlorophenyl | |
| 1297 | 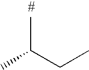 | H | H | H | 3,5-dichlorophenyl | |
| 1298 | 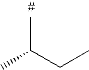 | H | H | H | 2,5-dichlorophenyl | |
| 1299 | 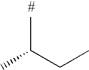 | H | H | H | 2,4,6-trichlorophenyl | |
| 1300 | 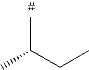 | H | H | H | 2-chloro-4-methylphenyl | |
| 1301 | 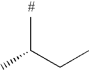 | H | H | H | 2-chloro-4-methoxy- phenyl | |
| 1302 | 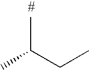 | H | H | H | 2,4-dimethylphenyl | |
| 1303 | 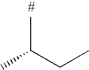 | H | H | H | 2,5-dimethylphenyl | |
| 1304 | 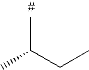 | H | H | H | 2,6-dimethylphenyl | |
| 1305 | 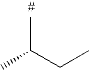 | H | H | H | 2,4,6-trimethylphenyl | |
| 1306 | 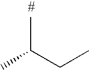 | CH₃ | H | H | phenyl | |
| 1307 | 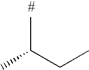 | CH₃ | H | H | 2-chlorophenyl | |
| 1308 | 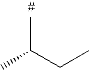 | CH₃ | H | H | 3-chlorophenyl | |
| 1309 | 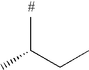 | CH₃ | H | H | 4-chlorophenyl | |
| 1310 | 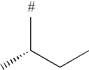 | CH₃ | H | H | 2-methylphenyl | |
| 1311 | 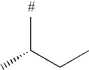 | CH₃ | H | H | 3-methylphenyl | |
| 1312 | 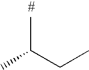 | CH₃ | H | H | 4-methylphenyl | |
| 1313 | 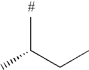 | CH₃ | H | H | 2-methoxyphenyl | |
| 1314 | 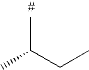 | CH₃ | H | H | 3-methoxyphenyl | |
| 1315 | 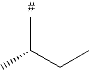 | CH₃ | H | H | 4-methoxyphenyl | |
| 1316 | 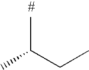 | CH₃ | H | H | 2,4-dichlorophenyl | |
| 1317 | 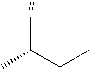 | CH₃ | H | H | 3,4-dichlorophenyl | |
| 1318 | 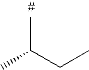 | CH₃ | H | H | 3,5-dichlorophenyl | |
| 1319 | 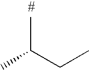 | CH₃ | H | H | 2,5-dichlorophenyl | |
| 1320 | 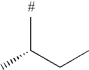 | CH₃ | H | H | 2,4,6-trichlorophenyl | |
| 1321 | 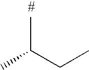 | CH₃ | H | H | 2-chloro-4-methylphenyl | |
| 1322 | 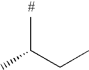 | CH₃ | H | H | 2-chloro-4-methoxy- phenyl | |
| 1323 | 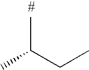 | CH₃ | H | H | 2,4-dimethylphenyl | |
| 1324 | 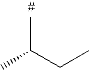 | CH₃ | H | H | 2,5-dimethylphenyl | |
| 1325 | 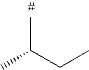 | CH₃ | H | H | 2,6-dimethylphenyl | |
| 1326 | 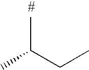 | CH₃ | H | H | 2,4,6-trimethylphenyl | |
| 1327 | 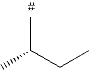 | CH₃ | H | H | 2-fluorophenyl | |
| 1328 | 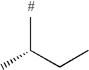 | CH₃ | H | H | 3-fluorophenyl | |
| 1329 | 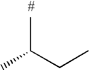 | CH₃ | H | H | 4-fluorophenyl | |
| 1330 | 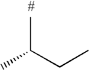 | CH₃ | H | H | 2-chloro-4-fluorophenyl | |
| 1331 | 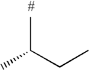 | CH₃ | H | H | 2,4-dimethoxyphenyl | |
| 1332 | 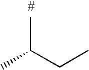 | CH₃ | H | H | 2,3-dimethoxyphenyl | |
| 1333 | 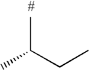 | CH₃ | H | H | 3,4-dimethoxyphenyl | |
| 1334 | 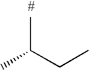 | CH₃ | H | H | 3,5-dimethoxyphenyl | |
| 1335 | 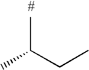 | CH₃ | H | H | 2,5-dimethoxyphenyl | |
| 1336 | 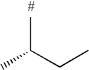 | CH₃ | H | H | 2-trifluoromethylphenyl | |
| 1337 | 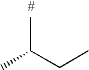 | CH₃ | H | H | 3-trifluoromethylphenyl | |
| 1338 | 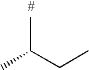 | CH₃ | H | H | 4-trifluoromethylphenyl | |
| 1339 | 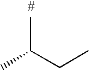 | CH₃ | H | H | 3-trifluoromethyl- 4-chlorophenyl | |
| 1340 | 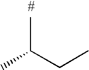 | CH₃ | H | H | 3-trifluoromethyl- 4-fluorophenyl | |
| 1341 | 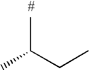 | CH₃ | H | H | 2-trifluoro- methoxyphenyl | |
| 1342 | 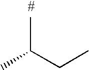 | CH₃ | H | H | 3-trifluoro- methoxyphenyl | |
| 1343 | 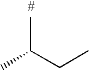 | CH₃ | H | H | 4-trifluoro- methoxyphenyl | |
| 1344 | 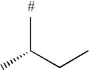 | CH₃ | H | H | 2-difluoro- methoxyphenyl | |
| 1345 | 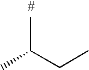 | CH₃ | H | H | 3-difluoro- methoxyphenyl | |
| 1346 | 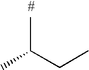 | CH₃ | H | H | 4-difluoro- methoxyphenyl | |
| 1347 | 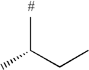 | CH₃ | H | H | 2-tert-butylphenyl | |
| 1348 | 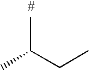 | CH₃ | H | H | 3-tert-butylphenyl | |
| 1349 | 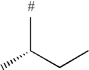 | CH₃ | H | H | 4-tert-butylphenyl | |
| 1350 | 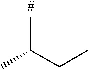 | CH₃ | H | H | 2-phenoxyphenyl | |
| 1351 | 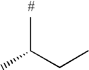 | CH₃ | H | H | 3-phenoxyphenyl | |
| 1352 | 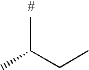 | CH₃ | H | H | 4-phenoxyphenyl | |
| 1353 | 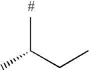 | CH₃ | H | H | 2-(4-methoxyphenoxy)- phenyl | |
| 1354 | 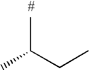 | CH₃ | H | H | 3-(4-methoxyphenoxy)- phenyl | |
| 1355 | 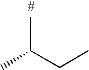 | CH₃ | H | H | 4-(4-methoxyphenoxy)- phenyl | |
| 1356 | 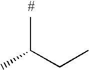 | CH₃ | H | H | 4-methanesulphonyl- phenyl | |
| 1357 | 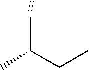 | CH₃ | H | H | 2-ethoxyphenyl | |
| 1358 | 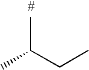 | CH₃ | H | H | 3-ethoxyphenyl | |
| 1359 | 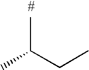 | CH₃ | H | H | 4-ethoxyphenyl | |
| 1360 | 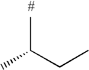 | CH₃ | H | H | methyl | |
| 1361 | 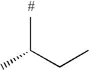 | CH₃ | H | H | ethyl | |
| 1362 | 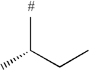 | CH₃ | H | H | n-propyl | |
| 1363 | 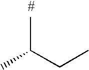 | CH₃ | H | H | n-butyl | |
| 1364 | 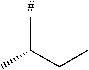 | CH₃ | H | H | n-pentyl | |
| 1365 | 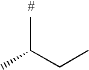 | CH₃ | H | H | benzyl | |
| 1366 | 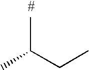 | CH₃ | H | H | 2-chlorobenzyl | |
| 1367 | 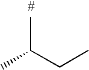 | CH₃ | H | H | 3-chlorobenzyl | |
| 1368 | 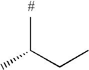 | CH₃ | H | H | 4-chlorobenzyl | |
| 1369 | 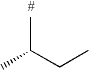 | CH₃ | H | H | 2-methylbenzyl | |
| 1370 | 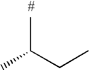 | CH₃ | H | H | 3-methylbenzyl | |
| 1371 | 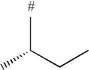 | CH₃ | H | H | 4-methylbenzyl | |
| 1372 | 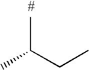 | CH₃ | H | H | 2-methoxybenzyl | |
| 1373 | 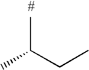 | CH₃ | H | H | 3-methoxybenzyl | |
| 1374 | 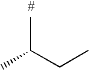 | CH₃ | H | H | 4-methoxybenzyl | |
| 1375 | 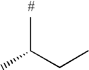 | CH₃ | H | H | 2,4-dichlorobenzyl | |
| 1376 | 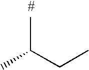 | CH₃ | H | H | 3,4-dichlorobenzyl | |
| 1377 | 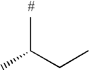 | CH₃ | H | H | 3,5-dichlorobenzyl | |
| 1378 | 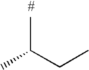 | CH₃ | H | H | 2,5-dichlorobenzyl | |
| 1379 | 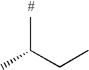 | CH₃ | H | H | 2,4,6-trichlorobenzyl | |
| 1380 | 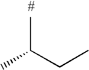 | CH₃ | H | H | 2-chloro-4-methylbenzyl | |
| 1381 | 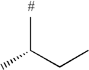 | CH₃ | H | H | 2-chloro-4-methoxy- benzyl | |
| 1382 | 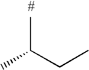 | CH₃ | H | H | 2,4-dimethylbenzyl | |
| 1383 | 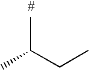 | CH₃ | H | H | 2,5-dimethylbenzyl | |
| 1384 | 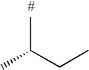 | CH₃ | H | H | 2,6-dimethylbenzyl | |
| 1385 | 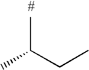 | CH₃ | H | H | 2,4,6-trimethylbenzyl | |
| 1386 | 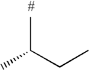 | CH₃ | H | H | 2-fluorobenzyl | |
| 1387 | 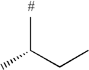 | CH₃ | H | H | 3-fluorobenzyl | |
| 1388 | 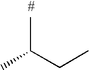 | CH₃ | H | H | 4-fluorobenzyl | |
| 1389 | 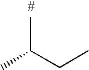 | CH₃ | H | H | 2-chloro-4-fluorobenzyl | |
| 1390 | 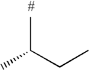 | CH₃ | H | H | 2,4-dimethoxybenzyl | |
| 1391 | 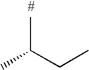 | CH₃ | H | H | 2,3-dimethoxybenzyl | |
| 1392 | 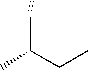 | CH₃ | H | H | 3,4-dimethoxybenzyl | |
| 1393 | 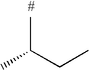 | CH₃ | H | H | 3,5-dimethoxybenzyl | |
| 1394 | 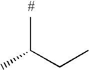 | CH₃ | H | H | 2,5-dimethoxybenzyl | |
| 1395 | 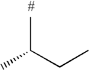 | CH₃ | H | H | 2-trifluoromethylbenzyl | |
| 1396 | 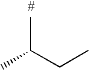 | CH₃ | H | H | 3-trifluoromethylbenzyl | |
| 1397 | 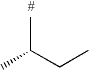 | CH₃ | H | H | 4-trifluoromethylbenzyl | |
| 1398 | 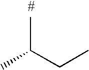 | CH₃ | H | H | 3-trifluoromethyl-4- chlorobenzyl | |
| 1399 | 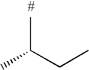 | CH₃ | H | H | 3-trifluoromethyl- 4-fluorobenzyl | |
| 1400 | 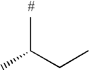 | CH₃ | H | H | 2-trifluoro- methoxybenzyl | |
| 1401 | 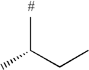 | CH₃ | H | H | 3-trifluoro- methoxybenzyl | |
| 1402 | 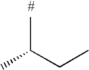 | CH₃ | H | H | 4-trifluoro- methoxybenzyl | |
| 1403 | 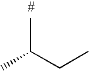 | CH₃ | H | H | 2-difluoro- methoxybenzyl | |
| 1404 | 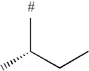 | CH₃ | H | H | 3-difluoro- methoxybenzyl | |
| 1405 | 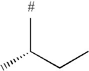 | CH₃ | H | H | 4-difluoro- methoxybenzyl | |
| 1406 | 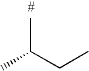 | CH₃ | H | H | 2-tert-butylbenzyl | |
| 1407 | 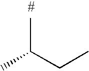 | CH₃ | H | H | 3-tert-butylbenzyl | |
| 1408 | 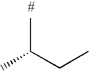 | CH₃ | H | H | 4-tert-butylbenzyl | |
| 1409 | 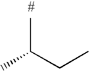 | CH₃ | H | H | 2-phenoxybenzyl | |
| 1410 | 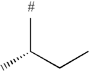 | CH₃ | H | H | 3-phenoxybenzyl | |
| 1411 | 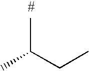 | CH₃ | H | H | 4-phenoxybenzyl | |
| 1412 | 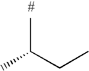 | CH₃ | H | H | 2-(4-methoxyphenoxy)- benzyl | |
| 1413 | 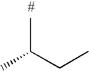 | CH₃ | H | H | 3-(4-methoxyphenoxy)- benzyl | |
| 1414 | 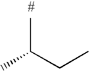 | CH₃ | H | H | 4-(4-methoxyphenoxy)- benzyl | |
| 1415 | 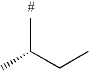 | CH₃ | H | H | 4-methanesulphonyl- benzyl | |
| 1416 | 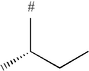 | CH₃ | H | H | 2-ethoxybenzyl | |
| 1417 | 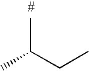 | CH₃ | H | H | 3-ethoxybenzyl | |
| 1418 | 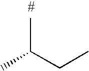 | CH₃ | H | H | 4-ethoxybenzyl | |
| 1419 | 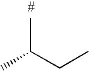 | CH₃ | H | H | phenethyl | |
| 1420 | 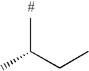 | CH₃ | H | H | 2-chlorophenethyl | |
| 1421 | 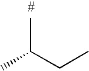 | CH₃ | H | H | 3-chlorophenethyl | |
| 1422 | 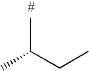 | CH₃ | H | H | 4-chlorophenethyl | |
| 1423 | 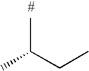 | CH₃ | H | H | 2-methylphenethyl | |
| 1424 | 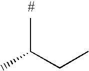 | CH₃ | H | H | 3-methylphenethyl | |
| 1425 | 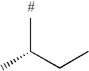 | CH₃ | H | H | 4-methylphenethyl | |
| 1426 | 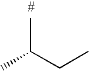 | CH₃ | H | H | 2-methoxyphenethyl | |
| 1427 | 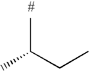 | CH₃ | H | H | 3-methoxyphenethyl | |
| 1428 | 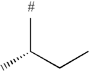 | CH₃ | H | H | 4-methoxyphenethyl | |
| 1429 | 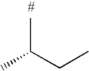 | CH₃ | H | H | 2,4-dichlorophenethyl | |
| 1430 | 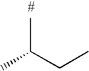 | CH₃ | H | H | 3,4-dichlorophenethyl | |
| 1431 | 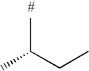 | CH₃ | H | H | 3,5-dichlorophenethyl | |
| 1432 | 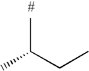 | CH₃ | H | H | 2,5-dichlorophenethyl | |
| 1433 | 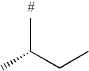 | CH₃ | H | H | 2,4,6-trichlorophenethyl | |
| 1434 | 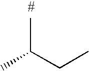 | CH₃ | H | H | 2-chloro- 4-methylphenethyl | |
| 1435 | 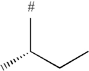 | CH₃ | H | H | 2-chloro-4-methoxy- phenethyl | |
| 1436 | 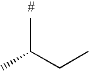 | CH₃ | H | H | 2,4-dimethylphenethyl | |
| 1437 | 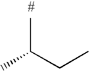 | CH₃ | H | H | 2,6-dimethylphenethyl | |
| 1438 | 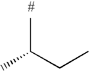 | CH₃ | H | H | 2,4,6-trimethylphenethyl | |
| 1439 | 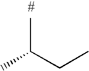 | CH₃ | H | H | 2-fluorophenethyl | |
| 1440 | 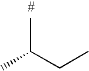 | CH₃ | H | H | 3-fluorophenethyl | |
| 1441 | 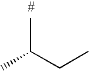 | CH₃ | H | H | 4-fluorophenethyl | |
| 1442 | 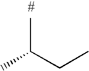 | CH₃ | H | H | 2-chloro- 4-fluorophenethyl | |
| 1443 | 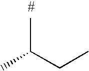 | CH₃ | H | H | 2,4-dimethoxyphenethyl | |
| 1444 | 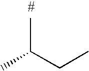 | CH₃ | H | H | 3,4-dimethoxyphenethyl | |
| 1445 | 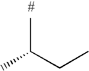 | CH₃ | H | H | 3,5-dimethoxyphenethyl | |
| 1446 | 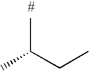 | CH₃ | H | H | 2,5-dimethoxyphenethyl | |
| 1447 | 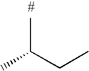 | CH₃ | H | H | 2-trifluoromethyl- phenethyl | |
| 1448 | 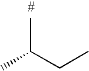 | CH₃ | H | H | 3-trifluoromethyl- phenethyl | |
| 1449 | 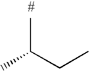 | CH₃ | H | H | 4-trifluoromethyl- phenethyl | |
| 1450 | 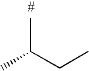 | CH₃ | H | H | 2-trifluoro- methoxyphenethyl | |
| 1451 | 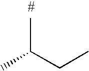 | CH₃ | H | H | 3-trifluoro- methoxyphenethyl | |
| 1452 | 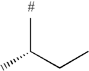 | CH₃ | H | H | 4-trifluoro- methoxyphenethyl | |
| 1453 | 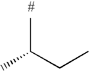 | CH₃ | H | H | 2-difluoro- methoxyphenethyl | |
| 1454 | 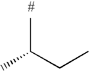 | CH₃ | H | H | 3-difluoro- methoxyphenethyl | |
| 1455 | 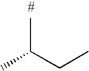 | CH₃ | H | H | 4-difluoro- methoxyphenethyl | |
| 1456 | 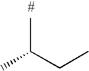 | CH₃ | H | H | 2-tert-butylphenethyl | |
| 1457 | 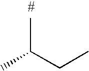 | CH₃ | H | H | 3-tert-butylphenethyl | |
| 1458 | 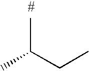 | CH₃ | H | H | 4-tert-butylphenethyl | |
| 1459 | 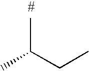 | CH₃ | H | H | tert-butoxycarbonyl | |
| 1460 | 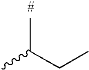 | CH₃ | H | H | phenyl | |
| 1461 | 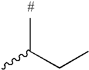 | CH₃ | H | H | 2-chlorophenyl | |
| 1462 | 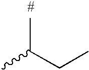 | CH₃ | H | H | 3-chlorophenyl | |
| 1463 | 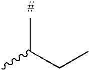 | CH₃ | H | H | 4-chlorophenyl | |
| 1464 | 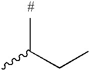 | CH₃ | H | H | 2-methylphenyl | |
| 1465 | 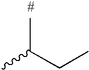 | CH₃ | H | H | 3-methylphenyl | |
| 1466 | 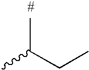 | CH₃ | H | H | 4-methylphenyl | |
| 1467 | 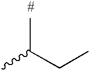 | CH₃ | H | H | 2-methoxyphenyl | |
| 1468 | 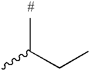 | CH₃ | H | H | 3-methoxyphenyl | |
| 1469 | 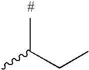 | CH₃ | H | H | 4-methoxyphenyl | |
| 1470 | 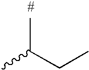 | CH₃ | H | H | 2,4-dichlorophenyl | |
| 1471 | 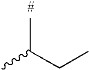 | CH₃ | H | H | 3,4-dichlorophenyl | |
| 1472 | 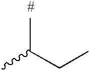 | CH₃ | H | H | 3,5-dichlorophenyl | |
| 1473 | 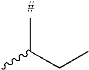 | CH₃ | H | H | 2,5-dichlorophenyl | |
| 1474 | 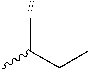 | CH₃ | H | H | 2,4,6-trichlorophenyl | |
| 1475 | 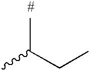 | CH₃ | H | H | 2-chloro-4-methylphenyl | |
| 1476 | 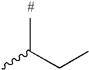 | CH₃ | H | H | 2-chloro-4-methoxy- phenyl | |
| 1477 | 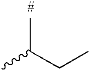 | CH₃ | H | H | 2,4-dimethylphenyl | |
| 1478 | 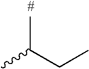 | CH₃ | H | H | 2,5-dimethylphenyl | |
| 1479 | 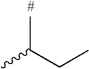 | CH₃ | H | H | 2,6-dimethylphenyl | |
| 1480 | 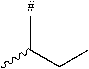 | CH₃ | H | H | 2,4,6-trimethylphenyl | |
| 1481 | 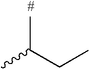 | CH₃ | H | H | 2-fluorophenyl | |
| 1482 | 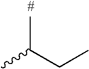 | CH₃ | H | H | 3-fluorophenyl | |
| 1483 | 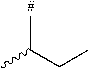 | CH₃ | H | H | 4-fluorophenyl | |
| 1484 | 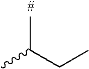 | CH₃ | H | H | 2-chloro-4-fluorophenyl | |
| 1485 | 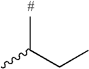 | CH₃ | H | H | 2,4-dimethoxyphenyl | |
| 1486 | 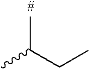 | CH₃ | H | H | 2,3-dimethoxyphenyl | |
| 1487 | 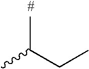 | CH₃ | H | H | 3,4-dimethoxyphenyl | |
| 1488 | 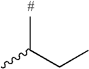 | CH₃ | H | H | 3,5-dimethoxyphenyl | |
| 1489 | 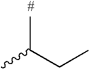 | CH₃ | H | H | 2,5-dimethoxyphenyl | |
| 1490 | 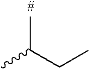 | CH₃ | H | H | 2-trifluoromethylphenyl | |
| 1491 | 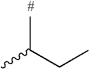 | CH₃ | H | H | 3-trifluoromethylphenyl | |
| 1492 | 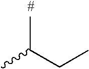 | CH₃ | H | H | 4-trifluoromethylphenyl | |
| 1493 | 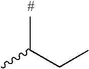 | CH₃ | H | H | 3-trifluoromethyl- 4-chlorophenyl | |
| 1494 | 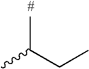 | CH₃ | H | H | 3-trifluoromethyl- 4-fluorophenyl | |
| 1495 | 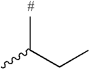 | CH₃ | H | H | 2-trifluoro- methoxyphenyl | |
| 1496 | 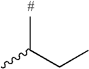 | CH₃ | H | H | 3-trifluoro- methoxyphenyl | |
| 1497 | 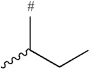 | CH₃ | H | H | 4-trifluoro- methoxyphenyl | |
| 1498 | 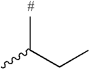 | CH₃ | H | H | 2.-difluoro- methoxyphenyl | |
| 1499 | 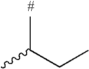 | CH₃ | H | H | 3-difluoro- methoxyphenyl | |
| 1500 | 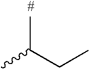 | CH₃ | H | H | 4-difluoro- methoxyphenyl | |
| 1501 | 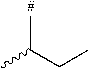 | CH₃ | H | H | 2-tert-butylphenyl | |
| 1502 | 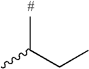 | CH₃ | H | H | 3-tert-butylphenyl | |
| 1503 | 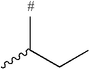 | CH₃ | H | H | 4-tert-butylphenyl | |
| 1504 | 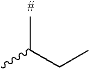 | CH₃ | H | H | 2-phenoxyphenyl | |
| 1505 | 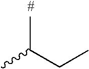 | CH₃ | H | H | 3-phenoxyphenyl | |
| 1506 | 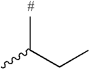 | CH₃ | H | H | 4-phenoxyphenyl | |
| 1507 | 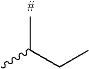 | CH₃ | H | H | 2-(4-methoxyphenoxy)- phenyl | |
| 1508 | 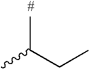 | CH₃ | H | H | 3-(4-methoxyphenoxy)- phenyl | |
| 1509 | 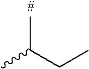 | CH₃ | H | H | 4-(4-methoxyphenoxy)- phenyl | |
| 1510 | 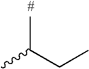 | CH₃ | H | H | 4-methanesulphonyl- phenyl | |
| 1511 | 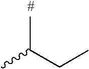 | CH₃ | H | H | 2-ethoxyphenyl | |
| 1512 | 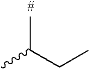 | CH₃ | H | H | 3-ethoxyphenyl | |
| 1513 | 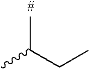 | CH₃ | H | H | 4-ethoxyphenyl | |
| 1514 | 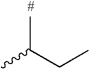 | CH₃ | H | H | methyl | |
| 1515 | 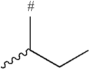 | CH₃ | H | H | ethyl | |
| 1516 | 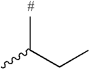 | CH₃ | H | H | n-propyl | |
| 1517 | 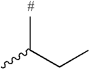 | CH₃ | H | H | n-butyl | |
| 1518 | 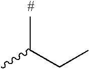 | CH₃ | H | H | n-pentyl | |
| 1519 | 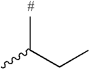 | CH₃ | H | H | benzyl | |
| 1520 | 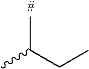 | CH₃ | H | H | 2-chlorobenzyl | |
| 1521 | 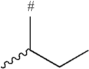 | CH₃ | H | H | 3-chlorobenzyl | |
| 1522 | 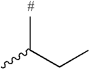 | CH₃ | H | H | 4-chlorobenzyl | |
| 1523 | 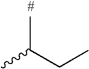 | CH₃ | H | H | 2-methylbenzyl | |
| 1524 | 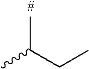 | CH₃ | H | H | 3-methylbenzyl | |
| 1525 | 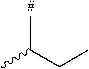 | CH₃ | H | H | 4-methylbenzyl | |
| 1526 | 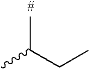 | CH₃ | H | H | 2-methoxybenzyl | |
| 1527 | 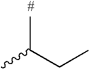 | CH₃ | H | H | 3-methoxybenzyl | |
| 1528 | 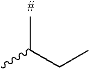 | CH₃ | H | H | 4-methoxybenzyl | |
| 1529 | 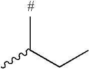 | CH₃ | H | H | 2,4-dichlorobenzyl | |
| 1530 | 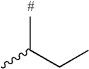 | CH₃ | H | H | 3,4-dichlorobenzyl | |
| 1531 | 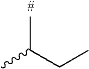 | CH₃ | H | H | 3,5-dichlorobenzyl | |
| 1532 | 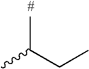 | CH₃ | H | H | 2,5-dichlorobenzyl | |
| 1533 | 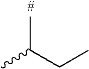 | CH₃ | H | H | 2,4,6-trichlorobenzyl | |
| 1534 | 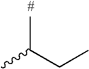 | CH₃ | H | H | 2-chloro-4-methylbenzyl | |
| 1535 | 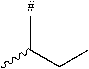 | CH₃ | H | H | 2-chloro-4-methoxy- benzyl | |
| 1536 | 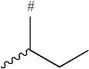 | CH₃ | H | H | 2,4-dimethylbenzyl | |
| 1537 | 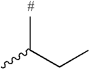 | CH₃ | H | H | 2,5-dimethylbenzyl | |
| 1538 | 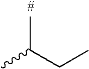 | CH₃ | H | H | 2,6-dimethylbenzyl | |
| 1539 | 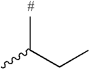 | CH₃ | H | H | 2,4,6-trimethylbenzyl | |
| 1540 | 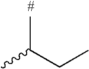 | CH₃ | H | H | 2-fluorobenzyl | |
| 1541 | 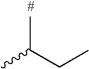 | CH₃ | H | H | 3-fluorobenzyl | |
| 1542 | 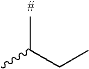 | CH₃ | H | H | 4-fluorobenzyl | |
| 1543 | 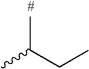 | CH₃ | H | H | 2-chloro-4-fluorobenzyl | |
| 1544 | 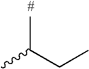 | CH₃ | H | H | 2,4-dimethoxybenzyl | |
| 1545 | 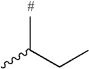 | CH₃ | H | H | 2,3-dimethoxybenzyl | |
| 1546 | 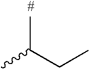 | CH₃ | H | H | 3,4-dimethoxybenzyl | |
| 1547 | 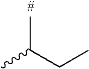 | CH₃ | H | H | 3,5-dimethoxybenzyl | |
| 1548 | 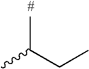 | CH₃ | H | H | 2,5-dimethoxybenzyl | |
| 1549 | 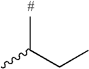 | CH₃ | H | H | 2-trifluoromethylbenzyl | |
| 1550 | 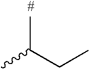 | CH₃ | H | H | 3-trifluoromethylbenzyl | |
| 1551 | 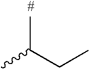 | CH₃ | H | H | 4-trifluoromethylbenzyl | |
| 1552 | 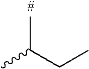 | CH₃ | H | H | 3-trifluoromethyl-4- chlorobenzyl | |
| 1553 | 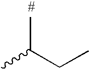 | CH₃ | H | H | 3-trifluoromethyl- 4-fluorobenzyl | |
| 1554 | 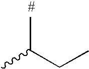 | CH₃ | H | H | 2-trifluoro- methoxybenzyl | |
| 1555 | 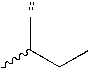 | CH₃ | H | H | 3-trifluoro- methoxybenzyl | |
| 1556 | 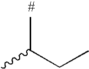 | CH₃ | H | H | 4-trifluoro- methoxybenzyl | |
| 1557 | 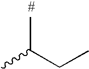 | CH₃ | H | H | 2-difluoro- methoxybenzyl | |
| 1558 | 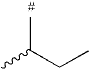 | CH₃ | H | H | 3-difluoro- methoxybenzyl | |
| 1559 | 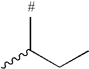 | CH₃ | H | H | 4-difluoro- methoxybenzyl | |
| 1560 | 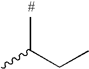 | CH₃ | H | H | 2-tert-butylbenzyl | |
| 1561 | 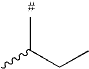 | CH₃ | H | H | 3-tert-butylbenzyl | |
| 1562 | 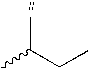 | CH₃ | H | H | 4-tert-butylbenzyl | |
| 1563 | 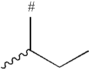 | CH₃ | H | H | 2-phenoxybenzyl | |
| 1564 | 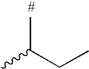 | CH₃ | H | H | 3-phenoxybenzyl | |
| 1565 | 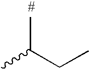 | CH₃ | H | H | 4-phenoxybenzyl | |
| 1566 | 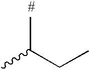 | CH₃ | H | H | 2-(4-methoxyphenoxy)- benzyl | |
| 1567 | 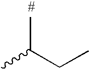 | CH₃ | H | H | 3-(4-methoxyphenoxy)- benzyl | |
| 1568 | 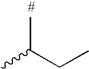 | CH₃ | H | H | 4-(4-methoxyphenoxy)- benzyl | |
| 1569 | 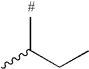 | CH₃ | H | H | 4-methanesulphonyl- benzyl | |
| 1570 | 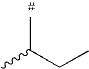 | CH₃ | H | H | 2-ethoxybenzyl | |
| 1571 | 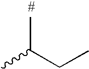 | CH₃ | H | H | 3-ethoxybenzyl | |
| 1572 | 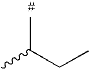 | CH₃ | H | H | 4-ethoxybenzyl | |
| 1573 | 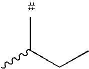 | CH₃ | H | H | phenethyl | |
| 1574 | 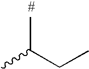 | CH₃ | H | H | 2-chlorophenethyl | |
| 1575 | 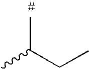 | CH₃ | H | H | 3-chlorophenethyl | |
| 1576 | 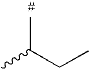 | CH₃ | H | H | 4-chlorophenethyl | |
| 1577 | 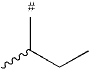 | CH₃ | H | H | 2-methylphenethyl | |
| 1578 | 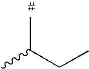 | CH₃ | H | H | 3-methylphenethyl | |
| 1579 | 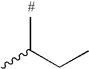 | CH₃ | H | H | 4-methylphenethyl | |
| 1580 | 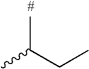 | CH₃ | H | H | 2-methoxyphenethyl | |
| 1581 | 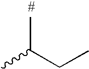 | CH₃ | H | H | 3-methoxyphenethyl | |
| 1582 | 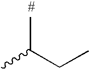 | CH₃ | H | H | 4-methoxyphenethyl | |
| 1583 | 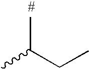 | CH₃ | H | H | 2,4-dichlorophenethyl | |
| 1584 | 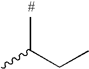 | CH₃ | H | H | 3,4-dichlorophenethyl | |
| 1585 | 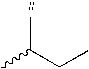 | CH₃ | H | H | 3,5-dichlorophenethyl | |
| 1586 | 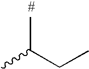 | CH₃ | H | H | 2,5-dichlorophenethyl | |
| 1587 | 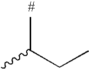 | CH₃ | H | H | 2,4,6-trichlorophenethyl | |
| 1588 | 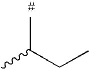 | CH₃ | H | H | 2-chloro- 4-methylphenethyl | |
| 1589 | 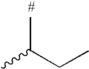 | CH₃ | H | H | 2-chloro-4-methoxy- phenethyl | |
| 1590 | 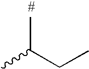 | CH₃ | H | H | 2,4-dimethylphenethyl | |
| 1591 | 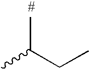 | CH₃ | H | H | 2,6-dimethylphenethyl | |
| 1592 | 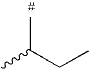 | CH₃ | H | H | 2,4,6-trimethylphenethyl | |
| 1593 | 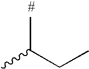 | CH₃ | H | H | 2-fluorophenethyl | |
| 1594 | 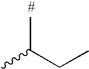 | CH₃ | H | H | 3-fluorophenethyl | |
| 1595 | 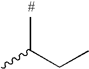 | CH₃ | H | H | 4-fluorophenethyl | |
| 1596 | 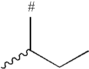 | CH₃ | H | H | 2-chloro- 4-fluorophenethyl | |
| 1597 | 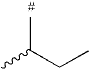 | CH₃ | H | H | 2,4-dimethoxyphenethyl | |
| 1598 | 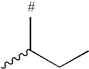 | CH₃ | H | H | 3,4-dimethoxyphenethyl | |
| 1599 | 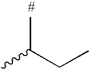 | CH₃ | H | H | 3,5-dimethoxyphenethyl | |
| 1600 | 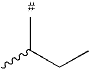 | CH₃ | H | H | 2,5-dimethoxyphenethyl | |
| 1601 | 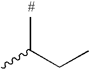 | CH₃ | H | H | 2-trifluoromethyl- phenethyl | |
| 1602 | 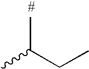 | CH₃ | H | H | 3-trifluoromethyl- phenethyl | |
| 1603 | 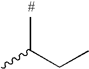 | CH₃ | H | H | 4-trifluoromethyl- phenethyl | |
| 1604 | 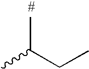 | CH₃ | H | H | 2-trifluoro- methoxyphenethyl | |
| 1605 | 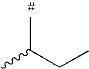 | CH₃ | H | H | 3-trifluoro- methoxyphenethyl | |
| 1606 | 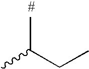 | CH₃ | H | H | 4-trifluoro- methoxyphenethyl | |
| 1607 | 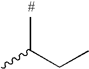 | CH₃ | H | H | 2-difluoro- methoxyphenethyl | |
| 1608 | 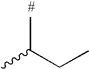 | CH₃ | H | H | 3-difluoro- methoxyphenethyl | |
| 1609 | 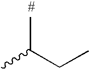 | CH₃ | H | H | 4-difluoro- methoxyphenethyl | |
| 1610 | 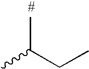 | CH₃ | H | H | 2-tert-butylphenethyl | |
| 1611 | 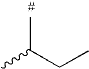 | CH₃ | H | H | 3-tert-butylphenethyl | |
| 1612 | 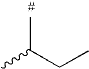 | CH₃ | H | H | 4-tert-butylphenethyl | |
| 1613 | cyclohexyl | CH₃ | H | H | phenyl | |
| 1614 | cyclohexyl | CH₃ | H | H | 2-chlorophenyl | |
| 1615 | cyclohexyl | CH₃ | H | H | 3-chlorophenyl | |
| 1616 | cyclohexyl | CH₃ | H | H | 4-chlorophenyl | |
| 1617 | cyclohexyl | CH₃ | H | H | 2-methylphenyl | |
| 1618 | cyclohexyl | CH₃ | H | H | 3-methylphenyl | |
| 1619 | cyclohexyl | CH₃ | H | H | 4-methylphenyl | |
| 1620 | cyclohexyl | CH₃ | H | H | 2-methoxyphenyl | |
| 1621 | cyclohexyl | CH₃ | H | H | 3-methoxyphenyl | |
| 1622 | cyclohexyl | CH₃ | H | H | 4-methoxyphenyl | |
| 1623 | cyclohexyl | CH₃ | H | H | 2,4-dichlorophenyl | |
| 1624 | cyclohexyl | CH₃ | H | H | 3,4-dichlorophenyl | |
| 1625 | cyclohexyl | CH₃ | H | H | 3,5-dichlorophenyl | |
| 1626 | cyclohexyl | CH₃ | H | H | 2,5-dichlorophenyl | |
| 1627 | cyclohexyl | CH₃ | H | H | 2,4,6-trichlorophenyl | |
| 1628 | cyclohexyl | CH₃ | H | H | 2-chloro-4-methylphenyl | |
| 1629 | cyclohexyl | CH₃ | H | H | 2-chloro-4-methoxy- | |
| | | | | | phenyl | |
| 1630 | cyclohexyl | CH₃ | H | H | 2,4-dimethylphenyl | |
| 1631 | cyclohexyl | CH₃ | H | H | 2,5-dimethylphenyl | |
| 1632 | cyclohexyl | CH₃ | H | H | 2,6-dimethylphenyl | |
| 1633 | cyclohexyl | CH₃ | H | H | 2,4,6-trimethylphenyl | |
| 1634 | cyclohexyl | CH₃ | H | H | 2-fluorophenyl | |
| 1635 | cyclohexyl | CH₃ | H | H | 3-fluorophenyl | |
| 1636 | cyclohexyl | CH₃ | H | H | 4-fluorophenyl | |
| 1637 | cyclohexyl | CH₃ | H | H | 2-chloro-4-fluorophenyl | |
| 1638 | cyclohexyl | CH₃ | H | H | 2,4-dimethoxyphenyl | |
| 1639 | cyclohexyl | CH₃ | H | H | 2,3-dimethoxyphenyl | |
| 1640 | cyclohexyl | CH₃ | H | H | 3,4-dimethoxyphenyl | |
| 1641 | cyclohexyl | CH₃ | H | H | 3,5-dimethoxyphenyl | |
| 1642 | cyclohexyl | CH₃ | H | H | 2,5-dimethoxyphenyl | |
| 1643 | cyclohexyl | CH₃ | H | H | 2-trifluoromethylphenyl | |
| 1644 | cyclohexyl | CH₃ | H | H | 3-trifluoromethylphenyl | |
| 1645 | cyclohexyl | CH₃ | H | H | 4-trifluoromethylphenyl | |
| 1646 | cyclohexyl | CH₃ | H | H | 3-trifluoromethyl- | |
| | | | | | 4-chlorophenyl | |
| 1647 | cyclohexyl | CH₃ | H | H | 3-trifluoromethyl- | |
| | | | | | 4-fluorophenyl | |
| 1648 | cyclohexyl | CH₃ | H | H | 2-trifluoro- | |
| | | | | | methoxyphenyl | |
| 1649 | cyclohexyl | CH₃ | H | H | 3-trifluoro- | |
| | | | | | methoxyphenyl | |
| 1650 | cyclohexyl | CH₃ | H | H | 4-trifluoro- | |
| | | | | | methoxyphenyl | |
| 1651 | cyclohexyl | CH₃ | H | H | 2-difluoro- | |
| | | | | | methoxyphenyl | |
| 1652 | cyclohexyl | CH₃ | H | H | 3-difluoro- | |
| | | | | | methoxyphenyl | |
| 1653 | cyclohexyl | CH₃ | H | H | 4-difluoro- | |
| | | | | | methoxyphenyl | |
| 1654 | cyclohexyl | CH₃ | H | H | 2-tert-butylphenyl | |
| 1655 | cyclohexyl | CH₃ | H | H | 3-tert-butylphenyl | |
| 1656 | cyclohexyl | CH₃ | H | H | 4-tert-butylphenyl | |
| 1657 | cyclohexyl | CH₃ | H | H | 2-phenoxyphenyl | |
| 1658 | cyclohexyl | CH₃ | H | H | 3-phenoxyphenyl | |
| 1659 | cyclohexyl | CH₃ | H | H | 4-phenoxyphenyl | |
| 1660 | cyclohexyl | CH₃ | H | H | 2-(4-methoxyphenoxy)- | |
| | | | | | phenyl | |
| 1661 | cyclohexyl | CH₃ | H | H | 3-(4-methoxyphenoxy)- | |
| | | | | | phenyl | |
| 1662 | cyclohexyl | CH₃ | H | H | 4-(4-methoxyphenoxy)- | |
| | | | | | phenyl | |
| 1663 | cyclohexyl | CH₃ | H | H | 4-methanesulphonyl- | |
| | | | | | phenyl | |
| 1664 | cyclohexyl | CH₃ | H | H | 2-ethoxyphenyl | |
| 1665 | cyclohexyl | CH₃ | H | H | 3-ethoxyphenyl | |
| 1666 | cyclohexyl | CH₃ | H | H | 4-ethoxyphenyl | |
| 1667 | cyclohexyl | CH₃ | H | H | methyl | |
| 1668 | cyclohexyl | CH₃ | H | H | ethyl | |
| 1669 | cyclohexyl | CH₃ | H | H | n-propyl | |
| 1670 | cyclohexyl | CH₃ | H | H | n-butyl | |
| 1671 | cyclohexyl | CH₃ | H | H | n-pentyl | |
| 1672 | cyclohexyl | CH₃ | H | H | benzyl | |
| 1673 | cyclohexyl | CH₃ | H | H | 2-chlorobenzyl | |
| 1674 | cyclohexyl | CH₃ | H | H | 3-chlorobenzyl | |
| 1675 | cyclohexyl | CH₃ | H | H | 4-chlorobenzyl | |
| 1676 | cyclohexyl | CH₃ | H | H | 2-methylbenzyl | |
| 1677 | cyclohexyl | CH₃ | H | H | 3-methylbenzyl | |
| 1678 | cyclohexyl | CH₃ | H | H | 4-methylbenzyl | |
| 1679 | cyclohexyl | CH₃ | H | H | 2-methoxybenzyl | |
| 1680 | cyclohexyl | CH₃ | H | H | 3-methoxybenzyl | |
| 1681 | cyclohexyl | CH₃ | H | H | 4-methoxybenzyl | |
| 1682 | cyclohexyl | CH₃ | H | H | 2,4-dichlorobenzyl | |
| 1683 | cyclohexyl | CH₃ | H | H | 3,4-dichlorobenzyl | |
| 1684 | cyclohexyl | CH₃ | H | H | 3,5-dichlorobenzyl | |
| 1685 | cyclohexyl | CH₃ | H | H | 2,5-dichlorobenzyl | |
| 1686 | cyclohexyl | CH₃ | H | H | 2,4,6-trichlorobenzyl | |
| 1687 | cyclohexyl | CH₃ | H | H | 2-chloro-4-methylbenzyl | |
| 1688 | cyclohexyl | CH₃ | H | H | 2-chloro-4-methoxy- | |
| | | | | | benzyl | |
| 1689 | cyclohexyl | CH₃ | H | H | 2,4-dimethylbenzyl | |
| 1690 | cyclohexyl | CH₃ | H | H | 2,5-dimethylbenzyl | |
| 1691 | cyclohexyl | CH₃ | H | H | 2,6-dimethylbenzyl | |
| 1692 | cyclohexyl | CH₃ | H | H | 2,4,6-trimethylbenzyl | |
| 1693 | cyclohexyl | CH₃ | H | H | 2-fluorobenzyl | |
| 1694 | cyclohexyl | CH₃ | H | H | 3-fluorobenzyl | |
| 1695 | cyclohexyl | CH₃ | H | H | 4-fluorobenzyl | |
| 1696 | cyclohexyl | CH₃ | H | H | 2-chloro-4-fluorobenzyl | |
| 1697 | cyclohexyl | CH₃ | H | H | 2,4-dimethoxybenzyl | |
| 1698 | cyclohexyl | CH₃ | H | H | 2,3-dimethoxybenzyl | |
| 1699 | cyclohexyl | CH₃ | H | H | 3,4-dimethoxybenzyl | |
| 1700 | cyclohexyl | CH₃ | H | H | 3,5-dimethoxybenzyl | |
| 1701 | cyclohexyl | CH₃ | H | H | 2,5-dimethoxybenzyl | |
| 1702 | cyclohexyl | CH₃ | H | H | 2-trifluoromethylbenzyl | |
| 1703 | cyclohexyl | CH₃ | H | H | 3-trifluoromethylbenzyl | |
| 1704 | cyclohexyl | CH₃ | H | H | 4-trifluoromethylbenzyl | |
| 1705 | cyclohexyl | CH₃ | H | H | 3-trifluoromethyl-4- | |
| | | | | | chlorobenzyl | |
| 1706 | cyclohexyl | CH₃ | H | H | 3-trifluoromethyl- | |
| | | | | | 4-fluorobenzyl | |
| 1707 | cyclohexyl | CH₃ | H | H | 2-trifluoro- | |
| | | | | | methoxybenzyl | |
| 1708 | cyclohexyl | CH₃ | H | H | 3-trifluoro- | |
| | | | | | methoxybenzyl | |
| 1709 | cyclohexyl | CH₃ | H | H | 4-trifluoro- | |
| | | | | | methoxybenzyl | |
| 1710 | cyclohexyl | CH₃ | H | H | 2-difluoro- | |
| | | | | | methoxybenzyl | |
| 1711 | cyclohexyl | CH₃ | H | H | 3-difluoro- | |
| | | | | | methoxybenzyl | |
| 1712 | cyclohexyl | CH₃ | H | H | 4-difluoro- | |
| | | | | | methoxybenzyl | |
| 1713 | cyclohexyl | CH₃ | H | H | 2-tert-butylbenzyl | |
| 1714 | cyclohexyl | CH₃ | H | H | 3-tert-butylbenzyl | |
| 1715 | cyclohexyl | CH₃ | H | H | 4-tert-butylbenzyl | |
| 1716 | cyclohexyl | CH₃ | H | H | 2-phenoxybenzyl | |
| 1717 | cyclohexyl | CH₃ | H | H | 3-phenoxybenzyl | |
| 1718 | cyclohexyl | CH₃ | H | H | 4-phenoxybenzyl | |
| 1719 | cyclohexyl | CH₃ | H | H | 2-(4-methoxyphenoxy)- | |
| | | | | | benzyl | |
| 1720 | cyclohexyl | CH₃ | H | H | 3-(4-methoxyphenoxy)- | |
| | | | | | benzyl | |
| 1721 | cyclohexyl | CH₃ | H | H | 4-(4-methoxyphenoxy)- | |
| | | | | | benzyl | |
| 1722 | cyclohexyl | CH₃ | H | H | 4-methanesulphonyl- | |
| | | | | | benzyl | |
| 1723 | cyclohexyl | CH₃ | H | H | 2-ethoxybenzyl | |
| 1724 | cyclohexyl | CH₃ | H | H | 3-ethoxybenzyl | |
| 1725 | cyclohexyl | CH₃ | H | H | 4-ethoxybenzyl | |
| 1726 | cyclohexyl | CH₃ | H | H | phenethyl | |
| 1727 | cyclohexyl | CH₃ | H | H | 2-chlorophenethyl | |
| 1728 | cyclohexyl | CH₃ | H | H | 3-chlorophenethyl | |
| 1729 | cyclohexyl | CH₃ | H | H | 4-chlorophenethyl | |
| 1730 | cyclohexyl | CH₃ | H | H | 2-methylphenethyl | |
| 1731 | cyclohexyl | CH₃ | H | H | 3-methylphenethyl | |
| 1732 | cyclohexyl | CH₃ | H | H | 4-methylphenethyl | |
| 1733 | cyclohexyl | CH₃ | H | H | 2-methoxyphenethyl | |
| 1734 | cyclohexyl | CH₃ | H | H | 3-methoxyphenethyl | |
| 1735 | cyclohexyl | CH₃ | H | H | 4-methoxyphenethyl | |
| 1736 | cyclohexyl | CH₃ | H | H | 2,4-dichlorophenethyl | |
| 1737 | cyclohexyl | CH₃ | H | H | 3,4-dichlorophenethyl | |
| 1738 | cyclohexyl | CH₃ | H | H | 3,5-dichlorophenethyl | |
| 1739 | cyclohexyl | CH₃ | H | H | 2,5-dichlorophenethyl | |
| 1740 | cyclohexyl | CH₃ | H | H | 2,4,6-trichlorophenethyl | |
| 1741 | cyclohexyl | CH₃ | H | H | 2-chloro- | |
| | | | | | 4-methylphenethyl | |
| 1742 | cyclohexyl | CH₃ | H | H | 2-chloro-4-methoxy- | |
| | | | | | phenethyl | |
| 1743 | cyclohexyl | CH₃ | H | H | 2,4-dimethylphenethyl | |
| 1744 | cyclohexyl | CH₃ | H | H | 2,6-dimethylphenethyl | |
| 1745 | cyclohexyl | CH₃ | H | H | 2,4,6-trimethylphenethyl | |
| 1746 | cyclohexyl | CH₃ | H | H | 2-fluorophenethyl | |
| 1747 | cyclohexyl | CH₃ | H | H | 3-fluorophenethyl | |
| 1748 | cyclohexyl | CH₃ | H | H | 4-fluorophenethyl | |
| 1749 | cyclohexyl | CH₃ | H | H | 2-chloro- | |
| | | | | | 4-fluorophenethyl | |
| 1750 | cyclohexyl | CH₃ | H | H | 2,4-dimethoxyphenethyl | |
| 1751 | cyclohexyl | CH₃ | H | H | 3,4-dimethoxyphenethyl | |
| 1752 | cyclohexyl | CH₃ | H | H | 3,5-dimethoxyphenethyl | |
| 1753 | cyclohexyl | CH₃ | H | H | 2,5-dimethoxyphenethyl | |
| 1754 | cyclohexyl | CH₃ | H | H | 2-trifluoromethyl- | |
| | | | | | phenethyl | |
| 1755 | cyclohexyl | CH₃ | H | H | 3-trifluoromethyl- | |
| | | | | | phenethyl | |
| 1756 | cyclohexyl | CH₃ | H | H | 4-trifluoromethyl- | |
| | | | | | phenethyl | |
| 1757 | cyclohexyl | CH₃ | H | H | 2-trifluoro- | |
| | | | | | methoxyphenethyl | |
| 1758 | cyclohexyl | CH₃ | H | H | 3-trifluoro- | |
| | | | | | methoxyphenethyl | |
| 1759 | cyclohexyl | CH₃ | H | H | 4-trifluoro- | |
| | | | | | methoxyphenethyl | |
| 1760 | cyclohexyl | CH₃ | H | H | 2-difluoro- | |
| | | | | | methoxyphenethyl | |
| 1761 | cyclohexyl | CH₃ | H | H | 3-difluoro- | |
| | | | | | methoxyphenethyl | |
| 1762 | cyclohexyl | CH₃ | H | H | 4-difluoro- | |
| | | | | | methoxyphenethyl | |
| 1763 | cyclohexyl | CH₃ | H | H | 2-tert-butylphenethyl | |
| 1764 | cyclohexyl | CH₃ | H | H | 3-tert-butylphenethyl | |
| 1765 | cyclohexyl | CH₃ | H | H | 4-tert-butylphenethyl | |
| 1766 | isopropyl | CH₃ | H | H | phenyl | |
| 1767 | isopropyl | CH₃ | H | H | 2-chlorophenyl | |
| 1768 | isopropyl | CH₃ | H | H | 3-chlorophenyl | |
| 1769 | isopropyl | CH₃ | H | H | 4-chlorophenyl | |
| 1770 | isopropyl | CH₃ | H | H | 2-methylphenyl | |
| 1771 | isopropyl | CH₃ | H | H | 3-methylphenyl | |
| 1772 | isopropyl | CH₃ | H | H | 4-methylphenyl | |
| 1773 | isopropyl | CH₃ | H | H | 2-methoxyphenyl | |
| 1774 | isopropyl | CH₃ | H | H | 3-methoxyphenyl | |
| 1775 | isopropyl | CH₃ | H | H | 4-methoxyphenyl | |
| 1776 | isopropyl | CH₃ | H | H | 2,4-dichlorophenyl | |
| 1777 | isopropyl | CH₃ | H | H | 3,4-dichlorophenyl | |
| 1778 | isopropyl | CH₃ | H | H | 3,5-dichlorophenyl | |
| 1779 | isopropyl | CH₃ | H | H | 2,5-dichlorophenyl | |
| 1780 | isopropyl | CH₃ | H | H | 2,4,6-trichlorophenyl | |
| 1781 | isopropyl | CH₃ | H | H | 2-chloro-4-methylphenyl | |
| 1782 | isopropyl | CH₃ | H | H | 2-chloro-4-methoxy- | |
| | | | | | phenyl | |
| 1783 | isopropyl | CH₃ | H | H | 2,4-dimethylphenyl | |
| 1784 | isopropyl | CH₃ | H | H | 2,5-dimethylphenyl | |
| 1785 | isopropyl | CH₃ | H | H | 2,6-dimethylphenyl | |
| 1786 | isopropyl | CH₃ | H | H | 2,4,6-trimethylphenyl | |
| 1787 | isopropyl | CH₃ | H | H | 2-fluorophenyl | |
| 1788 | isopropyl | CH₃ | H | H | 3-fluorophenyl | |
| 1789 | isopropyl | CH₃ | H | H | 4-fluorophenyl | |
| 1790 | isopropyl | CH₃ | H | H | 2-chloro-4-fluorophenyl | |
| 1791 | isopropyl | CH₃ | H | H | 2,4-dimethoxyphenyl | |
| 1792 | isopropyl | CH₃ | H | H | 2,3-dimethoxyphenyl | |
| 1793 | isopropyl | CH₃ | H | H | 3,4-dimethoxyphenyl | |
| 1794 | isopropyl | CH₃ | H | H | 3,5-dimethoxyphenyl | |
| 1795 | isopropyl | CH₃ | H | H | 2,5-dimethoxyphenyl | |
| 1796 | isopropyl | CH₃ | H | H | 2-trifluoromethylphenyl | |
| 1797 | isopropyl | CH₃ | H | H | 3-trifluoromethylphenyl | |
| 1798 | isopropyl | CH₃ | H | H | 4-trifluoromethylphenyl | |
| 1799 | isopropyl | CH₃ | H | H | 3-trifluoromethyl- | |
| | | | | | 4-chlorophenyl | |
| 1800 | isopropyl | CH₃ | H | H | 3-trifluoromethyl- | |
| | | | | | 4-fluorophenyl | |
| 1801 | isopropyl | CH₃ | H | H | 2-trifluoro- | |
| | | | | | methoxyphenyl | |
| 1802 | isopropyl | CH₃ | H | H | 3-trifluoro- | |
| | | | | | methoxyphenyl | |
| 1803 | isopropyl | CH₃ | H | H | 4-trifluoro- | |
| | | | | | methoxyphenyl | |
| 1804 | isopropyl | CH₃ | H | H | 2-difluoro- | |
| | | | | | methoxyphenyl | |
| 1805 | isopropyl | CH₃ | H | H | 3-difluoro- | |
| | | | | | methoxyphenyl | |
| 1806 | isopropyl | CH₃ | H | H | 4-difluoro- | |
| | | | | | methoxyphenyl | |
| 1807 | isopropyl | CH₃ | H | H | 2-tert-butylphenyl | |
| 1808 | isopropyl | CH₃ | H | H | 3-tert-butylphenyl | |
| 1809 | isopropyl | CH₃ | H | H | 4-tert-butylphenyl | |
| 1810 | isopropyl | CH₃ | H | H | 2-phenoxyphenyl | |
| 1811 | isopropyl | CH₃ | H | H | 3-phenoxyphenyl | |
| 1812 | isopropyl | CH₃ | H | H | 4-phenoxyphenyl | |
| 1813 | isopropyl | CH₃ | H | H | 2-(4-methoxyphenoxy)- | |
| | | | | | phenyl | |
| 1814 | isopropyl | CH₃ | H | H | 3-(4-methoxyphenoxy)- | |
| | | | | | phenyl | |
| 1815 | isopropyl | CH₃ | H | H | 4-(4-methoxyphenoxy)- | |
| | | | | | phenyl | |
| 1816 | isopropyl | CH₃ | H | H | 4-methanesulphonyl- | |
| | | | | | phenyl | |
| 1817 | isopropyl | CH₃ | H | H | 2-ethoxyphenyl | |
| 1818 | isopropyl | CH₃ | H | H | 3-ethoxyphenyl | |
| 1819 | isopropyl | CH₃ | H | H | 4-ethoxyphenyl | |
| 1820 | isopropyl | CH₃ | H | H | methyl | |
| 1821 | isopropyl | CH₃ | H | H | ethyl | |
| 1822 | isopropyl | CH₃ | H | H | n-propyl | |
| 1823 | isopropyl | CH₃ | H | H | n-butyl | |
| 1824 | isopropyl | CH₃ | H | H | n-pentyl | |
| 1825 | isopropyl | CH₃ | H | H | benzyl | |
| 1826 | isopropyl | CH₃ | H | H | 2-chlorobenzyl | |
| 1827 | isopropyl | CH₃ | H | H | 3-chlorobenzyl | |
| 1828 | isopropyl | CH₃ | H | H | 4-chlorobenzyl | |
| 1829 | isopropyl | CH₃ | H | H | 2-methylbenzyl | |
| 1830 | isopropyl | CH₃ | H | H | 3-methylbenzyl | |
| 1831 | isopropyl | CH₃ | H | H | 4-methylbenzyl | |
| 1832 | isopropyl | CH₃ | H | H | 2-methoxybenzyl | |
| 1833 | isopropyl | CH₃ | H | H | 3-methoxybenzyl | |
| 1834 | isopropyl | CH₃ | H | H | 4-methoxybenzyl | |
| 1835 | isopropyl | CH₃ | H | H | 2,4-dichlorobenzyl | |
| 1836 | isopropyl | CH₃ | H | H | 3,4-dichlorobenzyl | |
| 1837 | isopropyl | CH₃ | H | H | 3,5-dichlorobenzyl | |
| 1838 | isopropyl | CH₃ | H | H | 2,5-dichlorobenzyl | |
| 1839 | isopropyl | CH₃ | H | H | 2,4,6-trichlorobenzyl | |
| 1840 | isopropyl | CH₃ | H | H | 2-chloro-4-methylbenzyl | |
| 1841 | isopropyl | CH₃ | H | H | 2-chloro-4-methoxy- | |
| | | | | | benzyl | |
| 1842 | isopropyl | CH₃ | H | H | 2,4-dimethylbenzyl | |
| 1843 | isopropyl | CH₃ | H | H | 2,5-dimethylbenzyl | |
| 1844 | isopropyl | CH₃ | H | H | 2,6-dimethylbenzyl | |
| 1845 | isopropyl | CH₃ | H | H | 2,4,6-trimethylbenzyl | |
| 1846 | isopropyl | CH₃ | H | H | 2-fluorobenzyl | |
| 1847 | isopropyl | CH₃ | H | H | 3-fluorobenzyl | |
| 1848 | isopropyl | CH₃ | H | H | 4-fluorobenzyl | |
| 1849 | isopropyl | CH₃ | H | H | 2-chloro-4-fluorobenzyl | |
| 1850 | isopropyl | CH₃ | H | H | 2,4-dimethoxybenzyl | |
| 1851 | isopropyl | CH₃ | H | H | 2,3-dimethoxybenzyl | |
| 1852 | isopropyl | CH₃ | H | H | 3,4-dimethoxybenzyl | |
| 1853 | isopropyl | CH₃ | H | H | 3,5-dimethoxybenzyl | |
| 1854 | isopropyl | CH₃ | H | H | 2,5-dimethoxybenzyl | |
| 1855 | isopropyl | CH₃ | H | H | 2-trifluoromethylbenzyl | |
| 1856 | isopropyl | CH₃ | H | H | 3-trifluoromethylbenzyl | |
| 1857 | isopropyl | CH₃ | H | H | 4-trifluoromethylbenzyl | |
| 1858 | isopropyl | CH₃ | H | H | 3-trifluoromethyl-4- | |
| | | | | | chlorobenzyl | |
| 1859 | isopropyl | CH₃ | H | H | 3-trifluoromethyl- | |
| | | | | | 4-fluorobenzyl | |
| 1860 | isopropyl | CH₃ | H | H | 2-trifluoro- | |
| | | | | | methoxybenzyl | |
| 1861 | isopropyl | CH₃ | H | H | 3-trifluoro- | |
| | | | | | methoxybenzyl | |
| 1862 | isopropyl | CH₃ | H | H | 4-trifluoro- | |
| | | | | | methoxybenzyl | |
| 1863 | isopropyl | CH₃ | H | H | 2-difluoro- | |
| | | | | | methoxybenzyl | |
| 1864 | isopropyl | CH₃ | H | H | 3-difluoro- | |
| | | | | | methoxybenzyl | |
| 1865 | isopropyl | CH₃ | H | H | 4-difluoro- | |
| | | | | | methoxybenzyl | |
| 1866 | isopropyl | CH₃ | H | H | 2-tert-butylbenzyl | |
| 1867 | isopropyl | CH₃ | H | H | 3-tert-butylbenzyl | |
| 1868 | isopropyl | CH₃ | H | H | 4-tert-butylbenzyl | |
| 1869 | isopropyl | CH₃ | H | H | 2-phenoxybenzyl | |
| 1870 | isopropyl | CH₃ | H | H | 3-phenoxybenzyl | |
| 1871 | isopropyl | CH₃ | H | H | 4-phenoxybenzyl | |
| 1872 | isopropyl | CH₃ | H | H | 2-(4-methoxyphenoxy)- | |
| | | | | | benzyl | |
| 1873 | isopropyl | CH₃ | H | H | 3-(4-methoxyphenoxy)- | |
| | | | | | benzyl | |
| 1874 | isopropyl | CH₃ | H | H | 4-(4-methoxyphenoxy)- | |
| | | | | | benzyl | |
| 1875 | isopropyl | CH₃ | H | H | 4-methanesulphonyl- | |
| | | | | | benzyl | |
| 1876 | isopropyl | CH₃ | H | H | 2-ethoxybenzyl | |
| 1877 | isopropyl | CH₃ | H | H | 3-ethoxybenzyl | |
| 1878 | isopropyl | CH₃ | H | H | 4-ethoxybenzyl | |
| 1879 | isopropyl | CH₃ | H | H | phenethyl | |
| 1880 | isopropyl | CH₃ | H | H | 2-chlorophenethyl | |
| 1881 | isopropyl | CH₃ | H | H | 3-chlorophenethyl | |
| 1882 | isopropyl | CH₃ | H | H | 4-chlorophenethyl | |
| 1883 | isopropyl | CH₃ | H | H | 2-methylphenethyl | |
| 1884 | isopropyl | CH₃ | H | H | 3-methylphenethyl | |
| 1885 | isopropyl | CH₃ | H | H | 4-methylphenethyl | |
| 1886 | isopropyl | CH₃ | H | H | 2-methoxyphenethyl | |
| 1887 | isopropyl | CH₃ | H | H | 3-methoxyphenethyl | |
| 1888 | isopropyl | CH₃ | H | H | 4-methoxyphenethyl | |
| 1889 | isopropyl | CH₃ | H | H | 2,4-dichlorophenethyl | |
| 1890 | isopropyl | CH₃ | H | H | 3,4-dichlorophenethyl | |
| 1891 | isopropyl | CH₃ | H | H | 3,5-dichlorophenethyl | |
| 1892 | isopropyl | CH₃ | H | H | 2,5-dichlorophenethyl | |
| 1893 | isopropyl | CH₃ | H | H | 2,4,6-trichlorophenethyl | |
| 1894 | isopropyl | CH₃ | H | H | 2-chloro- | |
| | | | | | 4-methylphenethyl | |
| 1895 | isopropyl | CH₃ | H | H | 2-chloro-4-methoxy- | |
| | | | | | phenethyl | |
| 1896 | isopropyl | CH₃ | H | H | 2,4-dimethylphenethyl | |
| 1897 | isopropyl | CH₃ | H | H | 2,6-dimethylphenethyl | |
| 1898 | isopropyl | CH₃ | H | H | 2,4,6-trimethylphenethyl | |
| 1899 | isopropyl | CH₃ | H | H | 2-fluorophenethyl | |
| 1900 | isopropyl | CH₃ | H | H | 3-fluorophenethyl | |
| 1901 | isopropyl | CH₃ | H | H | 4-fluorophenethyl | |
| 1902 | isopropyl | CH₃ | H | H | 2-chloro- | |
| | | | | | 4-fluorophenethyl | |
| 1903 | isopropyl | CH₃ | H | H | 2,4-dimethoxyphenethyl | |
| 1904 | isopropyl | CH₃ | H | H | 3,4-dimethoxyphenethyl | |
| 1905 | isopropyl | CH₃ | H | H | 3,5-dimethoxyphenethyl | |
| 1906 | isopropyl | CH₃ | H | H | 2,5-dimethoxyphenethyl | |
| 1907 | isopropyl | CH₃ | H | H | 2-trifluoromethyl- | |
| | | | | | phenethyl | |
| 1908 | isopropyl | CH₃ | H | H | 3-trifluoromethyl- | |
| | | | | | phenethyl | |
| 1909 | isopropyl | CH₃ | H | H | 4-trifluoromethyl- | |
| | | | | | phenethyl | |
| 1910 | isopropyl | CH₃ | H | H | 2-trifluoro- | |
| | | | | | methoxyphenethyl | |
| 1911 | isopropyl | CH₃ | H | H | 3-trifluoro- | |
| | | | | | methoxyphenethyl | |
| 1912 | isopropyl | CH₃ | H | H | 4-trifluoro- | |
| | | | | | methoxyphenethyl | |
| 1913 | isopropyl | CH₃ | H | H | 2-difluoro- | |
| | | | | | methoxyphenethyl | |
| 1914 | isopropyl | CH₃ | H | H | 3-difluoro- | |
| | | | | | methoxyphenethyl | |
| 1915 | isopropyl | CH₃ | H | H | 4-difluoro- | |
| | | | | | methoxyphenethyl | |
| 1916 | isopropyl | CH₃ | H | H | 2-tert-butylphenethyl | |
| 1917 | isopropyl | CH₃ | H | H | 3-tert-butylphenethyl | |
| 1918 | isopropyl | CH₃ | H | H | 4-tert-butylphenethyl | |
| 1919 | isobutyl | CH₃ | H | H | phenyl | |
| 1920 | isobutyl | CH₃ | H | H | 2-chlorophenyl | |
| 1921 | isobutyl | CH₃ | H | H | 3-chlorophenyl | |
| 1922 | isobutyl | CH₃ | H | H | 4-chlorophenyl | |
| 1923 | isobutyl | CH₃ | H | H | 2-methylphenyl | |
| 1924 | isobutyl | CH₃ | H | H | 3-methylphenyl | |
| 1925 | isobutyl | CH₃ | H | H | 4-methylphenyl | |
| 1926 | isobutyl | CH₃ | H | H | 2-methoxyphenyl | |
| 1927 | isobutyl | CH₃ | H | H | 3-methoxyphenyl | |
| 1928 | isobutyl | CH₃ | H | H | 4-methoxyphenyl | |
| 1929 | isobutyl | CH₃ | H | H | 2,4-dichlorophenyl | |
| 1930 | isobutyl | CH₃ | H | H | 3,4-dichlorophenyl | |
| 1931 | isobutyl | CH₃ | H | H | 3,5-dichlorophenyl | |
| 1932 | isobutyl | CH₃ | H | H | 2,5-dichlorophenyl | |
| 1933 | isobutyl | CH₃ | H | H | 2,4,6-trichlorophenyl | |
| 1934 | isobutyl | CH₃ | H | H | 2-chloro-4-methylphenyl | |
| 1935 | isobutyl | CH₃ | H | H | 2-chloro-4-methoxy- | |
| | | | | | phenyl | |
| 1936 | isobutyl | CH₃ | H | H | 2,4-dimethylphenyl | |
| 1937 | isobutyl | CH₃ | H | H | 2,5-dimethylphenyl | |
| 1938 | isobutyl | CH₃ | H | H | 2,6-dimethylphenyl | |
| 1939 | isobutyl | CH₃ | H | H | 2,4,6-trimethylphenyl | |
| 1940 | isobutyl | CH₃ | H | H | 2-fluorophenyl | |
| 1941 | isobutyl | CH₃ | H | H | 3-fluorophenyl | |
| 1942 | isobutyl | CH₃ | H | H | 4-fluorophenyl | |
| 1943 | isobutyl | CH₃ | H | H | 2-chloro-4-fluorophenyl | |
| 1944 | isobutyl | CH₃ | H | H | 2,4-dimethoxyphenyl | |
| 1945 | isobutyl | CH₃ | H | H | 2,3-dimethoxyphenyl | |
| 1946 | isobutyl | CH₃ | H | H | 3,4-dimethoxyphenyl | |
| 1947 | isobutyl | CH₃ | H | H | 3,5-dimethoxyphenyl | |
| 1948 | isobutyl | CH₃ | H | H | 2,5-dimethoxyphenyl | |
| 1949 | isobutyl | CH₃ | H | H | 2-trifluoromethylphenyl | |
| 1950 | isobutyl | CH₃ | H | H | 3-trifluoromethylphenyl | |
| 1951 | isobutyl | CH₃ | H | H | 4-trifluoromethylphenyl | |
| 1952 | isobutyl | CH₃ | H | H | 3-trifluoromethyl- | |
| | | | | | 4-chlorophenyl | |
| 1953 | isobutyl | CH₃ | H | H | 3-trifluoromethyl- | |
| | | | | | 4-fluorophenyl | |
| 1954 | isobutyl | CH₃ | H | H | 2-trifluoro- | |
| | | | | | methoxyphenyl | |
| 1955 | isobutyl | CH₃ | H | H | 3-trifluoro- | |
| | | | | | methoxyphenyl | |
| 1956 | isobutyl | CH₃ | H | H | 4-trifluoro- | |
| | | | | | methoxyphenyl | |
| 1957 | isobutyl | CH₃ | H | H | 2-difluoro- | |
| | | | | | methoxyphenyl | |
| 1958 | isobutyl | CH₃ | H | H | 3-difluoro- | |
| | | | | | methoxyphenyl | |
| 1959 | isobutyl | CH₃ | H | H | 4-difluoro- | |
| | | | | | methoxyphenyl | |
| 1960 | isobutyl | CH₃ | H | H | 2-tert-butylphenyl | |
| 1961 | isobutyl | CH₃ | H | H | 3-tert-butylphenyl | |
| 1962 | isobutyl | CH₃ | H | H | 4-tert-butylphenyl | |
| 1963 | isobutyl | CH₃ | H | H | 2-phenoxyphenyl | |
| 1964 | isobutyl | CH₃ | H | H | 3-phenoxyphenyl | |
| 1965 | isobutyl | CH₃ | H | H | 4-phenoxyphenyl | |
| 1966 | isobutyl | CH₃ | H | H | 2-(4-methoxyphenoxy)- | |
| | | | | | phenyl | |
| 1967 | isobutyl | CH₃ | H | H | 3-(4-methoxyphenoxy)- | |
| | | | | | phenyl | |
| 1968 | isobutyl | CH₃ | H | H | 4-(4-methoxyphenoxy)- | |
| | | | | | phenyl | |
| 1969 | isobutyl | CH₃ | H | H | 4-methanesulphonyl- | |
| | | | | | phenyl | |
| 1970 | isobutyl | CH₃ | H | H | 2-ethoxyphenyl | |
| 1971 | isobutyl | CH₃ | H | H | 3-ethoxyphenyl | |
| 1972 | isobutyl | CH₃ | H | H | 4-ethoxyphenyl | |
| 1973 | isobutyl | CH₃ | H | H | methyl | |
| 1974 | isobutyl | CH₃ | H | H | ethyl | |
| 1975 | isobutyl | CH₃ | H | H | n-butyl | |
| 1976 | isobutyl | CH₃ | H | H | n-butyl | |
| 1977 | isobutyl | CH₃ | H | H | n-pentyl | |
| 1978 | isobutyl | CH₃ | H | H | benzyl | |
| 1979 | isobutyl | CH₃ | H | H | 2-chlorobenzyl | |
| 1980 | isobutyl | CH₃ | H | H | 3-chlorobenzyl | |
| 1981 | isobutyl | CH₃ | H | H | 4-chlorobenzyl | |
| 1982 | isobutyl | CH₃ | H | H | 2-methylbenzyl | |
| 1983 | isobutyl | CH₃ | H | H | 3-methylbenzyl | |
| 1984 | isobutyl | CH₃ | H | H | 4-methylbenzyl | |
| 1985 | isobutyl | CH₃ | H | H | 2-methoxybenzyl | |
| 1986 | isobutyl | CH₃ | H | H | 3-methoxybenzyl | |
| 1987 | isobutyl | CH₃ | H | H | 4-methoxybenzyl | |
| 1988 | isobutyl | CH₃ | H | H | 2,4-dichlorobenzyl | |
| 1989 | isobutyl | CH₃ | H | H | 3,4-dichlorobenzyl | |
| 1990 | isobutyl | CH₃ | H | H | 3,5-dichlorobenzyl | |
| 1991 | isobutyl | CH₃ | H | H | 2,5-dichlorobenzyl | |
| 1992 | isobutyl | CH₃ | H | H | 2,4,6-trichlorobenzyl | |
| 1993 | isobutyl | CH₃ | H | H | 2-chloro-4-methylbenzyl | |
| 1994 | isobutyl | CH₃ | H | H | 2-chloro-4-methoxy- | |
| | | | | | benzyl | |
| 1995 | isobutyl | CH₃ | H | H | 2,4-dimethylbenzyl | |
| 1996 | isobutyl | CH₃ | H | H | 2,5-dimethylbenzyl | |
| 1997 | isobutyl | CH₃ | H | H | 2,6-dimethylbenzyl | |
| 1998 | isobutyl | CH₃ | H | H | 2,4,6-trimethylbenzyl | |
| 1999 | isobutyl | CH₃ | H | H | 2-fluorobenzyl | |
| 2000 | isobutyl | CH₃ | H | H | 3-fluorobenzyl | |
| 2001 | isobutyl | CH₃ | H | H | 4-fluorobenzyl | |
| 2002 | isobutyl | CH₃ | H | H | 2-chloro-4-fluorobenzyl | |
| 2003 | isobutyl | CH₃ | H | H | 2,4-dimethoxybenzyl | |
| 2004 | isobutyl | CH₃ | H | H | 2,3-dimethoxybenzyl | |
| 2005 | isobutyl | CH₃ | H | H | 3,4-dimethoxybenzyl | |
| 2006 | isobutyl | CH₃ | H | H | 3,5-dimethoxybenzyl | |
| 2007 | isobutyl | CH₃ | H | H | 2,5-dimethoxybenzyl | |
| 2008 | isobutyl | CH₃ | H | H | 2-trifluoromethylbenzyl | |
| 2009 | isobutyl | CH₃ | H | H | 3-trifluoromethylbenzyl | |
| 2010 | isobutyl | CH₃ | H | H | 4-trifluoromethylbenzyl | |
| 2011 | isobutyl | CH₃ | H | H | 3-trifluoromethyl-4- | |
| | | | | | chlorobenzyl | |
| 2012 | isobutyl | CH₃ | H | H | 3-trifluoromethyl- | |
| | | | | | 4-fluorobenzyl | |
| 2013 | isobutyl | CH₃ | H | H | 2-trifluoro- | |
| | | | | | methoxybenzyl | |
| 2014 | isobutyl | CH₃ | H | H | 3-trifluoro- | |
| | | | | | methoxybenzyl | |
| 2015 | isobutyl | CH₃ | H | H | 4-trifluoro- | |
| | | | | | methoxybenzyl | |
| 2016 | isobutyl | CH₃ | H | H | 2-difluoro- | |
| | | | | | methoxybenzyl | |
| 2017 | isobutyl | CH₃ | H | H | 3-difluoro- | |
| | | | | | methoxybenzyl | |
| 2018 | isobutyl | CH₃ | H | H | 4-difluoro- | |
| | | | | | methoxybenzyl | |
| 2019 | isobutyl | CH₃ | H | H | 2-tert-butylbenzyl | |
| 2020 | isobutyl | CH₃ | H | H | 3-tert-butylbenzyl | |
| 2021 | isobutyl | CH₃ | H | H | 4-tert-butylbenzyl | |
| 2022 | isobutyl | CH₃ | H | H | 2-phenoxybenzyl | |
| 2023 | isobutyl | CH₃ | H | H | 3-phenoxybenzyl | |
| 2024 | isobutyl | CH₃ | H | H | 4-phenoxybenzyl | |
| 2025 | isobutyl | CH₃ | H | H | 2-(4-methoxyphenoxy)- | |
| | | | | | benzyl | |
| 2026 | isobutyl | CH₃ | H | H | 3-(4-methoxyphenoxy)- | |
| | | | | | benzyl | |
| 2027 | isobutyl | CH₃ | H | H | 4-(4-methoxyphenoxy)- | |
| | | | | | benzyl | |
| 2028 | isobutyl | CH₃ | H | H | 4-methanesulphonyl- | |
| | | | | | benzyl | |
| 2029 | isobutyl | CH₃ | H | H | 2-ethoxybenzyl | |
| 2030 | isobutyl | CH₃ | H | H | 3-ethoxybenzyl | |
| 2031 | isobutyl | CH₃ | H | H | 4-ethoxybenzyl | |
| 2032 | isobutyl | CH₃ | H | H | phenethyl | |
| 2033 | isobutyl | CH₃ | H | H | 2-chlorophenethyl | |
| 2034 | isobutyl | CH₃ | H | H | 3-chlorophenethyl | |
| 2035 | isobutyl | CH₃ | H | H | 4-chlorophenethyl | |
| 2036 | isobutyl | CH₃ | H | H | 2-methylphenethyl | |
| 2037 | isobutyl | CH₃ | H | H | 3-methylphenethyl | |
| 2038 | isobutyl | CH₃ | H | H | 4-methylphenethyl | |
| 2039 | isobutyl | CH₃ | H | H | 2-methoxyphenethyl | |
| 2040 | isobutyl | CH₃ | H | H | 3-methoxyphenethyl | |
| 2041 | isobutyl | CH₃ | H | H | 4-methoxyphenethyl | |
| 2042 | isobutyl | CH₃ | H | H | 2,4-dichlorophenethyl | |
| 2043 | isobutyl | CH₃ | H | H | 3,4-dichlorophenethyl | |
| 2044 | isobutyl | CH₃ | H | H | 3,5-dichlorophenethyl | |
| 2045 | isobutyl | CH₃ | H | H | 2,5-dichlorophenethyl | |
| 2046 | isobutyl | CH₃ | H | H | 2,4,6-trichlorophenethyl | |
| 2047 | isobutyl | CH₃ | H | H | 2-chloro- | |
| | | | | | 4-methylphenethyl | |
| 2048 | isobutyl | CH₃ | H | H | 2-chloro-4-methoxy- | |
| | | | | | phenethyl | |
| 2049 | isobutyl | CH₃ | H | H | 2,4-dimethylphenethyl | |
| 2050 | isobutyl | CH₃ | H | H | 2,6-dimethylphenethyl | |
| 2051 | isobutyl | CH₃ | H | H | 2,4,6-trimethylphenethyl | |
| 2052 | isobutyl | CH₃ | H | H | 2-fluorophenethyl | |
| 2053 | isobutyl | CH₃ | H | H | 3-fluorophenethyl | |
| 2054 | isobutyl | CH₃ | H | H | 4-fluorophenethyl | |
| 2055 | isobutyl | CH₃ | H | H | 2-chloro- | |
| | | | | | 4-fluorophenethyl | |
| 2056 | isobutyl | CH₃ | H | H | 2,4-dimethoxyphenethyl | |
| 2057 | isobutyl | CH₃ | H | H | 3,4-dimethoxyphenethyl | |
| 2058 | isobutyl | CH₃ | H | H | 3,5-dimethoxyphenethyl | |
| 2059 | isobutyl | CH₃ | H | H | 2,5-dimethoxyphenethyl | |
| 2060 | isobutyl | CH₃ | H | H | 2-trifluoromethyl- | |
| | | | | | phenethyl | |
| 2061 | isobutyl | CH₃ | H | H | 3-trifluoromethyl- | |
| | | | | | phenethyl | |
| 2062 | isobutyl | CH₃ | H | H | 4-trifluoromethyl- | |
| | | | | | phenethyl | |
| 2063 | isobutyl | CH₃ | H | H | 2-trifluoro- | |
| | | | | | methoxyphenethyl | |
| 2064 | isobutyl | CH₃ | H | H | 3-trifluoro- | |
| | | | | | methoxyphenethyl | |
| 2065 | isobutyl | CH₃ | H | H | 4-trifluoro- | |
| | | | | | methoxyphenethyl | |
| 2066 | isobutyl | CH₃ | H | H | 2-difluoro- | |
| | | | | | methoxyphenethyl | |
| 2067 | isobutyl | CH₃ | H | H | 3-difluoro- | |
| | | | | | methoxyphenethyl | |
| 2068 | isobutyl | CH₃ | H | H | 4-difluoro- | |
| | | | | | methoxyphenethyl | |
| 2069 | isobutyl | CH₃ | H | H | 2-tert-butylphenethyl | |
| 2070 | isobutyl | CH₃ | H | H | 3-tert-butylphenethyl | |
| 2071 | isobutyl | CH₃ | H | H | 4-tert-butylphenethyl | |
| 2072 | benzyl | CH₃ | H | H | phenyl | |
| 2073 | benzyl | CH₃ | H | H | 2-chlorophenyl | |
| 2074 | benzyl | CH₃ | H | H | 3-chlorophenyl | |
| 2075 | benzyl | CH₃ | H | H | 4-chlorophenyl | |
| 2076 | benzyl | CH₃ | H | H | 2-methylphenyl | |
| 2077 | benzyl | CH₃ | H | H | 3-methylphenyl | |
| 2078 | benzyl | CH₃ | H | H | 4-methylphenyl | |
| 2079 | benzyl | CH₃ | H | H | 2-methoxyphenyl | |
| 2080 | benzyl | CH₃ | H | H | 3-methoxyphenyl | |
| 2081 | benzyl | CH₃ | H | H | 4-methoxyphenyl | |
| 2082 | benzyl | CH₃ | H | H | 2,4-dichlorophenyl | |
| 2083 | benzyl | CH₃ | H | H | 3,4-dichlorophenyl | |
| 2084 | benzyl | CH₃ | H | H | 3,5-dichlorophenyl | |
| 2085 | benzyl | CH₃ | H | H | 2,5-dichlorophenyl | |
| 2086 | benzyl | CH₃ | H | H | 2,4,6-trichlorophenyl | |
| 2087 | benzyl | CH₃ | H | H | 2-chloro-4-methylphenyl | |
| 2088 | benzyl | CH₃ | H | H | 2-chloro-4-methoxy- | |
| | | | | | phenyl | |
| 2089 | benzyl | CH₃ | H | H | 2,4-dimethylphenyl | |
| 2090 | benzyl | CH₃ | H | H | 2,5-dimethylphenyl | |
| 2091 | benzyl | CH₃ | H | H | 2,6-dimethylphenyl | |
| 2092 | benzyl | CH₃ | H | H | 2,4,6-trimethylphenyl | |
| 2093 | benzyl | CH₃ | H | H | 2-fluorophenyl | |
| 2094 | benzyl | CH₃ | H | H | 3-fluorophenyl | |
| 2095 | benzyl | CH₃ | H | H | 4-fluorophenyl | |
| 2096 | benzyl | CH₃ | H | H | 2-chloro-4-fluorophenyl | |
| 2097 | benzyl | CH₃ | H | H | 2,4-dimethoxyphenyl | |
| 2098 | benzyl | CH₃ | H | H | 2,3-dimethoxyphenyl | |
| 2099 | benzyl | CH₃ | H | H | 3,4-dimethoxyphenyl | |
| 2100 | benzyl | CH₃ | H | H | 3,5-dimethoxyphenyl | |
| 2101 | benzyl | CH₃ | H | H | 2,5-dimethoxyphenyl | |
| 2102 | benzyl | CH₃ | H | H | 2-trifluoromethylphenyl | |
| 2103 | benzyl | CH₃ | H | H | 3-trifluoromethylphenyl | |
| 2104 | benzyl | CH₃ | H | H | 4-trifluoromethylphenyl | |
| 2105 | benzyl | CH₃ | H | H | 3-trifluoromethyl- | |
| | | | | | 4-chlorophenyl | |
| 2106 | benzyl | CH₃ | H | H | 3-trifluoromethyl- | |
| | | | | | 4-fluorophenyl | |
| 2107 | benzyl | CH₃ | H | H | 2-trifluoro- | |
| | | | | | methoxyphenyl | |
| 2108 | benzyl | CH₃ | H | H | 3-trifluoro- | |
| | | | | | methoxyphenyl | |
| 2109 | benzyl | CH₃ | H | H | 4-trifluoro- | |
| | | | | | methoxyphenyl | |
| 2110 | benzyl | CH₃ | H | H | 2-difluoro- | |
| | | | | | methoxyphenyl | |
| 2111 | benzyl | CH₃ | H | H | 3-difluoro- | |
| | | | | | methoxyphenyl | |
| 2112 | benzyl | CH₃ | H | H | 4-difluoro- | |
| | | | | | methoxyphenyl | |
| 2113 | benzyl | CH₃ | H | H | 2-tert-butylphenyl | |
| 2114 | benzyl | CH₃ | H | H | 3-tert-butylphenyl | |
| 2115 | benzyl | CH₃ | H | H | 4-tert-butylphenyl | |
| 2116 | benzyl | CH₃ | H | H | 2-phenoxyphenyl | |
| 2117 | benzyl | CH₃ | H | H | 3-phenoxyphenyl | |
| 2118 | benzyl | CH₃ | H | H | 4-phenoxyphenyl | |
| 2119 | benzyl | CH₃ | H | H | 2-(4-methoxyphenoxy)- | |
| | | | | | phenyl | |
| 2120 | benzyl | CH₃ | H | H | 3-(4-methoxyphenoxy)- | |
| | | | | | phenyl | |
| 2121 | benzyl | CH₃ | H | H | 4-(4-methoxyphenoxy)- | |
| | | | | | phenyl | |
| 2122 | benzyl | CH₃ | H | H | 4-methanesulphonyl- | |
| | | | | | phenyl | |
| 2123 | benzyl | CH₃ | H | H | 2-ethoxyphenyl | |
| 2124 | benzyl | CH₃ | H | H | 3-ethoxyphenyl | |
| 2125 | benzyl | CH₃ | H | H | 4-ethoxyphenyl | |
| 2126 | benzyl | CH₃ | H | H | methyl | |
| 2127 | benzyl | CH₃ | H | H | ethyl | |
| 2128 | benzyl | CH₃ | H | H | n-butyl | |
| 2129 | benzyl | CH₃ | H | H | n-butyl | |
| 2130 | benzyl | CH₃ | H | H | n-pentyl | |
| 2131 | benzyl | CH₃ | H | H | benzyl | |
| 2132 | benzyl | CH₃ | H | H | 2-chlorobenzyl | |
| 2133 | benzyl | CH₃ | H | H | 3-chlorobenzyl | |
| 2134 | benzyl | CH₃ | H | H | 4-chlorobenzyl | |
| 2135 | benzyl | CH₃ | H | H | 2-methylbenzyl | |
| 2136 | benzyl | CH₃ | H | H | 3-methylbenzyl | |
| 2137 | benzyl | CH₃ | H | H | 4-methylbenzyl | |
| 2138 | benzyl | CH₃ | H | H | 2-methoxybenzyl | |
| 2139 | benzyl | CH₃ | H | H | 3-methoxybenzyl | |
| 2140 | benzyl | CH₃ | H | H | 4-methoxybenzyl | |
| 2141 | benzyl | CH₃ | H | H | 2,4-dichlorobenzyl | |
| 2142 | benzyl | CH₃ | H | H | 3,4-dichlorobenzyl | |
| 2143 | benzyl | CH₃ | H | H | 3,5-dichlorobenzyl | |
| 2144 | benzyl | CH₃ | H | H | 2,5-dichlorobenzyl | |
| 2145 | benzyl | CH₃ | H | H | 2,4,6-trichlorobenzyl | |
| 2146 | benzyl | CH₃ | H | H | 2-chloro-4-methylbenzyl | |
| 2147 | benzyl | CH₃ | H | H | 2-chloro-4-methoxy- | |
| | | | | | benzyl | |
| 2148 | benzyl | CH₃ | H | H | 2,4-dimethylbenzyl | |
| 2149 | benzyl | CH₃ | H | H | 2,5-dimethylbenzyl | |
| 2150 | benzyl | CH₃ | H | H | 2,6-dimethylbenzyl | |
| 2151 | benzyl | CH₃ | H | H | 2,4,6-trimethylbenzyl | |
| 2152 | benzyl | CH₃ | H | H | 2-fluorobenzyl | |
| 2153 | benzyl | CH₃ | H | H | 3-fluorobenzyl | |
| 2154 | benzyl | CH₃ | H | H | 4-fluorobenzyl | |
| 2155 | benzyl | CH₃ | H | H | 2-chloro-4-fluorobenzyl | |
| 2156 | benzyl | CH₃ | H | H | 2,4-dimethoxybenzyl | |
| 2157 | benzyl | CH₃ | H | H | 2,3-dimethoxybenzyl | |
| 2158 | benzyl | CH₃ | H | H | 3,4-dimethoxybenzyl | |
| 2159 | benzyl | CH₃ | H | H | 3,5-dimethoxybenzyl | |
| 2160 | benzyl | CH₃ | H | H | 2,5-dimethoxybenzyl | |
| 2161 | benzyl | CH₃ | H | H | 2-trifluoromethylbenzyl | |
| 2162 | benzyl | CH₃ | H | H | 3-trifluoromethylbenzyl | |
| 2163 | benzyl | CH₃ | H | H | 4-trifluoromethylbenzyl | |
| 2164 | benzyl | CH₃ | H | H | 3-trifluoromethyl-4- | |
| | | | | | chlorobenzyl | |
| 2165 | benzyl | CH₃ | H | H | 3-trifluoromethyl- | |
| | | | | | 4-fluorobenzyl | |
| 2166 | benzyl | CH₃ | H | H | 2-trifluoro- | |
| | | | | | methoxybenzyl | |
| 2167 | benzyl | CH₃ | H | H | 3-trifluoro- | |
| | | | | | methoxybenzyl | |
| 2168 | benzyl | CH₃ | H | H | 4-trifluoro- | |
| | | | | | methoxybenzyl | |
| 2169 | benzyl | CH₃ | H | H | 2-difluoro- | |
| | | | | | methoxybenzyl | |
| 2170 | benzyl | CH₃ | H | H | 3-difluoro- | |
| | | | | | methoxybenzyl | |
| 2171 | benzyl | CH₃ | H | H | 4-difluoro- | |
| | | | | | methoxybenzyl | |
| 2172 | benzyl | CH₃ | H | H | 2-tert-butylbenzyl | |
| 2173 | benzyl | CH₃ | H | H | 3-tert-butylbenzyl | |
| 2174 | benzyl | CH₃ | H | H | 4-tert-butylbenzyl | |
| 2175 | benzyl | CH₃ | H | H | 2-phenoxybenzyl | |
| 2176 | benzyl | CH₃ | H | H | 3-phenoxybenzyl | |
| 2177 | benzyl | CH₃ | H | H | 4-phenoxybenzyl | |
| 2178 | benzyl | CH₃ | H | H | 2-(4-methoxyphenoxy)- | |
| | | | | | benzyl | |
| 2179 | benzyl | CH₃ | H | H | 3-(4-methoxyphenoxy)- | |
| | | | | | benzyl | |
| 2180 | benzyl | CH₃ | H | H | 4-(4-methoxyphenoxy)- | |
| | | | | | benzyl | |
| 2181 | benzyl | CH₃ | H | H | 4-methanesulphonyl- | |
| | | | | | benzyl | |
| 2182 | benzyl | CH₃ | H | H | 2-ethoxybenzyl | |
| 2183 | benzyl | CH₃ | H | H | 3-ethoxybenzyl | |
| 2184 | benzyl | CH₃ | H | H | 4-ethoxybenzyl | |
| 2185 | benzyl | CH₃ | H | H | phenethyl | |
| 2186 | benzyl | CH₃ | H | H | 2-chlorophenethyl | |
| 2187 | benzyl | CH₃ | H | H | 3-chlorophenethyl | |
| 2188 | benzyl | CH₃ | H | H | 4-chlorophenethyl | |
| 2189 | benzyl | CH₃ | H | H | 2-methylphenethyl | |
| 2190 | benzyl | CH₃ | H | H | 3-methylphenethyl | |
| 2191 | benzyl | CH₃ | H | H | 4-methylphenethyl | |
| 2192 | benzyl | CH₃ | H | H | 2-methoxyphenethyl | |
| 2193 | benzyl | CH₃ | H | H | 3-methoxyphenethyl | |
| 2194 | benzyl | CH₃ | H | H | 4-methoxyphenethyl | |
| 2195 | benzyl | CH₃ | H | H | 2,4-dichlorophenethyl | |
| 2196 | benzyl | CH₃ | H | H | 3,4-dichlorophenethyl | |
| 2197 | benzyl | CH₃ | H | H | 3,5-dichlorophenethyl | |
| 2198 | benzyl | CH₃ | H | H | 2,5-dichlorophenethyl | |
| 2199 | benzyl | CH₃ | H | H | 2,4,6-trichlorophenethyl | |
| 2200 | benzyl | CH₃ | H | H | 2-chloro- | |
| | | | | | 4-methylphenethyl | |
| 2201 | benzyl | CH₃ | H | H | 2-chloro-4-methoxy- | |
| | | | | | phenethyl | |
| 2202 | benzyl | CH₃ | H | H | 2,4-dimethylphenethyl | |
| 2203 | benzyl | CH₃ | H | H | 2,6-dimethylphenethyl | |
| 2204 | benzyl | CH₃ | H | H | 2,4,6-trimethylphenethyl | |
| 2205 | benzyl | CH₃ | H | H | 2-fluorophenethyl | |
| 2206 | benzyl | CH₃ | H | H | 3-fluorophenethyl | |
| 2207 | benzyl | CH₃ | H | H | 4-fluorophenethyl | |
| 2208 | benzyl | CH₃ | H | H | 2-chloro- | |
| | | | | | 4-fluorophenethyl | |
| 2209 | benzyl | CH₃ | H | H | 2,4-dimethoxyphenethyl | |
| 2210 | benzyl | CH₃ | H | H | 3,4-dimethoxyphenethyl | |
| 2211 | benzyl | CH₃ | H | H | 3,5-dimethoxyphenethyl | |
| 2212 | benzyl | CH₃ | H | H | 2,5-dimethoxyphenethyl | |
| 2213 | benzyl | CH₃ | H | H | 2-trifluoromethyl- | |
| | | | | | phenethyl | |
| 2214 | benzyl | CH₃ | H | H | 3-trifluoromethyl- | |
| | | | | | phenethyl | |
| 2215 | benzyl | CH₃ | H | H | 4-trifluoromethyl- | |
| | | | | | phenethyl | |
| 2216 | benzyl | CH₃ | H | H | 2-trifluoro- | |
| | | | | | methoxyphenethyl | |
| 2217 | benzyl | CH₃ | H | H | 3-trifluoro- | |
| | | | | | methoxyphenethyl | |
| 2218 | benzyl | CH₃ | H | H | 4-trifluoro- | |
| | | | | | methoxyphenethyl | |
| 2219 | benzyl | CH₃ | H | H | 2-difluoro- | |
| | | | | | methoxyphenethyl | |
| 2220 | benzyl | CH₃ | H | H | 3-difluoro- | |
| | | | | | methoxyphenethyl | |
| 2221 | benzyl | CH₃ | H | H | 4-difluoro- | |
| | | | | | methoxyphenethyl | |
| 2222 | benzyl | CH₃ | H | H | 2-tert-butylphenethyl | |
| 2223 | benzyl | CH₃ | H | H | 3-tert-butylphenethyl | |
| 2224 | benzyl | CH₃ | H | H | 4-tert-butylphenethyl | |
| 2225 | 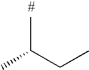 | CH₃ | H | CH₃ | phenyl | |
| 2226 | 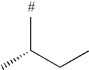 | CH₃ | H | CH₃ | 2-chlorophenyl | |
| 2227 | 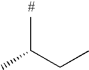 | CH₃ | H | CH₃ | 3-chlorophenyl | |
| 2228 | 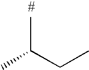 | CH₃ | H | CH₃ | 4-chlorophenyl | |
| 2229 | 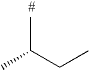 | CH₃ | H | CH₃ | 2-methylphenyl | |
| 2230 | 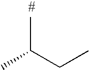 | CH₃ | H | CH₃ | 3-methylphenyl | |
| 2231 | 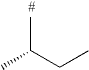 | CH₃ | H | CH₃ | 4-methylphenyl | |
| 2232 | 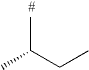 | CH₃ | H | CH₃ | 2-methoxyphenyl | |
| 2233 | 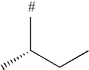 | CH₃ | H | CH₃ | 3-methoxyphenyl | |
| 2234 | 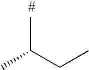 | CH₃ | H | CH₃ | 4-methoxyphenyl | |
| 2235 | 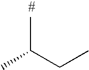 | CH₃ | H | CH₃ | 2,4-dichlorophenyl | |
| 2236 | 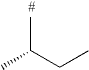 | CH₃ | H | CH₃ | 3,4-dichlorophenyl | |
| 2237 | 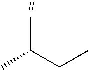 | CH₃ | H | CH₃ | 3,5-dichlorophenyl | |
| 2238 | 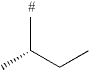 | CH₃ | H | CH₃ | 2,5-dichlorophenyl | |
| 2239 | 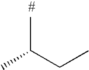 | CH₃ | H | CH₃ | 2,4,6-trichlorophenyl | |
| 2240 | 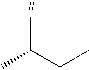 | CH₃ | H | CH₃ | 2-chloro-4-methylphenyl | |
| 2241 | 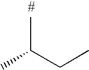 | CH₃ | H | CH₃ | 2-chloro-4-methoxy- phenyl | |
| 2242 | 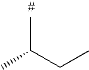 | CH₃ | H | CH₃ | 2,4-dimethylphenyl | |
| 2243 | 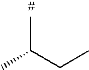 | CH₃ | H | CH₃ | 2,5-dimethylphenyl | |
| 2244 | 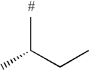 | CH₃ | H | CH₃ | 2,6-dimethylphenyl | |
| 2245 | 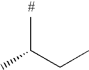 | CH₃ | H | CH₃ | 2,4,6-trimethylphenyl | |
| 2246 | 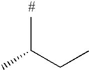 | CH₃ | H | CH₃ | 2-fluorophenyl | |
| 2247 | 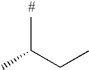 | CH₃ | H | CH₃ | 3-fluorophenyl | |
| 2248 | 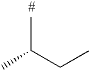 | CH₃ | H | CH₃ | 4-fluorophenyl | |
| 2249 | 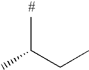 | CH₃ | H | CH₃ | 2-chloro-4-fluorophenyl | |
| 2250 | 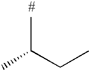 | CH₃ | H | CH₃ | 2,4-dimethoxyphenyl | |
| 2251 | 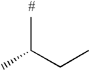 | CH₃ | H | CH₃ | 2,3-dimethoxyphenyl | |
| 2252 | 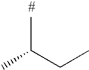 | CH₃ | H | CH₃ | 3,4-dimethoxyphenyl | |
| 2253 | 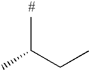 | CH₃ | H | CH₃ | 3,5-dimethoxyphenyl | |
| 2254 | 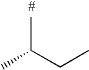 | CH₃ | H | CH₃ | 2,5-dimethoxyphenyl | |
| 2255 | 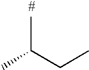 | CH₃ | H | CH₃ | 2-trifluoromethylphenyl | |
| 2256 | 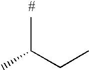 | CH₃ | H | CH₃ | 3-trifluoromethylphenyl | |
| 2257 | 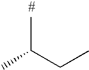 | CH₃ | H | CH₃ | 4-trifluoromethylphenyl | |
| 2258 | 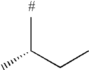 | CH₃ | H | CH₃ | 3-trifluoromethyl- 4-chlorophenyl | |
| 2259 | 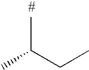 | CH₃ | H | CH₃ | 3-trifluoromethyl- 4-fluorophenyl | |
| 2260 | 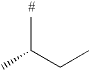 | CH₃ | H | CH₃ | 2-trifluoro- methoxyphenyl | |
| 2261 | 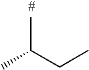 | CH₃ | H | CH₃ | 3-trifluoro- methoxyphenyl | |
| 2262 | 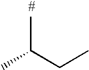 | CH₃ | H | CH₃ | 4-trifluoro- methoxyphenyl | |
| 2263 | 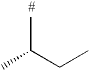 | CH₃ | H | CH₃ | 2-difluoro- methoxyphenyl | |
| 2264 | 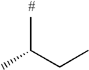 | CH₃ | H | CH₃ | 3-difluoro- methoxyphenyl | |
| 2265 | 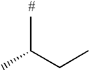 | CH₃ | H | CH₃ | 4-difluoro- methoxyphenyl | |
| 2266 | 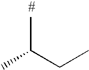 | CH₃ | H | CH₃ | 2-tert-butylphenyl | |
| 2267 | 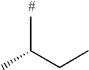 | CH₃ | H | CH₃ | 3-tert-butylphenyl | |
| 2268 | 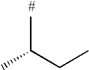 | CH₃ | H | CH₃ | 4-tert-butylphenyl | |
| 2269 | 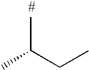 | CH₃ | H | CH₃ | 2-phenoxyphenyl | |
| 2270 | 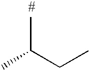 | CH₃ | H | CH₃ | 3-phenoxyphenyl | |
| 2271 | 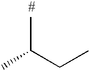 | CH₃ | H | CH₃ | 4-phenoxyphenyl | |
| 2272 | 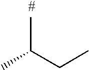 | CH₃ | H | CH₃ | 2-(4-methoxyphenoxy)- phenyl | |
| 2273 | 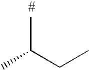 | CH₃ | H | CH₃ | 3-(4-methoxyphenoxy)- phenyl | |
| 2274 | 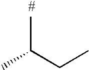 | CH₃ | H | CH₃ | 4-(4-methoxyphenoxy)- phenyl | |
| 2275 | 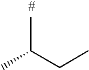 | CH₃ | H | CH₃ | 4-methanesulphonyl- phenyl | |
| 2276 | isopropyl | CH₃ | H | CH₃ | phenyl | |
| 2277 | isopropyl | CH₃ | H | CH₃ | 2-chlorophenyl | |
| 2278 | isopropyl | CH₃ | H | CH₃ | 3-chlorophenyl | |
| 2279 | isopropyl | CH₃ | H | CH₃ | 4-chlorophenyl | |
| 2280 | isopropyl | CH₃ | H | CH₃ | 2-methylphenyl | |
| 2281 | isopropyl | CH₃ | H | CH₃ | 3-methylphenyl | |
| 2282 | isopropyl | CH₃ | H | CH₃ | 4-methylphenyl | |
| 2283 | isopropyl | CH₃ | H | CH₃ | 2-methoxyphenyl | |
| 2284 | isopropyl | CH₃ | H | CH₃ | 3-methoxyphenyl | |
| 2285 | isopropyl | CH₃ | H | CH₃ | 4-methoxyphenyl | |
| 2286 | isopropyl | CH₃ | H | CH₃ | 2,4-dichlorophenyl | |
| 2287 | isopropyl | CH₃ | H | CH₃ | 3,4-dichlorophenyl | |
| 2288 | isopropyl | CH₃ | H | CH₃ | 3,5-dichlorophenyl | |
| 2289 | isopropyl | CH₃ | H | CH₃ | 2,5-dichlorophenyl | |
| 2290 | isopropyl | CH₃ | H | CH₃ | 2,4,6-trichlorophenyl | |
| 2291 | isopropyl | CH₃ | H | CH₃ | 2-chloro-4-methylphenyl | |
| 2292 | isopropyl | CH₃ | H | CH₃ | 2-chloro-4-methoxy- | |
| | | | | | phenyl | |
| 2293 | isopropyl | CH₃ | H | CH₃ | 2,4-dimethylphenyl | |
| 2294 | isopropyl | CH₃ | H | CH₃ | 2,5-dimethylphenyl | |
| 2295 | isopropyl | CH₃ | H | CH₃ | 2,6-dimethylphenyl | |
| 2296 | isopropyl | CH₃ | H | CH₃ | 2,4,6-trimethylphenyl | |
| 2297 | isopropyl | CH₃ | H | CH₃ | 2-fluorophenyl | |
| 2298 | isopropyl | CH₃ | H | CH₃ | 3-fluorophenyl | |
| 2299 | isopropyl | CH₃ | H | CH₃ | 4-fluorophenyl | |
| 2300 | isopropyl | CH₃ | H | CH₃ | 2-chloro-4-fluorophenyl | |
| 2301 | isopropyl | CH₃ | H | CH₃ | 2,4-dimethoxyphenyl | |
| 2302 | isopropyl | CH₃ | H | CH₃ | 2,3-dimethoxyphenyl | |
| 2303 | isopropyl | CH₃ | H | CH₃ | 3,4-dimethoxyphenyl | |
| 2304 | isopropyl | CH₃ | H | CH₃ | 3,5-dimethoxyphenyl | |
| 2305 | isopropyl | CH₃ | H | CH₃ | 2,5-dimethoxyphenyl | |
| 2306 | isopropyl | CH₃ | H | CH₃ | 2-trifluoromethylphenyl | |
| 2307 | isopropyl | CH₃ | H | CH₃ | 3-trifluoromethylphenyl | |
| 2308 | isopropyl | CH₃ | H | CH₃ | 4-trifluoromethylphenyl | |
| 2309 | isopropyl | CH₃ | H | CH₃ | 3-trifluoromethyl- | |
| | | | | | 4-chlorophenyl | |
| 2310 | isopropyl | CH₃ | H | CH₃ | 3-trifluoromethyl- | |
| | | | | | 4-fluorophenyl | |
| 2311 | isopropyl | CH₃ | H | CH₃ | 2-trifluoro- | |
| | | | | | methoxyphenyl | |
| 2312 | isopropyl | CH₃ | H | CH₃ | 3-trifluoro- | |
| | | | | | methoxyphenyl | |
| 2313 | isopropyl | CH₃ | H | CH₃ | 4-trifluoro- | |
| | | | | | methoxyphenyl | |
| 2314 | isobutyl | CH₃ | H | CH₃ | phenyl | |
| 2315 | isobutyl | CH₃ | H | CH₃ | 2-chlorophenyl | |
| 2316 | isobutyl | CH₃ | H | CH₃ | 3-chlorophenyl | |
| 2317 | isobutyl | CH₃ | H | CH₃ | 4-chlorophenyl | |
| 2318 | isobutyl | CH₃ | H | CH₃ | 2-methylphenyl | |
| 2319 | isobutyl | CH₃ | H | CH₃ | 3-methylphenyl | |
| 2320 | isobutyl | CH₃ | H | CH₃ | 4-methylphenyl | |
| 2321 | isobutyl | CH₃ | H | CH₃ | 2-methoxyphenyl | |
| 2322 | isobutyl | CH₃ | H | CH₃ | 3-methoxyphenyl | |
| 2323 | isobutyl | CH₃ | H | CH₃ | 4-methoxyphenyl | |
| 2324 | isobutyl | CH₃ | H | CH₃ | 2,4-dichlorophenyl | |
| 2325 | isobutyl | CH₃ | H | CH₃ | 3,4-dichlorophenyl | |
| 2326 | isobutyl | CH₃ | H | CH₃ | 3,5-dichlorophenyl | |
| 2327 | isobutyl | CH₃ | H | CH₃ | 2,5-dichlorophenyl | |
| 2328 | isobutyl | CH₃ | H | CH₃ | 2,4,6-trichlorophenyl | |
| 2329 | isobutyl | CH₃ | H | CH₃ | 2-chloro-4-methylphenyl | |
| 2330 | isobutyl | CH₃ | H | CH₃ | 2-chloro-4-methoxy- | |
| | | | | | phenyl | |
| 2331 | isobutyl | CH₃ | H | CH₃ | 2,4-dimethylphenyl | |
| 2332 | isobutyl | CH₃ | H | CH₃ | 2,5-dimethylphenyl | |
| 2333 | isobutyl | CH₃ | H | CH₃ | 2,6-dimethylphenyl | |
| 2334 | isobutyl | CH₃ | H | CH₃ | 2,4,6-trimethylphenyl | |
| 2335 | isobutyl | CH₃ | H | CH₃ | 2-fluorophenyl | |
| 2336 | isobutyl | CH₃ | H | CH₃ | 3-fluorophenyl | |
| 2337 | isobutyl | CH₃ | H | CH₃ | 4-fluorophenyl | |
| 2338 | isobutyl | CH₃ | H | CH₃ | 2-chloro-4-fluorophenyl | |
| 2339 | isobutyl | CH₃ | H | CH₃ | 2,4-dimethoxyphenyl | |
| 2340 | isobutyl | CH₃ | H | CH₃ | 2,3-dimethoxyphenyl | |
| 2341 | isobutyl | CH₃ | H | CH₃ | 3,4-dimethoxyphenyl | |
| 2342 | isobutyl | CH₃ | H | CH₃ | 3,5-dimethoxyphenyl | |
| 2343 | isobutyl | CH₃ | H | CH₃ | 2,5-dimethoxyphenyl | |
| 2344 | isobutyl | CH₃ | H | CH₃ | 2-trifluoromethylphenyl | |
| 2345 | isobutyl | CH₃ | H | CH₃ | 3-trifluoromethylphenyl | |
| 2346 | isobutyl | CH₃ | H | CH₃ | 4-trifluoromethylphenyl | |
| 2347 | isobutyl | CH₃ | H | CH₃ | 3-trifluoromethyl- | |
| | | | | | 4-chlorophenyl | |
| 2348 | isobutyl | CH₃ | H | CH₃ | 3-trifluoromethyl- | |
| | | | | | 4-fluorophenyl | |
| 2349 | isobutyl | CH₃ | H | CH₃ | 2-trifluoro- | |
| | | | | | methoxyphenyl | |
| 2350 | isobutyl | CH₃ | H | CH₃ | 3-trifluoro- | |
| | | | | | methoxyphenyl | |
| 2351 | isobutyl | CH₃ | H | CH₃ | 4-trifluoro- | |
| | | | | | methoxyphenyl | |
| 2352 | isobutyl | CH₃ | H | CH₃ | 2-difluoro- | |
| | | | | | methoxyphenyl | |
| 2353 | isobutyl | CH₃ | H | CH₃ | 3-difluoro- | |
| | | | | | methoxyphenyl | |
| 2354 | isobutyl | CH₃ | H | CH₃ | 4-difluoro- | |
| | | | | | methoxyphenyl | |
| 2355 | isobutyl | CH₃ | H | CH₃ | 2-tert-butylphenyl | |
| 2356 | isobutyl | CH₃ | H | CH₃ | 3-tert-butylphenyl | |
| 2357 | isobutyl | CH₃ | H | CH₃ | 4-tert-butylphenyl | |
| 2358 | isobutyl | CH₃ | H | CH₃ | 4-methanesulphonyl- | |
| | | | | | phenyl | |
| 2359 | 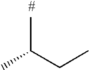 | H | H | H | 3,4,5-trimethoxyphenyl | 2.37¹⁾ |
| 2360 | 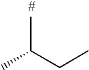 | H | H | H | 3-fluoro- 4-trifluoromethylphenyl | 3.34¹⁾ |
| 2361 | 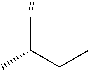 | H | H | H | 2-trifluoromethyl- 4-chlorophenyl | |
| 2362 | 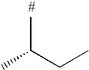 | H | H | H | 2-trifluoromethyl- 4-fluorophenyl | |
| 2363 | 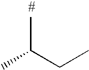 | H | H | H | 2-chloro- 4-trifluoromethylphenyl | |
| 2364 | 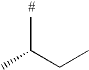 | H | H | H | 2-fluoro- 4-trifluoromethylphenyl | |
| 2365 | 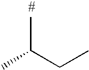 | H | H | H | 2-chloro- 4-trifluoromethoxyphenyl | |
| 2366 | 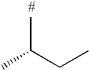 | H | H | H | 2-(4,6-bisbenzyloxy- [1,3,5]triazin-2-ylamino)methyl | |
| 2367 | 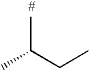 | H | H | H | 2-chloro- 4-trifluoromethylbenzyl | |
| 2368 | 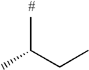 | H | H | H | 2-fluoro- 4-trifluoromethylbenzyl | |
| 2369 | 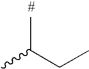 | H | H | H | 3,4,5-trimethoxyphenyl | |
| 2370 | 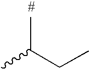 | H | H | H | 3-fluoro- 4-trifluoromethylphenyl | |
| 2371 | 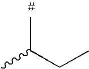 | H | H | H | 2-trifluoromethyl- 4-chlorophenyl | |
| 2372 | 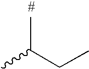 | H | H | H | 2-trifluoromethyl- 4-fluorophenyl | |
| 2373 | 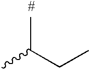 | H | H | H | 2-chloro- 4-trifluoromethylphenyl | |
| 2374 | 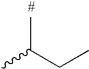 | H | H | H | 2-fluoro- 4-trifluoromethylphenyl | |
| 2375 | 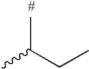 | H | H | H | 2-chloro- 4-trifluoromethoxyphenyl | |
| 2376 | 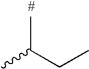 | H | H | H | 2-(4,6-bisbenzyloxy- [1,3,5]triazin-2- ylamino)methyl | |
| 2377 | 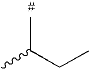 | H | H | H | 2-chloro- 4-trifluoromethylbenzyl | |
| 2378 | 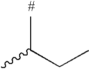 | H | H | H | 2-fluoro- 4-trifluoromethylbenzyl | |
| 2369 | cyclohexyl | H | H | H | 3,4,5-trimethoxyphenyl | |
| 2370 | cyclohexyl | H | H | H | 3-fluoro- | |
| | | | | | 4-trifluoromethylphenyl | |
| 2371 | cyclohexyl | H | H | H | 2-trifluoromethyl- | |
| | | | | | 4-chlorophenyl | |
| 2372 | cyclohexyl | H | H | H | 2-trifluoromethyl- | |
| | | | | | 4-fluorophenyl | |
| 2373 | cyclohexyl | H | H | H | 2-chloro- | |
| | | | | | 4-trifluoromethylphenyl | |
| 2374 | cyclohexyl | H | H | H | 2-fluoro- | |
| | | | | | 4-trifluoromethylphenyl | |
| 2375 | cyclohexyl | H | H | H | 2-chloro- | |
| | | | | | 4-trifluoromethoxyphenyl | |
| 2376 | cyclohexyl | H | H | H | 2-(4,6-bisbenzyloxy- | 4.07¹⁾ |
| | | | | | [1,3,5]triazin-2- | |
| | | | | | ylamino)methyl | |
| 2377 | cyclohexyl | H | H | H | 2-chloro- | |
| | | | | | 4-trifluoromethylbenzyl | |
| 2378 | cyclohexyl | H | H | H | 2-fluoro- | |
| | | | | | 4-trifluoromethylbenzyl | |
| 2379 | 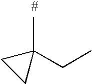 | H | H | H | phenyl | |
| 2380 | 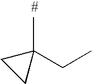 | H | H | H | 2-chlorophenyl | |
| 2381 | 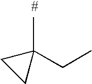 | H | H | H | 4-chlorophenyl | |
| 2382 | 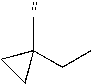 | H | H | H | 2-methylphenyl | |
| 2383 | 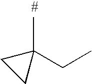 | H | H | H | 4-methylphenyl | |
| 2384 | 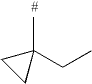 | H | H | H | 2-methoxyphenyl | |
| 2385 | 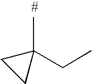 | H | H | H | 4-methoxyphenyl | |
| 2386 | 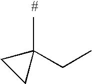 | H | H | H | 2,4-dichlorophenyl | |
| 2387 | 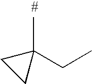 | H | H | H | 3,4-dichlorophenyl | |
| 2388 | 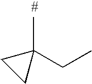 | H | H | H | 2,4-dimethylphenyl | |
| 2389 | 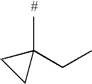 | H | H | H | 2,6-dimethylphenyl | |
| 2390 | 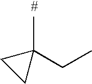 | H | H | H | 2-fluorophenyl | |
| 2391 | 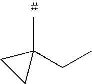 | H | H | H | 4-fluorophenyl | |
| 2392 | 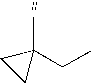 | H | H | H | 2-chloro-4-fluorophenyl | |
| 2393 | 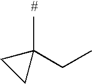 | H | H | H | 3,5-dimethoxyphenyl | 2.63¹⁾ |
| 2394 | 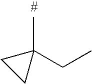 | H | H | H | 3,4-dimethoxyphenyl | |
| 2395 | 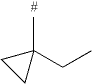 | H | H | H | 2,4-dimethoxyphenyl | |
| 2396 | 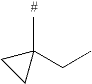 | H | H | H | 4-trifluoromethylphenyl | |
| 2397 | 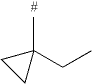 | H | H | H | 4-tert-butylphenyl | |

*)The HPLC retention times (Rt) and the logP values are determined by HPLC (gradient method) under the following conditions:
¹⁾logP determined as follows:
Instrument: HP1100 LC (UV/DAD)
Flow: 2 ml/min
Gradient (0.08% HCO2H):
90% water/10% acetonitrile to 5% water/95% acetonitrile (0-1.7 min)
5% water/95% acetonitrile (1 min)
Oven temperature: 40° C.
Column: Kromasil RP18. 3.5 μm, 23 × 3.1 mm
²⁾Retention times (Rt) determined as follows:
Instrument: HP1100 LC (UV)
Flow: 2 ml/min
Gradient (0.05% HCO2H):
95% water/5% acetonitrile to 5% water/95 acetonitrile (0-5.15 min)
5% water/95 acetonitrile (0.5 min)
Oven temperature: 40° C.
Column: Phenomenex, Synergie 2μ Fusion-RP 80A, 20 × 4.0 mm Mercury MS
³⁾the substance cannot de detected by UV: ¹H NMR (D3-acetonitrile, 400 MHz): δ = 0.92 (t, 3H), 1.01 (d, 3H), 1.28 (m, 1H), 1.44 (s, 9H), 1.61 (m, 1H), 1.99 (m, 1H), 3.65 (dd, 1H), 3.83 (d, 2H), 4.72 (d, 1H), 7.17 (bs, NH)
⁴⁾the substance cannot de detected by UV

Using methods known from the literature (Org. Lett., 2004, 6 (13), 2153-56), it is possible to obtain the beta-lactonecarboxylic acid derivatives of the formula

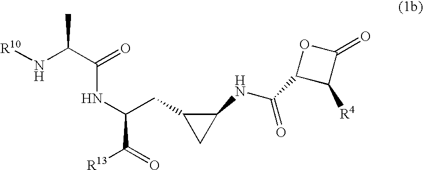

listed in Table 2 below.

TABLE 2

| Example | R⁴ | R¹⁰**) | R¹³ | logP*) |
|---|---|---|---|---|
| 2398 | 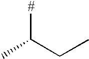 | H | OH | n.d.¹⁾ |
| 2399 | 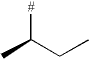 | H | OH | |
| 2400 | 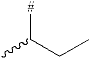 | H | OH | |
| 2401 | 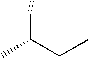 | Cbz | benzyloxy | n.d.¹⁾ |
| 2402 | 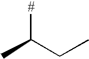 | Cbz | benzyloxy | |
| 2403 | 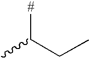 | Cbz | benzyloxy | |
| 2404 | isobutyl | H | OH | |
| 2405 | isobutyl | Cbz | benzyloxy | |
| 2406 | isopropyl | H | OH | |
| 2407 | isopropyl | Cbz | benzyloxy | |
| 2408 | benzyl | H | OH | |
| 2409 | benzyl | Cbz | benzyloxy | |
| 2410 | 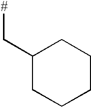 | H | OH | |
| 2411 | 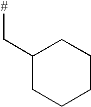 | Cbz | benzyloxy | |
| 2412 | 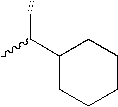 | H | OH | |
| 2413 | 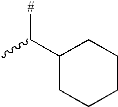 | Cbz | benzyloxy | |
| 2414 | 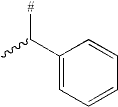 | H | OH | |
| 2415 | 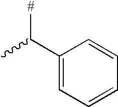 | Cbz | benzyloxy | |
| 2416 | 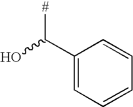 | H | OH | |
| 2417 | 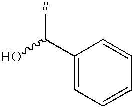 | Cbz | benzyloxy | |
| 2418 | 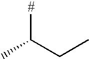 | Fmoc | benzyloxy | |
| 2419 | 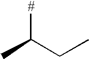 | Fmoc | benzyloxy | |
| 2420 | 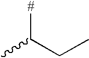 | Fmoc | benzyloxy | |
| 2421 | isobutyl | Fmoc | benzyloxy | |
| 2422 | isopropyl | Fmoc | benzyloxy | |
| 2423 | benzyl | Fmoc | benzyloxy | |
| 2424 | 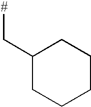 | Fmoc | benzyloxy | |
| 2425 | 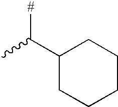 | Fmoc | benzyloxy | |
| 2426 | 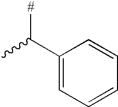 | Fmoc | benzyloxy | |
| 2427 | 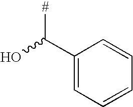 | Fmoc | benzyloxy | |

*) The compounds marked¹⁾ have already been described (EP1166781 or Org. Lett., 2004, 6(13), 2153-56). Accordingly, they are not characterized in more detail.
**) Cbz = benzyloxycarbonyl
Fmoc = 9H-fluoren-9-ylmethoxycarbonyl
Boc = tert-butoxycarbonyl

Using the methods described above (Org. Lett., 2004, 6 (13), 2153-56), it is also possible to obtain the beta-lactonecarboxylic acid derivatives of the formula

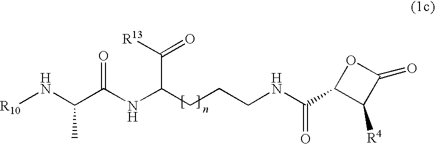

listed in Table 3 below.

TABLE 3

| Example | R⁴ | R¹⁰**) | R¹³**) | n | logP/Rt*) |
|---|---|---|---|---|---|
| 2428 | 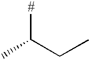 | H | OH | 1 | |
| 2429 | 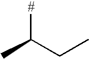 | H | OH | 1 | |
| 2430 | 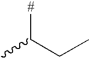 | H | OH | 1 | |
| 2431 | 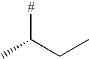 | Cbz | benzyloxy | 1 | n.d.¹⁾ |
| 2432 | 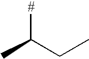 | Cbz | benzyloxy | 1 | |
| 2433 | 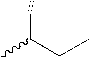 | Cbz | benzyloxy | 1 | |
| 2434 | isobutyl | H | OH | 1 | |
| 2435 | isobutyl | Cbz | benzyloxy | 1 | |
| 2436 | isopropyl | H | OH | 1 | |
| 2437 | isopropyl | Cbz | benzyloxy | 1 | |
| 2438 | benzyl | H | H | 1 | |
| 2439 | benzyl | Cbz | benzyloxy | 1 | |
| 2440 | 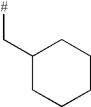 | H | OH | 1 | |
| 2441 | 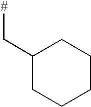 | Cbz | benzyloxy | 1 | |
| 2442 | 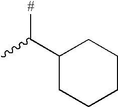 | H | OH | 1 | |
| 2443 | 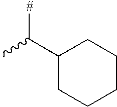 | Cbz | benzyloxy | 1 | |
| 2444 | 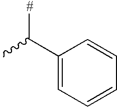 | H | OH | 1 | |
| 2445 | 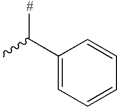 | Cbz | benzyloxy | 1 | |
| 2446 | 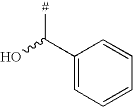 | H | OH | 1 | |
| 2447 | 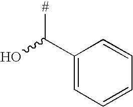 | Cbz | benzyloxy | 1 | |
| 2448 | 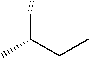 | Boc | OBoc | 1 | |
| 2449 | 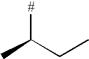 | Boc | OBoc | 1 | |
| 2450 | 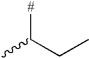 | Boc | OBoc | 1 | |
| 2451 | isobutyl | Boc | OBoc | 1 | |
| 2452 | isopropyl | Boc | OBoc | 1 | |
| 2453 | benzyl | Boc | OBoc | 1 | |
| 2454 | 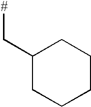 | Boc | OBoc | 1 | |
| 2455 | 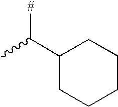 | Boc | OBoc | 1 | |
| 2456 | 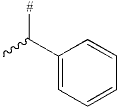 | Boc | OBoc | 1 | |
| 2457 | 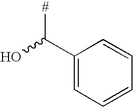 | Boc | OBoc | 1 | |
| 2458 | 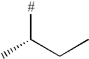 | H | OH | 2 | n.d.¹⁾ |
| 2459 | 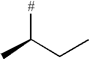 | H | OH | 2 | |
| 2460 | 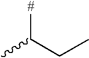 | H | OH | 2 | |
| 2461 | 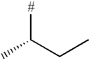 | Cbz | benzyloxy | 2 | n.d.¹⁾ |
| 2462 | 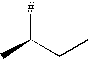 | Cbz | benzyloxy | 2 | |
| 2463 | 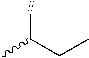 | Cbz | benzyloxy | 2 | |
| 2464 | isobutyl | H | OH | 2 | |
| 2465 | isobutyl | Cbz | benzyloxy | 2 | |
| 2466 | isopropyl | H | OH | 2 | |
| 2467 | isopropyl | Cbz | benzyloxy | 2 | |
| 2468 | benzyl | H | OH | 2 | |
| 2469 | benzyl | Cbz | benzyloxy | 2 | |
| 2470 | 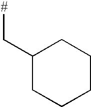 | H | OH | 2 | |
| 2471 | 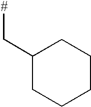 | Cbz | benzyloxy | 2 | |
| 2472 | 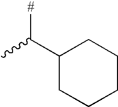 | H | OH | 2 | |
| 2473 | 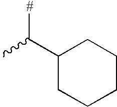 | Cbz | benzyloxy | 2 | |
| 2474 | 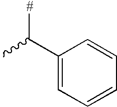 | H | OH | 2 | |
| 2475 | 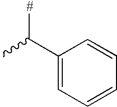 | Cbz | benzyloxy | 2 | |
| 2476 | 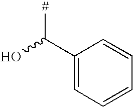 | H | OH | 2 | |
| 2477 | 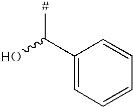 | Cbz | benzyloxy | 2 | |
| 2478 | 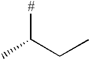 | Fmoc | benzyloxy | 2 | |
| 2479 | 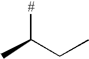 | Fmoc | benzyloxy | 2 | |
| 2480 | 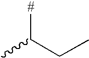 | Fmoc | benzyloxy | 2 | |
| 2481 | isobutyl | Fmoc | benzyloxy | 2 | |
| 2482 | isopropyl | Fmoc | benzyloxy | 2 | |
| 2483 | benzyl | Fmoc | benzyloxy | 2 | |
| 2484 | 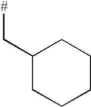 | Fmoc | benzyloxy | 2 | |
| 2485 | 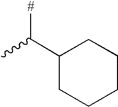 | Fmoc | benzyloxy | 2 | |
| 2486 | 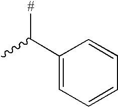 | Fmoc | benzyloxy | 2 | |
| 2487 | 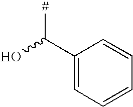 | Fmoc | benzyloxy | 2 | |
| 2488 | 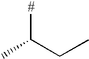 | Boc | OBoc | 2 | n.d.¹⁾ |
| 2489 | 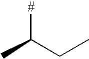 | Boc | OBoc | 2 | |
| 2490 | 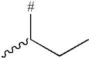 | Boc | OBoc | 2 | |
| 2491 | isobutyl | Boc | OBoc | 2 | |
| 2492 | isopropyl | Boc | OBoc | 2 | |
| 2493 | benzyl | Boc | OBoc | 2 | |
| 2494 | 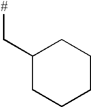 | Boc | OBoc | 2 | |
| 2495 | 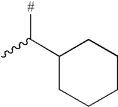 | Boc | OBoc | 2 | |
| 2496 | 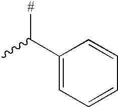 | Boc | OBoc | 2 | |
| 2497 | 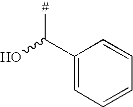 | Boc | OBoc | 2 | |

*) The compounds marked¹⁾ have already been described (EP1166781 or Org. Lett., 2004, 6(13), 2153-56). Accordingly, they are not characterized in more detail.
**) Cbz = benzyloxycarbonyl
Fmoc = 9H-fluoren-9-ylmethoxycarbonyl.
Boc = tert-butyoxycarbonyl

Using the methods stated, it is or was also possible to obtain the beta-lactonecarboxylic acid derivatives of the formula

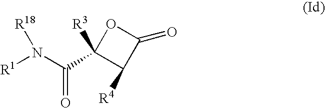

listed in Table 4 below.

TABLE 4

| Ex- | | | | | logP/ |
|---|---|---|---|---|---|
| ample | R⁴ | R³ | R¹⁸ | R¹ | Rt*) |
| 2498 | 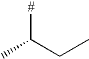 | H | H | benzyl | 2.53¹⁾ |
| 2499 | 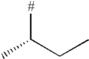 | H | H | phenyl | 2.67¹⁾ |
| 2500 | 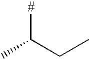 | H | H | 2-chlorophenyl | |
| 2501 | 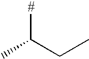 | H | H | 3-chlorophenyl | |
| 2502 | 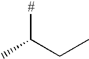 | H | H | 4-chlorophenyl | 3.29¹⁾ |
| 2503 | 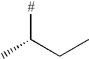 | H | H | 2-methylphenyl | |
| 2504 | 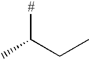 | H | H | 4-methylphenyl | |
| 2505 | 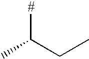 | H | H | 2-methoxyphenyl | |
| 2506 | 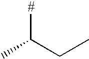 | H | H | 4-methoxyphenyl | |
| 2507 | 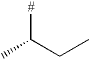 | H | H | 2,4-dichlorophenyl | 2.72¹⁾ |
| 2508 | 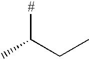 | H | H | 3,4-dichlorophenyl | |
| 2509 | 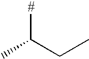 | H | H | 3,5-dichlorophenyl | 3.70¹⁾ |
| 2510 | 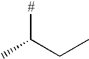 | H | H | 2,4-dimethylphenyl | |
| 2511 | 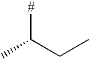 | H | H | 2,6-dimethylphenyl | |
| 2512 | 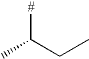 | H | H | 2-fluorophenyl | |
| 2513 | 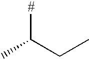 | H | H | 3-fluorophenyl | 3.00¹⁾ |
| 2514 | 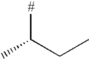 | H | H | 4-fluorophenyl | 2.87¹⁾ |
| 2515 | 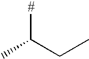 | H | H | 2-chloro-4-fluorophenyl | |
| 2516 | 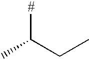 | H | H | 2,4-dimethoxyphenyl | |
| 2517 | 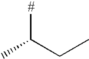 | H | H | 3,4-dimethoxyphenyl | |
| 2517a | 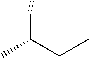 | H | H | 3,5-difluorophenyl | 3.29¹⁾ |
| 2518 | 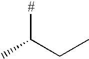 | H | H | 3,5-dimethoxyphenyl | |
| 2519 | 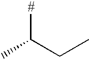 | H | H | 3,4,5-trimethoxyphenyl | 2.50¹⁾ |
| 2520 | 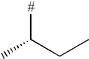 | H | H | 4-trifluoromethylphenyl | |
| 2521 | 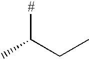 | H | H | 4-tert-butylphenyl | 3.93¹⁾ |
| 2522 | 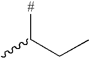 | H | H | phenyl | |
| 2523 | 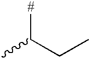 | H | H | 2-chlorophenyl | |
| 2524 | 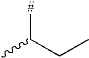 | H | H | 3-chlorophenyl | |
| 2525 | 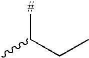 | H | H | 4-chlorophenyl | |
| 2526 | 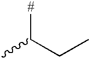 | H | H | 2-methylphenyl | |
| 2527 | 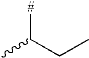 | H | H | 4-methylphenyl | |
| 2528 | 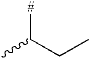 | H | H | 2-methoxyphenyl | |
| 2529 | 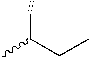 | H | H | 4-methoxyphenyl | |
| 2530 | 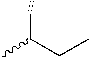 | H | H | 2,4-dichlorophenyl | |
| 2531 | 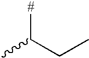 | H | H | 3,4-dichlorophenyl | |
| 2532 | 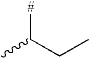 | H | H | 3,5-dichlorophenyl | |
| 2533 | 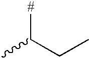 | H | H | 2,4-dimethylphenyl | |
| 2534 | 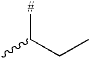 | H | H | 2,6-dimethylphenyl | |
| 2535 | 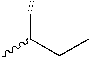 | H | H | 2-fluorophenyl | |
| 2536 | 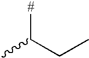 | H | H | 3-fluorophenyl | |
| 2537 | 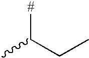 | H | H | 4-fluorophenyl | |
| 2538 | 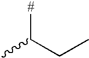 | H | H | 2-chloro-4-fluorophenyl | |
| 2539 | 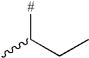 | H | H | 2,4-dimethoxyphenyl | |
| 2540 | 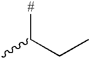 | H | H | 3,4-dimethoxyphenyl | |
| 2541 | 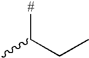 | H | H | 3,5-dimethoxyphenyl | |
| 2542 | 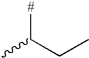 | H | H | 3,4,5-trimethoxyphenyl | |
| 2543 | 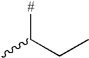 | H | H | 4-trifluoromethylphenyl | |
| 2544 | 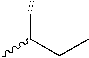 | H | H | 4-tert-butylphenyl | |
| 2545 | cyclohexyl | H | H | phenyl | |
| 2546 | cyclohexyl | H | H | 2-chlorophenyl | |
| 2547 | cyclohexyl | H | H | 3-chlorophenyl | |
| 2548 | cyclohexyl | H | H | 4-chlorophenyl | |
| 2549 | cyclohexyl | H | H | 2-methylphenyl | |
| 2550 | cyclohexyl | H | H | 4-methylphenyl | |
| 2551 | cyclohexyl | H | H | 2-methoxyphenyl | |
| 2552 | cyclohexyl | H | H | 4-methoxyphenyl | |
| 2553 | cyclohexyl | H | H | 2,4-dichlorophenyl | |
| 2554 | cyclohexyl | H | H | 3,4-dichlorophenyl | |
| 2555 | cyclohexyl | H | H | 3,5-dichlorophenyl | |
| 2556 | cyclohexyl | H | H | 2,4-dimethylphenyl | |
| 2557 | cyclohexyl | H | H | 2,6-dimethylphenyl | |
| 2558 | cyclohexyl | H | H | 2-fluorophenyl | |
| 2559 | cyclohexyl | H | H | 3-fluorophenyl | |
| 2560 | cyclohexyl | H | H | 4-fluorophenyl | |
| 2561 | cyclohexyl | H | H | 2-chloro-4-fluorophenyl | |
| 2562 | cyclohexyl | H | H | 2,4-dimethoxyphenyl | |
| 2563 | cyclohexyl | H | H | 3,4-dimethoxyphenyl | |
| 2564 | cyclohexyl | H | H | 3,5-dimethoxyphenyl | |
| 2565 | cyclohexyl | H | H | 3,4,5-trimethoxyphenyl | |
| 2566 | cyclohexyl | H | H | 4-trifluoromethylphenyl | |
| 2567 | cyclohexyl | H | H | 4-tert-butylphenyl | |
| 2568 | 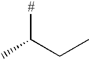 | H | H | cyclohexyl | |
| 2569 | 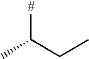 | H | H | 3-trifluoromethyl- cylcohexyl | 3.15¹⁾ |
| 2570 | 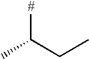 | H | H | 4-trifluoromethyl- cyclohexyl | 3.06¹⁾ |
| 2571 | 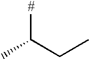 | H | H | 1-methyl-1-phenylethyl | |
| 2572 | 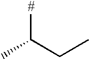 | H | H | 1-(3-chlorophenyl)- 1-methylethyl | |
| 2573 | 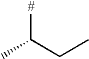 | H | H | 1-(3,5-dichlorophenyl)- 1-methylethyl | 4.04¹⁾ |
| 2574 | 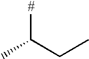 | H | H | 1-methyl-3-phenylpropyl | |
| 2575 | 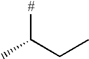 | H | H | 3-(3-chlorophenyl)- 1-methylpropyl | |
| 2576 | 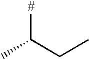 | H | H | 3-(4-chlorophenyl)- 1-methylpropyl | 3.78¹⁾ |
| 2577 | 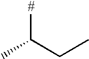 | H | H | 3-(4-methoxyphenyl)- 1-methylpropyl | 3.19¹⁾ |
| 2578 | 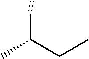 | H | H | 2-methyl-2-phenylpropyl | |
| 2579 | 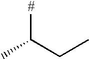 | H | H | 2-(2-chlorophenyl)- 2-methylpropyl | 3.61¹⁾ |
| 2580 | 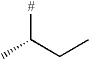 | H | H | 2-(3-chlorophenyl)- 2-methylpropyl | 3.68¹⁾ |
| 2581 | 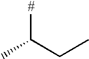 | H | H | 2-(4-chlorophenyl)- 2-methylpropyl | 3.73¹⁾ |
| 2582 | 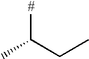 | H | H | 2-(3,4-dichlorophenyl)- 2-methylpropyl | 4.05¹⁾ |
| 2583 | 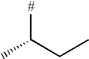 | H | H | 2-phenylbutyl | |
| 2584 | 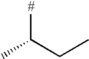 | H | H | 2-(3-chlorophenyl)butyl | |
| 2585 | 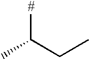 | H | H | 2-(4-chlorophenyl)butyl | |
| 2586 | 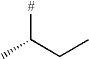 | H | H | 2-(2-methoxy- phenyl)butyl | 3.53¹⁾ |
| 2587 | 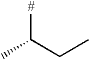 | H | H | 2-(3-methoxy- phenyl)butyl | |
| 2588 | 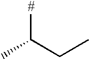 | H | H | 2-(4-methoxy- phenyl)butyl | |
| 2589 | 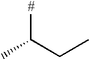 | H | H | 2-ethyl-2-phenylbutyl | |
| 2590 | 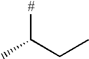 | H | H | 2-(3-chlorophenyl)- 2-ethylbutyl | 4.34¹⁾ |
| 2591 | 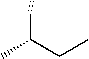 | H | H | 2-(4-chlorophenyl)- 2-ethylbutyl | 4.40¹⁾ |
| 2592 | 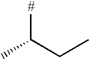 | H | H | 2-(2-methoxyphenyl)- 2-ethylbutyl | 4.23¹⁾ |
| 2593 | 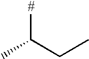 | H | H | 2-(3-methoxyphenyl)- 2-ethylbutyl | |
| 2594 | 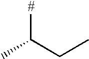 | H | H | 2-(4-methoxyphenyl)- 2-ethylbutyl | 3.89¹⁾ |
| 2595 | 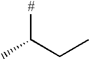 | H | H | 2-(3,4-dimethoxyphenyl)- 2-ethylbutyl | 3.46¹⁾ |

*) The HPLC retention times (Rt) and the logP values are determined by HPLC (gradient method) under the following conditions:
¹⁾logP determined as folllows:
Instrument: HP1100 LC (UV/DAD)
Flow: 2 ml/min
Gradient (0.08% HCO2H):
90% water/10% acetonitrile to 5% water/95% acetonitrile (0-1.7 min)
5% water/95% acetonitrile (1 min)
Oven temperature: 40° C.
Column: Kromasil RP18. 3.5 μm, 23 × 3.1 mm

### Biological Example 1

### Plasmopara Test (Grapevine)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Plasmopara viticola and then remain in an incubation cabin at about 20° C. and 100% relative atmospheric humidity for 1 day. The plants are then placed in a greenhouse at about 21° C. and about 90% atmospheric humidity for 4 days. The plants are then moistened and placed in an incubation cabin for 1 day.

Evaluation is carried out 6 days after the inoculation. 0% means an efficacy which corresponds to that of the control, whereas an efficacy of 100% means that no infection is observed.

In this test, the compounds according to the invention of Examples 1 and 2431 exhibit, at an active compound application rate of 100 ppm, an efficacy of 70% or more.

### Biological Example 2

### Venturia Test (Apple)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compound according to the invention of Example 1 exhibits, at an active compound concentration of 100 ppm, an efficacy of 70% or more.

### Biological Example 3

### Botrytis Test (Bean)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvents and emulsifier, and the concentrate is diluted with water to the desired concentration.

To test for protective activity, young plants are sprayed with the preparation of active compound at the stated application rate. After the spray coating has dried on, 2 small pieces of agar colonized by Botrytis cinerea are placed onto each leaf. The inoculated plants are placed in a dark chamber at about 20° C. and 100% relative atmospheric humidity.

2 days after the inoculation, the size of the infected areas on the leaves in assessed. 0% means an efficacy which corresponds to that of the control, whereas an efficacy of 100% means that no infection is observed.

In this test, the compounds according to the invention of Examples 1 and 2431 exhibit, at an active compound concentration of 500 ppm, an efficacy of 70% or more.

### Biological Example 4

### Alternaria Test (Tomato)/Protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young tomato plants are sprayed with the preparation of active compound at the stated application rate. 1 day after the treatment, the plants are inoculated with a spore suspension of Alternaria solani and then remain at 100% rel. humidity and 20° C. for 24 h. The plants then remain at 96% rel. atmospheric humidity and a temperature of 20° C.

Evaluation is carried out 7 days after the inoculation. 0% means an efficacy which corresponds to that of the control, whereas an efficacy of 100% means that no infection is observed.

In this test, the compounds according to the invention of Examples 1, 67, 96 and 154 exhibit, at an active compound concentration of 500 ppm, an efficacy of 70% or more.

In this test, the compounds according to the invention 2431, 2439, 2488, 1125, 489, 1173, 105, 68, 89, 113, 40, 111, 5, 2, 63, 117, 25, 24, 2360, 7, 12, 14, 1139, 10, 37 and 2517a also exhibit, at an active compound concentration of 500 ppm, an efficacy of 70% or more.

### Biological Example 5

### Blumeria Test (Tomato)/Protective

### Blumeria Test (Wheat)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate.

After the spray coating has dried on, the plants are dusted with spores of Blumeria graminis f.sp. tritici.

The plants are placed in a greenhouse at a temperature of about 20° C. and a relative atmospheric humidity of about 80% to promote the development of mildew pustules.

Evaluation is carried out 7 days after the inoculation. 0% means an efficacy which corresponds to that of the control, whereas an efficacy of 100% means that no infection is observed.

In this test, the compounds according to the invention 44, 2365, 2362, 2498, 25, 24, 11, 2360, 7, 12, 5a, 10, 37 and 144e exhibit, at an active compound concentration of 1000 ppm, an efficacy of 70% or more.

### Biological Example 6

### Phytophthora Test (Tomato)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Phytophthora infestans. The plants are then placed in an incubation cabin at about 20° C. and 100% relative atmospheric humidity.

Evaluation is carried out 3 days after the inoculation. 0% means an efficacy which corresponds to that of the control, whereas an efficacy of 100% means that no infection is observed.

In this test, the compounds according to the invention 2461, 1, 44, 2, 2365, 2362, 2361, 63, 117, 2513, 2509, 2498, 24, 11, 33a, 2360, 12, 14, 96, 1139, 10, 1133, 54a and 41 exhibit, at an active compound concentration of 100 ppm, an efficacy of 70% or more.

### Biological Example 7

### Puccinia Test (Wheat)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a conidia suspension of Puccinia triticina. The plants remain in an incubation cabin at 20° C. and 100% relative atmospheric humidity for 48 hours.

The plants are then placed in a greenhouse at a temperature of about 20° C. and a relative atmospheric humidity of 80% to promote the development of rust pustules.

Evaluation is carried out 10 days after the inoculation. 0% means an efficacy which corresponds to that of the control, whereas an efficacy of 100% means that no infection is observed.

In this test, the compounds according to the invention 1, 44, 2, 2365, 2362, 2361, 117, 2498, 25, 24, 11, 2360, 7, 12, 14, 10, 37 and 144e exhibit, at an active compound concentration of 1000 ppm, an efficacy of 70% or more.

1. A method of controlling phytopathogenic fungi and other microorganisms in or on plants, comprising applying to said phytopathogenic fungi, their habitat, or a combination thereof, a compound of formula (I) 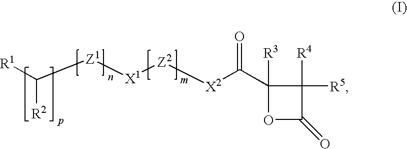
in which
m and n independently of one another are an integer from 0 to 5;
and
p is either 0 or 1;
and
R¹ is selected from the group consisting of H, alkyl, cycloalkyl, arylalkyl and aryl;
or
R¹ is NR⁶R⁷ in which
R⁶ is selected from the group consisting of H, alkyl, heterocyclyl and arylalkyl;
R⁷ is selected from the group consisting of H, alkyl, arylalkyl and CWR⁸;
R⁸ is selected from the group consisting of H, alkyl, alkylamino, alkoxy, aryl, heterocyclyl, arylalkyl, arylalkylamino and arylalkyloxy; and
W is either O or S;
or
R¹ is 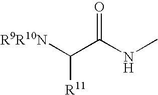
in which
R⁹ is selected from the group consisting of H, alkyl and arylalkyl; and
R¹⁰ is selected from the group consisting of H, alkyl, arylalkyl, CWR⁸, alkylsulphonyl, arylsulphonyl and PW(R¹²)₂,
and
R¹² may be identical or different and are selected from the group consisting of alkyl and aryl; or
R⁹ and R¹⁰ together are 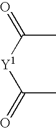
in which
Y¹ is alkylene or arylene; and
R¹¹ is selected from the group consisting of H, alkyl and arylalkyl;
and
R² is H, C₁-C₄-alkyl or COR¹³ in which
R¹³ is selected from the group consisting of OH, alkoxy, alkenyloxy, arylalkyloxy, cycloalkyloxy, aryloxyalkoxy and NR¹⁴R¹⁵ in which
R¹⁴ is selected from the group consisting of H, alkyl and aryl; and
R¹⁵ is selected from the group consisting of alkyl, arylalkyl, alkoxycarbonylalkyl, amino, alkylamino and arylamino; or
R¹⁴ and R¹⁵ together with the adjacent nitrogen atom form a heterocycle; or
R¹³ is CH₂OR¹⁶, in which
R¹⁶ is selected from the group consisting of H, alkyl and arylalkyl;
or
R² is selected from the group consisting of alkanoyl, arylcarbonyl and Si(R¹⁷)₃ in which
R¹⁷ are all identical or different and are selected from the group consisting of alkyl and aryl;
or
R¹ and R² together form a group of the formula 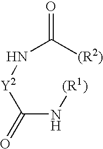
in which
Y² is alkylene;
and
R³, R⁴ and R⁵ independently of one another are selected from the group consisting of H, alkyl, alkenyl, alkynyl, cycloalkyl, cycloalkenyl, aryl, heterocyclyl and arylalkyl;
and
X¹ is selected from the group consisting of a bond, alkylene, cycloalkylene, alkenylene and arylene;
and
X² is selected from the group consisting of O, S, NR¹⁸, N(OR¹⁸), NR¹⁸O and NR¹⁸NH, in which
R¹⁸ is selected from the group consisting of
H, alkyl, C₁-C₄-alkylC(═O), C₁-C₄-alkylOC(═O), C₁-C₄-alkoxy(C₁-C₄)alkyl, C₁-C₆-alkynyl, C₁-C₆-alkylsulphinyl, C₁-C₆-alkylsulphonyl and C₃-C₈-cycloalkyl;
C₁-C₆-haloalkyl, C₁-C₄-haloalkylsulphinyl, C₁-C₄-haloalkylsulphonyl, halo-C₁-C₄-alkoxy-C₁-C₄-alkyl and C₃-C₈-halocycloalkyl having in each case 1 to 9 fluorine, chlorine, bromine atoms, or combinations thereof;
formyl, formyl-C₁-C₃-alkyl, (C₁-C₃-alkyl)carbonyl-C₁-C₃-alkyl and (C₁-C₃-alkoxy)carbonyl-C₁-C₃-alkyl;
halo-(C₁-C₃-alkyl)carbonyl-C₁-C₃-alkyl and halo-(C₁-C₃-alkoxy)carbonyl-C₁-C₃-alkyl having in each case 1 to 13 fluorine, chlorine, bromine atoms, or combinations thereof;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl and (C₃-C₈-cycloalkyl)carbonyl;
(C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl and (C₃-C₈-halocycloalkyl)carbonyl having in each case 1 to 9 fluorine, chlorine, bromine atoms, or combinations thereof; and
—CH₂—C≡C—R¹⁹, —CH₂—CH═CH—R¹⁹, —CH═C═CH—R¹⁹, —C(═O)C(═O)R¹⁹,
—CON(R¹⁹)₂, —CH₂N(R¹⁹)₂, C₁-C₄-trialkylsilyl, C₁-C₄-dialkylmonophenylsilyl and arylalkyl, in which
R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano;
and
Z¹ and Z² independently of one another are selected from the group consisting of CH₂, CHCH₃, CHF, CF₂, CHCl, CCl₂, CH₂—CH(CH₃) and CH(CH₃)—CH₂;
Z² is selected from the group consisting of —O—CH₂—CH₂, —O—CH(CH₃)—CH₂ and —O—CH₂—CHCH₃; or a combination thereof
or
of agrochemically active salts thereof.

2. A compound of the formula (I) as defined in claim 1 where
(a) R³ is selected from the group consisting of alkyl, alkenyl and arylalkyl, and
X² is NR¹⁸;
or
(b) R⁴ and R⁵ are selected from the group consisting of alkyl, alkenyl, alkynyl, cycloalkyl, cycloalkenyl, aryl, heterocyclyl and arylalkyl, and
X² is NR¹⁸;
or
(c) R¹ is selected from the group consisting of alkyl, cycloalkyl, arylalkyl and aryl, and
X² is NR¹⁸;
or
(d) X² is selected from the group consisting of S, N(OR¹⁸), NR¹⁸O and NR¹⁸NH;
or
(e) X² is oxygen, and
R⁴ comprises at least 2 carbon atoms, and
with the proviso that, if
(i) R¹=R³=R⁵=H, X¹ is a bond and m=n=p=0, then R⁴ is not n-propyl, sec-butyl, isobutyl or 2-methyl-1-propen-3-yl;
(ii) R¹=R³=H, R⁵=n-propyl, X¹ is a bond and m=n=p=0, then R⁴ is not n-propyl;
(iii) R¹=methyl, R³=R⁵=H, X¹ is a bond and m=n=p=0, then R⁴ is not ethyl;
(iv) R¹=ethyl, R³=trifluoromethyl, R⁵=H, X¹ is a bond and m=n=p=0, then R⁴ is not 1-phenylprop-3-yl;
(v) R¹=benzyl, R³=R⁵=H, X¹ is a bond and m=n=p=0, then R⁴ is not ethyl, isopropyl, isobutyl or 2-methyl-1-propen-3-yl;
(vi) R¹=tert-butyl, R³=R⁵=H, X¹ is a bond and m=n=p=0, then R⁴ is not sec-butyl, isobutyl, 1-penten-5-yl or naphth-2-ylmethyl;
(vii) R³=H, R⁴=1-propen-3-yl, R⁵=methyl, X¹ is a bond and m=n=p=0, then R¹ is not benzyl or n-butyl;
(viii) R¹=benzyl, R⁴=isobutyl, R⁵=H, X¹ is a bond and m=n=p=0, then R³ is not benzyloxycarbonyl or acetyloxymethyl;
(ix) R¹=methyl, R³=methoxycarbonyl, R⁵=H, X¹ is a bond and m=n=p=0, then R⁴ is not methoxycarbonylmethyl; or
(x) R¹=ethyl, R³=ethoxycarbonyl, R⁵=H, X¹ is a bond and m=n=p=0, then R⁴ is not ethoxycarbonylmethyl.

3. A process for preparing a compound of the formula (I) 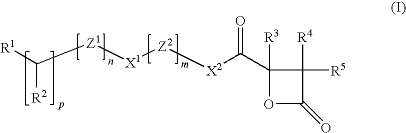
as defined in claim 1, which comprises reacting a compound of the formula (II) 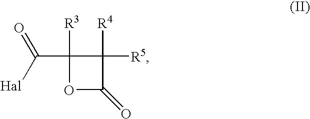
with a compound of the formula (IV) 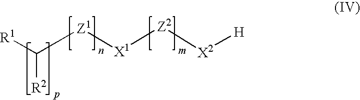
in the presence of a suitable base, where the symbols R¹, R², R³, R⁴, R⁵, X¹, X², Z¹, Z², m, n and p given in the formulae (II) and (IV) mentioned are as defined in claim 1, and Hal is selected from the group consisting of F, Cl, Br and I.

4. A process for preparing a compound of the formula (II) 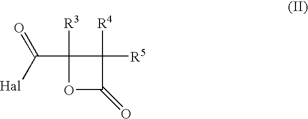
as defined in claim 3, which comprises reacting a compound of the formula (III) 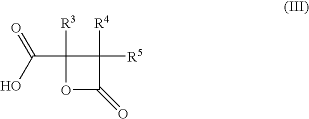
with a suitable halogenating agent, where the symbols R³, R⁴ and R⁵ given in formula (III) independently of one another are selected from the group consisting of H, alkyl, alkenyl, alkynyl, cycloalkyl, cycloalkenyl, aryl, heterocyclyl and arylalkyl.

5. A process for preparing a compound according to formula (I) 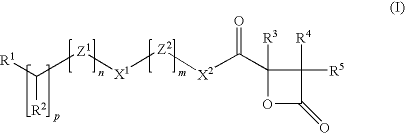
as defined in claim 1, which comprises reacting a compound according to formula (VIII) 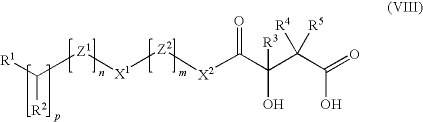
with a suitable condensing agent, where the symbols R¹, R², R³, R⁴, R⁵, X¹, X², Z¹, Z², m, n and p given in the formula (VIII) mentioned are as defined in claim 1.

6. A process for preparing a compound according to formula (VIII) 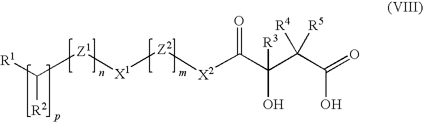
as defined in claim 5, which comprises reacting a compound according to formula (VII) 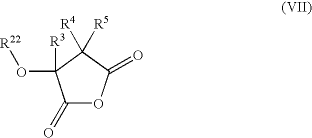
with a compound according to formula (IV) 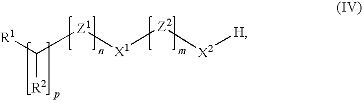
where,
m and n independently of one another are an integer from 0 to 5;
and
P is either 0 or 1;
and
R¹ is selected from the group consisting of H, alkyl, cycloalkyl, arylalkyl and aryl;
or
R¹ is NR⁶R⁷ in which
R⁶ is selected from the group consisting of H, alkyl, heterocyclyl and arylalkyl;
R⁷ is selected from the group consisting of H, alkyl, arylalkyl and CWR⁸;
R⁸ is selected from the group consisting of H, alkyl, alkylamino, alkoxy, aryl, heterocyclyl, arylalkyl, arylalkylamino and arylalkyloxy; and
W is either O or S;
or 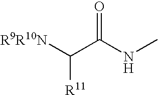
in which
R⁹ is selected from the group consisting of H, alkyl and arylalkyl; and
R¹⁰ is selected from the group consisting of H, alkyl, arylalkyl, CWR⁸, alkylsulphonyl, arylsulphonyl and PW(R¹²)₂, in which
W and R⁸ are as defined above; and
R¹² may be identical or different and are selected from the group consisting of alkyl and aryl; or
R⁹ and R¹⁰ together are 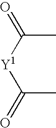
in which
Y¹ is alkylene or arylene; and
R¹¹ is selected from the group consisting of H, alkyl and arylalkyl;
and
R² is H, C₁-C₄-alkyl or COR¹³ in which R¹³ is selected from the group consisting of OH, alkoxy, alkenyloxy, arylalkyloxy, cycloalkyloxy, aryloxyalkoxy and NR¹⁴R¹⁵ in which
R¹⁴ is selected from the group consisting of H, alkyl and aryl; and
R¹⁵ is selected from the group consisting of alkyl, arylalkyl, alkoxycarbonylalkyl, amino, alkylamino and arylamino; or
R¹⁴ and R¹⁵ together with the adjacent nitrogen atom form a heterocycle; or
R¹³ is CH₂OR¹⁶, in which
R¹⁶ is selected from the group consisting of H, alkyl and arylalkyl;
Or
R² is selected from the group consisting of alkanoyl, arylcarbonyl and Si(R¹⁷)₃ in which
R¹⁷ are all identical or different and are selected from the group consisting of alkyl and aryl;
or
R¹ and R² together form a group of the formula 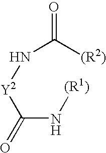
in which
Y² is alkylene;
and
R³, R⁴ and R⁵ independently of one another are selected from the group consisting of H, alkyl, alkenyl, alkynyl, cycloalkyl, cycloalkenyl, aryl, heterocyclyl and arylalkyl;
and
X¹ is selected from the group consisting of a bond, alkylene, cycloalkylene, alkenylene and arylene;
and
X² is selected from the group consisting of O, S, NR¹⁸, N(OR¹⁸), NR¹⁸O and NR¹⁸NH, in which
R¹⁸ is selected from the group consisting of
H, alkyl, C₁-C₄-alkylC(═O), C₁-C₄-alkylOC(═O), C₁-C₄-alkoxy(C₁-C₄)alkyl, C₁-C₆-alkylsulphinyl, C₁-C₆-alkylsulphonyl and C₃-C₈-cycloalkyl;
C₁-C₆-haloalkyl, C₁-C₄-halo alkyl sulphinyl, C₁-C₄-haloalkylsulphonyl, halo-C₁-C₄-alkoxy-C₁-C₄-alkyl and C₃-C₈-halocycloalkyl having in each case 1 to 9 fluorine, chlorine, bromine atoms, or combinations thereof;
formyl, (C₁-C₃-alkyl)carbonyl-C₁-C₃-alkyl and (C₁-C₃-alkoxy)carbonyl-C₁-C₃-alkyl;
halo-(C₁-C₃-alkyl)carbonyl-C₁-C₃-alkyl and halo-(C₁-C₃-alkoxy)carbonyl-C₁-C₃-alkyl having in each case 1 to 13 fluorine, chlorine, bromine atoms, or combinations thereof;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl and (C₃-C₈-cycloalkyl)carbonyl;
(C₁-C₆-haloalkyl)carbonyl, (C₁-C₆-haloalkylthio)carbonyl, (C₁-C₆-haloalkoxy)carbonyl, (C₃-C₆-haloalkenyloxy)carbonyl, (C₃-C₆-haloalkynyloxy)carbonyl, (halo-C₁-C₄-alkoxy-C₁-C₄-alkyl)carbonyl and (C₃-C₈-halocycloalkyl)carbonyl having in each case 1 to 9 fluorine, chlorine, bromine atoms, or combinations thereof; and
—CH₇—CH═CH—R¹⁹, —CH═C═CH—R¹⁹, —C(═O)C(═O)R¹⁹,
—CON(R¹⁹)₂, C₁-C₄-dialkylmonophenylsilyl and arylalkyl, in which
R¹⁹ are all identical or different and are selected from the group consisting of H, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl and cyano;
and
Z¹ and Z² independently of one another are selected from the group consisting of CH₂, CHCH₃, CHF, CF₂, CHCl, CCl₂, CH₂—CH(CH₃) and CH(CH₃)—CH₂;
Z² is selected from the group consisting of —O—CH₂—CH₂, —O—CH(CH₃)—CH₂ and —O—CH₂—CHCH₃; or a combination thereof,
and R²² is selected from the group consisting of H, SO₂R²³ or COR²³, where R²³ is unsubstituted or halogen-substituted (C₁-C₄)-alkyl, or unsubstituted or halogen-, (C₁-C₄)-alkyl- or (C₁-C₄)-haloalkyl-substituted aryl.

7. A process for preparing a compound according to formula (VII) 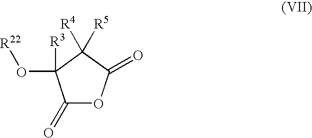
as defined in claim 6, which comprises reacting a compound according to formula (VI) 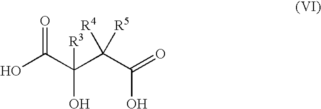
with an anhydride selected from the group consisting of ethyl chloroformate, methyl chloroformate, isopropyl chloroformate, isobutyl chloroformate, p-toluenesulphonyl chloride, methanesulphonyl chloride, acetic anhydride and trifluoroacetic anhydride, where the symbols R³, R⁴ and R⁵ given in the formula (VI) independently of one another are selected from the group consisting of H, alkyl, alkenyl, alkynyl, cycloalkyl, cycloalkenyl, aryl, heterocyclyl and arylalkyl.

8. A process for preparing a compound according to formula (VI) 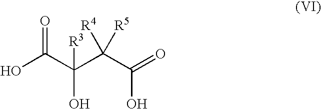
as defined in claim 7, which comprises reacting a compound according to formula (V) 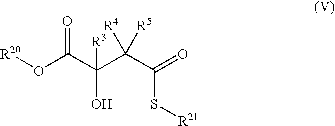
with a suitable base in an aqueous medium, where the symbols R³, R⁴ and R⁵ given in the formula (V) independently of one another are selected from the group consisting of H, alkyl, alkenyl, alkynyl, cycloalkyl, cycloalkenyl, aryl, heterocyclyl and arylalkyl, R²⁰ is selected from the group consisting of branched and straight-chain alkyl and arylalkyl radicals, and R²¹ is selected from the group consisting of branched and straight-chain alkyl, arylalkyl and aryl radicals.

9. A method for controlling phytopathogenic fungi and other microorganisms in or on plants, on seeds of plants, or combinations thereof, comprising applying to said phytopathogenic fungi or their habitat a mixture comprising at least one of the compounds defined in claim 1, agrochemically active salts thereof, or combinations thereof, and a further active compound selected from the group consisting of insecticides, attractants, sterilants, bactericides, acaricides, nematicides, fungicides, growth regulators, herbicides, safeners, fertilizers and semiochemicals.

10. (canceled)

11. (canceled)

12. A method for controlling phytopathogenic fungi and other microorganisms in or on plants, on seed of plants, or a combination thereof, comprising applying to said phytopathogenic fungi or their habitat a composition comprising at least one compound as defined in claim 1 an agrochemically active salt thereof, or a combination thereof, or a mixture comprising a compound as defined in claim 1 and a further active compound selected from the group consisting of insecticides, attractants, sterilants, bactericides, acaricides, nematicides, fungicides, growth regulators, herbicides, safeners, fertilizers and semiochemicals, and agrochemically customary auxiliaries, additives, or combinations thereof.

13. (canceled)

14. Seed treated with at least one compound as defined in claim 1, at least one agrochemically active salt thereof, or combinations thereof.

15. Seed according to claim 14, where the seed is seed of transgenic plants.
